(19) United States
(12) Patent Application Publication
(10) Pub. No.: US 2007/0184444 A1
(43) Pub. Date: Aug. 9, 2007

(54) Compositions and methods for the treatment of immune related diseases

(76) Inventors: Alexander Abbas, Belmont, CA (US); Hilary Clark, San Francisco, CA (US); Wenjun Ouyang, Foster City, CA (US); P. Mickey Williams, Half Moon Bay, CA (US); William I. Wood, Cupertino, CA (US); Thomas D. Wu, San Francisco, CA (US)

Correspondence Address: GENENTECH, INC., 1 DNA WAY, SOUTH SAN FRANCISCO, CA, 94080, US

(21) Appl. No.: 10/567,939

(22) PCT Filed: Aug. 11, 2004
(86) PCT No.: PCT/US04/26249

§ 371 Date: Dec. 4, 2006

Related U.S. Application Data
(60) Provisional application No. 60/493,546, filed on Aug. 11, 2003.

(51) Int. Cl. C12Q 1/68; G01N 33/567; C07K 14/74; C07K 16/28; C07H 21/04

(52) U.S. Cl. 435/6; 435/69.1; 435/320.1; 435/325; 530/350; 530/388.22; 514/12; 514/44; 536/23.5; 435/7.2

(57) ABSTRACT

The present invention relates to composition containing novel proteins and method of using those compositions for the dignosis and treatment of immune related diseases.

### PRIORTY

This application claims priority to U.S. Provisional Application No. 60/493,546 filed Aug. 11, 2003, to which U.S. Provisional Applications claim priority under 35 U.S.C. §119, the entire disclosure of which is hereby incorporated by reference in its entirety.

### FIELD OF THE INVENTION

The present invention relates to compositions and methods useful for the diagnosis and treatment of immune related diseases.

### BACKGROUND OF THE INVENTION

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

CD4 T helper cells play central role in regulating immune system. Under different pathogenic challenges, naive CD4 T cells can differentiate to two different subsets. T helper 1 (Th1) cells produce IFN-gamma, TNF-alpha and LT. Th1 cells and cytokines they produced are important for cellular immunity and critical for clearance of intracellular pathogen invasions. IFN-gamma produced by Th1 cells also helps antibody isotype switch to IgG2a, while the cytokines produced by Th1 cells activate macrophages and promote CTL reaction. In contrast, T helper 2 (Th2) CD4 cells mainly mediate humoral immunity. Th2 cells secrete IL-4, IL-5, IL-6, and IL-13. These cytokines play central in role in promotion of eosinophil development and mast cell activation. Th2 cells also help in B cell development antibody isotype switching to IgE and IgA. Th2 cells and their cytokines are critical for helminthes clearance.

Although Th1 and Th2 cells are necessary for the immune system to fight with various pathogenic invasion, unregulated Th1 and Th2 differentiation could play a role in autoimmune diseases. For example, uncontrolled Th2 differentiation has been demonstrated to be involved in immediate hypersensitivity, allergic reaction and asthma. Th1 cells have been shown to present in diabetes, MS, psoriasis, and lupus. Currently, IL-12 and IL-4 have been identified to be the key cytokines initiating the development of the Th1 and Th2 cells, respectively. Upon binding to its receptor, IL-12 activates Stat4, which then forms a homodimer, migrates into the nucleus and initiates down stream transcription events for Th1 development. IL-4 activates a different Stat molecule, Stat6, which induces transcription factor GATA3 expression. GATA-3 will then promote downstream differentiation of Th2 cells. The differentiation of Th1 and Th2 cells are a dynamic process, at each stage, there are different molecular events happening and different gene expression profiles. For example, at the early stage naive T cells are sensitive to environment stimuli, such as cytokines and costimulatory signals. If they receive the Th2 priming signal, they will quickly shut down the expression of the IL-12 receptor b2 chain expression and block further Th1 development. However, at the late stage of Th1 development, applying Th2 differentiation cytokines will fail to switch cells to a Th2 type. In this experiment, we mapped the gene expression profiles during the whole process of Th1 and Th2 development. We isolated naive CD4 T cells from normal human donors. Th1 cells were generated by stimulation of T cells with anti-CD3 and CD-28 plus IL-12, and anti-IL-4 antibody. Th2 cells were generated by similar TCR stimulation plus IL-4, anti-IL12, and anti-IFN-g antibodies. The undifferentiated T cells were generated by TCR stimulation, and neutralizing antibodies for IL-12, IL-4 and IFN-gamma. T cells were expanded on day 3 of primary activation with 5 volumes of fresh media. The fully differentiated Th1 and Th2 cells were then restimulated by anti-CD3 and anti-CD28. RNA was purified at different stages of T cell development, and RNA isolated for gene chip based expression analysis. Comparing gene expression profiles enabled us to identified genes preferentially expressed in Th1 or Th2 cell at different stages. These genes could play very important roles in the initiation of Th1/T2 differentiation, maintenance of Th1/Th2 phenotype, activation of Th1/Th2 cells, and effector functions, such as cytokine production, of Th1/Th2 cells. These genes could also serve as molecular markers to identify and target specific Th1 and Th2 subsets. Thus, these genes are potential therapeutic targets for many autoimmune diseases.

Autoimmune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

Despite the above identified advances in T cell research, there is a great need for additional diagnostic and therapeutic agents capable of detecting the presence of a T cell mediated disorders in a mammal and for effectively reducing these disorders. Accordingly, it is an objective of the present invention to identify polypeptides that are overexpressed in activated T cells as compared to resting T cells, and to use those polypeptides, and their encoding nucleic acids, to produce compositions of matter useful in the therapeutic treatment and diagnostic detection of T cell mediated disorders in mammals.

### SUMMARY OF THE INVENTION

A. Embodiments

The present invention concerns compositions and methods useful for the diagnosis and treatment of immune related disease in mammals, including humans. The present invention is based on the identification of proteins (including agonist and antagonist antibodies) which are a result of stimulation of the immune response in mammals. Immune related diseases can be treated by suppressing or enhancing the immune response. Molecules that enhance the immune response stimulate or potentiate the immune response to an antigen. Molecules which stimulate the immune response can be used therapeutically where enhancement of the immune response would be beneficial. Alternatively, molecules that suppress the immune response attenuate or reduce the immune response to an antigen (e.g., neutralizing antibodies) can be used therapeutically where attenuation of the immune response would be beneficial (e.g., inflammation). Accordingly, the PRO polypeptides, agonists and antagonists thereof are also useful to prepare medicines and medicaments for the treatment of immune-related and inflammatory diseases. In a specific aspect, such medicines and medicaments comprise a therapeutically effective amount of a PRO polypeptide, agonist or antagonist thereof with a pharmaceutically acceptable carrier. Preferably, the admixture is sterile.

In a further embodiment, the invention concerns a method of identifying agonists or antagonists to a PRO polypeptide which comprises contacting the PRO polypeptide with a candidate molecule and monitoring a biological activity mediated by said PRO polypeptide. Preferably, the PRO polypeptide is a native sequence PRO polypeptide. In a specific aspect, the PRO agonist or antagonist is an anti-PRO antibody.

In another embodiment, the invention concerns a composition of matter comprising a PRO polypeptide or an agonist or antagonist antibody which binds the polypeptide in admixture with a carrier or excipient. In one aspect, the composition comprises a therapeutically effective amount of the polypeptide or antibody. In another aspect, when the composition comprises an immune stimulating molecule, the composition is useful for: (a) increasing infiltration of inflammatory cells into a tissue of a mammal in need thereof, (b) stimulating or enhancing an immune response in a mammal in need thereof, (c) increasing the proliferation of T-lymphocytes in a mammal in need thereof in response to an antigen, (d) stimulating the activity of T-lymphocytes or (e) increasing the vascular permeability. In a further aspect, when the composition comprises an immune inhibiting molecule, the composition is useful for: (a) decreasing infiltration of inflammatory cells into a tissue of a mammal in need thereof, (b) inhibiting or reducing an immune response in a mammal in need thereof, (c) decreasing the activity of T-lymphocytes or (d) decreasing the proliferation of T-lymphocytes in a mammal in need thereof in response to an antigen. In another aspect, the composition comprises a further active ingredient, which may, for example, be a further antibody or a cytotoxic or chemotherapeutic agent. Preferably, the composition is sterile.

In another embodiment, the invention concerns a method of treating an immune related disorder in a mammal in need thereof, comprising administering to the mammal an effective amount of a PRO polypeptide, an agonist thereof, or an antagonist thereto. In a preferred aspect, the immune related disorder is selected from the group consisting of: systemic lupus erythematosis, rheumatoid arthritis, osteoarthritis, juvenile chronic arthritis, spondyloarthropathies, systemic sclerosis, idiopathic inflammatory myopathies, Sjögren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In another embodiment, the invention provides an antibody which specifically binds to any of the above or below described polypeptides. Optionally, the antibody is a monoclonal antibody, humanized antibody, antibody fragment or single-chain antibody. In one aspect, the present invention concerns an isolated antibody which binds a PRO polypeptide. In another aspect, the antibody mimics the activity of a PRO polypeptide (an agonist antibody) or conversely the antibody inhibits or neutralizes the activity of a PRO polypeptide (an antagonist antibody). In another aspect, the antibody is a monoclonal antibody, which preferably has nonhuman complementarity determining region (CDR) residues and human framework region (FR) residues. The antibody may be labeled and may be immobilized on a solid support. In a further aspect, the antibody is an antibody fragment, a monoclonal antibody, a single-chain antibody, or an anti-idiotypic antibody.

In yet another embodiment, the present invention provides a composition comprising an anti-PRO antibody in admixture with a pharmaceutically acceptable carrier. In one aspect, the composition comprises a therapeutically effective amount of the antibody. Preferably, the composition is sterile. The composition may be administered in the form of a liquid pharmaceutical formulation, which may be preserved to achieve extended storage stability. Alternatively, the antibody is a monoclonal antibody, an antibody fragment, a humanized antibody, or a single-chain antibody.

In a further embodiment, the invention concerns an article of manufacture, comprising:

(a) a composition of matter comprising a PRO polypeptide or agonist or antagonist thereof;

(b) a container containing said composition; and

(c) a label affixed to said container, or a package insert included in said container referring to the use of said PRO polypeptide or agonist or antagonist thereof in the treatment of an immune related disease. The composition may comprise a therapeutically effective amount of the PRO polypeptide or the agonist or antagonist thereof.

In yet another embodiment, the present invention concerns a method of diagnosing an immune related disease in a mammal, comprising detecting the level of expression of a gene encoding a PRO polypeptide (a) in a test sample of tissue cells obtained from the mammal, and (b) in a control sample of known normal tissue cells of the same cell type, wherein a higher or lower expression level in the test sample as compared to the control sample indicates the presence of immune related disease in the mammal from which the test tissue cells were obtained.

In another embodiment, the present invention concerns a method of diagnosing an immune disease in a mammal, comprising (a) contacting an anti-PRO antibody with a test sample of tissue cells obtained from the mammal, and (b) detecting the formation of a complex between the antibody and a PRO polypeptide, in the test sample; wherein the formation of said complex is indicative of the presence or absence of said disease. The detection may be qualitative or quantitative, and may be performed in comparison with monitoring the complex formation in a control sample of known normal tissue cells of the same cell type. A larger quantity of complexes formed in the test sample indicates the presence or absence of an immune disease in the mammal from which the test tissue cells were obtained. The antibody preferably carries a detectable label. Complex formation can be monitored, for example, by light microscopy, flow cytometry, fluorimetry, or other techniques known in the art. The test sample is usually obtained from an individual suspected of having a deficiency or abnormality of the immune system.

In another embodiment, the invention provides a method for determining the presence of a PRO polypeptide in a sample comprising exposing a test sample of cells suspected of containing the PRO polypeptide to an anti-PRO antibody and determining the binding of said antibody to said cell sample. In a specific aspect, the sample comprises a cell suspected of containing the PRO polypeptide and the antibody binds to the cell. The antibody is preferably detectably labeled and/or bound to a solid support.

In another embodiment, the present invention concerns an immune-related disease diagnostic kit, comprising an anti-PRO antibody and a carrier in suitable packaging. The kit preferably contains instructions for using the antibody to detect the presence of the PRO polypeptide. Preferably the carrier is pharmaceutically acceptable.

In another embodiment, the present invention concerns a diagnostic kit, containing an anti-PRO antibody in suitable packaging. The kit preferably contains instructions for using the antibody to detect the PRO polypeptide.

In another embodiment, the invention provides a method of diagnosing an immune-related disease in a mammal which comprises detecting the presence or absence or a PRO polypeptide in a test sample of tissue cells obtained from said mammal, wherein the presence or absence of the PRO polypeptide in said test sample is indicative of the presence of an immune-related disease in said mammal.

In another embodiment, the present invention concerns a method for identifying an agonist of a PRO polypeptide comprising:

(a) contacting cells and a test compound to be screened under conditions suitable for the induction of a cellular response normally induced by a PRO polypeptide; and

(b) determining the induction of said cellular response to determine if the test compound is an effective agonist, wherein the induction of said cellular response is indicative of said test compound being an effective agonist.

In another embodiment, the invention concerns a method for identifying a compound capable of inhibiting the activity of a PRO polypeptide comprising contacting a candidate compound with a PRO polypeptide under conditions and for a time sufficient to allow these two components to interact and determining whether the activity of the PRO polypeptide is inhibited. In a specific aspect, either the candidate compound or the PRO polypeptide is immobilized on a solid support. In another aspect, the non- immobilized component carries a detectable label. In a preferred aspect, this method comprises the steps of:

(a) contacting cells and a test compound to be screened in the presence of a PRO polypeptide under conditions suitable for the induction of a cellular response normally induced by a PRO polypeptide; and

(b) determining the induction of said cellular response to determine if the test compound is an effective antagonist.

In another embodiment, the invention provides a method for identifying a compound that inhibits the expression of a PRO polypeptide in cells that normally express the polypeptide, wherein the method comprises contacting the cells with a test compound and determining whether the expression of the PRO polypeptide is inhibited. In a preferred aspect, this method comprises the steps of:

(a) contacting cells and a test compound to be screened under conditions suitable for allowing expression of the PRO polypeptide; and

(b) determining the inhibition of expression of said polypeptide.

In yet another embodiment, the present invention concerns a method for treating an immune-related disorder in a mammal that suffers therefrom comprising administering to the mammal a nucleic acid molecule that codes for either (a) a PRO polypeptide, (b) an agonist of a PRO polypeptide or (c) an antagonist of a PRO polypeptide, wherein said agonist or antagonist may be an anti-PRO antibody. In a preferred embodiment, the mammal is human. In another preferred embodiment, the nucleic acid is administered via ex vivo gene therapy. In a further preferred embodiment, the nucleic acid is comprised within a vector, more preferably an adenoviral, adeno-associated viral, lentiviral or retroviral vector.

In yet another aspect, the invention provides a recombinant viral particle comprising a viral vector consisting essentially of a promoter, nucleic acid encoding (a) a PRO polypeptide, (b) an agonist polypeptide of a PRO polypeptide, or (c) an antagonist polypeptide of a PRO polypeptide, and a signal sequence for cellular secretion of the polypeptide, wherein the viral vector is in association with viral structural proteins. Preferably, the signal sequence is from a mammal, such as from a native PRO polypeptide.

In a still further embodiment, the invention concerns an ex vivo producer cell comprising a nucleic acid construct that expresses retroviral structural proteins and also comprises a retroviral vector consisting essentially of a promoter, nucleic acid encoding (a) a PRO polypeptide, (b) an agonist polypeptide of a PRO polypeptide or (c) an antagonist polypeptide of a PRO polypeptide, and a signal sequence for cellular secretion of the polypeptide, wherein said producer cell packages the retroviral vector in association with the structural proteins to produce recombinant retroviral particles.

In a still further embodiment, the invention provides a method of increasing the activity of T-lymphocytes in a mammal comprising administering to said mammal (a) a PRO polypeptide, (b) an agonist of a PRO polypeptide, or (c) an antagonist of a PRO polypeptide, wherein the activity of T-lymphocytes in the mammal is increased.

In a still further embodiment, the invention provides a method of decreasing the activity of T-lymphocytes in a mammal comprising administering to said mammal (a) a PRO polypeptide, (b) an agonist of a PRO polypeptide, or (c) an antagonist of a PRO polypeptide, wherein the activity of T-lymphocytes in the mammal is decreased.

In a still further embodiment, the invention provides a method of increasing the proliferation of T-lymphocytes in a mammal comprising administering to said mammal (a) a PRO polypeptide, (b) an agonist of a PRO polypeptide, or (c) an antagonist of a PRO polypeptide, wherein the proliferation of T-lymphocytes in the mammal is increased.

In a still further embodiment, the invention provides a method of decreasing the proliferation of T-lymphocytes in a mammal comprising administering to said mammal (a) a PRO polypeptide, (b) an agonist of a PRO polypeptide, or (c) an antagonist of a PRO polypeptide, wherein the proliferation of T-lymphocytes in the mammal is decreased.

B. Additional Embodiments

In other embodiments of the present invention, the invention provides vectors comprising DNA encoding any of the herein described polypeptides. Host cell comprising any such vector are also provided. By way of example, the host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which specifically binds to any of the above or below described polypeptides. Optionally, the antibody is a monoclonal antibody, humanized antibody, antibody fragment or single-chain antibody.

In yet other embodiments, the invention provides oligonucleotide probes useful for isolating genomic and cDNA nucleotide sequences or as antisense probes, wherein those probes may be derived from any of the above or below described nucleotide sequences.

In other embodiments, the invention provides an isolated nucleic acid molecule comprising a nucleotide sequence that encodes a PRO polypeptide.

In one aspect, the isolated nucleic acid molecule comprises a nucleotide sequence having at least about 80% nucleic acid sequence identity, alternatively at least about 81 % nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81 % nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91 % nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO polypeptide cDNA as disclosed herein, the coding sequence of a PRO polypeptide lacking the signal peptide as disclosed herein, the coding sequence of an extracellular domain of a transmembrane PRO polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs as disclosed herein, or (b) the complement of the DNA molecule of (a).

Another aspect the invention provides an isolated nucleic acid molecule comprising a nucleotide sequence encoding a PRO polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO polypeptides are contemplated.

Another embodiment is directed to fragments of a PRO polypeptide coding sequence, or the complement thereof, that may find use as, for example, hybridization probes, for encoding fragments of a PRO polypeptide that may optionally encode a polypeptide comprising a binding site for an anti-PRO antibody or as antisense oligonucleotide probes. Such nucleic acid fragments are usually at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length and alternatively at least about 1000 nucleotides in length, wherein in this context the term “about” means the referenced nucleotide sequence length plus or minus 10% of that referenced length. It is noted that novel fragments of a PRO polypeptide-encoding nutcleotide sequence may be determined in a routine manner by aligning the PRO polypeptide-encoding nucleotide sequence with other known nucleotide sequences using any of a number of well known sequence alignment programs and determining which PRO polypeptide-encoding nucleotide sequence fragment(s) are novel. All of such PRO polypeptide-encoding nucleotide sequences are contemplated herein. Also contemplated are the PRO polypeptide fragments encoded by these nucleotide molecule fragments, preferably those PRO polypeptide fragments that comprise a binding site for an anti-PRO antibody.

In another embodiment, the invention provides isolated PRO polypeptide encoded by any of the isolated nucleic acid sequences herein above identified.

In a certain aspect, the invention concerns an isolated PRO polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs as disclosed herein.

In a specific aspect, the invention provides an isolated PRO polypeptide without the N-terminal signal sequence and/or the initiating methionine and is encoded by a nucleotide sequence that encodes such an amino acid sequence as herein before described. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

Another aspect the invention provides an isolated PRO polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

In yet another embodiment, the invention concerns agonists and antagonists of a native PRO polypeptide as defined herein. In a particular embodiment, the agonist or antagonist is an anti-PRO antibody or a small molecule.

In a further embodiment, the invention concerns a method of identifying agonists or antagonists to a PRO polypeptide which comprise contacting the PRO polypeptide with a candidate molecule and monitoring a biological activity mediated by said PRO polypeptide. Preferably, the PRO polypeptide is a native PRO polypeptide.

In a still further embodiment, the invention concerns a composition of matter comprising a PRO polypeptide, or an agonist or antagonist of a PRO polypeptide as herein described, or an anti-PRO antibody, in combination with a carrier. Optionally, the carrier is a pharmaceutically acceptable carrier.

Another embodiment of the present invention is directed to the use of a PRO polypeptide, or an agonist or antagonist thereof as herein before described, or an anti-PRO antibody, for the preparation of a medicament useful in the treatment of a condition which is responsive to the PRO polypeptide, an agonist or antagonist thereof Or an anti-PRO antibody.

### BRIEF DESCRIPTION OF THE DRAWINGS

SEQ ID NOs 1-6464 show the nucleic acids of the invention and their encoded PRO polypeptides. Also included, for convenience is a List of Figures attached hereto as Appendix A, in which each Figure number corresponds to the same number SEQ ID NO: in the sequence listing. For example, FIG. 1 equals SEQ ID NO: 1 of the sequence listing.

### DETAILED DESCRIPTION OF THE PREFERRED EMBODIMENTS

### I. Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number potypeptide” and “PRO/number” wherein the term “number” is provided as an actual numerical designation as used herein encompass native sequence polypeptides and polypeptide variants (which are further defined herein). The PRO polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO polypeptide” comprises a polypeptide having the same amino acid sequence as the corresponding PRO polypeptide derived from nature. Such native sequence PRO polypeptides can be isolated from nature or can be produced by recombinant or synthetic means. The term “native sequence PRO polypeptide” specifically encompasses naturally-occurring truncated or secreted forms of the specific PRO polypeptide (e.g., an extracellular domain sequence), naturally-occurring variant forms (e.g., alternatively spliced forms) and naturally-occurring allelic variants of the polypeptide. In various embodiments of the invention, the native sequence PRO polypeptides disclosed herein are mature or full-length native sequence polypeptides comprising the full-length amino acids sequences shown in the accompanying figures. Start and stop codons are shown in bold font and underlined in the figures. However, while the PRO polypeptide disclosed in the accompanying figures are shown to begin with methionine residues designated herein as amino acid position I in the figures, it is conceivable and possible that other methionine residues located either upstream or downstream from the amino acid position 1 in the figures may be employed as the starting amino acid residue for the PRO polypeptides.

The PRO polypeptide “extracellular domain” or “ECD” refers to a form of the PRO polypeptide which is essentially free of the transmembrane and cytoplasmic domains. Ordinarily, a PRO polypeptide ECD will have less than 1% of such transmembrane and/or cytoplasmic domains and preferably, will have less than 0.5% of such domains. It will be understood that any transmembrane domains identified for the PRO polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various PRO polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO polypeptide variant” means an active PRO polypeptide as defined above or below having at least about 80% amino acid sequence identity with a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Such PRO polypeptide variants include, for instance, PRO polypeptides wherein one or more amino acid residues are added, or deleted, at the N- or C-terminus of the full-length native amino acid sequence. Ordinarily, a PRO polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other specifically defined fragment of a full-length PRO polypeptide sequence as disclosed herein. Ordinarily, PRO variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the PRO polypeptide sequences identified herein is defined as the percentage of amino acid residues in a candidate sequence that are identical with the amino acid residues in the specific PRO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table I below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PRO variant polynucleotide” or “PRO variant nucleic acid sequence” means a nucleic acid molecule which encodes an active PRO polypeptide as defined below and which has at least about 80% nucleic acid sequence identity with a nucleotide acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Ordinarily, a PRO variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal sequence, as disclosed herein or any other fragment of a full-length PRO polypeptide sequence as disclosed herein. Variants do not encompass the native nucleotide sequence.

Ordinarily, PRO variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to PRO-encoding nucleic acid sequences identified herein is defined as the percentage of nucleotides in a candidate sequence that are identical with the nucleotides in the PRO nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the WU-BLAST-2 computer program (Altschul et al., Methods in Enzymology 266:460480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PRO variant polynucleotides are nucleic acid molecules that encode an active PRO polypeptide and which are capable of hybridizing, preferably under stringent hybridization and wash conditions, to nucleotide sequences encoding a full-length PRO polypeptide as disclosed herein. PRO variant polypeptides may be those that are encoded by a PRO variant polynucleotide.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO polypeptide natural environment will not be present. Ordinarily, however, isolated polypeptide will be prepared by at least one purification step.

An “isolated” PRO polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO monoclonal antibodies (including agonist, antagonist, and neutralizing antibodies), anti-PRO antibody compositions with polyepitopic specificity, single chain anti-PRO antibodies, and fragments of anti-PRO antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and %SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a PRO polypeptide which retain a biological and/or an immunological activity of native or naturally-occurring PRO, wherein “biological” activity refers to a biological function (either inhibitory or stimulatory) caused by a native or naturally-occurring PRO other than the ability to induce the production of an antibody against an antigenic epitope possessed by a native or naturally-occurring PRO and an “immunological” activity refers to the ability to induce the production of an antibody against an antigenic epitope possessed by a native or naturally-occurring PRO.

The term “antagonist” is used in the broadest sense, and includes any molecule that partially or fully blocks, inhibits, or neutralizes a biological activity of a native PRO polypeptide disclosed herein. In a similar manner, the term “agonist” is used in the broadest sense and includes any molecule that mimics a biological activity of a native PRO polypeptide disclosed herein. Suitable agonist or antagonist molecules specifically include agonist or antagonist antibodies or antibody fragments, fragments or amino acid sequence variants of native PRO polypeptides, peptides, antisense oligonucleotides, small organic molecules, etc. Methods for identifying agonists or antagonists of a PRO polypeptide may comprise contacting a PRO polypeptide with a candidate agonist or antagonist molecule and measuring a detectable change in one or more biological activities normally associated with the PRO polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time.

“Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fᵥ comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

The term “effective amount” is a concentration or amount of a PRO polypeptide and/or agonist/antagonist which results in achieving a particular stated purpose. An “effective amount” of a PRO polypeptide or agonist or antagonist thereof may be determined empirically. Furthermore, a “therapeutically effective amount” is a concentration or amount of a PRO polypeptide and/or agonist/antagonist which is effective for achieving a stated therapeutic effect. This amount may also be determined empirically.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhöne-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo n inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (W B Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ, colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

As used herein, the term “inflammatory cells” designates cells that enhance the inflammatory response such as mononuclear cells, eosinophils, macrophages, and polymorphonuclear neutrophils (PMN).

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

### II. Compositions and Methods of the Invention

A. Full-Length PRO Polypeptides

The present invention provides newly identified and isolated nucleotide sequences encoding polypeptides referred to in the present application as PRO polypeptides. In particular, cDNAs encoding various PRO polypeptides have been identified and isolated, as disclosed in further detail in the Examples below. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been disclosed. The predicted amino acid sequence can be determined from the nucleotide sequence using routine skill. For the PRO polypeptides and encoding nucleic acids described herein, Applicants have identified what is believed to be the reading frame best identifiable with the sequence information available at the time.

B. PRO Polypeptide Variants

In addition to the full-length native sequence PRO polypeptides described herein, it is contemplated that PRO variants can be prepared. PRO variants can be prepared by introducing appropriate nucleotide changes into the PRO DNA, and/or by synthesis of the desired PRO polypeptide. Those skilled in the art will appreciate that amino acid changes may alter post-translational processes of the PRO, such as changing the number or position of glycosylation sites or altering the membrane anchoring characteristics.

Variations in the native full-length sequence PRO or in various domains of the PRO described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO that results in a change in the amino acid sequence of the PRO as compared with the native sequence PRO. Optionally, the variation is by substitution of at least one amino acid with any other amino acid in one or more of the domains of the PRO. Guidance in determining which amino acid residue may be inserted, substituted or deleted without adversely affecting the desired activity may be found by comparing the sequence of the PRO with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO polypeptide.

PRO fragments may be prepared by any of a number of conventional techniques. Desired peptide fragments may be chemically synthesized. An alternative approach involves generating PRO fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO polypeptide fragments share at least one biological and/or immunological activity with the native PRO polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in function or immunological identity of the PRO polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the PRO variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of PRO

Covalent modifications of PRO are included within the scope of this invention. One type of covalent modification includes reacting targeted amino acid residues of a PRO polypeptide with an organic derivatizing agent that is capable of reacting with selected side chains or the N- or C- terminal residues of the PRO. Derivatization with bifunctional agents is useful, for instance, for crosslinking PRO to a water-insoluble support matrix or surface for use in the method for purifying anti-PRO antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO polypeptide included within the scope of this invention comprises altering the native glycosylation pattern of the polypeptide. “Altering the native glycosylation pattern” is intended for purposes herein to mean deleting one or more carbohydrate moieties found in native sequence PRO (either by removing the underlying glycosylation site or by deleting the glycosylation by chemical and/or enzymatic means), and/or adding one or more glycosylation sites that are not present in the native sequence PRO. In addition, the phrase includes qualitative changes in the glycosylation of the native proteins, involving a change in the nature and proportions of the various carbohydrate moieties present.

Addition of glycosylation sites to the PRO polypeptide may be accomplished by altering the amino acid sequence. The alteration may be made, for example, by the addition of, or substitution by, one or more serine or threonine residues to the native sequence PRO (for O-linked glycosylation sites). The PRO amino acid sequence may optionally be altered through changes at the DNA level, particularly by mutating the DNA encoding the PRO polypeptide at preselected bases such that codons are generated that will translate into the desired amino acids.

Another means of increasing the number of carbohydrate moieties on the PRO polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO comprises linking the PRO polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The PRO of the present invention may also be modified in a way to form a chimeric molecule comprising PRO fused to another, heterologous polypeptide or amino acid sequence.

In one embodiment, such a chimeric molecule comprises a fusion of the PRO with a tag polypeptide which provides an epitope to which an anti-tag antibody can selectively bind. The epitope tag is generally placed at the amino- or carboxyl- terminus of the PRO. The presence of such epitope-tagged forms of the PRO can be detected using an antibody against the tag polypeptide. Also, provision of the epitope tag enables the PRO to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evans et al., Molecular and Cellular Biology, 5:3610-3616 (1985)]; and the Herpes Simplex virus glycoprotein D (gD) tag and its antibody [Paborsky et al., Protein Enigineerig, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the PRO with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of PRO

The description below relates primarily to production of PRO by culturing cells transformed or transfected with a vector containing PRO nucleic acid. It is, of course, contemplated that alternative methods, which are well known in the art, may be employed to prepare PRO. For instance, the PRO sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO may be chemically synthesized separately and combined using chemical or enzymatic methods to produce the full-length PRO.

### 1. Isolation of DNA Encoding PRO

DNA encoding PRO may be obtained from a cDNA library prepared from tissue believed to possess the PRO mRNA and to express it at a detectable level. Accordingly, human PRO DNA can be conveniently obtained from a cDNA library prepared from human tissue, such as described in the Examples. The PRO-encoding gene may also be obtained from a genomic library or by known synthetic procedures (e.g., automated nucleic acid synthesis).

Libraries can be screened with probes (such as antibodies to the PRO or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### 2. Selection and Transformation of Host Cells

Host cells are transfected or transformed with expression or cloning vectors described herein for PRO production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact. 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyomithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia EP 402,226); Pichia pastoris (EP 183,070; Sreekrishna et al., J. Basic Microbiol., 28:265-278 [1988]); Candida; Trichodenna reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

### 3. Selection and Use of a Replicable Vector

The nucleic acid (e.g., cDNA or genomic DNA) encoding PRO may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO-encoding DNA that is inserted into the vector. The signal sequence may be a prokaryotic signal sequence selected, for example, from the group of the alkaline phosphatase, penicillinase, 1pp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 (Jones, Genetics, 85:12 (1977)].

Expression and cloning vectors usually contain a promoter operably linked to the PRO-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO.

Still other methods, vectors, and host cells suitable for adaptation to the synthesis of PRO in recombinant vertebrate cell culture are described in Gething et al., Nature, 293:620-625 (1981); Mantei et al., Nature, 281:40-46 (1979); EP 117,060; and EP 117,058.

### 4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO polypeptide or against a synthetic peptide based on the DNA sequences provided herein or against exogenous sequence fused to PRO DNA and encoding a specific antibody epitope.

### 5. Purification of Polypeptide

Forms of PRO may be recovered from culture medium or from host cell lysates. If membrane-bound, it can be released from the membrane using a suitable detergent solution (e.g. Triton-X 100) or by enzymatic cleavage. Cells employed in expression of PRO can be disrupted by various physical or chemical means, such as freeze-thaw cycling, sonication, mechanical disruption, or cell lysing agents.

It may be desired to purify PRO from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO produced.

E. Tissue Distribution

The location of tissues expressing the PRO can be identified by determining mRNA expression in various human tissues. The location of such genes provides information about which tissues are most likely to be affected by the stimulating and inhibiting activities of the PRO polypeptides. The location of a gene in a specific tissue also provides sample tissue for the activity blocking assays discussed below.

As noted before, gene expression in various tissues may be measured by conventional Southern blotting, Northern blotting to quantitate the transcription of mRNA (Thomas, Proc. Natl. Acad. Sci USA, 77:5201-5205 [1980]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes.

Gene expression in various tissues, alternatively, may be measured by immunological methods, such as immunohistochemical staining of tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence of a PRO polypeptide or against a synthetic peptide based on the DNA sequences encoding the PRO polypeptide or against an exogenous sequence fused to a DNA encoding a PRO polypeptide and encoding a specific antibody epitope. General techniques for generating antibodies, and special protocols for Northern blotting and in situ hybridization are provided below.

F. Antibody Binding Studies

The activity of the PRO polypeptides can be further verified by antibody binding studies, in which the ability of anti-PRO antibodies to inhibit the effect of the PRO polypeptides, respectively, on tissue cells is tested. Exemplary antibodies include polyclonal, monoclonal, humanized, bispecific, and heteroconjugate antibodies, the preparation of which will be described hereinbelow.

Antibody binding studies may be carried out in any known assay method, such as competitive binding assays, direct and indirect sandwich assays, and immunoprecipitation assays. Zola, Monoclonal Antibodies: A Manual of Techniques, pp.147-158 (CRC Press, Inc., 1987).

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tissue sample may be fresh or frozen or may be embedded in paraffin and fixed with a preservative such as formalin, for example.

G. Cell-Based Assays

Cell-based assays and animal models for immune related diseases can be used to further understand the relationship between the genes and polypeptides identified herein and the development and pathogenesis of immune related disease.

In a different approach, cells of a cell type known to be involved in a particular immune related disease are transfected with the cDNAs described herein, and the ability of these cDNAs to stimulate or inhibit immune function is analyzed. Suitable cells can be transfected with the desired gene, and monitored for immune function activity. Such transfected cell lines can then be used to test the ability of poly- or monoclonal antibodies or antibody compositions to inhibit or stimulate immune function, for example to modulate T-cell proliferation or inflammatory cell infiltration. Cells transfected with the coding sequences of the genes identified herein can further be used to identify drug candidates for the treatment of immune related diseases.

In addition, primary cultures derived from transgenic animals (as described below) can be used in the cell-based assays herein, although stable cell lines are preferred. Techniques to derive continuous cell lines from transgenic animals are well known in the art (see, e.g., Small et al., Mol. Cell. Biol. 5: 642-648 [1985]).

One suitable cell based assay is the mixed lymphocyte reaction (MLR). Current Protocols in Immunology, unit 3.12; edited by J E Coligan, A M Kruisbeek, D H Marglies, E M Shevach, W Strober, National Institutes of Health, Published by John Wiley & Sons, Inc. In this assay, the ability of a test compound to stimulate or inhibit the proliferation of activated T cells is assayed. A suspension of responder T cells is cultured with allogeneic stimulator cells and the proliferation of T cells is measured by uptake of tritiated thymidine. This assay is a general measure of T cell reactivity. Since the majority of T cells respond to and produce IL-2 upon activation, differences in responsiveness in this assay in part reflect differences in IL-2 production by the responding cells. The MLR results can be verified by a standard lymphokine (IL-2) detection assay. Current Protocols in Immunology, above, 3.15, 6.3.

A proliferative T cell response in an MLR assay may be due to direct mitogenic properties of an assayed molecule or to external antigen induced activation. Additional verification of the T cell stimulatory activity of the PRO polypeptides can be obtained by a costimulation assay. T cell activation requires an antigen specific signal mediated through the T-cell receptor (TCR) and a costimulatory signal mediated through a second ligand binding interaction, for example, the B7 (CD80, CD86)/CD28 binding interaction. CD28 crosslinking increases lymphokine secretion by activated T cells. T cell activation has both negative and positive controls through the binding of ligands which have a negative or positive effect. CD28 and CTLA-4 are related glycoproteins in the Ig superfamily which bind to B7. CD28 binding to B7 has a positive costimulation effect of T cell activation; conversely, CTLA-4 binding to B7 has a T cell deactivating effect. Chambers, C. A. and Allison, J. P., Curr. Opin. Immunol. (1997) 9:396. Schwartz, R. H., Cell (1992) 71:1065; Linsey, P. S. and Ledbetter, J. A., Annu. Rev. Immunol. (1993) 11:191; June, C. H. et al, Immunol. Today (1994) 15:321; Jenkins, M. K., Immunity (1994) 1:405. In a costimulation assay, the PRO polypeptides are assayed for T cell costimulatory or inhibitory activity.

Direct use of a stimulating compound as in the invention has been validated in experiments with 4-1BB glycoprotein, a member of the tumor necrosis factor receptor family, which binds to a ligand (4-1BBL) expressed on primed T cells and signals T cell activation and growth. Alderson, M. E. et al., J. Immunol. (1994) 24:2219.

The use of an agonist stimulating compound has also been validated experimentally. Activation of 4-1BB by treatment with an agonist anti-4-1BB antibody enhances eradication of tumors. Hellstrom, I. and Hellstrom, K. E., Crit. Rev. Immunol. (1998) 18:1. Immunoadjuvant therapy for treatment of tumors, described in more detail below, is another example of the use of the stimulating compounds of the invention.

Alternatively, an immune stimulating or enhancing effect can also be achieved by administration of a PRO which has vascular permeability enhancing properties. Enhanced vascular permeability would be beneficial to disorders which can be attenuated by local infiltration of immune cells (e.g., monocytes, eosinophils, PMNs) and inflammation.

On the other hand, PRO polypeptides, as well as other compounds of the invention, which are direct inhibitors of T cell proliferation/activation, lymphokine secretion, and/or vascular permeability can be directly used to suppress the immune response. These compounds are useful to reduce the degree of the immune response and to treat immune related diseases characterized by a hyperactive, superoptimal, or autoimmune response. This use of the compounds of the invention has been validated by the experiments described above in which CTLA-4 binding to receptor B7 deactivates T cells. The direct inhibitory compounds of the invention function in an analogous manner. The use of compound which suppress vascular permeability would be expected to reduce inflammation. Such uses would be beneficial in treating conditions associated with excessive inflammation.

Alternatively, compounds, e.g., antibodies, which bind to stimulating PRO polypeptides and block the stimulating effect of these molecules produce a net inhibitory effect and can be used to suppress the T cell mediated immune response by inhibiting T cell proliferation/activation and/or lymphokine secretion. Blocking the stimulating effect of the polypeptides suppresses the immune response of the mammal. This use has been validated in experiments using an anti-IL2 antibody. In these experiments, the antibody binds to IL2 and blocks binding of IL2 to its receptor thereby achieving a T cell inhibitory effect.

H. Animal Models

The results of the cell based in vitro assays can be further verified using in vivo animal models and assays for T-cell function. A variety of well known animal models can be used to further understand the role of the genes identified herein in the development and pathogenesis of immune related disease, and to test the efficacy of candidate therapeutic agents, including antibodies, and other antagonists of the native polypeptides, including small molecule antagonists. The in vivo nature of such models makes them predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g., subcutaneous injection, tail vein injection, spleen implantation, intraperitoneal implantation, implantation under the renal capsule, etc.

Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. Graft-versus-host disease models provide a means of assessing T cell reactivity against MHC antigens and minor transplant antigens. A suitable procedure is described in detail in Current Protocols in Immunology, above, unit 4.3.

An animal model for skin allograft rejection is a means of testing the ability of T cells to mediate in vivo tissue destruction and a measure of their role in transplant rejection. The most common and accepted models use murine tail-skin grafts. Repeated experiments have shown that skin allograft rejection is mediated by T cells, helper T cells and killer-effector T cells, and not antibodies. Auchincloss, H. Jr. and Sachs, D. H., Fundamental Immunology, 2nd ed., W. E. Paul ed., Raven Press, NY, 1989, 889-992. A suitable procedure is described in detail in Current Protocols in Immunology, above, unit 4.4. Other transplant rejection models which can be used to test the compounds of the invention are the allogeneic heart transplant models described by Tanabe, M. et al, Transplantation (1994) 58:23 and Tinubu, S. A. et al, J. Immunol. (1994) 4330-4338.

Animal models for delayed type hypersensitivity provides an assay of cell mediated immune function as well. Delayed type hypersensitivity reactions are a T cell mediated in vivo immune response characterized by inflammation which does not reach a peak until after a period of time has elapsed after challenge with an antigen. These reactions also occur in tissue specific autoimmune diseases such as multiple sclerosis (MS) and experimental autoimmune encephalomyelitis (EAE, a model for MS). A suitable procedure is described in detail in Current Protocols in Immunology, above, unit 4.5.

EAE is a T cell mediated autoimmune disease characterized by T cell and mononuclear cell inflammation and subsequent demyelination of axons in the central nervous system. EAE is generally considered to be a relevant animal model for MS in humans. Bolton, C., Multiple Sclerosis (1995) 1:143. Both acute and relapsing-remitting models have been developed. The compounds of the invention can be tested for T cell stimulatory or inhibitory activity against immune mediated demyelinating disease using the protocol described in Current Protocols in Immunology, above, units 15.1 and 15.2. See also the models for myelin disease in which oligodendrocytes or Schwann cells are grafted into the central nervous system as described in Duncan, I. D. et al, Molec. Med. Today (1997) 554-561.

Contact hypersensitivity is a simple delayed type hypersensitivity in vivo assay of cell mediated immune function. In this procedure, cutaneous exposure to exogenous haptens which gives rise to a delayed type hypersensitivity reaction which is measured and quantitated. Contact sensitivity involves an initial sensitizing phase followed by an elicitation phase. The elicitation phase occurs when the T lymphocytes encounter an antigen to which they have had previous contact. Swelling and inflammation occur, making this an excellent model of human allergic contact dermatitis. A suitable procedure is described in detail in Current Protocols in Immunology, Eds. J. E. Cologan, A. M. Kruisbeek, D. H. Margulies, E. M. Shevach and W. Strober, John Wiley & Sons, Inc., 1994, unit 4.2. See also Grabbe, S. and Schwarz, T, Immun. Today 19 (1): 37-44 (1998).

An animal model for arthritis is collagen-induced arthritis. This model shares clinical, histological and immunological characteristics of human autoimmune rheumatoid arthritis and is an acceptable model for human autoimmune arthritis. Mouse and rat models are characterized by synovitis, erosion of cartilage and subchondral bone. The compounds of the invention can be tested for activity against autoimmune arthritis using the protocols described in Current Protocols in Immunology, above, units 15.5. See also the model using a monoclonal antibody to CD18 and VLA-4 integrins described in Issekutz, A. C. et al., Immunology (1996) 88:569.

A model of asthma has been described in which antigen-induced airway hyper-reactivity, pulmonary eosinophilia and inflammation are induced by sensitizing an animal with ovalbumin and then challenging the animal with the same protein delivered by aerosol. Several animal models (guinea pig, rat, non-human primate) show symptoms similar to atopic asthma in humans upon challenge with aerosol antigens. Murine models have many of the features of human asthma. Suitable procedures to test the compounds of the invention for activity and effectiveness in the treatment of asthma are described by Wolyniec, W. W. et al., Am. J. Respir. Cell Mol. Biol. (1998) 18:777 and the references cited therein.

Additionally, the compounds of the invention can be tested on animal models for psoriasis like diseases. Evidence suggests a T cell pathogenesis for psoriasis. The compounds of the invention can be tested in the scid/scid mouse model described by Schon, M. P. et al, Nat. Med. (1997) 3:183, in which the mice demonstrate histopathologic skin lesions resembling psoriasis. Another suitable model is the human skin/scid mouse chimera prepared as described by Nickoloff, B. J. et al, Am. J. Path. (1995) 146:580.

Recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA 82, 6148-615 [1985]); gene targeting in embryonic stem cells (Thompson et al., Cell 56, 313-321 [1989]); electroporation of embryos Val (V) ile; leu; met; phe; ala; norleucine leu (Lo, Mol. Cel. Biol. 3, 1803-1814 [1983]); sperm-mediated gene transfer (Lavitrano et al., Cell 57, 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA 89 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry.

The animals may be further examined for signs of immune disease pathology, for example by histological examination to determine infiltration of immune cells into specific tissues. Blocking experiments can also be performed in which the transgenic animals are treated with the compounds of the invention to determine the extent of the T cell proliferation stimulation or inhibition of the compounds. In these experiments, blocking antibodies which bind to the PRO polypeptide, prepared as described above, are administered to the animal and the effect on immune function is determined.

Alternatively, “knock out” animals can be constructed which have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

I. ImmunoAdjuvant Therapy

In one embodiment, the immunostimulating compounds of the invention can be used in immunoadjuvant therapy for the treatment of tumors (cancer). It is now well established that T cells recognize human tumor specific antigens. One group of tumor antigens, encoded by the MAGE, BAGE and GAGE families of genes, are silent in all adult normal tissues, but are expressed in significant amounts in tumors, such as melanomas, lung tumors, head and neck tumors, and bladder carcinomas DeSmet et al., (1996) Proc. Natl. Acad. Sci. USA, 93:7149. It has been shown that costimulation of T cells induces tumor regression and an antitumor response both in vitro and in vivo. Melero, I. et al., Nature Medicine (1997) 3:682; Kwon, E. D. et al., Proc. Natl. Acad. Sci. USA (1997) 94: 8099; Lynch, D. H. et al, Nature Medicine (1997) 3:625; Finn, O. J. and Lotze, M. T., J. Immunol. (1998) 21:114. The stimulatory compounds of the invention can be administered as adjuvants, alone or together with a growth regulating agent, cytotoxic agent or chemotherapeutic agent, to stimulate T cell proliferation/activation and an antitumor response to tumor antigens. The growth regulating, cytotoxic, or chemotherapeutic agent may be administered in conventional amounts using known administration regimes. Immunostimulating activity by the compounds of the invention allows reduced amounts of the growth regulating, cytotoxic, or chemotherapeutic agents thereby potentially lowering the toxicity to the patient.

J. Screening Assays for Drug Candidates

Screening assays for drug candidates are designed to identify compounds that bind to or complex with the polypeptides encoded by the genes identified herein or a biologically active fragment thereof, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art. All assays are common in that they call for contacting the drug candidate with a polypeptide encoded by a nucleic acid identified herein under conditions and for a time sufficient to allow these two components to interact.

In binding assays, the interaction is binding and the complex formed can be isolated or detected in the reaction mixture. In a particular embodiment, the polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, erg., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex.

If the candidate compound interacts with but does not bind to a particular protein encoded by a gene identified herein, its interaction with that protein can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature (London) 340, 245-246 (1989); Chien et al., Proc. Natl. Acad. Sci. USA 88, 9578-9582 (1991)] as disclosed by Chevray and Nathans, Proc. Natl. Acad. Sci. USA 89, 5789-5793 (1991). Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GALA-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

In order to find compounds that interfere with the interaction of a gene identified herein and other intra- or extracellular components can be tested, a reaction mixture is usually prepared containing the product of the gene and the intra- or extracellular component under conditions and for a time allowing for the interaction and binding of the two products. To test the ability of a test compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described above. The formation of a complex in the control reaction(s) but not in the reaction mixture containing the test compound indicates that the test compound interferes with the interaction of the test compound and its reaction partner.

K. Compositions and Methods for the Treatment of Immune Related Diseases

The compositions useful in the treatment of immune related diseases include, without limitation, proteins, antibodies, small organic molecules, peptides, phosphopeptides, antisense and ribozyme molecules, triple helix molecules, etc. that inhibit or stimulate immune function, for example, T cell proliferation/activation, lymphokine release, or immune cell infiltration.

For example, antisense RNA and RNA molecules act to directly block the translation of mRNA by hybridizing to targeted mRNA and preventing protein translation. When antisense DNA is used, oligodeoxyribonucleotides derived from the translation initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4, 469-471 (1994), and PCT publication No. WO 97/33551 (published Sep. 18, 1997).

Nucleic acid molecules in triple helix formation used to inhibit transcription should be single-stranded and composed of deoxynucleotides. The base composition of these oligonucleotides is designed such that it promotes triple helix formation via Hoogsteen base pairing rules, which generally require sizeable stretches of purines or pyrimidines on one strand of a duplex. For further details see, e.g., PCT publication No. WO 97/33551, supra.

These molecules can be identified by any or any combination of the screening assays discussed above and/or by any other screening techniques well known for those skilled in the art.

L. Anti-PRO Antibodies

The present invention further provides anti-PRO antibodies. Exemplary antibodies include polyclonal, monoclonal, humanized, bispecific, and heteroconjugate antibodies.

### 1. Polyclonal Antibodies

The anti-PRO antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

### 2. Monoclonal Antibodies

The anti-PRO antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the PRO polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

### 3. Human and Humanized Antibodies

The anti-PRO antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably times, even more preferably 20 or 30 times greater than the starting antibody (generally murine, humanized or human) from which the matured antibody is prepared.

### 4. Bispecific Antibodies

Bispecific antibodies are monoclonal, preferably human or humanized, antibodies that have binding specificities for at least two different antigens. In the present case, one of the binding specificities is for the PRO, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given PRO polypeptide herein. Alternatively, an anti-PRO polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular PRO polypeptide. Bispecific antibodies may also be used to localize cytotoxic agents to cells which express a particular PRO polypeptide. These antibodies possess a PRO-binding arm and an arm which binds a cytotoxic agent or a radionuclide chelator, such as EOTUBE, DPTA, DOTA, or TETA. Another bispecific antibody of interest binds the PRO polypeptide and further binds tissue factor (TF).

### 5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### 6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design, 3: 219-230 (1989).

### 7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, A leurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

### 8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81 (19): 1484 (1989).

M. Pharmaceutical Compositions

The active PRO molecules of the invention (e.g., PRO polypeptides, anti-PRO antibodies, and/or variants of each) as well as other molecules identified by the screening assays disclosed above, can be administered for the treatment of immune related diseases, in the form of pharmaceutical compositions.

Therapeutic formulations of the active PRO molecule, preferably a polypeptide or antibody of the invention, are prepared for storage by mixing the active molecule having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Compounds identified by the screening assays disclosed herein can be formulated in an analogous manner, using standard techniques well known in the art.

Lipofections or liposomes can also be used to deliver the PRO molecule into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90, 7889-7893 [1993]).

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active PRO molecules may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations or the PRO molecules may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

N. Methods of Treatment

It is contemplated that the polypeptides, antibodies and other active compounds of the present invention may be used to treat various immune related diseases and conditions, such as T cell mediated diseases, including those characterized by infiltration of inflammatory cells into a tissue, stimulation of T-cell proliferation, inhibition of T-cell proliferation, increased or decreased vascular permeability or the inhibition thereof.

Exemplary conditions or disorders to be treated with the polypeptides, antibodies and other compounds of the invention, include, but are not limited to systemic lupus erythematosis, rheumatoid arthritis, juvenile chronic arthritis, osteoarthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In systemic lupus erythematosus, the central mediator of disease is the production of auto-reactive antibodies to self proteins/tissues and the subsequent generation of immune-mediated inflammation. Antibodies either directly or indirectly mediate tissue injury. Though T lymphocytes have not been shown to be directly involved in tissue damage, T lymphocytes are required for the development of auto-reactive antibodies. The genesis of the disease is thus T lymphocyte dependent. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjögren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barrë syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+ T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus-Host-Disease (GVHD) are T lymphocyte-dependent; inhibition of T lymphocyte function is ameliorative. Other diseases in which intervention of the immune and/or inflammatory response have benefit are infectious disease including but not limited to viral infection (including but not limited to AIDS, hepatitis A, B, C, D, E and herpes) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (ie., as from chemotherapy) immunodeficiency, and neoplasia.

It has been demonstrated that some human cancer patients develop an antibody and/or T lymphocyte response to antigens on neoplastic cells. It has also been shown in animal models of neoplasia that enhancement of the immune response can result in rejection or regression of that particular neoplasm. Molecules that enhance the T lymphocyte response in the MLR have utility in vivo in enhancing the immune response against neoplasia. Molecules which enhance the T lymphocyte proliferative response in the MLR (or small molecule agonists or antibodies that affected the same receptor in an agonistic fashion) can be used therapeutically to treat cancer. Molecules that inhibit the lymphocyte response in the MLR also function in vivo during neoplasia to suppress the immune response to a neoplasm; such molecules can either be expressed by the neoplastic cells themselves or their expression can be induced by the neoplasm in other cells. Antagonism of such inhibitory molecules (either with antibody, small molecule antagonists or other means) enhances immune-mediated tumor rejection.

Additionally, inhibition of molecules with proinflammatory properties may have therapeutic benefit in reperfusion injury; stroke; myocardial infarction; atherosclerosis; acute lung injury; hemorrhagic shock; burn; sepsis/septic shock; acute tubular necrosis; endometriosis; degenerative joint disease and pancreatis.

The compounds of the present invention, e.g., polypeptides or antibodies, are administered to a mammal, preferably a human, in accord with known methods, such as intravenous administration as a bolus or by continuous inftusion over a period of time, by intramuscular, intraperitoneal, intracerobrospinal, subcutaneous, intra-articular, intrasynovial, intrathecal, oral, topical, or inhalation (intranasal, intrapulmonary) routes. Intravenous or inhaled administration of polypeptides and antibodies is preferred.

In immunoadjuvant therapy, other therapeutic regimens, such administration of an anti-cancer agent, may be combined with the administration of the proteins, antibodies or compounds of the instant invention. For example, the patient to be treated with a the immunoadjuvant of the invention may also receive an anti-cancer agent (chemotherapeutic agent) or radiation therapy. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the immunoadjuvant or may be given simultaneously therewith. Additionally, an anti-estrogen compound such as tamoxifen or an anti-progesterone such as onapristone (see, EP 616812) may be given in dosages known for such molecules.

It may be desirable to also administer antibodies against other immune disease associated or tumor associated antigens, such as antibodies which bind to CD20, CD11a, CD18, ErbB2, EGFR, ErbB3, ErbB4, or vascular endothelial factor (VEGF). Alternatively, or in addition, two or more antibodies binding the same or two or more different antigens disclosed herein may be coadministered to the patient. Sometimes, it may be beneficial to also administer one or more cytokines to the patient. In one embodiment, the PRO polypeptides are coadministered with a growth inhibitory agent. For example, the growth inhibitory agent may be administered first, followed by a PRO polypeptide. However, simultaneous administration or administration first is also contemplated. Suitable dosages for the growth inhibitory agent are those presently used and may be lowered due to the combined action (synergy) of the growth inhibitory agent and the PRO polypeptide.

For the treatment or reduction in the severity of immune related disease, the appropriate dosage of an a compound of the invention will depend on the type of disease to be treated, as defined above, the severity and course of the disease, whether the agent is administered for preventive or therapeutic purposes, previous therapy, the patient's clinical history and response to the compound, and the discretion of the anending physician. The compound is suitably administered to the patient at one time or over a series of treatments.

For example, depending on the type and severity of the disease, about 1 μg/kg to 15 mg/kg (e.g., 0.1-20 mg/kg) of polypeptide or antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

O. Articles of Manufacture

In another embodiment of the invention, an article of manufacture containing materials (e.g., comprising a PRO molecule) useful for the diagnosis or treatment of the disorders described above is provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually a polypeptide or an antibody of the invention. An instruction or label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

P. Diagnosis and Prognosis of Immune Related Disease

Cell surface proteins, such as proteins which are overexpressed in certain immune related diseases, are excellent targets for drug candidates or disease treatment. The same proteins along with secreted proteins encoded by the genes amplified in immune related disease states find additional use in the diagnosis and prognosis of these diseases. For example, antibodies directed against the protein products of genes amplified in multiple sclerosis, rheumatoid arthritis, or another immune related disease, can be used as diagnostics or prognostics.

For example, antibodies, including antibody fragments, can be used to qualitatively or quantitatively detect the expression of proteins encoded by amplified or overexpressed genes (“marker gene products”). The antibody preferably is equipped with a detectable, e.g., fluorescent label, and binding can be monitored by light microscopy, flow cytometry, fluorimetry, or other techniques known in the art. These techniques are particularly suitable, if the overexpressed gene encodes a cell surface protein Such binding assays are performed essentially as described above.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Microarray Analysis of Stimulated T-cells

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (for example, activated CD4+ T cells) sample is greater than hybridization signal of a probe from a control (for example, non-stimulated CD4+ T cells) sample, the gene or genes overexpressed in the test tissue are identified. The implication of this result is that an overexpressed protein in a test tissue is useful not only as a diagnostic marker for the presence of a disease condition, but also as a therapeutic target for treatment of a disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

When CD4+ T cells mature from thymus and enter into the peripheral lymph system, they usually maintain their naive phenotype before encountering antigens specific for their T cell receptor [Sprent et al., Annu Rev Immunol. (2002); 20:551-79]. The binding to specific antigens presented by APC, causes T cell activation. Depending on the environment and cytokine stimulation, CD4+ T cells differentiate into a Th1 or Th2 phenotype and become effector or memory cells [Sprent et al., Annu Rev Immunol. (2002); 20:551-79 and Murphy et al., Nat Rev Immunol. (2002) December; 2(12):933-44). This process is known as primary activation. Having undergone primary activation, CD4+ T cells become effector or memory cells, they maintain their phenotype as Th1 or Th2. Once these cells encounter antigen again, they undergo secondary activation, but this time the response to antigen will be quicker than the primary activation and results in the production of effector cytokines as determined by the primary activation [Sprent et al., Annu Rev Immunol. (2002); 20:551-79 and Murphy et al., Annu Rev Immunol. 2000;18:451-94].

Studies have found during the primary and secondary activation of CD4+ T cells the expression of certain genes is variable [Rogge et al., Nature Genetics. 25, 96-101 (2000) and Ouyang et al., Proc Natl Acad Sci USA. (1999) Mar. 30; 96(7):3888-931. The present study represents a model to identify differentially expressed genes during the primary and secondary activation response in vitro.

For primary activation conditions, naive T cells were activated by anti-CD3, anti-CD28 and specific cytokines (experimental conditions are described below). This primary activation was termed condition (a). RNA isolated from cells in this condition can provide information about what genes are differentially regulated during the primary activation, and what cytokines affect gene expression during Th1 and Th2 development. After primary activation, the CD4+ T cells were maintained in culture for a week. However, as the previous activation and cytokine treatment has been imprinted into these cells and they have become either effector or memory cells. During this period, because there are no APCs or antigens, the CD4+ T cells enter a resting stage. This resting stage, termed condition (b) (with experimental conditions described below), provides information about the differences between naive vs. memory cells, and resting memory Th1 vs. resting memory Th2 cells. The resting memory Th1 and Th2 cells then undergo secondary activation under condition (c) and condition (d), with both conditions being described below. These conditions provide information about the differences between activated naive and activated memory T cells, and the differences between activated memory Th1 vs. activated memory Th2 cells. This study demonstrates differential gene expression during different stages of CD4 T cell activation and differentiation. As we know, many autoimmune diseases are caused by memory Th1 and Th2 cells. The data now provide us opportunity to find markers to identify these cells and specifically target these cells as a new therapeutic approach.

In this experiment, CD4+ T cells were purified from a single donor using the RossetteSep™ protocol (Stem Cell Technologies, Vancouver BC) which contains anti-CD8, anti-CD16, anti-CD19, anti-CD36 and anti-CD56 antibodies used to produce a population of isolated CD4+ T cells with the modification to the protocol of using 1.3 ml reagent/25 ml blood. The isolated CD4+ T cells were washed by PBS (0.5% BSA) twice and counted. Naive CD4+ T cells were further isolated by Miltenyi CD45RO beads (Miltenyi Biotec) through the autoMACS™ depletion program and the purity of the cells was determined by FACS analysis. Experiments proceeded only with >90% cell pure CD4+ T cells. At this point RNA was extracted from 50×10ˆ6 CD4+ T cells for use as a baseline control. The remainder of the cells were stimulated by plate bound anti-CD3 and anti-CD28 at 20×10ˆ6 cells/6 ml T cell media/well of a 6 well plate.

On Day 1, to induce Th1 differentiation, IL-12 (1 ng/ml) and anti-IL4 (1 μ/ml)were added. For Th2 differentiation, IL4 (5 ng/ml), anti-IL-12 (0.5 μg/ml), and anti-IFN-g were added. For Th0 cells, anti-IL-12 (0.5 μg/ml), anti-IL4 (1 μg/ml) and anti-IFN-gamma (0.1 μg/ml) were added. All reagents were from R&D Systems (R & D Systems Inc. Minneapolis, Minn.).

On Day 2, cells from one well per condition were harvested for RNA purification to obtain a 48 hr time point (condition (a)). On Day 3, the cells were expanded 4 fold by removing the media used for differentiation, and adding fresh media plus IL-2 and cultured for 4 days. On Day 7, the cells were washed and counted, and the cytokine profiles were examined by intracellular cytokine staining and ELISA to determine if differentiation was complete. Half of the cells were harvested and RNA purified to determine the expression of genes in the resting state (condition (b)). IL4 and IFN-gamma producing cells were enriched for by using the Miltenyi™ cytokine assay kit. The isolated IL-4 or IFN-gamma producing cells were expanded for two more weeks by using similar conditions as above.

On Day 21, cells were harvested and subject to intracellular cytokine staining and ELISA for cytokine production analysis. The remainder of the cells were re-stimulated by anti-CD3 and anti-CD28 (secondary activation). Cells were harvested at 12 hr (condition (c)) and 48 hr (condition (d)) for RNA purification. From the different conditions, RNA was extracted and analysis run on Affimax (Affymetrix Inc. Santa Clara, Calif.) microarray chips. Non-stimulated cells harvested immediately after purification, were subjected to the same analysis. Genes were compared whose expression was upregulated or downregulated at the different activated conditions vs. resting cells.

Below are the results of these experiments, demonstrating that various PRO polypeptides of the present invention are significantly upregulated or downregulated in isolated stimulated CD4+ T helper cells as compared to unstimulated CD4+ T helper cells or isolated resting CD4+ T helper cells. As Th1 and Th2 cells play a role in normal immune defense during infection, and play a role in immune disorders, this data demonstrate that the PRO polypeptides of the present invention are useful not only as diagnostic markers for the presence of one or more immune disorders, but also serve as therapeutic targets for the treatment of those immune disorders.

SEQ ID NOs 1-6464 show nucleic acids and their encoded proteins show differential expression at (condition (c)) or (condition (d)) vs. unstimulated cells as a normal control, cells that have undergone primary activation, or primary activated cells that had been in resting for 7 days. SEQ ID NO:2955, SEQ ID NO:2855, SEQ ID NO:3487, SEQ ID NO:3088, SEQ ID NO:1319, SEQ ID NO:1629, SEQ ID NO:1733, SEQ ID NO: 1561, and SEQ ID NO: 1699 are highly overexpressed at (condtion (c)) or (condition (d)) vs. unstimulated cells as a normal control , cells that have undergone primary activation, or primary activated cells that had been in resting for 7 days.

### Example 2

### Use of PRO as a Hybridization Probe

The following method describes use of a nucleotide sequence encoding PRO as a hybridization probe.

DNA comprising the coding sequence of full-length or mature PRO as disclosed herein is employed as a probe to screen for homologous DNAs (such as those encoding naturally-occurring variants of PRO) in human tissue cDNA libraries or human tissue genomic libraries.

Hybridization and washing of filters containing either library DNAs is performed under the following high stringency conditions. Hybridization of radiolabeled PRO-derived probe to the filters is performed in a solution of 50% formamide, 5×SSC, 0.1% SDS, 0.1% sodium pyrophosphate, 50 mM sodium phosphate, pH 6.8, 2×Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PRO can then be identified using standard techniques known in the art.

### Example 3

### Expression of PRO in E. coli

This example illustrates preparation of an unglycosylated form of PRO by recombinant expression in E. coli.

The DNA sequence encoding PRO is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO protein can then be purified using a metal chelating column under conditions that allow tight binding of the protein.

PRO may be expressed in E. coli in a poly-His tagged form, using the following procedure. The DNA encoding PRO is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon gale rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 MM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

Many of the PRO polypeptides disclosed herein were successfully expressed as described above.

### Example 4

### Expression of PRO in Mammalian Cells

This example illustrates preparation of a potentially glycosylated form of PRO by recombinant expression in mammalian cells.

The vector, pRK5 (see EP 307,247, published Mar. 15, 1989), is employed as the expression vector. Optionally, the PRO DNA is ligated into pRK5 with selected restriction enzymes to allow insertion of the PRO DNA using ligation methods such as described in Sambrook et al., supra. The resulting vector is called pRK5-PRO.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO polypeptide. The cultures containing transfected cells may undergo further incubation (in serum free medium) and the medium is tested in selected bioassays.

In an alternative technique, PRO may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO can then be concentrated and purified by any selected method, such as dialysis and/or column chromatography.

In another embodiment, PRO can be expressed in CHO cells. The pRK5-PRO can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO polypeptide, the culture medium may be replaced with serum free medium. Preferably, the cultures are incubated for about 6 days, and then the conditioned medium is harvested. The medium containing the expressed PRO can then be concentrated and purified by any selected method.

Epitope-tagged PRO may also be expressed in host CHO cells. The PRO may be subcloned out of the pRK5 vector. The subclone insert can undergo PCR to fuse in frame with a selected epitope tag such as a poly-his tag into a Baculovirus expression vector. The poly-his tagged PRO insert can then be subcloned into a SV40 promoter/enhancer containing vector containing a selection marker such as DHFR for selection of stable clones. Finally, the CHO cells can be transfected (as described above) with the SV40 promoter/enhancer containing vector. Labeling may be performed, as described above, to verify expression. The culture medium containing the expressed poly-His tagged PRO can then be concentrated and purified by any selected method, such as by Ni²⁺-chelate affinity chromatography.

PRO may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mL of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μl of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Many of the PRO polypeptides disclosed herein were successfully expressed as described above.

### Example 5

### Expression of PRO in Yeast

The following method describes recombinant expression of PRO in yeast.

First, yeast expression vectors are constructed for intracellular production or secretion of PRO from the ADH2/GAPDH promoter. DNA encoding PRO and the promoter is inserted into suitable restriction enzyme sites in the selected plasmid to direct intracellular expression of PRO. For secretion, DNA encoding PRO can be cloned into the selected plasmid, together with DNA encoding the ADH2/GAPDH promoter, a native PRO signal peptide or other mammalian signal peptide, or, for example, a yeast alpha-factor or invertase secretory signal/leader sequence, and linker sequences (if needed) for expression of PRO.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO can subsequently be isolated and purified by removing the yeast cells from the fermentation medium by centrifugation and then concentrating the medium using selected cartridge filters. The concentrate containing PRO may further be purified using selected column chromatography resins.

Many of the PRO polypeptides disclosed herein were successfully expressed as described above.

### Example 6

### Expression of PRO in Baculovirus-Infected Insect Cells

The following method describes recombinant expression of PRO in Baculovirus-infected insect cells.

The sequence coding for PRO is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO or the desired portion of the coding sequence of PRO such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO are pooled and dialyzed against loading buffer.

Alternatively, purification of the IgG tagged (or Fc tagged) PRO can be performed using known chromatography techniques, including for instance, Protein A or protein G column chromatography.

Many of the PRO polypeptides disclosed herein were successfully expressed as described above.

### Example 7

### Preparation of Antibodies that Bind PRO

This example illustrates preparation of monoclonal antibodies which can specifically bind PRO.

Techniques for producing the monoclonal antibodies are known in the art and are described, for instance, in Goding, supra. Immunogens that may be employed include purified PRO, fusion proteins containing PRO, and cells expressing recombinant PRO on the cell surface. Selection of the immunogen can be made by the skilled artisan without undue experimentation.

Mice, such as Balb/c, are immunized with the PRO immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO antibodies.

After a suitable antibody titer has been detected, the animals “positive” for antibodies can be injected with a final intravenous injection of PRO. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO. Determination of “positive” hybridoma cells secreting the desired monoclonal antibodies against PRO is within the skill in the art.

The positive hybridoma cells can be injected intraperitoneally into syngeneic Balb/c mice to produce ascites containing the anti-PRO monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

### Purification of PRO Polypeptides Using Specific Antibodies

Native or recombinant PRO polypeptides may be purified by a variety of standard techniques in the art of protein purification. For example, pro-PRO polypeptide, mature PRO polypeptide, or pre-PRO polypeptide is purified by immunoaffinity chromatography using antibodies specific for the PRO polypeptide of interest. In general, an immunoaffinity column is constructed by covalently coupling the anti-PRO polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO polypeptide by preparing a fraction from cells containing PRO polypeptide in a soluble form. This preparation is derived by solubilization of the whole cell or of a subcellular fraction obtained via differential centrifugation by the addition of detergent or by other methods well known in the art. Alternatively, soluble PRO polypeptide containing a signal sequence may be secreted in useful quantity into the medium in which the cells are grown.

A soluble PRO polypeptide-containing preparation is passed over the immunoaffinity column, and the column is washed under conditions that allow the preferential absorbance of PRO polypeptide (e.g., high ionic strength buffers in the presence of detergent). Then, the column is eluted under conditions that disrupt antibody/PRO polypeptide binding (e.g., a low pH buffer such as approximately pH 2-3, or a high concentration of a chaotrope such as urea or thiocyanate ion), and PRO polypeptide is collected.

### Example 9

### Drug Screening

This invention is particularly useful for screening compounds by using PRO polypeptides or binding fragment thereof in any of a variety of drug screening techniques. The PRO polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO polypeptide or fragment. Drugs are screened against such transformed cells in competitive binding assays. Such cells, either in viable or fixed form, can be used for standard binding assays. One may measure, for example, the formation of complexes between PRO polypeptide or a fragment and the agent being tested. Alternatively, one can examine the diminution in complex formation between the PRO polypeptide and its target cell or target receptors caused by the agent being tested.

Thus, the present invention provides methods of screening for drugs or any other agents which can affect a PRO polypeptide-associated disease or disorder. These methods comprise contacting such an agent with an PRO polypeptide or fragment thereof and assaying (I) for the presence of a complex between the agent and the PRO polypeptide or fragment, or (ii) for the presence of a complex between the PRO polypeptide or fragment and the cell, by methods well known in the art. In such competitive binding assays, the PRO polypeptide or fragment is typically labeled. After suitable incubation, free PRO polypeptide or fragment is separated from that present in bound form, and the amount of free or uncomplexed label is a measure of the ability of the particular agent to bind to PRO polypeptide or to interfere with the PRO polypeptidelcell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO polypeptide, the peptide test compounds are reacted with PRO polypeptide and washed. Bound PRO polypeptide is detected by methods well known in the art. Purified PRO polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO polypeptide specifically compete with a test compound for binding to PRO polypeptide or fragments thereof. In this manner, the antibodies can be used to detect the presence of any peptide which shares one or more antigenic determinants with PRO polypeptide.

### Example 10

### Rational Drug Design

The goal of rational drug design is to produce structural analogs of biologically active polypeptide of interest (i.e., a PRO polypeptide) or of small molecules with which they interact, e.g., agonists, antagonists, or inhibitors. Any of these examples can be used to fashion drugs which are more active or stable forms of the PRO polypeptide or which enhance or interfere with the function of the PRO polypeptide in vivo (c.f., Hodgson, Bio/Technology, 9: 19-21 (1991)).

In one approach, the three-dimensional structure of the PRO polypeptide, or of a PRO polypeptide-inhibitor complex, is determined by x-ray crystallography, by computer modeling or, most typically, by a combination of the two approaches. Both the shape and charges of the PRO polypeptide must be ascertained to elucidate the structure and to determine active site(s) of the molecule. Less often, useful information regarding the structure of the PRO polypeptide may be gained by modeling based on the structure of homologous proteins. In both cases, relevant structural information is used to design analogous PRO polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al, J. Biochem., 13:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO polypeptide may be made available to perform such analytical studies as X-ray crystallography. In addition, knowledge of the PRO polypeptide amino acid sequence provided herein will provide guidance to those employing computer modeling techniques in place of or in addition to x-ray crystallography.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

APPENDIX A
List of Figures

FIG. 1: DNA344243, U25789, 200012_x_at
FIG. 2: PRO94991
FIG. 3: DNA326466, NP_004530.1, 200027_at
FIG. 4: PRO60800
FIG. 5: DNA326324, NP_000972.1, 200029_at
FIG. 6: PRO4738
FIG. 7: DNA344244, NP_006324.1, 200056_s_at
FIG. 8: PRO61385
FIG. 9: DNA304680, NP_031381.2, 200064_at
FIG. 10: PRO71106
FIG. 11: DNA325222, NP_000967.1, 200088_x_at
FIG. 12: PRO62236
FIG. 13: DNA270963, NP_003326.1, 1294_at
FIG. 14: PRO59293
FIG. 15: DNA188207, NP_005371.1, 37005_at
FIG. 16: PRO21719
FIG. 17: DNA333633, NP_055697.1, 38149_at
FIG. 18: PRO88275
FIG. 19: DNA254127, NP_008925.1, 38241_at
FIG. 20: PRO49242
FIG. 21A-B: DNA329908, BAA13246.1, 38892_at
FIG. 22: PRO85225
FIG. 23: DNA327523, NP_004916.1, 39248_at
FIG. 24: PRO38028
FIG. 25: DNA328357, 1452321.2, 39582_at
FIG. 26: PRO84217
FIG. 27A-B: DNA273398, NP_056383.1, 41577_at
FIG. 28: PRO61398
FIG. 29: DNA327526, NP_065727.2, 45288_at
FIG. 30: PRO83574
FIG. 31: DNA344245, AF177331, 47069_at
FIG. 32: PRO94992
FIG. 33A-B: DNA335121, NP_066300.1, 47550_at
FIG. 34: PRO89524
FIG. 35: DNA344246, NP_009093.1, 50221_at
FIG. 36: PRO94993
FIG. 37A-B: DNA226870, NP_000782.1, 48808_at
FIG. 38: PRO37333
FIG. 39A-B: DNA194778, NP_055545.1, 200617_at
FIG. 40: PRO24056
FIG. 41: DNA287245, NP_004175.1, 200628_s_at
FIG. 42: PRO69520
FIG. 43: DNA287245, NM_004184, 200629_at
FIG. 44: PRO69520
FIG. 45: DNA327532, NP_002056.2, 200648_s_at
FIG. 46: PRO71134
FIG. 47: DNA226063, X05130, 200656_s_at
FIG. 48: PRO36526
FIG. 49: DNA274759, NP_005611.1, 200660_at
FIG. 50: PRO62529
FIG. 51: DNA324276, NP_000985.1, 200674_s_at
FIG. 52: PRO80959
FIG. 53: DNA304669, NP_002119.1, 200679_x_at
FIG. 54: PRO71096
FIG. 55A-B: DNA344247, 7684654.2, 200690_at
FIG. 56: PRO94994
FIG. 57: DNA344248, NP_004125.3, 200691_s_at
FIG. 58: PRO94995
FIG. 59: DNA344249, NM_004134, 200692_s_at
FIG. 60: PRO94996
FIG. 61: DNA324897, NP_006845.1, 200700_s_at
FIG. 62: PRO12468
FIG. 63: DNA328375, NP_002071.1, 200708_at
FIG. 64: PRO80880
FIG. 65: DNA327114, NP_006004.1, 200725_x_at
FIG. 66: PRO62466
FIG. 67: DNA323943, NP_001021.1, 200741_s_at
FIG. 68: PRO80676
FIG. 69: DNA344250, NP_000382.3, 200742_s_at
FIG. 70: PRO94997
FIG. 71: DNA304659, NP_002023.1, 200748_s_at
FIG. 72: PRO71086
FIG. 73: DNA344251, 7762050.6, 200749_at
FIG. 74: PRO94998
FIG. 75: DNA287207, NP_006316.1, 200750_s_at
FIG. 76: PRO39268
FIG. 77A-B: DNA344252, NP_001377.1, 200762_at
FIG. 78: PRO62709
FIG. 79: DNA225584, NP_001145.1, 200782_at
FIG. 80: PRO36047
FIG. 81: DNA226262, NP_005554.1, 200783_s_at
FIG. 82: PRO36725
FIG. 83: DNA324060, NP_002530.1, 200790_at
FIG. 84: PRO80773
FIG. 85: DNA287211, NP_002147.1, 200806_s_at
FIG. 86: PRO69492
FIG. 87: DNA287211, NM_002156, 200807_s_at
FIG. 88: PRO69492
FIG. 89: DNA325222, NM_000976, 200809_x_at
FIG. 90: PRO62236
FIG. 91: DNA269874, NP_001271.1, 200810_s_at
FIG. 92: PRO58272
FIG. 93: DNA269874, NM_001280, 200811_at
FIG. 94: PRO58272
FIG. 95: DNA227795, NP_006420.1, 200812_at
FIG. 96: PRO38258
FIG. 97: DNA189687, NP_000843.1, 200824_at
FIG. 98: PRO25845
FIG. 99A-B: DNA255281, NP_006380.1,
200825_s_at
FIG. 100: PRO50357
FIG. 101: DNA88165, M14221, 200838_at
FIG. 102: PRO2678
FIG. 103: DNA196817, L16510, 200839_s_at
FIG. 104: PRO3344
FIG. 105: DNA326615, NP_000971.1, 200869_at
FIG. 106: PRO82971
FIG. 107: DNA226112, NP_002769.1, 200871_s_at
FIG. 108: PRO36575
FIG. 109: DNA254537, NP_002957.1, 200872_at
FIG. 110: PRO49642
FIG. 111: DNA254572, NP_006576.1, 200873_s_at
FIG. 112: PRO49675
FIG. 113: DNA271030, NP_006383.1, 200875_s_at
FIG. 114: PRO59358
FIG. 115: DNA324107, NP_006421.1, 200877_at
FIG. 116: PRO80814
FIG. 117: DNA328379, BC015869, 200878_at
FIG. 118: PRO84234
FIG. 119: DNA329099, 1164406.9, 200880_at
FIG. 120: PRO60127
FIG. 121: DNA271847, NP_001530.1, 200881_s_at
FIG. 122: PRO60127
FIG. 123: DNA226124, NP_003135.1, 200890_s_at
FIG. 124: PRO36587
FIG. 125: DNA325584, NP_002005.1, 200894_s_at
FIG. 126: PRO59262
FIG. 127: DNA325584, NM_002014, 200895_s_at
FIG. 128: PRO59262
FIG. 129: DNA272961, NP_004485.1, 200896_x_at
FIG. 130: PRO61041
FIG. 131A-B: DNA329018, NP_057165.2,
200897_s_at
FIG. 132: PRO84693
FIG. 133: DNA328380, X64879, 200904_at
FIG. 134A-B: DNA329018, NM_016081,
200907_s_at
FIG. 135: PRO84693
FIG. 136: DNA304665, NP_000995.1, 200909_s_at
FIG. 137: PRO71092
FIG. 138: DNA272974, NP_005989.1, 200910_at
FIG. 139: PRO61054
FIG. 140: DNA272695, NP_001722.1, 200920_s_at
FIG. 141: PRO60817
FIG. 142: DNA272695, NM_001731, 200921_s_at
FIG. 143: PRO60817
FIG. 144A-B: DNA270430, NP_054706.1,
200931_s_at
FIG. 145: PRO58810
FIG. 146: DNA325153, NP_150644.1, 200936_at
FIG. 147: PRO22907
FIG. 148: DNA329925, NP_001528.1, 200942_s_at
FIG. 149: PRO85239
FIG. 150A-B: DNA287217, NP_001750.1,
200951_s_at
FIG. 151: PRO36766
FIG. 152A-B: DNA287217, NM_001759,
200952_s_at
FIG. 153: PRO36766
FIG. 154A-B: DNA226303, D13639, 200953_s_at
FIG. 155: PRO36766
FIG. 156: DNA324149, NP_000984.1, 200963_x_at
FIG. 157: PRO11197
FIG. 158A-C: DNA344253, NP_002304.2,
200965_s_at
FIG. 159: PRO94999
FIG. 160: DNA344254, AL137335, 200992_at
FIG. 161: DNA325778, NP_006816.2, 200998_s_at
FIG. 162: PRO82248
FIG. 163: DNA325778, NM_006825, 200999_s_at
FIG. 164: PRO82248
FIG. 165: DNA275408, NP_001596.1, 201000_at
FIG. 166: PRO63068
FIG. 167: DNA328387, NP_001760.1, 201005_at
FIG. 168: PRO4769
FIG. 169: DNA304713, NP_006463.2, 201008_s_at
FIG. 170: PRO71139
FIG. 171: DNA304713, NM_006472, 201009_s_at
FIG. 172: PRO71139
FIG. 173: DNA304713, S73591, 201010_s_at
FIG. 174: PRO71139
FIG. 175: DNA89242, NP_000691.1, 201012_at
FIG. 176: PRO2907
FIG. 177: DNA328388, NP_006443.1, 201014_s_at
FIG. 178: PRO84240
FIG. 179A-B: DNA344255, 1327792.5, 201016_at
FIG. 180: PRO95001
FIG. 181: DNA328389, NP_006861.1, 201022_s_at
FIG. 182: PRO84241
FIG. 183: DNA344256, NP_005633.2, 201023_at
FIG. 184: PRO95002
FIG. 185A-B: DNA329101, NP_056988.2,
201024_x_at
FIG. 186: PRO84751
FIG. 187: DNA196628, NP_005318.1, 201036_s_at
FIG. 188: PRO25105
FIG. 189: DNA328391, NP_004408.1, 201041_s_at
FIG. 190: PRO84242
FIG. 191: DNA344257, NP_006296.1, 201043_s_at
FIG. 192: PRO95003
FIG. 193: DNA103208, NP_004090.3, 201061_s_at
FIG. 194: PRO4538
FIG. 195: DNA344258, NP_003810.1, 201064_s_at
FIG. 196: PRO62717
FIG. 197: DNA344259, NP_001907.2, 201066_at
FIG. 198: PRO95004
FIG. 199: DNA151675, NP_004791.1, 201078_at
FIG. 200: PRO11975
FIG. 201: DNA274743, NP_002850.1, 201087_at
FIG. 202: PRO62517
FIG. 203: DNA254725, NP_002257.1, 201088_at
FIG. 204: PRO49824
FIG. 205: DNA304719, NP_002296.1, 201105_at
FIG. 206: PRO71145
FIG. 207: DNA344260, NP_003312.2, 201113_at
FIG. 208: PRO95005
FIG. 209: DNA326273, NP_001961.1, 201123_s_at
FIG. 210: PRO82678
FIG. 211: DNA271185, NP_002397.1, 201126_s_at
FIG. 212: PRO59502
FIG. 213: DNA344261, NP_062543.1, 201132_at
FIG. 214: PRO95006
FIG. 215A-B: DNA227128, NP_055634.1,
201133_s_at
FIG. 216: PRO37591
FIG. 217: DNA329104, NP_004085.1, 201144_s_at
FIG. 218: PRO69550
FIG. 219: DNA344262, NP_000959.2, 201154_x_at
FIG. 220: PRO95007
FIG. 221A-B: DNA326365, NP_066565.1, 201158_at
FIG. 222: PRO82761
FIG. 223: DNA334099, NP_003642.2, 201161_s_at
FIG. 224: PRO85244
FIG. 225: DNA151802, NP_003661.1, 201169_s_at
FIG. 226: PRO12890
FIG. 227: DNA151802, NM_003670, 201170_s_at
FIG. 228: PRO12890
FIG. 229: DNA329091, NP_003936.1, 201171_at
FIG. 230: PRO11997
FIG. 231: DNA323783, NP_006591.1, 201173_x_at
FIG. 232: PRO80535
FIG. 233A-B: DNA344263, NP_003477.2,
201195_s_at
FIG. 234: PRO49192
FIG. 235: DNA328400, NP_003842.1, 201200_at
FIG. 236: PRO1409
FIG. 237: DNA103488, NP_002583.1, 201202_at
FIG. 238: PRO4815
FIG. 239: DNA344264, NP_005023.2, 201215_at
FIG. 240: PRO83378
FIG. 241: DNA326974, NP_000958.1, 201217_x_at
FIG. 242: PRO83285
FIG. 243: DNA327544, NP_002865.1, 201222_s_at
FIG. 244: PRO70357
FIG. 245: DNA344265, NP_006754.1, 201235_s_at
FIG. 246: PRO80725
FIG. 247: DNA275049, NP_004930.1, 201241_at
FIG. 248: PRO62770
FIG. 249: DNA226615, NP_001668.1, 201242_s_at
FIG. 250: PRO37078
FIG. 251: DNA226615, NM_001677, 201243_s_at
FIG. 252: PRO37078
FIG. 253: DNA287331, NP_002645.1, 201251_at
FIG. 254: PRO69595
FIG. 255: DNA324525, NP_000997.1, 201257_x_at
FIG. 256: PRO81179
FIG. 257: DNA227416, NP_006745.1, 201259_s_at
FIG. 258: PRO37879
FIG. 259: DNA227416, NM_006754, 201260_s_at
FIG. 260: PRO37879
FIG. 261: DNA270950, NP_003182.1, 201263_at
FIG. 262: PRO59281
FIG. 263: DNA97290, NP_002503.1, 201268_at
FIG. 264: PRO3637
FIG. 265: DNA344266, AF267863, 201276_at
FIG. 266: PRO95008
FIG. 267: DNA328405, NP_112556.1, 201277_s_at
FIG. 268: PRO84252
FIG. 269: DNA331290, NP_038474.1, 201285_at
FIG. 270: PRO86391
FIG. 271: DNA270526, NP_001166.1, 201288_at
FIG. 272: PRO58903
FIG. 273A-B: DNA327545, NP_001058.2,
201291_s_at
FIG. 274: PRO82731
FIG. 275A-B: DNA327545, NM_001067, 201292_at
FIG. 276: PRO82731
FIG. 277A-B: DNA344267, NM_134264,
201294_s_at
FIG. 278: PRO95009
FIG. 279A-B: DNA226778, AL110269, 201295_s_at
FIG. 280: PRO37241
FIG. 281: DNA333423, NP_001144.1, 201301_s_at
FIG. 282: PRO61325
FIG. 283: DNA333423, NM_001153, 201302_at
FIG. 284: PRO61325
FIG. 285: DNA329106, NP_003013.1, 201311_s_at
FIG. 286: PRO83360
FIG. 287: DNA329106, NM_003022, 201312_s_at
FIG. 288: PRO83360
FIG. 289: DNA255078, NP_006426.1, 201315_x_at
FIG. 290: PRO50165
FIG. 291: DNA274745, NP_006815.1, 201323_at
FIG. 292: PRO62518
FIG. 293: DNA150781, NP_001414.1, 201324_at
FIG. 294: PRO12467
FIG. 295: DNA150781, NM_001423, 201325_s_at
FIG. 296: PRO12467
FIG. 297: DNA329002, NP_001753.1, 201326_at
FIG. 298: PRO4912
FIG. 299: DNA329002, NM_001762, 201327_s_at
FIG. 300: PRO4912
FIG. 301A-C: DNA271656, NP_056128.1,
201334_s_at
FIG. 302: PRO59943
FIG. 303: DNA329107, NP_008818.3, 201367_s_at
FIG. 304: PRO84754
FIG. 305A-B: DNA329108, 1383643.16, 201368_at
FIG. 306: PRO84755
FIG. 307: DNA329107, NM_006887, 201369_s_at
FIG. 308: PRO84754
FIG. 309: DNA329218, NP_055227.1, 201381_x_at
FIG. 310: PRO84829
FIG. 311: DNA344268, NP_002800.2, 201388_at
FIG. 312: PRO63269
FIG. 313: DNA326116, NP_057376.1, 201391_at
FIG. 314: PRO82542
FIG. 315: DNA331447, NP_006614.2, 201397_at
FIG. 316: PRO85247
FIG. 317: DNA328410, NP_004519.1, 201403_s_at
FIG. 318: PRO60174
FIG. 319: DNA327072, NP_066357.1, 201406_at
FIG. 320: PRO10723
FIG. 321: DNA344269, NP_077007.1, 201420_s_at
FIG. 322: PRO95010
FIG. 323: DNA272286, NP_001743.1, 201432_at
FIG. 324: PRO60544
FIG. 325A-C: DNA88140, NP_004360.1, 201438_at
FIG. 326: PRO2670
FIG. 327: DNA344270, NP_071505.1, 201450_s_at
FIG. 328: PRO95011
FIG. 329: DNA326736, NP_006657.1, 201459_at
FIG. 330: PRO83076
FIG. 331: DNA226359, NP_002219.1, 201464_x_at
FIG. 332: PRO36822
FIG. 333: DNA226359, NM_002228, 201466_s_at
FIG. 334: PRO36822
FIG. 335: DNA328414, NP_003891.1, 201471_s_at
FIG. 336: PRO81346
FIG. 337: DNA103320, NP_002220.1, 201473_at
FIG. 338: PRO4650
FIG. 339: DNA325704, NP_004981.2, 201475_x_at
FIG. 340: PRO82188
FIG. 341: DNA327551, NP_001024.1, 201476_s_at
FIG. 342: PRO59289
FIG. 343: DNA327551, NM_001033, 201477_s_at
FIG. 344: PRO59289
FIG. 345: DNA254783, NP_001354.1, 201478_s_at
FIG. 346: PRO49881
FIG. 347: DNA254783, NM_001363, 201479_at
FIG. 348: PRO49881
FIG. 349: DNA329940, NP_001805.1, 201487_at
FIG. 350: PRO2679
FIG. 351: DNA304459, NP_005720.1, 201489_at
FIG. 352: PRO37073
FIG. 353: DNA304459, NM_005729, 201490_s_at
FIG. 354: PRO37073
FIG. 355: DNA325920, NP_036243.1, 201491_at
FIG. 356: PRO82373
FIG. 357: DNA253807, NP_065390.1, 201502_s_at
FIG. 358: PRO49210
FIG. 359: DNA329941, NP_001543.1, 201508_at
FIG. 360: PRO85249
FIG. 361: DNA323741, NP_003123.1, 201516_at
FIG. 362: PRO80498
FIG. 363: DNA344271, NP_073719.1, 201522_x_at
FIG. 364: PRO62659
FIG. 365: DNA328418, NP_003398.1, 201531_at
FIG. 366: PRO84261
FIG. 367: DNA329943, NP_009037.1, 201534_s_at
FIG. 368: PRO85251
FIG. 369: DNA329943, NM_007106, 201535_at
FIG. 370: PRO85251
FIG. 371: DNA329553, NP_064535.1, 201543_s_at
FIG. 372: PRO38313
FIG. 373: DNA344272, NP_004121.2, 201554_x_at
FIG. 374: PRO95012
FIG. 375: DNA272171, NP_002379.2, 201555_at
FIG. 376: PRO60438
FIG. 377: DNA226291, NP_055047.1, 201557_at
FIG. 378: PRO36754
FIG. 379A-B: DNA290226, NP_039234.1,
201559_s_at
FIG. 380: PRO70317
FIG. 381A-B: DNA290226, NM_013943, 201560_at
FIG. 382: PRO70317
FIG. 383: DNA227478, NP_002157.1, 201565_s_at
FIG. 384: PRO37941
FIG. 385: DNA150986, D13891, 201566_x_at
FIG. 386: PRO0
FIG. 387: DNA344273, M75715, 201573_s_at
FIG. 388: PRO95013
FIG. 389A-B: DNA270995, NP_004721.1, 201574_at
FIG. 390: PRO59324
FIG. 391: DNA227071, NP_000260.1, 201577_at
FIG. 392: PRO37534
FIG. 393A-B: DNA329944, AB032988, 201581_at
FIG. 394: DNA227013, NP_001560.1, 201587_s_at
FIG. 395: PRO37476
FIG. 396: DNA150990, NP_003632.1, 201601_x_at
FIG. 397: PRO12570
FIG. 398: DNA290280, NP_004359.1, 201605_x_at
FIG. 399: PRO70425
FIG. 400: DNA329947, NP_536806.1, 201613_s_at
FIG. 401: PRO37674
FIG. 402: DNA188207, NM_005380, 201621_at
FIG. 403: PRO21719
FIG. 404: DNA329114, NP_001340.1, 201623_s_at
FIG. 405: PRO84759
FIG. 406: DNA329114, NM_001349, 201624_at
FIG. 407: PRO84759
FIG. 408: DNA344274, 7698185.18, 201626_at
FIG. 409: PRO95014
FIG. 410A-D: DNA344275, U96876, 201627_s_at
FIG. 411: DNA344276, NM_004300, 201629_s_at
FIG. 412: PRO89350
FIG. 413: DNA329115, NP_434702.1, 201631_s_at
FIG. 414: PRO84760
FIG. 415: DNA326193, NP_085056.1, 201634_s_at
FIG. 416: PRO82609
FIG. 417: DNA287240, NP_004326.1, 201641_at
FIG. 418: PRO29371
FIG. 419: DNA88410, NP_005525.1, 201642_at
FIG. 420: PRO2778
FIG. 421A-B: DNA220748, NP_000201.1, 201656_at
FIG. 422: PRO34726
FIG. 423: DNA328423, NP_003245.1, 201666_at
FIG. 424: PRO2121
FIG. 425: DNA344277, NP_683877.1, 201676_x_at
FIG. 426: PRO81959
FIG. 427: DNA324742, NP_001751.1, 201700_at
FIG. 428: PRO81367
FIG. 429: DNA270883, NP_001061.1, 201714_at
FIG. 430: PRO59218
FIG. 431A-B: DNA151806, NP_001422.1,
201718_s_at
FIG. 432: PRO12768
FIG. 433A-B: DNA151806, NM_001431,
201719_s_at
FIG. 434: PRO12768
FIG. 435: DNA273759, NP_006014.1, 201725_at
FIG. 436: PRO61721
FIG. 437: DNA344278, NP_005618.2, 201739_at
FIG. 438: PRO86741
FIG. 439: DNA326373, NP_008855.1, 201742_x_at
FIG. 440: PRO82769
FIG. 441A-B: DNA344279, 345309.13, 201749_at
FIG. 442: PRO95015
FIG. 443: DNA287167, NP_006627.1, 201761_at
FIG. 444: PRO59136
FIG. 445A-B: DNA150444, NP_055589.1,
201778_s_at
FIG. 446: PRO12253
FIG. 447A-B: DNA103387, NP_002287.1, 201795_at
FIG. 448: PRO4716
FIG. 449A-B: DNA272263, NP_006286.1,
201797_s_at
FIG. 450: PRO70138
FIG. 451: DNA151017, NP_004835.1, 201810_s_at
FIG. 452: PRO12841
FIG. 453: DNA151017, NM_004844, 201811_x_at
FIG. 454: PRO12841
FIG. 455: DNA324015, NP_006326.1, 201821_s_at
FIG. 456: PRO80735
FIG. 457: DNA329952, NP_005854.2, 201830_s_at
FIG. 458: PRO85256
FIG. 459: DNA304710, NP_001531.1, 201841_s_at
FIG. 460: PRO71136
FIG. 461: DNA88450, NP_000226.1, 201847_at
FIG. 462: PRO2795
FIG. 463: DNA254350, NP_004043.2, 201849_at
FIG. 464: PRO49461
FIG. 465: DNA150725, NP_001738.1, 201850_at
FIG. 466: PRO12792
FIG. 467: DNA329118, NP_068660.1, 201853_s_at
FIG. 468: PRO83123
FIG. 469A-B: DNA103553, NP_000167.1,
201865_x_at
FIG. 470: PRO4880
FIG. 471: DNA272066, NP_002931.1, 201872_s_at
FIG. 472: PRO60337
FIG. 473A-B: DNA331295, NP_002710.1,
201877_s_at
FIG. 474: PRO86394
FIG. 475: DNA150805, NP_055703.1, 201889_at
FIG. 476: PRO11583
FIG. 477: DNA344280, BC028932, 201890_at
FIG. 478: DNA329956, NP_000875.1, 201892_s_at
FIG. 479: PRO85260
FIG. 480: DNA328431, NP_001817.1, 201897_s_at
FIG. 481: PRO45093
FIG. 482: DNA324310, NP_003356.1, 201903_at
FIG. 483: PRO80988
FIG. 484: DNA305191, NP_000999.1, 201909_at
FIG. 485: PRO71295
FIG. 486: DNA275385, NP_002085.1, 201912_s_at
FIG. 487: PRO63048
FIG. 488: DNA254978, NP_060625.1, 201917_s_at
FIG. 489: PRO50067
FIG. 490: DNA103328, NP_005406.2, 201920_at
FIG. 491: PRO4658
FIG. 492: DNA329057, NP_004116.2, 201921_at
FIG. 493: PRO84719
FIG. 494: DNA227112, NP_006397.1, 201923_at
FIG. 495: PRO37575
FIG. 496: DNA83046, NP_000565.1, 201925_s_at
FIG. 497: PRO2569
FIG. 498: DNA83046, NM_000574, 201926_s_at
FIG. 499: PRO2569
FIG. 500A-B: DNA344281, NP_005906.2, 201930_at
FIG. 501: PRO62927
FIG. 502: DNA329119, NP_004633.1, 201938_at
FIG. 503: PRO4550
FIG. 504A-B: DNA329120, NP_002560.1, 201945_at
FIG. 505: PRO2752
FIG. 506: DNA274167, NP_0006422.1, 201946_s_at
FIG. 507: PRO62097
FIG. 508: DNA274167, NM_006431, 201947_s_at
FIG. 509: PRO62097
FIG. 510A-B: DNA327563, NP_066945.1, 201963_at
FIG. 511: PRO83592
FIG. 512: DNA344282, NP_002624.2, 201968_s_at
FIG. 513: PRO95016
FIG. 514: DNA344283, NP_751896.1, 201970_s_at
FIG. 515: PRO95017
FIG. 516: DNA344284, NP_002393.1, 202016_at
FIG. 517: PRO95018
FIG. 518: DNA328437, NP_005792.1, 202021_x_at
FIG. 519: PRO84271
FIG. 520: DNA300776, NP_000990.1, 202029_x_at
FIG. 521: PRO70900
FIG. 522: DNA344285, NP_005521.1, 202069_s_at
FIG. 523: PRO83596
FIG. 524: DNA226116, NP_002990.1, 202071_at
FIG. 525: PRO36579
FIG. 526: DNA344286, AF070533, 202073_at
FIG. 527: PRO95019
FIG. 528: DNA289522, NP_004994.1, 202077_at
FIG. 529: PRO70276
FIG. 530A-B: DNA270923, NP_004808.1, 202085_at
FIG. 531: PRO59256
FIG. 532: DNA327568, NP_002453.1, 202086_at
FIG. 533: PRO57922
FIG. 534: DNA271404, NP_001542.1, 202105_at
FIG. 535: PRO59703
FIG. 536: DNA328440, NP_004517.1, 202107_s_at
FIG. 537: PRO84274
FIG. 538: DNA344287, NP_003822.2, 202129_s_at
FIG. 539: PRO95020
FIG. 540: DNA324895, NP_006294.2, 202138_x_at
FIG. 541: PRO81501
FIG. 542A-B: DNA304479, NP_057124.2, 202194_at
FIG. 543: PRO733
FIG. 544: DNA329121, NP_079471.1, 202241_at
FIG. 545: PRO84763
FIG. 546: DNA325711, NP_000066.1, 202246_s_at
FIG. 547: PRO4873
FIG. 548: DNA294794, NP_002861.1, 202252_at
FIG. 549: PRO70754
FIG. 550: DNA256533, NP_006105.1, 202264_s_at
FIG. 551: PRO51565
FIG. 552: DNA150808, NP_002044.1, 202269_x_at
FIG. 553: PRO12478
FIG. 554: DNA150808, NM_002053, 202270_at
FIG. 555: PRO12478
FIG. 556: DNA304716, NP_510867.1, 202284_s_at
FIG. 557: PRO71142
FIG. 558: DNA328274, NP_055706.1, 202290_at
FIG. 559: PRO12912
FIG. 560: DNA331450, NP_004381.2, 202295_s_at
FIG. 561: PRO2682
FIG. 562: DNA344288, NP_000584.2, 202307_s_at
FIG. 563: PRO36996
FIG. 564A-B: DNA329970, NP_000910.2,
202336_s_at
FIG. 565: PRO85272
FIG. 566: DNA325115, NP_001435.1, 202345_s_at
FIG. 567: PRO81689
FIG. 568: DNA344289, NP_002807.1, 202352_s_at
FIG. 569: PRO58880
FIG. 570A-B: DNA254188, NP_004913.1, 202361_at
FIG. 571: PRO49300
FIG. 572: DNA331297, NP_005953.2, 202364_at
FIG. 573: PRO86396
FIG. 574A-B: DNA227353, NP_055637.1, 202375_at
FIG. 575: PRO37816
FIG. 576: DNA344290, 1096863.3, 202377_at
FIG. 577: PRO95021
FIG. 578: DNA103246, NP_059996.1, 202378_s_at
FIG. 579: PRO4576
FIG. 580: DNA328449, NP_005462.1, 202382_s_at
FIG. 581: PRO60304
FIG. 582: DNA150514, NP_065203.1, 202418_at
FIG. 583: PRO12304
FIG. 584A-C: DNA270933, NP_006757.1, 202423_at
FIG. 585: PRO59265
FIG. 586A-B: DNA335104, NP_000935.1,
202429_s_at
FIG. 587: PRO49644
FIG. 588: DNA227121, NP_066928.1, 202430_s_at
FIG. 589: PRO37584
FIG. 590: DNA66487, NP_002458.1, 202431_s_at
FIG. 591: PRO1213
FIG. 592A-B: DNA327576, NP_000095.1,
202435_s_at
FIG. 593: PRO83600
FIG. 594A-B: DNA327576, NM_000104,
202436_s_at
FIG. 595: PRO83600
FIG. 596A-D: DNA270871, U56438, 202437_s_at
FIG. 597A-B: DNA344291, 7685287.117,
202438_x_at
FIG. 598: PRO2328
FIG. 599A-B: DNA335104, NM_000944,
202457_s_at
FIG. 600: PRO49644
FIG. 601A-B: DNA329973, NP_055461.1,
202459_s_at
FIG. 602: PRO82824
FIG. 603A-B: DNA269642, NP_004557.1,
202464_s_at
FIG. 604: PRO58054
FIG. 605: DNA227921, NP_003789.1, 202468_s_at
FIG. 606: PRO38384
FIG. 607A-B: DNA329122, NP_067675.1, 202478_at
FIG. 608: PRO84764
FIG. 609A-B: DNA329122, NM_021643,
202479_s_at
FIG. 610: PRO84764
FIG. 611: DNA329123, NP_002873.1, 202483_s_at
FIG. 612: PRO84765
FIG. 613: DNA344292, NP_003918.1, 202484_s_at
FIG. 614: PRO95022
FIG. 615: DNA324925, NP_036544.1, 202487_s_at
FIG. 616: PRO61812
FIG. 617A-B: DNA103449, NP_008862.1,
202498_s_at
FIG. 618: PRO4776
FIG. 619: DNA328451, NP_000007.1, 202502_at
FIG. 620: PRO62139
FIG. 621: DNA234442, NP_055551.1, 202503_s_at
FIG. 622: PRO38852
FIG. 623A-B: DNA277809, NP_055582.1,
202523_s_at
FIG. 624: PRO64556
FIG. 625A-B: DNA277809, NM_014767,
202524_s_at
FIG. 626: PRO64556
FIG. 627A-B: DNA226870, NM_000791,
202534_x_at
FIG. 628: PRO37333
FIG. 629: DNA328453, NP_003752.2, 202546_at
FIG. 630: PRO84281
FIG. 631A-B: DNA344293, NP_008879.2, 202557_at
FIG. 632: PRO95023
FIG. 633: DNA344294, NP_004166.1, 202567_at
FIG. 634: PRO83257
FIG. 635: DNA325587, NP_068772.1, 202580_x_at
FIG. 636: PRO82083
FIG. 637: DNA329979, NP_001062.1, 202589_at
FIG. 638: PRO82821
FIG. 639: DNA326078, NP_057725.1, 202593_s_at
FIG. 640: PRO38464
FIG. 641: DNA329125, NP_056159.1, 202594_at
FIG. 642: PRO84767
FIG. 643: DNA329125, NM_015344, 202595_s_at
FIG. 644: PRO84767
FIG. 645: DNA274881, NP_001896.1, 202613_at
FIG. 646: PRO62626
FIG. 647A-B: DNA329980, 1134366.16, 202615_at
FIG. 648: PRO85278
FIG. 649A-C: DNA344295, NP_036427.1,
202624_s_at
FIG. 650: PRO95024
FIG. 651A-B: DNA344296, 441144.12, 202625_at
FIG. 652: PRO95025
FIG. 653: DNA103245, NP_002341.1, 202626_s_at
FIG. 654: PRO4575
FIG. 655: DNA329126, NP_005025.1, 202635_s_at
FIG. 656: PRO84768
FIG. 657: DNA59763, NP_000192.1, 202638_s_at
FIG. 658: PRO160
FIG. 659: DNA289528, NP_004302.1, 202641_at
FIG. 660: PRO70286
FIG. 661A-B: DNA344297, NP_006281.1,
202643_s_at
FIG. 662: PRO12904
FIG. 663A-B: DNA344298, NM_006290,
202644_s_at
FIG. 664: PRO12904
FIG. 665: DNA254129, NP_006001.1, 202655_at
FIG. 666: PRO49244
FIG. 667A-B: DNA333747, 099914.40, 202663_at
FIG. 668: PRO88372
FIG. 669: DNA344299, NP_001665.1, 202672_s_at
FIG. 670: PRO95026
FIG. 671: DNA272801, NP_004483.1, 202678_at
FIG. 672: PRO60906
FIG. 673: DNA335588, NP_003801.1, 202687_s_at
FIG. 674: PRO1096
FIG. 675: DNA335588, NM_003810, 202688_at
FIG. 676: PRO1096
FIG. 677: DNA344300, NP_008869.1, 202690_s_at
FIG. 678: PRO41946
FIG. 679A-B: DNA150467, NP_055513.1,
202699_s_at
FIG. 680: PRO12272
FIG. 681: DNA330776, NP_005740.1, 202704_at
FIG. 682: PRO58014
FIG. 683: DNA326000, NP_004692.1, 202705_at
FIG. 684: PRO82442
FIG. 685A-B: DNA328459, NP_004332.2, 202715_at
FIG. 686: PRO84285
FIG. 687A-B: DNA270254, NP_002006.2,
202724_s_at
FIG. 688: PRO58642
FIG. 689: DNA331298, NP_055271.2, 202730_s_at
FIG. 690: PRO81909
FIG. 691: DNA344301, NM_145341, 202731_at
FIG. 692: PRO95027
FIG. 693A-B: DNA344302, BC035058, 202741_at
FIG. 694: PRO95028
FIG. 695: DNA271973, NP_002722.1, 202742_s_at
FIG. 696: PRO60248
FIG. 697: DNA344303, BC040437, 202746_at
FIG. 698: PRO1189
FIG. 699: DNA327192, NP_004858.1, 202747_s_at
FIG. 700: PRO1189
FIG. 701: DNA227164, Y12478, 202749_at
FIG. 702: PRO37627
FIG. 703A-C: DNA329129, NP_009134.1,
202759_s_at
FIG. 704: PRO84288
FIG. 705A-B: DNA344304, NM_147150,
202760_s_at
FIG. 706: PRO95029
FIG. 707A-B: DNA256782, AL080133, 202761_s_at
FIG. 708: PRO51715
FIG. 709A-B: DNA328464, 977954.20, 202769_at
FIG. 710: PRO84290
FIG. 711: DNA226578, NP_004345.1, 202770_s_at
FIG. 712: PRO37041
FIG. 713: DNA273346, NP_055316.1, 202779_s_at
FIG. 714: PRO61349
FIG. 715: DNA275337, NP_037365.1, 202786_at
FIG. 716: PRO63011
FIG. 717: DNA344305, 345245.28, 202789_at
FIG. 718: PRO95030
FIG. 719: DNA329986, NP_006454.1, 202811_at
FIG. 720: PRO61895
FIG. 721: DNA328465, NP_005639.1, 202824_s_at
FIG. 722: PRO84291
FIG. 723: DNA269828, NP_006691.1, 202837_at
FIG. 724: PRO58230
FIG. 725: DNA329988, NP_036460.1, 202842_s_at
FIG. 726: PRO1471
FIG. 727: DNA329988, NM_012328, 202843_at
FIG. 728: PRO1471
FIG. 729: DNA328466, NP_004554.1, 202847_at
FIG. 730: PRO84292
FIG. 731: DNA227063, NP_002849.1, 202850_at
FIG. 732: PRO37526
FIG. 733: DNA103394, NP_004198.1, 202855_s_at
FIG. 734: PRO4722
FIG. 735: DNA103394, NM_004207, 202856_s_at
FIG. 736: PRO4722
FIG. 737: DNA344306, NP_000575.1, 202859_x_at
FIG. 738: PRO74
FIG. 739: DNA275144, NP_000128.1, 202862_at
FIG. 740: PRO62852
FIG. 741: DNA328467, NP_003104.2, 202864_s_at
FIG. 742: PRO84293
FIG. 743: DNA287289, NP_058132.1, 202869_at
FIG. 744: PRO69559
FIG. 745: DNA273060, NP_001246.1, 202870_s_at
FIG. 746: PRO61125
FIG. 747: DNA325334, NP_061931.1, 202887_s_at
FIG. 748: PRO81877
FIG. 749A-B: DNA333705, NP_004070.3,
202901_x_at
FIG. 750: PRO88334
FIG. 751A-B: DNA333705, NM_004079,
202902_s_at
FIG. 752: PRO88334
FIG. 753: DNA332688, NP_510966.1, 202910_s_at
FIG. 754: PRO2030
FIG. 755A-B: DNA275066, NP_000170.1, 202911_at
FIG. 756: PRO62786
FIG. 757: DNA83008, NP_001115.1, 202912_at
FIG. 758: PRO2032
FIG. 759A-B: DNA344307, 7762119.3, 202934_at
FIG. 760: PRO95031
FIG. 761: DNA344308, NP_056518.2, 202937_x_at
FIG. 762: PRO95032
FIG. 763: DNA304681, NP_066552.1, 202941_at
FIG. 764: PRO71107
FIG. 765: DNA269481, NP_001976.1, 202942_at
FIG. 766: PRO57901
FIG. 767: DNA273320, NP_008950.1, 202954_at
FIG. 768: PRO61327
FIG. 769: DNA344309, X73427, 202988_s_at
FIG. 770: PRO95033
FIG. 771: DNA329136, NP_057475.1, 203023_at
FIG. 772: PRO84772
FIG. 773: DNA270174, NP_000092.1, 203028_s_at
FIG. 774: PRO58563
FIG. 775A-B: DNA83163, U66702, 203029_s_at
FIG. 776: PRO2611
FIG. 777A-B: DNA344310, NP_055566.1,
203037_s_at
FIG. 778: PRO95034
FIG. 779A-B: DNA344311, NP_002835.2, 203038_at
FIG. 780: PRO95035
FIG. 781A-B: DNA304464, NP_055733.1, 203044_at
FIG. 782: PRO71042
FIG. 783A-B: DNA328358, NP_005981.1, 203047_at
FIG. 784: PRO84218
FIG. 785A-B: DNA227821, NP_055666.1, 203068_at
FIG. 786: PRO38284
FIG. 787: DNA329137, NP_005892.1, 203077_s_at
FIG. 788: PRO12879
FIG. 789A-B: DNA339385, NP_055568.1, 203082_at
FIG. 790: PRO91190
FIG. 791: DNA344312, 1386457.26, 203086_at
FIG. 792: PRO95036
FIG. 793: DNA329138, NP_004511.1, 203087_s_at
FIG. 794: PRO84773
FIG. 795: DNA344313, AF026030, 203092_at
FIG. 796: PRO95037
FIG. 797A-B: DNA227949, NP_055062.1,
203096_s_at
FIG. 798: PRO38412
FIG. 799: DNA329992, NP_002399.1, 203102_s_at
FIG. 800: PRO59267
FIG. 801: DNA272867, NP_003960.1, 203109_at
FIG. 802: PRO60960
FIG. 803: DNA150430, NP_006387.1, 203114_at
FIG. 804: PRO12770
FIG. 805: DNA329994, NP_004707.2, 203118_at
FIG. 806: PRO85286
FIG. 807: DNA287417, NP_077003.1, 203119_at
FIG. 808: PRO69674
FIG. 809A-B: DNA226395, NP_000312.1, 203132_at
FIG. 810: PRO36858
FIG. 811A-B: DNA344314, NP_620309.1, 203140_at
FIG. 812: PRO12790
FIG. 813: DNA269433, NP_005877.1, 203163_at
FIG. 814: PRO57856
FIG. 815: DNA340116, NP_000146.2, 203179_at
FIG. 816: PRO91615
FIG. 817A-B: DNA331303, NP_003129.1,
203182_s_at
FIG. 818: PRO86399
FIG. 819: DNA304720, NP_062427.1, 203186_s_at
FIG. 820: PRO71146
FIG. 821A-B: DNA270861, NP_001371.1, 203187_at
FIG. 822: PRO59198
FIG. 823A-B: DNA344315, AAL56659.1,
203194_s_at
FIG. 824: PRO95038
FIG. 825: DNA329997, NP_031396.1, 203209_at
FIG. 826: PRO61115
FIG. 827A-B: DNA328481, NP_057240.1,
203211_s_at
FIG. 828: PRO84307
FIG. 829: DNA327588, 995529.4, 203213_at
FIG. 830: PRO83607
FIG. 831: DNA334914, NP_001777.1, 203214_x_at
FIG. 832: PRO58324
FIG. 833A-C: DNA274481, NP_000323.1,
203231_s_at
FIG. 834: PRO62384
FIG. 835A-C: DNA274481, NM_000332,
203232_s_at
FIG. 836: PRO62384
FIG. 837: DNA76514, NP_000409.1, 203233_at
FIG. 838: PRO2540
FIG. 839: DNA334781, NP_006448.1, 203242_s_at
FIG. 840: PRO89234
FIG. 841: DNA334781, NM_006457, 203243_s_at
FIG. 842: PRO89234
FIG. 843: DNA330000, NP_036277.1, 203270_at
FIG. 844: PRO85289
FIG. 845: DNA270963, NM_003335, 203281_s_at
FIG. 846: PRO59293
FIG. 847: DNA225675, NP_005561.1, 203293_s_at
FIG. 848: PRO36138
FIG. 849: DNA225675, NM_005570, 203294_s_at
FIG. 850: PRO36138
FIG. 851: DNA328489, NP_006511.1, 203303_at
FIG. 852: PRO84314
FIG. 853: DNA344316, NP_233796.1, 203313_s_at
FIG. 854: PRO95039
FIG. 855: DNA271740, NP_003085.1, 203316_s_at
FIG. 856: PRO60024
FIG. 857A-B: DNA330003, NP_005532.1,
203331_s_at
FIG. 858: PRO85291
FIG. 859A-B: DNA330003, NM_005541,
203332_s_at
FIG. 860: PRO85291
FIG. 861: DNA330004, NP_055785.2, 203333_at
FIG. 862: PRO85292
FIG. 863: DNA324514, NP_002349.1, 203362_s_at
FIG. 864: PRO81169
FIG. 865: DNA328493, NP_008957.1, 203367_at
FIG. 866: PRO84317
FIG. 867: DNA151022, NP_001336.1, 203385_at
FIG. 868: PRO12096
FIG. 869A-B: DNA344317, 232388.2, 203386_at
FIG. 870: PRO95040
FIG. 871A-B: DNA341155, NP_055647.1,
203387_s_at
FIG. 872: PRO91654
FIG. 873: DNA331200, NP_004304.1, 203388_at
FIG. 874: PRO86322
FIG. 875: DNA88324, M65128, 203391_at
FIG. 876: PRO2748
FIG. 877A-B: DNA254616, NP_004473.1,
203397_s_at
FIG. 878: PRO49718
FIG. 879: DNA270134, NP_000098.1, 203409_at
FIG. 880: PRO58523
FIG. 881: DNA344318, NP_733821.1, 203411_s_at
FIG. 882: PRO95041
FIG. 883: DNA28759, NP_006150.1, 203413_at
FIG. 884: PRO2520
FIG. 885A-B: DNA256807, NP_057339.1, 203420_at
FIG. 886: PRO51738
FIG. 887: DNA327808, NP_002961.1, 203455_s_at
FIG. 888: PRO83769
FIG. 889: DNA269591, NP_002655.1, 203471_s_at
FIG. 890: PRO58004
FIG. 891: DNA150959, NP_005813.1, 203498_at
FIG. 892: PRO11599
FIG. 893A-C: DNA331461, NP_005493.2,
203504_s_at
FIG. 894: PRO86511
FIG. 895A-C: DNA328498, AF285167, 203505_at
FIG. 896: PRO84320
FIG. 897A-B: DNA333708, NP_001057.1, 203508_at
FIG. 898: PRO21928
FIG. 899A-B: DNA331462, NP_003096.1, 203509_at
FIG. 900: PRO86512
FIG. 901: DNA344319, 474053.9, 203510_at
FIG. 902: PRO95042
FIG. 903A-C: DNA344320, BAB47469.2, 203513_at
FIG. 904: PRO95043
FIG. 905: DNA272911, NP_006545.1, 203517_at
FIG. 906: PRO60997
FIG. 907A-D: DNA333617, NP_000072.1,
203518_at
FIG. 908: PRO88260
FIG. 909A-B: DNA272399, NP_001197.1,
203542_s_at
FIG. 910: PRO60653
FIG. 911A-B: DNA272399, NM_001206,
203543_s_at
FIG. 912: PRO60653
FIG. 913: DNA344321, NP_003464.1, 203544_s_at
FIG. 914: PRO62698
FIG. 915: DNA324684, NP_004210.1, 203554_x_at
FIG. 916: PRO81319
FIG. 917A-B: DNA339392, NP_055758.1, 203556_at
FIG. 918: PRO91197
FIG. 919: DNA327594, NP_003869.1, 203560_at
FIG. 920: PRO83611
FIG. 921: DNA332919, NP_005094.1, 203562_at
FIG. 922: PRO60597
FIG. 923: DNA344322, NP_006346.1, 203567_s_at
FIG. 924: PRO85303
FIG. 925A-B: DNA340123, NP_003602.1,
203569_s_at
FIG. 926: PRO91622
FIG. 927: DNA329033, NP_005375.1, 203574_at
FIG. 928: PRO84700
FIG. 929: DNA344323, NP_054763.2, 203583_at
FIG. 930: PRO95044
FIG. 931A-B: DNA270323, NP_036552.1,
203595_s_at
FIG. 932: PRO58710
FIG. 933A-B: DNA344324, NP_733936.1, 203608_at
FIG. 934: PRO95045
FIG. 935: DNA344325, NM_006355, 203610_s_at
FIG. 936: PRO85303
FIG. 937: DNA287246, NP_004044.2, 203612_at
FIG. 938: PRO69521
FIG. 939: DNA344326, NP_002681.1, 203616_at
FIG. 940: PRO95046
FIG. 941: DNA330018, NP_064528.1, 203622_s_at
FIG. 942: PRO85304
FIG. 943A-B: DNA270264, DNA270264, 203633_at
FIG. 944A-B: DNA327597, NP_075261.1,
203639_s_at
FIG. 945: PRO83613
FIG. 946: DNA254642, NP_004100.1, 203646_at
FIG. 947: PRO49743
FIG. 948: DNA328507, NP_006395.1, 203650_at
FIG. 949: PRO4761
FIG. 950: DNA151752, NP_002124.1, 203665_at
FIG. 951: PRO12886
FIG. 952: DNA88352, NP_002067.1, 203676_at
FIG. 953: PRO2759
FIG. 954A-B: DNA227646, NP_000288.1, 203688_at
FIG. 955: PRO38109
FIG. 956A-B: DNA330021, NP_001940.1,
203692_s_at
FIG. 957: PRO85306
FIG. 958A-B: DNA330021, NM_001949,
203693_s_at
FIG. 959: PRO85306
FIG. 960A-B: DNA344327, NP_002591.1, 203708_at
FIG. 961: PRO10691
FIG. 962A-C: DNA331467, NP_002213.1, 203710_at
FIG. 963: PRO86516
FIG. 964: DNA329144, NM_014878, 203712_at
FIG. 965: PRO84779
FIG. 966: DNA324183, NP_001926.2, 203716_s_at
FIG. 967: PRO80881
FIG. 968: DNA330023, NP_001915.1, 203725_at
FIG. 969: PRO85308
FIG. 970A-B: DNA344328, NP_003613.1,
203736_s_at
FIG. 971: PRO95047
FIG. 972A-B: DNA325369, NP_055877.2,
203737_s_at
FIG. 973: PRO81905
FIG. 974: DNA344329, AL834427, 203738_at
FIG. 975A-B: DNA274324, NP_006517.1, 203739_at
FIG. 976: PRO62242
FIG. 977A-B: DNA150748, NP_001105.1,
203741_s_at
FIG. 978: PRO12446
FIG. 979: DNA344330, 197185.7, 203745_at
FIG. 980: PRO58198
FIG. 981A-B: DNA325972, NP_001202.3, 203755_at
FIG. 982: PRO82417
FIG. 983: DNA328509, NP_006739.1, 203761_at
FIG. 984: PRO57996
FIG. 985: DNA344331, NP_057092.1, 203762_s_at
FIG. 986: PRO95049
FIG. 987: DNA344332, NM_016008, 203763_at
FIG. 988: PRO95050
FIG. 989: DNA330025, NP_055565.2, 203764_at
FIG. 990: PRO85310
FIG. 991: DNA330027, NP_036578.1, 203787_at
FIG. 992: PRO85312
FIG. 993: DNA274125, NP_071739.1, 203830_at
FIG. 994: PRO62061
FIG. 995A-B: DNA331113, NP_005914.1,
203836_s_at
FIG. 996: PRO60244
FIG. 997A-B: DNA344333, U67156, 203837_at
FIG. 998: PRO60244
FIG. 999A-B: DNA344334, 435717.6, 203843_at
FIG. 1000: PRO95051
FIG. 1001A-B: DNA325529, NP_536739.1,
203853_s_at
FIG. 1002: PRO82037
FIG. 1003: DNA275339, NP_005685.1, 203880_at
FIG. 1004: PRO63012
FIG. 1005: DNA328513, NM_016283, 203893_at
FIG. 1006: PRO37815
FIG. 1007: DNA151820, NP_000851.1, 203914_x_at
FIG. 1008: PRO12194
FIG. 1009: DNA82376, NP_002407.1, 203915_at
FIG. 1010: PRO1723
FIG. 1011: DNA344335, NP_004258.2, 203921_at
FIG. 1012: PRO77044
FIG. 1013: DNA271676, NP_002052.1, 203925_at
FIG. 1014: PRO59961
FIG. 1015: DNA344336, NP_002940.2, 203931_s_at
FIG. 1016: PRO95052
FIG. 1017: DNA88035, NP_002517.1, 203939_at
FIG. 1018: PRO2135
FIG. 1019: DNA327606, NP_001163.1, 203945_at
FIG. 1020: PRO57873
FIG. 1021: DNA327606, NM_001172, 203946_s_at
FIG. 1022: PRO57873
FIG. 1023: DNA344337, NP_005186.2, 203973_s_at
FIG. 1024: PRO95053
FIG. 1025: DNA227239, NP_003497.1, 203987_at
FIG. 1026: PRO37702
FIG. 1027: DNA344338, NP_004471.1, 203988_s_at
FIG. 1028: PRO95054
FIG. 1029: DNA226133, NP_001983.1, 203989_x_at
FIG. 1030: PRO36596
FIG. 1031A-B: DNA333574, NP_002820.2,
203997_at
FIG. 1032: PRO88221
FIG. 1033A-B: DNA344339, BC010502,
204009_s_at
FIG. 1034: PRO95055
FIG. 1035: DNA328516, NP_005833.1, 204011_at
FIG. 1036: PRO12323
FIG. 1037: DNA344340, NP_001385.1, 204014_at
FIG. 1038: PRO49185
FIG. 1039: DNA329145, NM_057158, 204015_s_at
FIG. 1040: PRO84780
FIG. 1041: DNA330033, NP_056492.1, 204019_s_at
FIG. 1042: PRO85318
FIG. 1043: DNA328271, NP_008988.2, 204026_s_at
FIG. 1044: PRO81868
FIG. 1045: DNA344341, NP_055390.1, 204030_s_at
FIG. 1046: PRO95056
FIG. 1047: DNA344342, 7698646.3, 204057_at
FIG. 1048: PRO95057
FIG. 1049A-B: DNA336315, NP_005035.1,
204060_s_at
FIG. 1050: PRO90466
FIG. 1051: DNA226737, NP_004576.1, 204070_at
FIG. 1052: PRO37200
FIG. 1053A-C: DNA333515, NP_075463.1,
204072_s_at
FIG. 1054: PRO88167
FIG. 1055: DNA344343, NP_003586.1, 204079_at
FIG. 1056: PRO61375
FIG. 1057: DNA344344, NP_006186.1, 204082_at
FIG. 1058: PRO22518
FIG. 1059: DNA270476, NP_003591.1, 204092_s_at
FIG. 1060: PRO58855
FIG. 1061: DNA216689, NP_002975.1, 204103_at
FIG. 1062: PRO34276
FIG. 1063: DNA328522, NP_001769.2, 204118_at
FIG. 1064: PRO2696
FIG. 1065: DNA304489, NP_003495.1, 204126_s_at
FIG. 1066: PRO71058
FIG. 1067: DNA325824, NP_002906.1, 204128_s_at
FIG. 1068: PRO82290
FIG. 1069: DNA103333, NP_055705.1, 204135_at
FIG. 1070: PRO4663
FIG. 1071: DNA344345, NP_006470.1, 204146_at
FIG. 1072: PRO61659
FIG. 1073A-B: DNA344346, 7698815.10, 204156_at
FIG. 1074: PRO95058
FIG. 1075: DNA330040, NP_523240.1, 204159_at
FIG. 1076: PRO59546
FIG. 1077: DNA273694, NP_006092.1, 204162_at
FIG. 1078: PRO61661
FIG. 1079A-B: DNA254376, NP_055778.1,
204166_at
FIG. 1080: PRO49486
FIG. 1081: DNA272655, NP_001818.1, 204170_s_at
FIG. 1082: PRO60781
FIG. 1083: DNA330041, NP_000088.2, 204172_at
FIG. 1084: PRO85324
FIG. 1085: DNA328529, NP_001620.2, 204174_at
FIG. 1086: PRO49814
FIG. 1087: DNA226380, NP_001765.1, 204192_at
FIG. 1088: PRO4695
FIG. 1089A-B: DNA290230, NP_004341.1,
204197_s_at
FIG. 1090: PRO70325
FIG. 1091: DNA151798, NP_001797.1, 204203_at
FIG. 1092: PRO12186
FIG. 1093: DNA271778, NP_068594.1, 204205_at
FIG. 1094: PRO60062
FIG. 1095: DNA333754, NP_004868.1, 204220_at
FIG. 1096: PRO88379
FIG. 1097: DNA150812, NP_006842.1, 204222_s_at
FIG. 1098: PRO12481
FIG. 1099A-B: DNA287273, NP_006435.1,
204240_s_at
FIG. 1100: PRO69545
FIG. 1101: DNA330043, NP_001789.2, 204252_at
FIG. 1102: PRO85326
FIG. 1103A-B: DNA103527, NP_000367.1,
204254_s_at
FIG. 1104: PRO4854
FIG. 1105A-B: DNA103527, NP_000376,
204255_s_at
FIG. 1106: PRO4854
FIG. 1107: DNA228132, NP_076995.1, 204256_at
FIG. 1108: PRO38595
FIG. 1109: DNA273802, NP_066950.1, 204285_s_at
FIG. 1110: PRO61763
FIG. 1111: DNA273802, NM_021127, 204286_s_at
FIG. 1112: PRO61763
FIG. 1113: DNA344347, NP_002916.1, 204319_s_at
FIG. 1114: PRO63255
FIG. 1115: DNA330136, X76717, 204326_x_at
FIG. 1116: PRO82583
FIG. 1117: DNA327613, NP_005971.1, 204351_at
FIG. 1118: PRO83622
FIG. 1119A-D: DNA339387, NP_055625.2,
204373_s_at
FIG. 1120: PRO91192
FIG. 1121: DNA344348, NP_004477.2, 204384_at
FIG. 1122: PRO95059
FIG. 1123: DNA334269, NP_000231.1, 204388_s_at
FIG. 1124: PRO59228
FIG. 1125: DNA334269, NM_000240, 204389_at
FIG. 1126: PRO59228
FIG. 1127: DNA344349, NP_002241.1, 204401_at
FIG. 1128: PRO4787
FIG. 1129: DNA255402, NP_055288.1, 204405_x_at
FIG. 1130: PRO50469
FIG. 1131A-B: DNA254135, NP_060066.1,
204411_at
FIG. 1132: PRO49250
FIG. 1133: DNA327616, NP_075011.1, 204415_at
FIG. 1134: PRO83624
FIG. 1135: DNA327617, NP_006811.1, 204439_at
FIG. 1136: PRO83625
FIG. 1137A-B: DNA330049, NP_004514.2,
204444_at
FIG. 1138: PRO85330
FIG. 1139: DNA270496, NP_001316.1, 204459_at
FIG. 1140: PRO58875
FIG. 1141: DNA331075, NP_000601.2, 204489_s_at
FIG. 1142: PRO86231
FIG. 1143: DNA331075, NM_000610, 204490_s_at
FIG. 1144: PRO86231
FIG. 1145A-C: DNA344350, 418805.19, 204491_s_at
FIG. 1146: PRO95060
FIG. 1147: DNA194652, NP_001187.1, 204493_at
FIG. 1148: PRO23974
FIG. 1149A-B: DNA331311, NP_056054.1,
204500_s_at
FIG. 1150: PRO86405
FIG. 1151: DNA297387, NP_003494.1, 204510_at
FIG. 1152: PRO58394
FIG. 1153: DNA330051, NP_003431.1, 204523_at
FIG. 1154: PRO85332
FIG. 1155A-B: DNA272298, NP_055544.1,
204529_s_at
FIG. 1156: PRO60555
FIG. 1157: DNA82362, NP_001556.1, 204533_at
FIG. 1158: PRO1718
FIG. 1159: DNA225993, NP_000646.1, 204563_at
FIG. 1160: PRO36456
FIG. 1161: DNA151910, NP_004906.2, 204567_s_at
FIG. 1162: PRO12754
FIG. 1163: DNA328266, NP_005993.1, 204616_at
FIG. 1164: PRO12125
FIG. 1165: DNA344351, NP_006177.1, 204621_s_at
FIG. 1166: PRO12850
FIG. 1167: DNA344352, NM_173173, 204622_x_at
FIG. 1168: PRO95061
FIG. 1169: DNA226079, NP_001602.1, 204638_at
FIG. 1170: PRO36542
FIG. 1171: DNA226699, NP_000013.1, 204639_at
FIG. 1172: PRO37162
FIG. 1173: DNA254470, NP_002488.1, 204641_at
FIG. 1174: PRO49578
FIG. 1175A-B: DNA227097, NP_000101.1,
204646_at
FIG. 1176: PRO37560
FIG. 1177: DNA52729, M21121, 204655_at
FIG. 1178: PRO91
FIG. 1179: DNA344353, M11867, 204670_x_at
FIG. 1180: PRO95062
FIG. 1181: DNA327521, NP_002192.2, 204698_at
FIG. 1182: PRO58320
FIG. 1183: DNA271179, NP_004280.3, 204702_s_at
FIG. 1184: PRO59497
FIG. 1185A-B: DNA344354, NP_612565.1,
204709_s_at
FIG. 1186: PRO95063
FIG. 1187A-B: DNA335768, NP_000121.1,
204714_s_at
FIG. 1188: PRO90077
FIG. 1189A-B: DNA273690, NP_055602.1,
204720_s_at
FIG. 1190: PRO61657
FIG. 1191: DNA328698, NP_006144.1, 204725_s_at
FIG. 1192: PRO12168
FIG. 1193A-B: DNA83176, NP_003234.1, 204731_at
FIG. 1194: PRO2620
FIG. 1195A-B: DNA344355, NP_006193.1,
204735_at
FIG. 1196: PRO95064
FIG. 1197A-B: DNA325192, NP_038203.1,
204744_s_at
FIG. 1198: PRO81753
FIG. 1199: DNA330057, NP_005941.1, 204745_x_at
FIG. 1200: PRO85337
FIG. 1201: DNA287178, NP_001540.1, 204747_at
FIG. 1202: PRO69467
FIG. 1203A-B: DNA226070, NP_000954.1,
204748_at
FIG. 1204: PRO36533
FIG. 1205: DNA330058, NP_004529.2, 204749_at
FIG. 1206: PRO85338
FIG. 1207A-B: DNA270601, NP_002117.1,
204753_s_at
FIG. 1208: PRO58973
FIG. 1209: DNA329153, NP_001259.1, 204759_at
FIG. 1210: PRO84786
FIG. 1211: DNA328541, NP_004503.1, 204773_at
FIG. 1212: PRO4843
FIG. 1213: DNA328542, NP_055025.1, 204774_at
FIG. 1214: PRO2577
FIG. 1215: DNA227033, NP_002362.1, 204777_s_at
FIG. 1216: PRO37496
FIG. 1217: DNA332667, NP_000034.1, 204780_s_at
FIG. 1218: PRO1207
FIG. 1219: DNA344356, NM_152877, 204781_s_at
FIG. 1220: PRO95065
FIG. 1221: DNA344357, NP_000865.2, 204786_s_at
FIG. 1222: PRO1011
FIG. 1223: DNA253585, NP_004409.1, 204794_at
FIG. 1224: PRO49183
FIG. 1225A-B: DNA329907, NP_036423.1,
204817_at
FIG. 1226: PRO85224
FIG. 1227: DNA254127, NM_006994, 204820_s_at
FIG. 1228: PRO49242
FIG. 1229: DNA254127, U90548, 204821_at
FIG. 1230: PRO49242
FIG. 1231A-B: DNA269878, M86699, 204822_at
FIG. 1232: PRO58276
FIG. 1233: DNA255289, NP_055606.1, 204825_at
FIG. 1234: PRO50363
FIG. 1235: DNA344358, NP_002175.2, 204863_s_at
FIG. 1236: PRO85478
FIG. 1237: DNA344359, NM_175767, 204864_s_at
FIG. 1238: PRO95066
FIG. 1239: DNA333633, NM_014882, 204882_at
FIG. 1240: PRO88275
FIG. 1241: DNA330065, NP_055079.2, 204887_s_at
FIG. 1242: PRO85345
FIG. 1243: DNA226195, NP_000949.1, 204896_s_at
FIG. 1244: PRO36658
FIG. 1245: DNA344360, 334072.2, 204897_at
FIG. 1246: PRO95067
FIG. 1247: DNA329157, NP_004271.1, 204905_s_at
FIG. 1248: PRO62861
FIG. 1249A-B: DNA344361, NP_001549.1,
204912_at
FIG. 1250: PRO2536
FIG. 1251: DNA228014, NP_002153.1, 204949_at
FIG. 1252: PRO38477
FIG. 1253: DNA150427, NP_005599.1, 204960_at
FIG. 1254: PRO12243
FIG. 1255: DNA330067, NP_001800.1, 204962_s_at
FIG. 1256: PRO60368
FIG. 1257: DNA287399, NP_058197.1, 204972_at
FIG. 1258: PRO69656
FIG. 1259: DNA329158, NP_077013.1, 204985_s_at
FIG. 1260: PRO84788
FIG. 1261: DNA272427, NP_004799.1, 205005_s_at
FIG. 1262: PRO60679
FIG. 1263: DNA272427, NM_004808, 205006_s_at
FIG. 1264: PRO60679
FIG. 1265: DNA344362, NP_000666.2, 205013_s_at
FIG. 1266: PRO4938
FIG. 1267: DNA329534, NP_004615.2, 205019_s_at
FIG. 1268: PRO2904
FIG. 1269: DNA272312, NP_005188.1, 205022_s_at
FIG. 1270: PRO60569
FIG. 1271: DNA330069, NP_002866.2, 205024_s_at
FIG. 1272: PRO85348
FIG. 1273: DNA328297, NP_477097.1, 205034_at
FIG. 1274: PRO59418
FIG. 1275: DNA324992, NP_597680.1, 205047_s_at
FIG. 1276: PRO81586
FIG. 1277: DNA328551, NP_003823.1, 205048_s_at
FIG. 1278: PRO84351
FIG. 1279A-B: DNA83118, NP_000213.1,
205051_s_at
FIG. 1280: PRO2598
FIG. 1281: DNA254214, NP_001689.1, 205052_at
FIG. 1282: PRO49326
FIG. 1283A-B: DNA220750, NP_002199.2,
205055_at
FIG. 1284: PRO34728
FIG. 1285: DNA329025, NP_006199.1, 205066_s_at
FIG. 1286: PRO4860
FIG. 1287: DNA327632, NP_001302.1, 205081_at
FIG. 1288: PRO83635
FIG. 1289A-B: DNA344363, NP_005482.1,
205088_at
FIG. 1290: PRO95068
FIG. 1291: DNA344364, 331306.1, 205098_at
FIG. 1292: PRO4949
FIG. 1293: DNA226177, NP_001286.1, 205099_s_at
FIG. 1294: PRO36640
FIG. 1295: DNA192060, NP_002974.1, 205114_s_at
FIG. 1296: PRO21960
FIG. 1297: DNA344365, NP_008924.1, 205129_at
FIG. 1298: PRO95069
FIG. 1299: DNA299899, NP_002148.1, 205133_s_at
FIG. 1300: PRO62760
FIG. 1301: DNA328554, NP_038202.1, 205147_x_at
FIG. 1302: PRO84354
FIG. 1303A-B: DNA329160, NP_002821.1,
205171_at
FIG. 1304: PRO84789
FIG. 1305: DNA328810, NP_001770.1, 205173_x_at
FIG. 1306: PRO2557
FIG. 1307: DNA344366, NP_004476.1, 205184_at
FIG. 1308: PRO59080
FIG. 1309: DNA272443, NP_055531.1, 205213_at
FIG. 1310: PRO60693
FIG. 1311: DNA273535, NP_004217.1, 205214_at
FIG. 1312: PRO61515
FIG. 1313: DNA188333, NP_006410.1, 205242_at
FIG. 1314: PRO21708
FIG. 1315: DNA227447, NP_003193.1, 205254_x_at
FIG. 1316: PRO37910
FIG. 1317: DNA227447, NM_003202, 205255_x_at
FIG. 1318: PRO37910
FIG. 1319A-B: DNA188301, NP_002300.1,
205266_at
FIG. 1320: PRO21834
FIG. 1321: DNA332739, NP_006226.1, 205267_at
FIG. 1322: PRO87518
FIG. 1323: DNA227173, NP_001456.1, 205285_s_at
FIG. 1324: PRO37636
FIG. 1325A-B: DNA331483, NM_003672,
205288_at
FIG. 1326: PRO86528
FIG. 1327: DNA43320, DNA43320, 205289_at
FIG. 1328: PRO313
FIG. 1329: DNA219011, NP_001191.1, 205290_s_at
FIG. 1330: PRO34479
FIG. 1331A-B: DNA331484, NP_000869.1,
205291_at
FIG. 1332: PRO3276
FIG. 1333: DNA327019, NP_001406.1, 205321_at
FIG. 1334: PRO83323
FIG. 1335A-B: DNA269546, NP_055612.1,
205340_at
FIG. 1336: PRO57962
FIG. 1337: DNA326497, NM_000156, 205354_at
FIG. 1338: PRO58046
FIG. 1339: DNA336844, NP_003857.1, 205376_at
FIG. 1340: PRO90913
FIG. 1341A-C: DNA332571, NP_065209.1,
205390_s_at
FIG. 1342: PRO12143
FIG. 1343: DNA325568, NP_001265.1, 205393_s_at
FIG. 1344: PRO12187
FIG. 1345: DNA325568, NM_001274, 205394_at
FIG. 1346: PRO12187
FIG. 1347: DNA151830, NP_005893.1, 205397_x_at
FIG. 1348: PRO62998
FIG. 1349: DNA151830, NM_005902, 205398_s_at
FIG. 1350: PRO62998
FIG. 1351: DNA329010, NP_004942.1, 205419_at
FIG. 1352: PRO23370
FIG. 1353: DNA335207, NP_057531.2, 205429_s_at
FIG. 1354: PRO89594
FIG. 1355: DNA287337, NP_002096.1, 205436_s_at
FIG. 1356: PRO69600
FIG. 1357: DNA272221, NP_037431.1, 205449_at
FIG. 1358: PRO60483
FIG. 1359: DNA88194, NP_000724.1, 205456_at
FIG. 1360: PRO2220
FIG. 1361: DNA188355, NP_004582.1, 205476_at
FIG. 1362: PRO21885
FIG. 1363: DNA287224, NP_005092.1, 205483_s_at
FIG. 1364: PRO69503
FIG. 1365: DNA330084, NP_055265.1, 205484_at
FIG. 1366: PRO9895
FIG. 1367A-E: DNA334058, NP_000531.1,
205485_at
FIG. 1368: PRO88622
FIG. 1369: DNA225959, NP_006135.1, 205488_at
FIG. 1370: PRO36422
FIG. 1371: DNA226043, NP_006424.2, 205495_s_at
FIG. 1372: PRO36506
FIG. 1373A-B: DNA344367, NP_005392.1,
205503_at
FIG. 1374: PRO24022
FIG. 1375: DNA344368, NP_001481.2, 205505_at
FIG. 1376: PRO95070
FIG. 1377: DNA328566, NP_060446.1, 205511_at
FIG. 1378: PRO84363
FIG. 1379A-B: DNA334718, NP_004923.1,
205532_s_at
FIG. 1380: PRO2196
FIG. 1381: DNA344369, NP_036581.1, 205542_at
FIG. 1382: PRO28528
FIG. 1383: DNA344370, NP_006797.3, 205548_s_at
FIG. 1384: PRO95071
FIG. 1385: DNA331486, NM_002534, 205552_s_at
FIG. 1386: PRO69559
FIG. 1387: DNA256257, NP_055213.1, 205569_at
FIG. 1388: PRO51301
FIG. 1389A-B: DNA227714, NP_000852.1,
205579_at
FIG. 1390: PRO38177
FIG. 1391A-B: DNA327643, NP_055712.1,
205594_at
FIG. 1392: PRO83644
FIG. 1393: DNA344371, NP_073576.1, 205596_s_at
FIG. 1394: PRO95072
FIG. 1395: DNA329013, NP_005649.1, 205599_at
FIG. 1396: PRO20128
FIG. 1397: DNA90631, NP_000747.1, 205630_at
FIG. 1398: PRO2519
FIG. 1399: DNA88076, NP_001628.1, 205639_at
FIG. 1400: PRO2640
FIG. 1401: DNA344372, NP_003780.1, 205641_s_at
FIG. 1402: PRO95073
FIG. 1403A-B: DNA196641, NP_002340.1,
205668_at
FIG. 1404: PRO25114
FIG. 1405: DNA344373, NP_076992.1, 205673_s_at
FIG. 1406: PRO95074
FIG. 1407: DNA328570, NP_004040.1, 205681_at
FIG. 1408: PRO37843
FIG. 1409: DNA327644, NP_060395.2, 205684_s_at
FIG. 1410: PRO83645
FIG. 1411: DNA344374, NP_061989.1, 205687_at
FIG. 1412: PRO95075
FIG. 1413: DNA226234, NP_001766.1, 205692_s_at
FIG. 1414: PRO36697
FIG. 1415: DNA150621, NP_036595.1, 205704_s_at
FIG. 1416: PRO12374
FIG. 1417: DNA331817, NP_055154.3, 205707_at
FIG. 1418: PRO86240
FIG. 1419: DNA220761, NP_000880.1, 205718_at
FIG. 1420: PRO34739
FIG. 1421: DNA326483, NP_060346.1, 205748_s_at
FIG. 1422: PRO82861
FIG. 1423: DNA331318, NP_003636.1, 205768_s_at
FIG. 1424: PRO51139
FIG. 1425: DNA331318, NM_003645, 205769_at
FIG. 1426: PRO51139
FIG. 1427: DNA330091, NP_057461.1, 205771_s_at
FIG. 1428: PRO85362
FIG. 1429: DNA344375, NP_002176.2, 205798_at
FIG. 1430: PRO95076
FIG. 1431A-B: DNA344376, NP_733772.1,
205801_s_at
FIG. 1432: PRO95077
FIG. 1433: DNA194766, NP_079504.1, 205804_s_at
FIG. 1434: PRO24046
FIG. 1435: DNA344377, NP_064512.1, 205807_s_at
FIG. 1436: PRO95078
FIG. 1437: DNA103440, NP_031386.1, 205821_at
FIG. 1438: PRO4767
FIG. 1439: DNA75526, NP_001758.1, 205831_at
FIG. 1440: PRO2013
FIG. 1441A-B: DNA328574, NP_004963.1,
205841_at
FIG. 1442: PRO84368
FIG. 1443A-B: DNA328574, NM_004972,
205842_s_at
FIG. 1444: PRO84368
FIG. 1445A-B: DNA220746, NP_000876.1,
205884_at
FIG. 1446: PRO34724
FIG. 1447: DNA330095, NP_004732.1, 205895_s_at
FIG. 1448: PRO85366
FIG. 1449: DNA328576, NP_001328.1, 205898_at
FIG. 1450: PRO4940
FIG. 1451: DNA103307, NP_000238.1, 205904_at
FIG. 1452: PRO4637
FIG. 1453A-B: DNA339322, NP_003408.1,
205917_at
FIG. 1454: PRO91128
FIG. 1455A-B: DNA255292, NP_056374.1,
205933_at
FIG. 1456: PRO50365
FIG. 1457A-B: DNA270867, NP_006217.1,
205934_at
FIG. 1458: PRO59203
FIG. 1459: DNA329047, NP_006390.1, 205965_at
FIG. 1460: PRO58425
FIG. 1461: DNA196439, NP_003865.1, 205988_at
FIG. 1462: PRO24934
FIG. 1463A-B: DNA227747, NP_005798.1,
206007_at
FIG. 1464: PRO38210
FIG. 1465: DNA103281, NP_002899.1, 206036_s_at
FIG. 1466: PRO4611
FIG. 1467: DNA344378, NP_073715.1, 206042_x_at
FIG. 1468: PRO95079
FIG. 1469: DNA275181, NP_003081.1, 206055_s_at
FIG. 1470: PRO62882
FIG. 1471: DNA330096, NP_057051.1, 206060_s_at
FIG. 1472: PRO37163
FIG. 1473A-B: DNA344379, NP_006246.2,
206099_at
FIG. 1474: PRO95080
FIG. 1475: DNA83063, NP_004429.1, 206114_at
FIG. 1476: PRO2068
FIG. 1477A-B: DNA151420, NP_004421.1,
206115_at
FIG. 1478: PRO12876
FIG. 1479: DNA329006, NP_003142.1, 206118_at
FIG. 1480: PRO12865
FIG. 1481: DNA331657, NP_001707.1, 206126_at
FIG. 1482: PRO23970
FIG. 1483: DNA344380, NP_004953.1, 206159_at
FIG. 1484: PRO2562
FIG. 1485: DNA329005, NP_003028.1, 206181_at
FIG. 1486: PRO12612
FIG. 1487A-B: DNA344381, NP_055604.1,
206188_at
FIG. 1488: PRO95081
FIG. 1489A-B: DNA274141, NP_006460.2,
206245_s_at
FIG. 1490: PRO62077
FIG. 1491: DNA334388, NP_055141.2, 206324_s_at
FIG. 1492: PRO88904
FIG. 1493: DNA88224, NP_001829.1, 206337_at
FIG. 1494: PRO2236
FIG. 1495: DNA336220, NM_006123, 206342_x_at
FIG. 1496: PRO91049
FIG. 1497: DNA227700, NP_004769.1, 206361_at
FIG. 1498: PRO38163
FIG. 1499: DNA227208, NP_005351.2, 206363_at
FIG. 1500: PRO37671
FIG. 1501A-B: DNA330100, NP_055690.1,
206364_at
FIG. 1502: PRO85369
FIG. 1503: DNA329169, NP_002986.1, 206365_at
FIG. 1504: PRO1610
FIG. 1505: DNA329169, NM_002995, 206366_x_at
FIG. 1506: PRO1610
FIG. 1507A-B: DNA335332, NP_002640.2,
206369_s_at
FIG. 1508: PRO89706
FIG. 1509A-E: DNA333253, NP_066267.1,
206385_s_at
FIG. 1510: PRO87958
FIG. 1511: DNA326727, NP_001527.1, 206445_s_at
FIG. 1512: PRO83069
FIG. 1513: DNA153751, NP_005942.1, 206461_x_at
FIG. 1514: PRO12925
FIG. 1515: DNA288243, NP_002277.3, 206486_at
FIG. 1516: PRO36451
FIG. 1517: DNA268333, NP_001260.1, 206499_s_at
FIG. 1518: PRO57322
FIG. 1519: DNA344382, NP_003826.1, 206518_s_at
FIG. 1520: PRO95082
FIG. 1521A-B: DNA334589, NP_055073.1,
206546_at
FIG. 1522: PRO89073
FIG. 1523: DNA327663, NP_006771.1, 206565_x_at
FIG. 1524: PRO83654
FIG. 1525: DNA330103, NP_056179.1, 206584_at
FIG. 1526: PRO19671
FIG. 1527: DNA329172, NP_005254.1, 206589_at
FIG. 1528: PRO84796
FIG. 1529: DNA344383, NP_003846.1, 206618_at
FIG. 1530: PRO4778
FIG. 1531A-C: DNA328331, NP_004645.1,
206624_at
FIG. 1532: PRO84195
FIG. 1533: DNA227709, NP_000947.1, 206631_at
FIG. 1534: PRO38172
FIG. 1535: DNA335452, NP_004891.3, 206632_s_at
FIG. 1536: PRO89808
FIG. 1537: DNA327666, 7688312.1, 206653_at
FIG. 1538: PRO83656
FIG. 1539: DNA88374, NP_002095.1, 206666_at
FIG. 1540: PRO2768
FIG. 1541: DNA334470, NP_536859.1, 206687_s_at
FIG. 1542: PRO88974
FIG. 1543: DNA328590, NP_056948.2, 206707_x_at
FIG. 1544: PRO84375
FIG. 1545: DNA340145, NP_036439.1, 206710_s_at
FIG. 1546: PRO91644
FIG. 1547: DNA340152, NP_055300.1, 206726_at
FIG. 1548: PRO91651
FIG. 1549: DNA226427, NP_002251.1, 206785_s_at
FIG. 1550: PRO36890
FIG. 1551: DNA88195, NP_000064.1, 206804_at
FIG. 1552: PRO2693
FIG. 1553: DNA272165, NP_003319.1, 206828_at
FIG. 1554: PRO60433
FIG. 1555: DNA339650, NP_079465.1, 206829_x_at
FIG. 1556: PRO91399
FIG. 1557: DNA256561, NP_062550.1, 206914_at
FIG. 1558: PRO51592
FIG. 1559: DNA344384, NP_005659.1, 206925_at
FIG. 1560: PRO59592
FIG. 1561: DNA83130, NP_002665.1, 206942_s_at
FIG. 1562: PRO2096
FIG. 1563: DNA93439, NP_006555.1, 206974_at
FIG. 1564: PRO4515
FIG. 1565: DNA35629, NP_000586.2, 206975_at
FIG. 1566: PRO7
FIG. 1567: DNA331493, NP_000638.1, 206978_at
FIG. 1568: PRO84690
FIG. 1569: DNA188346, NP_001450.1, 206980_s_at
FIG. 1570: PRO21766
FIG. 1571A-B: DNA227659, NP_000570.1,
206991_s_at
FIG. 1572: PRO38122
FIG. 1573A-B: DNA344385, NP_001550.1,
206999_at
FIG. 1574: PRO23394
FIG. 1575: DNA328295, NP_004154.2, 207017_at
FIG. 1576: PRO84168
FIG. 1577: DNA344386, NP_003830.1, 207037_at
FIG. 1578: PRO20114
FIG. 1579: DNA344387, NP_003844.1, 207072_at
FIG. 1580: PRO36013
FIG. 1581: DNA334102, NM_020481, 207087_x_at
FIG. 1582: PRO88662
FIG. 1583: DNA344388, NM_000594, 207113_s_at
FIG. 1584: PRO6
FIG. 1585: DNA344389, NP_060113.1, 207115_x_at
FIG. 1586: PRO95083
FIG. 1587A-B: DNA327674, NP_002739.1,
207121_s_at
FIG. 1588: PRO83661
FIG. 1589: DNA331323, NP_001250.1, 207143_at
FIG. 1590: PRO86412
FIG. 1591: DNA344390, NP_000873.2, 207160_at
FIG. 1592: PRO82
FIG. 1593: DNA103418, NP_036616.1, 207165_at
FIG. 1594: PRO4746
FIG. 1595: DNA344391, NP_004450.1, 207186_s_at
FIG. 1596: PRO95084
FIG. 1597A-B: DNA151879, NP_055463.1,
207231_at
FIG. 1598: PRO12743
FIG. 1599A-B: DNA151879, NM_014648,
207232_s_at
FIG. 1600: PRO12743
FIG. 1601: DNA330024, NP_058521.1, 207266_x_at
FIG. 1602: PRO85309
FIG. 1603: DNA226045, NP_006728.1, 207313_x_at
FIG. 1604: PRO36508
FIG. 1605: DNA226045, NM_006737, 207314_x_at
FIG. 1606: PRO36508
FIG. 1607: DNA227751, NP_006557.1, 207315_at
FIG. 1608: PRO38214
FIG. 1609A-B: DNA226536, NP_003225.1,
207332_s_at
FIG. 1610: PRO36999
FIG. 1611: DNA88656, NP_003233.3, 207334_s_at
FIG. 1612: PRO2461
FIG. 1613: DNA331497, NP_002332.1, 207339_s_at
FIG. 1614: PRO11604
FIG. 1615: DNA330117, NP_003966.1, 207351_s_at
FIG. 1616: PRO85379
FIG. 1617: DNA225961, NP_005308.1, 207460_at
FIG. 1618: PRO36424
FIG. 1619: DNA274829, NP_003653.1, 207469_s_at
FIG. 1620: PRO62588
FIG. 1621: DNA344392, AK000231, 207474_at
FIG. 1622: PRO95085
FIG. 1623: DNA344393, Y07827, 207485_x_at
FIG. 1624: PRO95086
FIG. 1625A-B: DNA344394, NP_777613.1,
207521_s_at
FIG. 1626: PRO95087
FIG. 1627A-B: DNA344395, NM_174954,
207522_s_at
FIG. 1628: PRO95088
FIG. 1629: DNA216508, NP_002972.1, 207533_at
FIG. 1630: PRO34260
FIG. 1631: DNA344396, NP_001552.2, 207536_s_at
FIG. 1632: PRO2023
FIG. 1633: DNA344397, NP_000580.1, 207538_at
FIG. 1634: PRO68
FIG. 1635: DNA344398, NM_000589, 207539_s_at
FIG. 1636: PRO68
FIG. 1637: DNA344399, NP_523353.1, 207551_s_at
FIG. 1638: PRO95089
FIG. 1639: DNA328600, NP_0004839.1, 207571_x_at
FIG. 1640: PRO84383
FIG. 1641: DNA328601, NP_056490.1, 207574_s_at
FIG. 1642: PRO84384
FIG. 1643: DNA330121, NP_004171.2, 207616_s_at
FIG. 1644: PRO85383
FIG. 1645: DNA228010, NP_003679.1, 207620_s_at
FIG. 1646: PRO38473
FIG. 1647: DNA344400, NP_005683.2, 207622_s_at
FIG. 1648: PRO36800
FIG. 1649: DNA227606, NP_001872.2, 207630_s_at
FIG. 1650: PRO38069
FIG. 1651: DNA196426, NP_037440.1, 207651_at
FIG. 1652: PRO24924
FIG. 1653: DNA328554, NM_013416, 207677_s_at
FIG. 1654: PRO84354
FIG. 1655: DNA227752, NP_001495.1, 207681_at
FIG. 1656: PRO38215
FIG. 1657: DNA328763, NP_001219.2, 207686_s_at
FIG. 1658: PRO84511
FIG. 1659: DNA336246, NP_001767.2, 207691_x_at
FIG. 1660: PRO90415
FIG. 1661A-B: DNA226405, NP_006525.1,
207700_s_at
FIG. 1662: PRO36868
FIG. 1663: DNA333631, NP_031359.1, 207723_s_at
FIG. 1664: PRO88273
FIG. 1665: DNA329064, NP_060301.1, 207735_at
FIG. 1666: PRO84724
FIG. 1667: DNA325654, NP_054752.1, 207761_s_at
FIG. 1668: PRO4348
FIG. 1669A-B: DNA329179, NP_056958.1,
207785_s_at
FIG. 1670: PRO84802
FIG. 1671: DNA329180, NP_004428.1, 207793_s_at
FIG. 1672: PRO84803
FIG. 1673: DNA329000, NM_000648, 207794_at
FIG. 1674: PRO84690
FIG. 1675: DNA227722, NP_002253.1, 207795_s_at
FIG. 1676: PRO38185
FIG. 1677: DNA329181, NM_007334, 207796_x_at
FIG. 1678: PRO84804
FIG. 1679: DNA227494, NP_002158.1, 207826_s_at
FIG. 1680: PRO37957
FIG. 1681A-C: DNA335409, NP_057427.2,
207828_s_at
FIG. 1682: PRO89771
FIG. 1683: DNA329182, NP_065385.2, 207838_x_at
FIG. 1684: PRO84805
FIG. 1685: DNA330123, NP_008984.1, 207840_at
FIG. 1686: PRO35080
FIG. 1687: DNA344401, NP_002179.2, 207844_at
FIG. 1688: PRO95090
FIG. 1689: DNA217244, U25676, 207849_at
FIG. 1690: PRO34286
FIG. 1691: DNA330124, NP_002981.2, 207861_at
FIG. 1692: PRO34107
FIG. 1693: DNA109234, NP_000065.1, 207892_at
FIG. 1694: PRO6517
FIG. 1695: DNA344402, NP_002978.1, 207900_at
FIG. 1696: PRO1717
FIG. 1697A-B: DNA150910, NP_005566.1,
207904_s_at
FIG. 1698: PRO12536
FIG. 1699: DNA344403, NP_000579.2, 207906_at
FIG. 1700: PRO95091
FIG. 1701: DNA344404, NP_000870.1, 207952_at
FIG. 1702: PRO69
FIG. 1703: DNA227067, X06318, 207957_s_at
FIG. 1704: PRO37530
FIG. 1705A-B: DNA344405, NP_008912.1,
207978_s_at
FIG. 1706: PRO85386
FIG. 1707A-C: DNA254145, NP_004329.1,
207996_s_at
FIG. 1708: PRO49260
FIG. 1709A-B: DNA226403, NP_000711.1,
207998_s_at
FIG. 1710: PRO36866
FIG. 1711: DNA344406, NM_012411, 208010_s_at
FIG. 1712: PRO95092
FIG. 1713: DNA324249, NM_004510, 208012_x_at
FIG. 1714: PRO80933
FIG. 1715: DNA333763, NM_021708, 208071_s_at
FIG. 1716: PRO88387
FIG. 1717A-C: DNA331500, NP_003307.2,
208073_x_at
FIG. 1718: PRO86537
FIG. 1719: DNA331501, D84212, 208079_s_at
FIG. 1720: PRO58855
FIG. 1721A-B: DNA344407, NP_110384.1,
208082_x_at
FIG. 1722: PRO95093
FIG. 1723: DNA344408, NP_112182.1, 208103_s_at
FIG. 1724: PRO80638
FIG. 1725A-B: DNA335356, NP_000952.1,
208131_s_at
FIG. 1726: PRO25026
FIG. 1727: DNA325329, NP_004719.1, 208152_s_at
FIG. 1728: PRO81872
FIG. 1729: DNA344409, NP_002177.1, 208164_s_at
FIG. 1730: PRO64957
FIG. 1731: DNA210622, NP_057009.1, 208190_s_at
FIG. 1732: PRO35016
FIG. 1733: DNA36717, NP_000581.1, 208193_at
FIG. 1734: PRO72
FIG. 1735: DNA328611, NP_005816.2, 208206_s_at
FIG. 1736: PRO84393
FIG. 1737: DNA344410, NP_071431.2, 208303_s_at
FIG. 1738: PRO28725
FIG. 1739: DNA196361, NP_001828.1, 208304_at
FIG. 1740: PRO24864
FIG. 1741: DNA344411, X12544, 208306_x_at
FIG. 1742: PRO95094
FIG. 1743A-B: DNA344412, NP_006776.1,
208309_s_at
FIG. 1744: PRO9824
FIG. 1745A-C: DNA344413, NP_006729.3,
208325_s_at
FIG. 1746: PRO95095
FIG. 1747: DNA344414, NP_003813.1, 208337_s_at
FIG. 1748: PRO62964
FIG. 1749: DNA344415, NM_003822, 208343_s_at
FIG. 1750: PRO62964
FIG. 1751: DNA329576, NM_002745, 208351_s_at
FIG. 1752: PRO64127
FIG. 1753: DNA344416, NM_020480, 208353_x_at
FIG. 1754: PRO95096
FIG. 1755: DNA344417, NP_008999.2, 208382_s_at
FIG. 1756: PRO95097
FIG. 1757: DNA324250, NP_536349.1, 208392_x_at
FIG. 1758: PRO80934
FIG. 1759A-B: DNA344418, NP_005723.2,
208393_s_at
FIG. 1760: PRO86236
FIG. 1761: DNA344419, NP_004801.1, 208406_s_at
FIG. 1762: PRO12190
FIG. 1763A-B: DNA331315, NP_004622.1,
208433_s_at
FIG. 1764: PRO70090
FIG. 1765: DNA327690, NP_004022.1, 208436_s_at
FIG. 1766: PRO83673
FIG. 1767A-C: DNA331504, NP_000042.2,
208442_s_at
FIG. 1768: PRO86540
FIG. 1769: DNA331327, NP_036382.2, 208456_s_at
FIG. 1770: PRO86414
FIG. 1771: DNA326738, NP_004315.1, 208478_s_at
FIG. 1772: PRO38101
FIG. 1773: DNA344420, NM_006260, 208499_s_at
FIG. 1774: PRO11602
FIG. 1775: DNA344421, NP_005281.1, 208524_at
FIG. 1776: PRO54695
FIG. 1777: DNA344422, NP_619527.1, 208536_s_at
FIG. 1778: PRO95098
FIG. 1779: DNA330045, NP_005943.1, 208581_x_at
FIG. 1780: PRO82583
FIG. 1781: DNA225836, NP_006716.1, 208602_x_at
FIG. 1782: PRO36299
FIG. 1783: DNA344423, NP_066301.1, 208608_s_at
FIG. 1784: PRO23346
FIG. 1785: DNA281431, NP_004550.1, 208628_s_at
FIG. 1786: PRO66271
FIG. 1787: DNA324641, NP_005608.1, 208646_at
FIG. 1788: PRO10849
FIG. 1789: DNA344424, NP_006007.2, 208653_s_at
FIG. 1790: PRO95099
FIG. 1791: DNA344425, U87954, 208676_s_at
FIG. 1792: PRO95100
FIG. 1793: DNA304686, NP_002565.1, 208680_at
FIG. 1794: PRO71112
FIG. 1795A-B: DNA328619, BC001188, 208691_at
FIG. 1796: PRO84401
FIG. 1797: DNA287189, NP_002038.1, 208693_s_at
FIG. 1798: PRO69475
FIG. 1799: DNA344426, NP_036205.1, 208696_at
FIG. 1800: PRO81195
FIG. 1801: DNA325127, NP_001559.1, 208697_s_at
FIG. 1802: PRO81699
FIG. 1803A-B: DNA325944, NP_001960.2,
208708_x_at
FIG. 1804: PRO82391
FIG. 1805: DNA344427, NP_061899.1, 208716_s_at
FIG. 1806: PRO177
FIG. 1807: DNA344428, NP_003899.1, 208726_s_at
FIG. 1808: PRO95101
FIG. 1809: DNA344429, NP_004879.1, 208737_at
FIG. 1810: PRO61194
FIG. 1811: DNA344430, NM_006476, 208745_at
FIG. 1812: PRO95102
FIG. 1813: DNA287285, NP_005794.1, 208748_s_at
FIG. 1814: PRO69556
FIG. 1815: DNA344431, NP_631946.1, 208754_s_at
FIG. 1816: PRO71113
FIG. 1817: DNA324217, NP_004035.2, 208758_at
FIG. 1818: PRO80908
FIG. 1819: DNA344432, NP_060877.1, 208767_s_at
FIG. 1820: PRO37687
FIG. 1821: DNA344433, NP_002806.2, 208777_s_at
FIG. 1822: PRO95103
FIG. 1823: DNA287219, NP_110379.1, 208778_s_at
FIG. 1824: PRO69498
FIG. 1825: DNA329189, NP_009139.1, 208787_at
FIG. 1826: PRO4911
FIG. 1827: DNA225671, NP_001822.1, 208791_at
FIG. 1828: PRO36134
FIG. 1829A-B: DNA344434, NP_055818.2,
208798_x_at
FIG. 1830: PRO95104
FIG. 1831: DNA330145, NP_002788.1, 208799_at
FIG. 1832: PRO84403
FIG. 1833A-C: DNA330146, 1397486.26, 208806_at
FIG. 1834: PRO85404
FIG. 1835: DNA273521, NP_002070.1, 208813_at
FIG. 1836: PRO61502
FIG. 1837: DNA327699, BAA75062.1, 208815_x_at
FIG. 1838: PRO83682
FIG. 1839: DNA344435, NP_002789.1, 208827_at
FIG. 1840: PRO82662
FIG. 1841A-B: DNA83031, NP_001737.1,
208852_s_at
FIG. 1842: PRO2564
FIG. 1843: DNA227874, NP_003320.1, 208864_s_at
FIG. 1844: PRO38337
FIG. 1845: DNA344436, NP_113600.1, 208869_s_at
FIG. 1846: PRO95105
FIG. 1847: DNA328624, BC003562, 208891_at
FIG. 1848: PRO59076
FIG. 1849: DNA270713, NP_001937.1, 208892_s_at
FIG. 1850: PRO59076
FIG. 1851: DNA328625, NM_022652, 208893_s_at
FIG. 1852: PRO84404
FIG. 1853: DNA329221, NP_061984.1, 208894_at
FIG. 1854: PRO4555
FIG. 1855A-B: DNA324910, NP_061820.1,
208905_at
FIG. 1856: PRO81514
FIG. 1857: DNA326260, NP_001203.1, 208910_s_at
FIG. 1858: PRO82667
FIG. 1859: DNA226500, NP_005619.1, 208916_at
FIG. 1860: PRO36963
FIG. 1861: DNA325473, NP_006353.2, 208922_s_at
FIG. 1862: PRO81996
FIG. 1863: DNA329552, NP_063948.1, 208925_at
FIG. 1864: PRO85097
FIG. 1865: DNA326233, NP_000968.2, 208929_x_at
FIG. 1866: PRO82645
FIG. 1867: DNA327702, NP_006490.2, 208934_s_at
FIG. 1868: PRO83684
FIG. 1869: DNA327702, NM_006499, 208936_x_at
FIG. 1870: PRO83684
FIG. 1871: DNA344437, NP_036379.1, 208941_s_at
FIG. 1872: PRO70339
FIG. 1873A-B: DNA344438, D50683, 208944_at
FIG. 1874: PRO95106
FIG. 1875: DNA325900, NP_002297.1, 208949_s_at
FIG. 1876: PRO82356
FIG. 1877: DNA327661, NP_005522.1, 208966_x_at
FIG. 1878: PRO83652
FIG. 1879A-B: DNA344439, NP_002256.2,
208974_x_at
FIG. 1880: PRO82739
FIG. 1881A-B: DNA330153, L38951, 208975_s_at
FIG. 1882: PRO82739
FIG. 1883: DNA328629, NP_006079.1, 208977_x_at
FIG. 1884: PRO84407
FIG. 1885: DNA329522, NP_000433.2, 208981_at
FIG. 1886: PRO85080
FIG. 1887: DNA330155, 7692317.2, 208982_at
FIG. 1888: PRO85407
FIG. 1889: DNA329522, NM_000442, 208983_s_at
FIG. 1890: PRO85080
FIG. 1891: DNA330156, NP_003749.1, 208985_s_at
FIG. 1892: PRO85408
FIG. 1893: DNA344440, NP_644805.1, 208991_at
FIG. 1894: PRO95107
FIG. 1895: DNA331514, NM_003150, 208992_s_at
FIG. 1896: PRO86548
FIG. 1897: DNA227552, NP_003346.2, 208997_s_at
FIG. 1898: PRO38015
FIG. 1899A-B: DNA344441, AAG09407.1,
208999_at
FIG. 1900: PRO95108
FIG. 1901: DNA328630, NP_036293.1, 209004_s_at
FIG. 1902: PRO84408
FIG. 1903: DNA328631, AK027318, 209006_s_at
FIG. 1904: PRO84409
FIG. 1905: DNA328632, NP_064713.2, 209007_s_at
FIG. 1906: PRO84410
FIG. 1907: DNA328633, NP_004784.2, 209017_s_at
FIG. 1908: PRO84411
FIG. 1909: DNA327706, NP_006363.3, 209024_s_at
FIG. 1910: PRO83688
FIG. 1911: DNA344442, AF279899, 209034_at
FIG. 1912: PRO95109
FIG. 1913: DNA274967, AF233453, 209049_s_at
FIG. 1914: PRO62700
FIG. 1915A-C: DNA344443, NP_579890.1,
209052_s_at
FIG. 1916: PRO81109
FIG. 1917A-B: DNA331518, NM_133336,
209053_s_at
FIG. 1918: PRO86550
FIG. 1919A-B: DNA226405, NM_006534,
209060_x_at
FIG. 1920: PRO36868
FIG. 1921A-C: DNA344444, 1394903.34, 209061_at
FIG. 1922: PRO95110
FIG. 1923A-B: DNA226405, AF036892,
209062_x_at
FIG. 1924: PRO36868
FIG. 1925: DNA330160, NP_006285.1, 209066_x_at
FIG. 1926: PRO85412
FIG. 1927: DNA329194, NP_112740.1, 209067_s_at
FIG. 1928: PRO84814
FIG. 1929A-B: DNA324473, NP_002904.2,
209084_s_at
FIG. 1930: PRO81135
FIG. 1931A-B: DNA273483, AB007960,
209090_s_at
FIG. 1932: DNA324318, NP_006755.2, 209100_at
FIG. 1933: PRO80995
FIG. 1934: DNA330118, NP_036389.2, 209102_s_at
FIG. 1935: PRO85380
FIG. 1936: DNA330163, NP_060308.1, 209104_s_at
FIG. 1937: PRO85415
FIG. 1938A-B: DNA344445, 104805.26, 209105_at
FIG. 1939: PRO95111
FIG. 1940: DNA344446, NP_004055.1, 209112_at
FIG. 1941: PRO95112
FIG. 1942: DNA344447, BC005127, 209122_at
FIG. 1943: PRO95113
FIG. 1944: DNA344448, NM_176895, 209147_s_at
FIG. 1945: PRO95114
FIG. 1946: DNA330166, NP_004688.2, 209161_at
FIG. 1947: PRO85418
FIG. 1948: DNA344449, 1448768.1, 209163_at
FIG. 1949: PRO95115
FIG. 1950: DNA344450, NP_001906.1, 209164_s_at
FIG. 1951: PRO57071
FIG. 1952A-C: DNA270403, NM_016343,
209172_s_at
FIG. 1953: PRO58786
FIG. 1954: DNA329196, NP_004573.2, 209181_s_at
FIG. 1955: PRO84815
FIG. 1956A-B: DNA344451, NP_733765.1,
209186_at
FIG. 1957: PRO84419
FIG. 1958: DNA189700, NP_005243.1, 209189_at
FIG. 1959: PRO25619
FIG. 1960: DNA226176, NP_003458.1, 209201_x_at
FIG. 1961: PRO36639
FIG. 1962: DNA326267, NP_004861.1, 209208_at
FIG. 1963: PRO82674
FIG. 1964: DNA103439, NP_001111.2, 209215_at
FIG. 1965: PRO4766
FIG. 1966: DNA330168, NP_006322.1, 209233_at
FIG. 1967: PRO85420
FIG. 1968: DNA344452, NM_007189, 209247_s_at
FIG. 1969: PRO95116
FIG. 1970: DNA344453, BC004949, 209251_x_at
FIG. 1971: PRO84424
FIG. 1972: DNA255255, NP_071437.3, 209267_s_at
FIG. 1973: PRO50332
FIG. 1974: DNA328650, DNA328650, 209286_at
FIG. 1975: PRO84425
FIG. 1976A-B: DNA344454, NP_006440.2,
209288_s_at
FIG. 1977: PRO95117
FIG. 1978: DNA328651, AF087853, 209304_x_at
FIG. 1979: PRO82889
FIG. 1980: DNA344455, BC024654, 209305_s_at
FIG. 1981: PRO95118
FIG. 1982: DNA344456, NP_001216.1, 209310_s_at
FIG. 1983: PRO37559
FIG. 1984: DNA344457, U65585, 209312_x_at
FIG. 1985: PRO95119
FIG. 1986A-B: DNA344458, NP_006611.1,
209316_s_at
FIG. 1987: PRO12057
FIG. 1988: DNA344459, U94829, 209325_s_at
FIG. 1989: PRO95120
FIG. 1990: DNA329200, NP_005040.1, 209336_at
FIG. 1991: PRO84817
FIG. 1992: DNA275106, NP_005058.2, 209339_at
FIG. 1993: PRO62821
FIG. 1994: DNA328655, 346677.3, 209341_s_at
FIG. 1995: PRO84429
FIG. 1996: DNA227208, NM_005360, 209347_s_at
FIG. 1997: PRO37671
FIG. 1998A-B: DNA328658, AF055376,
209348_s_at
FIG. 1999: PRO84432
FIG. 2000: DNA330170, AF109161, 209357_at
FIG. 2001: PRO84807
FIG. 2002A-B: DNA344460, NP_001745.2,
209360_s_at
FIG. 2003: PRO95121
FIG. 2004A-C: DNA344461, NP_061872.1,
209379_s_at
FIG. 2005: PRO95122
FIG. 2006: DNA330173, NP_006200.2, 209392_at
FIG. 2007: PRO85423
FIG. 2008: DNA339326, NP_004273.1, 209406_at
FIG. 2009: PRO91131
FIG. 2010: DNA330175, NP_006836.1, 209408_at
FIG. 2011: PRO59681
FIG. 2012A-B: DNA344462, NM_133650,
209447_at
FIG. 2013: PRO95123
FIG. 2014: DNA330121, NM_004180, 209451_at
FIG. 2015: PRO85383
FIG. 2016: DNA344463, NP_065737.1, 209459_s_at
FIG. 2017: PRO95124
FIG. 2018: DNA344464, NM_020686, 209460_at
FIG. 2019: PRO95125
FIG. 2020: DNA287304, AAH00040.1, 209461_x_at
FIG. 2021: PRO69571
FIG. 2022A-B: DNA344465, 347965.2, 209473_at
FIG. 2023: PRO95126
FIG. 2024: DNA336246, NM_001776, 209474_s_at
FIG. 2025: PRO90415
FIG. 2026: DNA324976, NP_005828.1, 209482_at
FIG. 2027: PRO81571
FIG. 2028: DNA324899, NP_002938.1, 209507_at
FIG. 2029: PRO81503
FIG. 2030: DNA274027, NP_004571.2, 209514_s_at
FIG. 2031: PRO61971
FIG. 2032A-B: DNA344466, NM_144767,
209534_x_at
FIG. 2033: PRO95127
FIG. 2034: DNA344467, NM_139265, 209536_s_at
FIG. 2035: PRO82426
FIG. 2036: DNA274949, NP_008904.1, 209538_at
FIG. 2037: PRO62684
FIG. 2038A-B: DNA344468, NP_004831.1,
209539_at
FIG. 2039: PRO83388
FIG. 2040A-C: DNA335383, NP_000609.1,
209540_at
FIG. 2041: PRO19618
FIG. 2042A-C: DNA335383, NM_000618,
209541_at
FIG. 2043: PRO19618
FIG. 2044: DNA329201, NP_055984.1, 209567_at
FIG. 2045: PRO84818
FIG. 2046: DNA344469, NP_003788.2, 209572_s_at
FIG. 2047: PRO40888
FIG. 2048A-C: DNA254145, NM_004338,
209573_s_at
FIG. 2049: PRO49260
FIG. 2050: DNA344470, NP_002060.3, 209576_at
FIG. 2051: PRO95128
FIG. 2052: DNA304797, NP_005935.3, 209582_s_at
FIG. 2053: PRO71209
FIG. 2054: DNA304797, NM_005944, 209583_s_at
FIG. 2055: PRO71209
FIG. 2056: DNA344471, NP_004119.1, 209595_at
FIG. 2057: PRO95129
FIG. 2058: DNA270689, NP_002042.1, 209602_s_at
FIG. 2059: PRO59053
FIG. 2060: DNA344472, 412986.6, 209603_at
FIG. 2061: PRO95130
FIG. 2062: DNA270689, NM_002051, 209604_s_at
FIG. 2063: PRO59053
FIG. 2064: DNA330186, NP_004327.1, 209642_at
FIG. 2065: PRO85434
FIG. 2066: DNA323856, NP_056455.1, 209669_s_at
FIG. 2067: PRO80599
FIG. 2068A-B: DNA344473, NP_008927.1,
209681_at
FIG. 2069: PRO23299
FIG. 2070A-B: DNA344474, NM_170662,
209682_at
FIG. 2071: PRO95131
FIG. 2072: DNA328264, NP_005183.2, 209714_s_at
FIG. 2073: PRO12087
FIG. 2074A-B: DNA328594, M37435, 209716_at
FIG. 2075: PRO84379
FIG. 2076A-C: DNA254412, NP_005656.2,
209717_at
FIG. 2077: PRO49522
FIG. 2078: DNA227124, NP_005118.1, 209732_at
FIG. 2079: PRO37587
FIG. 2080: DNA344475, AF113682, 209753_s_at
FIG. 2081: PRO95132
FIG. 2082: DNA344476, U09088, 209754_s_at
FIG. 2083: PRO95133
FIG. 2084: DNA324250, NM_080424, 209761_s_at
FIG. 2085: PRO80934
FIG. 2086A-B: DNA328675, NM_033274,
209765_at
FIG. 2087: PRO84447
FIG. 2088: DNA329178, NP_008979.2, 209770_at
FIG. 2089: PRO84801
FIG. 2090: DNA275195, NP_001025.1, 209773_s_at
FIG. 2091: PRO62893
FIG. 2092A-B: DNA255050, NP_065165.1,
209780_at
FIG. 2093: PRO50138
FIG. 2094A-B: DNA344477, AF222340,
209788_s_at
FIG. 2095: PRO95134
FIG. 2096: DNA336284, NP_001217.2, 209790_s_at
FIG. 2097: PRO90442
FIG. 2098: DNA226436, NP_001772.1, 209795_at
FIG. 2099: PRO36899
FIG. 2100: DNA327731, NP_003302.1, 209803_s_at
FIG. 2101: PRO83707
FIG. 2102: DNA271384, AAA61110.1, 209813_x_at
FIG. 2103: PRO59683
FIG. 2104: DNA326100, NP_006444.2, 209820_s_at
FIG. 2105: PRO82528
FIG. 2106: DNA225992, NP_003374.1, 209822_s_at
FIG. 2107: PRO36455
FIG. 2108: DNA344478, M17955, 209823_x_at
FIG. 2109: PRO95135
FIG. 2110: DNA336282, NP_001169.2, 209824_s_at
FIG. 2111: PRO61686
FIG. 2112: DNA327732, NP_036606.2, 209825_s_at
FIG. 2113: PRO61801
FIG. 2114A-B: DNA196499, AB002384, 209829_at
FIG. 2115: PRO24988
FIG. 2116: DNA344479, L05424, 209835_x_at
FIG. 2117: DNA344480, AAH35133.1, 209840_s_at
FIG. 2118: PRO95136
FIG. 2119: DNA329207, NM_018334, 209841_s_at
FIG. 2120: PRO220
FIG. 2121: DNA344481, BC012398, 209845_at
FIG. 2122: PRO95137
FIG. 2123: DNA324805, NP_008978.1, 209846_s_at
FIG. 2124: PRO81419
FIG. 2125: DNA272753, NP_005780.1, 209853_s_at
FIG. 2126: PRO60864
FIG. 2127: DNA344482, NP_006829.1, 209861_s_at
FIG. 2128: PRO61513
FIG. 2129A-B: DNA325767, NP_476510.1,
209876_at
FIG. 2130: PRO82238
FIG. 2131: DNA226120, NP_002997.1, 209879_at
FIG. 2132: PRO36583
FIG. 2133A-C: DNA194808, NP_003606.2,
209884_s_at
FIG. 2134: PRO24078
FIG. 2135A-B: DNA344483, NP_056305.1,
209889_at
FIG. 2136: PRO95138
FIG. 2137: DNA334335, NP_065726.1, 209891_at
FIG. 2138: PRO80882
FIG. 2139: DNA254936, NP_009164.1, 209917_s_at
FIG. 2140: PRO50026
FIG. 2141: DNA299884, AB040875, 209921_at
FIG. 2142: PRO70858
FIG. 2143: DNA226887, NP_002529.1, 209925_at
FIG. 2144: PRO37350
FIG. 2145: DNA150133, AAD01646.1, 209933_s_at
FIG. 2146: PRO12219
FIG. 2147: DNA336245, AF005775, 209939_x_at
FIG. 2148: PRO91070
FIG. 2149: DNA344484, NM_139266, 209969_s_at
FIG. 2150: PRO83711
FIG. 2151: DNA344485, AF116615, 209971_x_at
FIG. 2152: DNA226658, NP_003736.1, 209999_x_at
FIG. 2153: PRO37121
FIG. 2154: DNA226658, NM_003745, 210001_s_at
FIG. 2155: PRO37121
FIG. 2156A-B: DNA344486, NM_173844,
210017_at
FIG. 2157: PRO95140
FIG. 2158A-B: DNA344487, NM_006785,
210018_x_at
FIG. 2159: PRO9824
FIG. 2160: DNA255921, NP_000725.1, 210031_at
FIG. 2161: PRO50974
FIG. 2162: DNA344488, NP_002159.1, 210046_s_at
FIG. 2163: PRO82489
FIG. 2164: DNA326809, NP_036244.2, 210052_s_at
FIG. 2165: PRO83142
FIG. 2166: DNA328285, NP_002745.1, 210059_s_at
FIG. 2167: PRO84161
FIG. 2168: DNA344489, NP_057580.1, 210075_at
FIG. 2169: PRO50605
FIG. 2170: DNA334812, NP_002028.1, 210105_s_at
FIG. 2171: PRO4624
FIG. 2172A-C: DNA344490, 348003.19, 210108_at
FIG. 2173: PRO95141
FIG. 2174: DNA254310, NP_055226.1, 210109_at
FIG. 2175: PRO49421
FIG. 2176: DNA270010, NP_002342.1, 210116_at
FIG. 2177: PRO58405
FIG. 2178: DNA344491, 7763479.63, 210136_at
FIG. 2179: PRO95142
FIG. 2180: DNA333697, NP_003641.2, 210140_at
FIG. 2181: PRO88328
FIG. 2182: DNA256015, NP_002182.1, 210141_s_at
FIG. 2183: PRO51063
FIG. 2184: DNA344492, NP_077734.1, 210145_at
FIG. 2185: PRO90384
FIG. 2186: DNA340737, NM_172390, 210162_s_at
FIG. 2187: PRO92688
FIG. 2188: DNA330202, NP_005400.1, 210163_at
FIG. 2189: PRO19838
FIG. 2190: DNA287620, NP_004122.1, 210164_at
FIG. 2191: PRO2081
FIG. 2192: DNA335084, 233354.1, 210174_at
FIG. 2193: PRO89492
FIG. 2194: DNA330203, NP_003755.1, 210190_at
FIG. 2195: PRO85449
FIG. 2196: DNA186230, NP_006599.1, 210191_s_at
FIG. 2197: PRO21476
FIG. 2198: DNA344493, NP_003773.1, 210205_at
FIG. 2199: PRO1756
FIG. 2200: DNA344494, NP_000749.2, 210229_s_at
FIG. 2201: PRO2055
FIG. 2202: DNA344495, NM_134470, 210233_at
FIG. 2203: PRO88491
FIG. 2204: DNA328690, NP_524145.1, 210240_s_at
FIG. 2205: PRO59660
FIG. 2206: DNA287333, NP_005283.1, 210279_at
FIG. 2207: PRO69597
FIG. 2208A-B: DNA270015, NP_003444.1,
210281_s_at
FIG. 2209: PRO58410
FIG. 2210A-C: DNA194808, NM_003615,
210286_s_at
FIG. 2211: PRO24078
FIG. 2212: DNA272137, NP_000309.1, 210296_s_at
FIG. 2213: PRO60406
FIG. 2214A-B: DNA188419, NP_002011.1,
210316_at
FIG. 2215: PRO21767
FIG. 2216: DNA329213, NP_219491.1, 210321_at
FIG. 2217: PRO2313
FIG. 2218: DNA225528, NP_000610.1, 210354_at
FIG. 2219: PRO35991
FIG. 2220: DNA330207, BC001131, 210387_at
FIG. 2221: PRO85451
FIG. 2222A-B: DNA330208, AF164622,
210425_x_at
FIG. 2223: PRO85452
FIG. 2224: DNA344496, NP_599022.1, 210426_x_at
FIG. 2225: PRO95143
FIG. 2226: DNA329215, NP_036224.1, 210439_at
FIG. 2227: PRO7424
FIG. 2228: DNA344497, NP_002552.2, 210448_s_at
FIG. 2229: PRO95144
FIG. 2230: DNA344498, NM_133484, 210458_s_at
FIG. 2231: PRO86554
FIG. 2232: DNA326589, NP_060192.1, 210463_x_at
FIG. 2233: PRO82947
FIG. 2234: DNA323856, NM_015640, 210466_s_at
FIG. 2235: PRO80599
FIG. 2236A-B: DNA274461, M37712, 210473_s_at
FIG. 2237: PRO62367
FIG. 2238: DNA344499, NM_134262, 210479_s_at
FIG. 2239: PRO95145
FIG. 2240: DNA256385, NP_004470.1, 210506_at
FIG. 2241: PRO51426
FIG. 2242: DNA344500, NP_003367.2, 210512_s_at
FIG. 2243: PRO84827
FIG. 2244: DNA344501, NP_002118.1, 210514_x_at
FIG. 2245: PRO50891
FIG. 2246: DNA270066, AF078844, 210524_x_at
FIG. 2247: PRO58459
FIG. 2248: DNA344502, AF010447, 210528_at
FIG. 2249: PRO95146
FIG. 2250: DNA344503, NP_003769.1, 210540_s_at
FIG. 2251: PRO1109
FIG. 2252A-B: DNA344504, NP_004546.1,
210555_s_at
FIG. 2253: PRO82622
FIG. 2254A-B: DNA344505, NM_173164,
210556_at
FIG. 2255: PRO95147
FIG. 2256: DNA344506, NM_172211, 210557_x_at
FIG. 2257: PRO95148
FIG. 2258: DNA344507, NM_033379, 210559_s_at
FIG. 2259: PRO70806
FIG. 2260: DNA344508, U97075, 210563_x_at
FIG. 2261: PRO95149
FIG. 2262: DNA329217, AAH03406.1, 210571_s_at
FIG. 2263: PRO84828
FIG. 2264: DNA344509, AF241788, 210574_s_at
FIG. 2265: PRO95150
FIG. 2266: DNA327808, NM_002970, 210592_s_at
FIG. 2267: PRO83769
FIG. 2268: DNA227722, NM_002262, 210606_x_at
FIG. 2269: PRO38185
FIG. 2270: DNA330210, U03858, 210607_at
FIG. 2271: PRO126
FIG. 2272: DNA150511, AF000425, 210629_x_at
FIG. 2273: PRO11557
FIG. 2274: DNA344510, NP_003692.1, 210643_at
FIG. 2275: PRO1292
FIG. 2276: DNA227153, NP_002278.1, 210644_s_at
FIG. 2277: PRO37616
FIG. 2278A-C: DNA330214, D83077, 210645_s_at
FIG. 2279: PRO12135
FIG. 2280: DNA290260, NP_036555.1, 210646_x_at
FIG. 2281: PRO70385
FIG. 2282: DNA256521, NP_038459.1, 210690_at
FIG. 2283: PRO51556
FIG. 2284: DNA329218, NM_014412, 210691_s_at
FIG. 2285: PRO84829
FIG. 2286A-B: DNA335356, NM_000961,
210702_s_at
FIG. 2287: PRO25026
FIG. 2288: DNA329023, NP_066925.1, 210715_s_at
FIG. 2289: PRO209
FIG. 2290: DNA344511, BC015818, 210732_s_at
FIG. 2291: PRO95151
FIG. 2292: DNA103245, NM_002350, 210754_s_at
FIG. 2293: PRO4575
FIG. 2294: DNA194819, NP_667341.1, 210763_x_at
FIG. 2295: PRO24086
FIG. 2296: DNA344512, NP_001307.2, 210766_s_at
FIG. 2297: PRO83174
FIG. 2298: DNA103572, D14705, 210844_x_at
FIG. 2299: PRO4896
FIG. 2300: DNA344513, Y09392, 210847_x_at
FIG. 2301A-C: DNA329220, NM_000051,
210858_x_at
FIG. 2302: PRO84830
FIG. 2303: DNA188234, NP_000630.1, 210865_at
FIG. 2304: PRO21942
FIG. 2305: DNA228132, NM_024090, 210868_s_at
FIG. 2306: PRO38595
FIG. 2307: DNA344514, AF098641, 210916_s_at
FIG. 2308: PRO95153
FIG. 2309: DNA344515, NP_000061.1, 210944_s_at
FIG. 2310: PRO38022
FIG. 2311: DNA344516, NM_003711, 210946_at
FIG. 2312: PRO95154
FIG. 2313: DNA344517, AF294627, 210948_s_at
FIG. 2314: PRO95155
FIG. 2315: DNA344518, NP_004453.1, 210950_s_at
FIG. 2316: PRO81644
FIG. 2317: DNA274027, NM_004580, 210951_x_at
FIG. 2318: PRO61971
FIG. 2319: DNA336282, NM_001178, 210971_s_at
FIG. 2320: PRO61686
FIG. 2321A-B: DNA344519, NP_000595.1,
210973_s_at
FIG. 2322: PRO34231
FIG. 2323: DNA344520, U47674, 210980_s_at
FIG. 2324: PRO95156
FIG. 2325: DNA269888, NP_002073.1, 210981_s_at
FIG. 2326: PRO58286
FIG. 2327: DNA329221, NM_019111, 210982_s_at
FIG. 2328: PRO4555
FIG. 2329: DNA238565, NP_005907.2, 210983_s_at
FIG. 2330: PRO39210
FIG. 2331: DNA151825, NP_005891.1, 210993_s_at
FIG. 2332: PRO12900
FIG. 2333: DNA344521, NM_002184, 211000_s_at
FIG. 2334: PRO85478
FIG. 2335: DNA150135, NP_055202.1, 211005_at
FIG. 2336: PRO12232
FIG. 2337: DNA273498, L12723, 211015_s_at
FIG. 2338: PRO61480
FIG. 2339: DNA344522, BC002526, 211016_x_at
FIG. 2340: PRO95157
FIG. 2341A-C: DNA344523, NP_000480.2,
211022_s_at
FIG. 2342: PRO95158
FIG. 2343: DNA287198, NP_006073.1, 211058_x_at
FIG. 2344: PRO69484
FIG. 2345: DNA328698, NM_006153, 211063_s_at
FIG. 2346: PRO12168
FIG. 2347: DNA326974, NM_000967, 211073_x_at
FIG. 2348: PRO83285
FIG. 2349A-B: DNA235639, NP_000206.1,
211108_s_at
FIG. 2350: PRO38866
FIG. 2351: DNA304765, M30894, 211144_x_at
FIG. 2352: PRO71178
FIG. 2353: DNA196439, NM_003874, 211190_x_at
FIG. 2354: PRO24934
FIG. 2355: DNA344524, U96627, 211192_s_at
FIG. 2356: PRO95159
FIG. 2357: DNA330221, NP_056071.1, 211207_s_at
FIG. 2358: PRO85460
FIG. 2359: DNA270010, NM_002351, 211209_x_at
FIG. 2360: PRO58405
FIG. 2361: DNA344525, AF100539, 211210_x_at
FIG. 2362: PRO95160
FIG. 2363: DNA344526, AF100542, 211211_x_at
FIG. 2364: PRO95161
FIG. 2365: DNA151022, NM_001345, 211272_s_at
FIG. 2366: PRO12096
FIG. 2367: DNA344527, NM_004130, 211275_s_at
FIG. 2368: PRO95162
FIG. 2369A-B: DNA344528, NM_002600,
211302_s_at
FIG. 2370: PRO10691
FIG. 2371A-C: DNA328811, NM_002222,
211323_s_at
FIG. 2372: PRO84551
FIG. 2373A-B: DNA339333, NP_005537.3,
211339_s_at
FIG. 2374: PRO91137
FIG. 2375: DNA103395, U80737, 211352_s_at
FIG. 2376: PRO4723
FIG. 2377: DNA327754, NP_150634.1, 211367_s_at
FIG. 2378: PRO4526
FIG. 2379A-B: DNA339371, NP_054742.1,
211383_s_at
FIG. 2380: PRO91176
FIG. 2381: DNA327755, NP_115957.1, 211458_s_at
FIG. 2382: PRO83725
FIG. 2383: DNA93439, NM_006564, 211469_s_at
FIG. 2384: PRO4515
FIG. 2385: DNA324183, NM_001935, 211478_s_at
FIG. 2386: PRO80881
FIG. 2387: DNA344529, BC001173, 211501_s_at
FIG. 2388: PRO62214
FIG. 2389: DNA344530, NM_003376, 211527_x_at
FIG. 2390: PRO69153
FIG. 2391: DNA344531, NP_001005.1, 211542_x_at
FIG. 2392: PRO95163
FIG. 2393: DNA269888, NM_002082, 211543_s_at
FIG. 2394: PRO58286
FIG. 2395: DNA226578, NM_004354, 211559_s_at
FIG. 2396: PRO37041
FIG. 2397: DNA329031, NP_004890.2, 211566_x_at
FIG. 2398: PRO84699
FIG. 2399: DNA226255, NP_003047.1, 211576_s_at
FIG. 2400: PRO36718
FIG. 2401: DNA331572, AF000426, 211581_x_at
FIG. 2402: PRO86585
FIG. 2403: DNA196752, AF031136, 211583_x_at
FIG. 2404: PRO25202
FIG. 2405: DNA344532, NP_631958.1, 211597_s_at
FIG. 2406: PRO95164
FIG. 2407: DNA275389, M30448, 211623_s_at
FIG. 2408: PRO63052
FIG. 2409: DNA344533, M24668, 211633_x_at
FIG. 2410: PRO95165
FIG. 2411: DNA344534, L06101, 211641_x_at
FIG. 2412: DNA344535, M17565, 211654_x_at
FIG. 2413A-B: DNA103553, NM_000176,
211671_s_at
FIG. 2414: PRO4880
FIG. 2415A-B: DNA255619, AF054589,
211675_s_at
FIG. 2416: PRO50682
FIG. 2417: DNA188293, NP_000407.1, 211676_s_at
FIG. 2418: PRO21787
FIG. 2419: DNA327760, NP_114430.1, 211685_s_at
FIG. 2420: PRO83729
FIG. 2421: DNA88515, L41270, 211688_x_at
FIG. 2422: PRO2390
FIG. 2423: DNA344536, NM_000968, 211710_x_at
FIG. 2424: PRO95168
FIG. 2425: DNA344537, NM_178014, 211714_x_at
FIG. 2426: PRO10347
FIG. 2427A-B: DNA274117, NP_612356.1,
211721_s_at
FIG. 2428: PRO62054
FIG. 2429: DNA329225, NP_006486.2, 211742_s_at
FIG. 2430: PRO84833
FIG. 2431: DNA344538, NM_148976, 211746_x_at
FIG. 2432: PRO81959
FIG. 2433: DNA344539, NP_036454.1, 211747_s_at
FIG. 2434: PRO95169
FIG. 2435: DNA344540, BC021088, 211750_x_at
FIG. 2436: PRO84424
FIG. 2437: DNA324147, NP_005774.2, 211758_x_at
FIG. 2438: PRO80848
FIG. 2439: DNA344541, BC005974, 211760_s_at
FIG. 2440: PRO95170
FIG. 2441: DNA254725, NM_002266, 211762_s_at
FIG. 2442: PRO49824
FIG. 2443: DNA340145, NM_012307, 211776_s_at
FIG. 2444: PRO91644
FIG. 2445: DNA344542, NM_001561, 211786_at
FIG. 2446: PRO2023
FIG. 2447: DNA344543, NP_003627.1, 211791_s_at
FIG. 2448: PRO62306
FIG. 2449: DNA331536, AAA60662.1, 211796_s_at
FIG. 2450: PRO86563
FIG. 2451: DNA344544, NM_052827, 211804_s_at
FIG. 2452: PRO95171
FIG. 2453A-B: DNA225940, NP_000144.1,
211810_s_at
FIG. 2454: PRO36403
FIG. 2455A-B: DNA328707, AAF03782.1,
211828_s_at
FIG. 2456: PRO84466
FIG. 2457: DNA344545, NM_138763, 211833_s_at
FIG. 2458: PRO95172
FIG. 2459: DNA344546, NP_757351.1, 211839_s_at
FIG. 2460: PRO95173
FIG. 2461A-B: DNA188192, NP_006130.1,
211856_x_at
FIG. 2462: PRO21704
FIG. 2463A-B: DNA188192, NM_006139,
211861_x_at
FIG. 2464: PRO21704
FIG. 2465: DNA225836, NM_006725, 211893_x_at
FIG. 2466: PRO36299
FIG. 2467: DNA344547, U6614.6, 211900_x_at
FIG. 2468: PRO95174
FIG. 2469: DNA226176, NM_003467, 211919_s_at
FIG. 2470: PRO36639
FIG. 2471: DNA272286, NM_001752, 211922_s_at
FIG. 2472: PRO60544
FIG. 2473: DNA344548, 7762146.13, 211929_at
FIG. 2474: PRO95175
FIG. 2475A-B: DNA272195, D21262, 211951_at
FIG. 2476: DNA325941, NP_005339.1, 211969_at
FIG. 2477: PRO82388
FIG. 2478: DNA344549, 474771.15, 211974_x_at
FIG. 2479: PRO95176
FIG. 2480A-B: DNA344550, BC047523, 211984_at
FIG. 2481: PRO4904
FIG. 2482A-B: DNA344551, 7698619.16,
211985_s_at
FIG. 2483: PRO95177
FIG. 2484A-C: DNA327765, 1390535.1, 211986_at
FIG. 2485: PRO83732
FIG. 2486: DNA344552, NP_291032.1, 211990_at
FIG. 2487: PRO85469
FIG. 2488: DNA324768, NM_033554, 211991_s_at
FIG. 2489: PRO4884
FIG. 2490: DNA326406, NP_005315.1, 211999_at
FIG. 2491: PRO11403
FIG. 2492: DNA287433, NP_006810.1, 212009_s_at
FIG. 2493: PRO69690
FIG. 2494: DNA88197, X66733, 212014_x_at
FIG. 2495: PRO2694
FIG. 2496A-D: DNA103461, NP_002408.2,
212020_s_at
FIG. 2497: PRO4788
FIG. 2498A-D: DNA103461, NM_002417,
212022_s_at
FIG. 2499: PRO4788
FIG. 2500A-D: DNA226463, X65551, 212023_s_at
FIG. 2501: PRO36926
FIG. 2502: DNA328709, BC004151, 212048_s_at
FIG. 2503: PRO37676
FIG. 2504A-B: DNA344553, 7697666.18, 212063_at
FIG. 2505: PRO95178
FIG. 2506A-D: DNA344554, BAA25496.2,
212065_s_at
FIG. 2507: PRO95179
FIG. 2508: DNA344555, NP_065800.1, 212096_s_at
FIG. 2509: PRO95180
FIG. 2510: DNA325009, NP_001744.2, 212097_at
FIG. 2511: PRO81600
FIG. 2512: DNA344556, AF055029, 212098_at
FIG. 2513: PRO95181
FIG. 2514: DNA344557, 7763517.13, 212099_at
FIG. 2515: PRO95182
FIG. 2516A-B: DNA150956, BAA06685.1,
212110_at
FIG. 2517: PRO12560
FIG. 2518: DNA344558, AF070622, 212124_at
FIG. 2519: PRO95183
FIG. 2520: DNA151008, BC014044, 212125_at
FIG. 2521: PRO12837
FIG. 2522: DNA330242, BC007034, 212185_x_at
FIG. 2523: PRO85477
FIG. 2524: DNA330243, NP_006207.1, 212190_at
FIG. 2525: PRO2584
FIG. 2526: DNA326233, NM_000977, 212191_x_at
FIG. 2527: PRO82645
FIG. 2528A-C: DNA330244, 253946.17, 212195_at
FIG. 2529: PRO85478
FIG. 2530: DNA328437, NM_005801, 212227_x_at
FIG. 2531: PRO84271
FIG. 2532: DNA151120, M61906, 212240_s_at
FIG. 2533: PRO12179
FIG. 2534A-B: DNA329229, 1345070.7, 212249_at
FIG. 2535: PRO84835
FIG. 2536: DNA329182, NM_020524, 212259_s_at
FIG. 2537: PRO84805
FIG. 2538A-B: DNA344559, 332723.7, 212290_at
FIG. 2539: PRO95184
FIG. 2540: DNA344560, AL833829, 212291_at
FIG. 2541: DNA328719, BC012895, 212295_s_at
FIG. 2542: PRO84475
FIG. 2543A-B: DNA344561, AL832633, 212299_at
FIG. 2544: PRO95186
FIG. 2545A-B: DNA344562, 319543.9, 212314_at
FIG. 2546: PRO95187
FIG. 2547A-B: DNA124122, NP_005602.2,
212331_at
FIG. 2548: PRO6323
FIG. 2549A-B: DNA124122, NM_005611,
212332_at
FIG. 2550: PRO6323
FIG. 2551: DNA287190, CAB43217.1, 212333_at
FIG. 2552: PRO69476
FIG. 2553: DNA344563, BC017742, 212334_at
FIG. 2554: PRO95188
FIG. 2555A-B: DNA344564, 254170.1, 212335_at
FIG. 2556: PRO2759
FIG. 2557A-B: DNA255527, D50525, 212337_at
FIG. 2558: DNA344565, BC040726, 212359_s_at
FIG. 2559A-B: DNA269762, BAA25456.1,
212368_at
FIG. 2560: PRO58171
FIG. 2561A-B: DNA344566, BAA25518.1,
212370_x_at
FIG. 2562: PRO95190
FIG. 2563A-C: DNA330249, AAA99177.1,
212372_at
FIG. 2564: PRO85482
FIG. 2565A-C: DNA344567, 020294.13, 212386_at
FIG. 2566: PRO95191
FIG. 2567A-C: DNA328725, AB007923, 212390_at
FIG. 2568A-B: DNA328549, NP_002897.1,
212397_at
FIG. 2569: PRO84350
FIG. 2570A-B: DNA328549, NM_002906,
212398_at
FIG. 2571: PRO84350
FIG. 2572A-B: DNA344568, AK074108, 212400_at
FIG. 2573A-B: DNA330250, NP_060727.1,
212406_s_at
FIG. 2574: PRO85483
FIG. 2575: DNA254828, NP_056417.1, 212408_at
FIG. 2576: PRO49923
FIG. 2577: DNA344569, 1454838.10, 212412_at
FIG. 2578: PRO95192
FIG. 2579: DNA330251, NP_059965.1, 212430_at
FIG. 2580: PRO85484
FIG. 2581: DNA304655, NP_079472.1, 212434_at
FIG. 2582: PRO71082
FIG. 2583A-B: DNA344570, 481983.1, 212446_s_at
FIG. 2584: PRO95193
FIG. 2585: DNA344571, AF052178, 212458_at
FIG. 2586: PRO95194
FIG. 2587: DNA151348, DNA151348, 212463_at
FIG. 2588: PRO11726
FIG. 2589: DNA344572, 226098.35, 212472_at
FIG. 2590: PRO95195
FIG. 2591A-B: DNA330252, NP_055447.1,
212473_s_at
FIG. 2592: PRO85485
FIG. 2593A-B: DNA344573, D26069, 212476_at
FIG. 2594A-C: DNA344574, NP_597677.1,
212483_at
FIG. 2595: PRO95197
FIG. 2596: DNA344575, 7762745.4, 212498_at
FIG. 2597: PRO95198
FIG. 2598: DNA344576, NP_005185.2, 212501_at
FIG. 2599: PRO91094
FIG. 2600A-B: DNA344577, NP_116193.1,
212502_at
FIG. 2601: PRO84485
FIG. 2602: DNA344578, 1307005.1, 212511_at
FIG. 2603: PRO95199
FIG. 2604A-B: DNA344579, BC036190, 212522_at
FIG. 2605: PRO95200
FIG. 2606: DNA328733, AF038183, 212527_at
FIG. 2607: PRO84486
FIG. 2608: DNA344580, AL080111, 212530_at
FIG. 2609: PRO95201
FIG. 2610A-C: DNA344581, NP_056111.1,
212538_at
FIG. 2611: PRO95202
FIG. 2612: DNA65407, DNA65407, 212558_at
FIG. 2613: PRO1276
FIG. 2614A-D: DNA328737, 148650.1, 212560_at
FIG. 2615: PRO84490
FIG. 2616A-B: DNA254958, AL117448, 212561_at
FIG. 2617: DNA344582, NP_056016.1, 212563_at
FIG. 2618: PRO81715
FIG. 2619: DNA344583, BC039084, 212568_s_at
FIG. 2620: PRO95203
FIG. 2621A-C: DNA331128, NP_065892.1,
212582_at
FIG. 2622: PRO84841
FIG. 2623A-B: DNA333749, NP_002829.2,
212587_s_at
FIG. 2624: PRO88374
FIG. 2625: DNA275100, DNA275100, 212589_at
FIG. 2626: DNA331327, NM_012250, 212590_at
FIG. 2627: PRO86414
FIG. 2628: DNA331298, NM_014456, 212593_s_at
FIG. 2629: PRO81909
FIG. 2630: DNA272928, NP_055579.1, 212595_s_at
FIG. 2631: PRO61012
FIG. 2632: DNA344584, 253648.3, 212613_at
FIG. 2633: PRO95204
FIG. 2634A-B: DNA330258, BAA22955.2,
212619_at
FIG. 2635: PRO85490
FIG. 2636A-B: DNA344585, AL833311, 212621_at
FIG. 2637: PRO95205
FIG. 2638: DNA194679, BAA05062.1, 212623_at
FIG. 2639: PRO23989
FIG. 2640: DNA344586, AL050082, 212637_s_at
FIG. 2641: PRO95206
FIG. 2642A-C: DNA344587, NP_006725.2,
212641_at
FIG. 2643: PRO95207
FIG. 2644A-C: DNA344588, NM_006734,
212642_s_at
FIG. 2645: PRO95208
FIG. 2646: DNA329031, NM_004899, 212645_x_at
FIG. 2647: PRO84699
FIG. 2648: DNA344589, NP_000568.1, 212657_s_at
FIG. 2649: PRO83789
FIG. 2650A-B: DNA344590, D87076, 212660_at
FIG. 2651: DNA344591, L34089, 212671_s_at
FIG. 2652A-D: DNA344592, 032872.20, 212672_at
FIG. 2653: PRO84830
FIG. 2654: DNA344593, AF515797, 212681_at
FIG. 2655A-B: DNA329901, BAA32291.2,
212683_at
FIG. 2656: PRO85218
FIG. 2657: DNA272355, L38935, 212697_at
FIG. 2658: DNA326234, NM_033251, 212734_x_at
FIG. 2659: PRO82646
FIG. 2660: DNA290267, NP_005000.1, 212739_s_at
FIG. 2661: PRO70399
FIG. 2662A-B: DNA327779, 363462.9, 212741_at
FIG. 2663: PRO83744
FIG. 2664A-B: DNA273398, NM_015568,
212750_at
FIG. 2665: PRO61398
FIG. 2666A-B: DNA344594, NP_751911.1,
212757_s_at
FIG. 2667: PRO95212
FIG. 2668: DNA344595, AAH34232.1, 212771_at
FIG. 2669: PRO95213
FIG. 2670A-C: DNA344596, AB029032, 212779_at
FIG. 2671: DNA290260, NM_012423, 212790_x_at
FIG. 2672: PRO70385
FIG. 2673A-B: DNA150479, BAA74900.1,
212792_at
FIG. 2674: PRO12281
FIG. 2675A-B: DNA344597, NP_055894.1,
212796_s_at
FIG. 2676: PRO95215
FIG. 2677: DNA328750, 7689361.1, 212812_at
FIG. 2678: PRO84500
FIG. 2679A-C: DNA336121, AB020663, 212820_at
FIG. 2680A-B: DNA344598, BAB84995.1,
212823_s_at
FIG. 2681: PRO95216
FIG. 2682: DNA330171, CAA34971.1, 212827_at
FIG. 2683: PRO85421
FIG. 2684: DNA344599, 234498.36, 212847_at
FIG. 2685: PRO95217
FIG. 2686: DNA344600, AL713742, 212886_at
FIG. 2687: PRO95218
FIG. 2688: DNA344601, 989341.96, 212906_at
FIG. 2689: PRO85986
FIG. 2690: DNA271630, DNA271630, 212907_at
FIG. 2691: DNA272939, NP_064582.1, 212922_s_at
FIG. 2692: PRO61023
FIG. 2693: DNA344602, BC045715, 212923_s_at
FIG. 2694A-B: DNA344603, AB011164,
212929_s_at
FIG. 2695A-B: DNA272008, BAA06684.1,
212932_at
FIG. 2696: PRO60283
FIG. 2697: DNA344604, NP_056156.2, 212949_at
FIG. 2698: PRO80842
FIG. 2699: DNA255330, AL359588, 212959_s_at
FIG. 2700: DNA344605, U66042, 212961_x_at
FIG. 2701: PRO50485
FIG. 2702: DNA325417, NP_001742.1, 212971_at
FIG. 2703: PRO69635
FIG. 2704A-B: DNA344606, 474311.10, 212985_at
FIG. 2705: PRO95220
FIG. 2706: DNA344607, NM_147156, 212989_at
FIG. 2707: PRO50467
FIG. 2708: DNA344608, BC038387, 213010_at
FIG. 2709A-C: DNA327783, DNA327783,
213015_at
FIG. 2710: PRO83747
FIG. 2711A-B: DNA253815, BAA20833.2,
213035_at
FIG. 2712: PRO49218
FIG. 2713A-B: DNA344609, NM_174953,
213036_x_at
FIG. 2714: PRO95221
FIG. 2715: DNA344610, NP_699172.1, 213038_at
FIG. 2716: PRO95222
FIG. 2717A-B: DNA329242, BAA76857.1,
213056_at
FIG. 2718: PRO84847
FIG. 2719: DNA323879, NP_003991.1, 213060_s_at
FIG. 2720: PRO80622
FIG. 2721A-C: DNA328757, 475076.9, 213069_at
FIG. 2722: PRO84506
FIG. 2723: DNA150837, CAA06743.1, 213083_at
FIG. 2724: PRO12495
FIG. 2725: DNA344611, NP_000975.2, 213084_x_at
FIG. 2726: PRO95223
FIG. 2727A-B: DNA331353, BAA76818.1,
213092_x_at
FIG. 2728: PRO60758
FIG. 2729: DNA270466, M12996, 213093_at
FIG. 2730A-B: DNA339968, BAA76825.1,
213111_at
FIG. 2731: PRO91476
FIG. 2732: DNA330215, NP_060081.1, 213113_s_at
FIG. 2733: PRO24295
FIG. 2734: DNA326217, NP_004474.1, 213129_s_at
FIG. 2735: PRO82630
FIG. 2736: DNA344612, NM_006806, 213134_x_at
FIG. 2737: PRO95224
FIG. 2738: DNA287230, AAA36325.1, 213138_at
FIG. 2739: PRO69509
FIG. 2740: DNA330277, CAB45152.1, 213142_x_at
FIG. 2741: PRO85506
FIG. 2742A-B: DNA344613, 1330122.30, 213164_at
FIG. 2743: PRO95225
FIG. 2744: DNA344614, X17568, 213175_s_at
FIG. 2745: PRO95226
FIG. 2746: DNA344615, AF279370, 213186_at
FIG. 2747: DNA344616, NP_705833.1, 213188_s_at
FIG. 2748: PRO95227
FIG. 2749: DNA339710, NP_116167.3, 213189_at
FIG. 2750: PRO91439
FIG. 2751: DNA344617, K02885, 213193_x_at
FIG. 2752: DNA344618, 1501943.6, 213206_at
FIG. 2753: PRO95229
FIG. 2754: DNA344619, 1398007.8, 213226_at
FIG. 2755: PRO95230
FIG. 2756A-B: DNA344620, NP_065186.2,
213238_at
FIG. 2757: PRO95231
FIG. 2758A-B: DNA194850, BAA25458.1,
213243_at
FIG. 2759: PRO24112
FIG. 2760A-C: DNA344621, BAA20800.2,
213261_at
FIG. 2761: PRO59767
FIG. 2762A-B: DNA344622, AY217548, 213281_at
FIG. 2763: PRO4671
FIG. 2764: DNA260974, NP_006065.1, 213293_s_at
FIG. 2765: PRO54720
FIG. 2766A-B: DNA329248, BAA20816.1,
213302_at
FIG. 2767: PRO84850
FIG. 2768A-B: DNA331295, NM_002719,
213305_s_at
FIG. 2769: PRO86394
FIG. 2770A-B: DNA344623, NP_055999.1,
213309_at
FIG. 2771: PRO95232
FIG. 2772: DNA344624, AY074889, 213315_x_at
FIG. 2773: PRO95233
FIG. 2774: DNA344625, BC020923, 213317_at
FIG. 2775: PRO95234
FIG. 2776: DNA344626, AAH19339.1, 213320_at
FIG. 2777: PRO95235
FIG. 2778A-B: DNA344627, AF022789,
213327_s_at
FIG. 2779: DNA287433, NM_006819, 213330_s_at
FIG. 2780: PRO69690
FIG. 2781A-B: DNA274793, BAA96028.1,
213365_at
FIG. 2782: PRO62559
FIG. 2783: DNA324853, NP_001007.2, 213377_x_at
FIG. 2784: PRO81462
FIG. 2785: DNA344628, 222320.2, 213385_at
FIG. 2786: PRO95237
FIG. 2787A-B: DNA344629, 7697344.6, 213416_at
FIG. 2788: PRO95238
FIG. 2789A-B: DNA331398, DNA331398,
213457_at
FIG. 2790: PRO83924
FIG. 2791A-B: DNA330285, 241020.1, 213469_at
FIG. 2792: PRO85513
FIG. 2793A-B: DNA344630, NP_055917.1,
213471_at
FIG. 2794: PRO95239
FIG. 2795: DNA328766, NP_006077.1, 213476_x_at
FIG. 2796: PRO84514
FIG. 2797A-B: DNA344631, NM_002265,
213507_s_at
FIG. 2798: PRO82739
FIG. 2799: DNA326639, NP_001229.1, 213523_at
FIG. 2800: PRO82992
FIG. 2801: DNA324005, NP_056529.1, 213524_s_at
FIG. 2802: PRO11582
FIG. 2803: DNA344632, BC022977, 213530_at
FIG. 2804A-B: DNA344633, 062042.23,
213531_s_at
FIG. 2805: PRO95240
FIG. 2806: DNA254264, NP_689960.1, 213546_at
FIG. 2807: PRO49375
FIG. 2808: DNA344634, NM_144781, 213581_at
FIG. 2809: PRO95241
FIG. 2810: DNA344635, AAH15899.1, 213587_s_at
FIG. 2811: PRO95242
FIG. 2812: DNA326426, NP_004300.1, 213606_s_at
FIG. 2813: PRO61246
FIG. 2814A-C: DNA330292, NP_056045.2,
213618_at
FIG. 2815: PRO85519
FIG. 2816: DNA344636, BC045542, 213623_at
FIG. 2817: PRO95243
FIG. 2818: DNA344637, NP_005940.1, 213629_x_at
FIG. 2819: PRO95244
FIG. 2820: DNA326239, NP_006752.1, 213655_at
FIG. 2821: PRO39530
FIG. 2822: DNA325704, NM_004990, 213671_s_at
FIG. 2823: PRO82188
FIG. 2824: DNA344638, AK057596, 213703_at
FIG. 2825: PRO95245
FIG. 2826: DNA328629, NM_006088, 213726_x_at
FIG. 2827: PRO84407
FIG. 2828: DNA334387, NP_075563.2, 213727_x_at
FIG. 2829: PRO88903
FIG. 2830A-B: DNA344639, NP_036467.2,
213733_at
FIG. 2831: PRO95246
FIG. 2832: DNA326273, NM_001970, 213757_at
FIG. 2833: PRO82678
FIG. 2834: DNA327804, AF442151, 213797_at
FIG. 2835: PRO69493
FIG. 2836A-B: DNA344640, 7684018.188,
213803_at
FIG. 2837: PRO95247
FIG. 2838: DNA344641, 233172.5, 213852_at
FIG. 2839: PRO95248
FIG. 2840: DNA344642, 026641.16, 213888_s_at
FIG. 2841: PRO95249
FIG. 2842: DNA272347, NP_001011.1, 213890_x_at
FIG. 2843: PRO60603
FIG. 2844: DNA151041, X66087, 213906_at
FIG. 2845: DNA333671, NP_005592.1, 213915_at
FIG. 2846: PRO37543
FIG. 2847: DNA327806, 242985.1, 213929_at
FIG. 2848: PRO83767
FIG. 2849: DNA344643, 1454455.7, 213931_at
FIG. 2850: PRO95250
FIG. 2851A-D: DNA339387, NM_014810,
213956_at
FIG. 2852: PRO91192
FIG. 2853: DNA344644, BC033755, 213958_at
FIG. 2854: PRO95251
FIG. 2855: DNA226014, NP_000230.1, 213975_s_at
FIG. 2856: PRO36477
FIG. 2857: DNA344645, AL050290, 213988_s_at
FIG. 2858: PRO95252
FIG. 2859: DNA344646, AF305069, 213996_at
FIG. 2860: PRO86433
FIG. 2861: DNA329136, NM_016391, 214011_s_at
FIG. 2862: PRO84772
FIG. 2863: DNA150990, NM_003641, 214022_s_at
FIG. 2864: PRO12570
FIG. 2865: DNA344647, BC013297, 214049_x_at
FIG. 2866: PRO84853
FIG. 2867: DNA330298, NP_005403.2, 214095_at
FIG. 2868: PRO83772
FIG. 2869: DNA330298, NM_005412, 214096_s_at
FIG. 2870: PRO83772
FIG. 2871: DNA344648, L43578, 214112_s_at
FIG. 2872: DNA344649, NP_005096.1, 214113_s_at
FIG. 2873: PRO37600
FIG. 2874: DNA344650, 127586.127, 214129_at
FIG. 2875: PRO95254
FIG. 2876: DNA344651, 1500085.15, 214163_at
FIG. 2877: PRO95255
FIG. 2878: DNA344652, 236569.38, 214169_at
FIG. 2879: PRO95256
FIG. 2880: DNA329182, BC016852, 214177_s_at
FIG. 2881: PRO84805
FIG. 2882A-B: DNA269826, NP_003195.1,
214179_s_at
FIG. 2883: PRO58228
FIG. 2884: DNA344653, NM_000391, 214196_s_at
FIG. 2885: PRO95257
FIG. 2886: DNA331361, NP_003318.1, 214228_x_at
FIG. 2887: PRO2398
FIG. 2888: DNA344654, 264912.4, 214241_at
FIG. 2889: PRO95258
FIG. 2890: DNA344655, 202212.8, 214329_x_at
FIG. 2891: PRO95259
FIG. 2892: DNA344656, NP_203524.1, 214352_s_at
FIG. 2893: PRO95260
FIG. 2894: DNA304680, NM_007355, 214359_s_at
FIG. 2895: PRO71106
FIG. 2896: DNA273138, NP_005495.1, 214390_s_at
FIG. 2897: PRO61182
FIG. 2898: DNA344657, AK097004, 214402_s_at
FIG. 2899: PRO95261
FIG. 2900: DNA287630, NP_000160.1, 214430_at
FIG. 2901: PRO2154
FIG. 2902: DNA344658, BC039858, 214435_x_at
FIG. 2903: PRO12184
FIG. 2904A-B: DNA344659, NP_036213.1,
214446_at
FIG. 2905: PRO37794
FIG. 2906: DNA331744, NP_001326.2, 214450_at
FIG. 2907: PRO1574
FIG. 2908: DNA327812, NP_006408.2, 214453_s_at
FIG. 2909: PRO83773
FIG. 2910: DNA150971, NP_002249.1, 214470_at
FIG. 2911: PRO12564
FIG. 2912: DNA329253, NP_006128.1, 214551_s_at
FIG. 2913: PRO84853
FIG. 2914: DNA80218, U23772, 214567_s_at
FIG. 2915: PRO1610
FIG. 2916: DNA344660, AF001892, 214657_s_at
FIG. 2917: PRO95262
FIG. 2918: DNA330303, BAA05499.1, 214662_at
FIG. 2919: PRO85528
FIG. 2920: DNA328785, NP_004062.1, 214683_s_at
FIG. 2921: PRO84531
FIG. 2922: DNA344661, NP_006622.1, 214686_at
FIG. 2923: PRO95263
FIG. 2924A-B: DNA344662, AB002326,
214707_x_at
FIG. 2925: DNA344663, AB046861, 214723_x_at
FIG. 2926A-B: DNA334132, BAB21826.1,
214724_at
FIG. 2927: PRO88686
FIG. 2928A-B: DNA344664, 350410.3, 214787_at
FIG. 2929: PRO95266
FIG. 2930: DNA339733, NP_612411.2, 214791_at
FIG. 2931: PRO91461
FIG. 2932A-B: DNA344665, AAH42045.1,
214855_s_at
FIG. 2933: PRO95267
FIG. 2934A-E: DNA344666, L39064, 214950_at
FIG. 2935: DNA344667, NP_009198.3, 214958_s_at
FIG. 2936: PRO95269
FIG. 2937A-B: DNA344668, NP_003023.1,
214971_s_at
FIG. 2938: PRO54745
FIG. 2939: DNA344669, NP_003819.1, 214975_s_at
FIG. 2940: PRO95270
FIG. 2941: DNA327532, NM_002065, 215001_s_at
FIG. 2942: PRO71134
FIG. 2943: DNA344670, U90551, 215071_s_at
FIG. 2944: PRO85534
FIG. 2945: DNA344671, 212023.3, 215100_at
FIG. 2946: PRO23679
FIG. 2947: DNA344672, 350922.19, 215133_s_at
FIG. 2948: PRO95271
FIG. 2949: DNA344673, AAH20773.1, 215136_s_at
FIG. 2950: PRO84861
FIG. 2951: DNA273371, NP_000364.1, 215165_x_at
FIG. 2952: PRO61373
FIG. 2953: DNA324015, NM_006335, 215171_s_at
FIG. 2954: PRO80735
FIG. 2955: DNA344674, NP_056420.1, 215172_at
FIG. 2956: PRO95272
FIG. 2957A-B: DNA150496, AB023212, 215175_at
FIG. 2958: DNA324269, NP_006345.1, 215273_s_at
FIG. 2959: PRO80952
FIG. 2960A-B: DNA255050, NM_020432,
215286_s_at
FIG. 2961: PRO50138
FIG. 2962: DNA254588, AL049782, 215318_at
FIG. 2963: DNA344675, 7763519.36, 215338_s_at
FIG. 2964: PRO95273
FIG. 2965: DNA336791, BC027954, 215345_x_at
FIG. 2966: PRO90861
FIG. 2967: DNA327831, NP_076956.1, 215380_s_at
FIG. 2968: PRO83783
FIG. 2969: DNA331570, AAH15794.1, 215440_s_at
FIG. 2970: PRO84545
FIG. 2971: DNA344676, NM_152876, 215719_x_at
FIG. 2972: PRO95274
FIG. 2973: DNA273821, X98258, 215731_s_at
FIG. 2974: DNA344677, NP_000944.1, 215894_at
FIG. 2975: PRO95275
FIG. 2976: DNA330324, NP_002720.1, 215933_s_at
FIG. 2977: PRO58034
FIG. 2978: DNA344678, 1452291.4, 216133_at
FIG. 2979: PRO23844
FIG. 2980: DNA344679, AAA61033.1, 216191_s_at
FIG. 2981: PRO95276
FIG. 2982A-B: DNA344680, NM_015184,
216218_s_at
FIG. 2983: PRO95277
FIG. 2984: DNA344681, NM_173172, 216248_s_at
FIG. 2985: PRO95278
FIG. 2986: DNA326994, NP_055955.1, 216251_s_at
FIG. 2987: PRO83301
FIG. 2988: DNA344682, NM_152873, 216252_x_at
FIG. 2989: PRO95279
FIG. 2990A-C: DNA270933, NM_006766,
216361_s_at
FIG. 2991: PRO59265
FIG. 2992: DNA344683, X80821, 216563_at
FIG. 2993: DNA287243, NP_004452.1, 216602_s_at
FIG. 2994: PRO69518
FIG. 2995A-C: DNA150435, NP_055444.1,
216620_s_at
FIG. 2996: PRO12247
FIG. 2997: DNA226699, NM_000022, 216705_s_at
FIG. 2998: PRO37162
FIG. 2999: DNA344684, BC026029, 216804_s_at
FIG. 3000: PRO95280
FIG. 3001: DNA329135, NP_002913.2, 216834_at
FIG. 3002: PRO58102
FIG. 3003: DNA227597, NP_000627.1, 216841_s_at
FIG. 3004: PRO38060
FIG. 3005: DNA344685, L76665, 216907_x_at
FIG. 3006: PRO95281
FIG. 3007: DNA328810, NM_001779, 216942_s_at
FIG. 3008: PRO2557
FIG. 3009A-C: DNA103378, U23850, 216944_s_at
FIG. 3010: PRO4708
FIG. 3011: DNA275181, NM_303090, 216977_x_at
FIG. 3012: PRO62882
FIG. 3013: DNA344686, NP_543157.1, 217025_s_at
FIG. 3014: PRO95282
FIG. 3015: DNA331366, L06797, 217028_at
FIG. 3016: PRO4516
FIG. 3017: DNA329073, NP_004830.1, 217080_s_at
FIG. 3018: PRO84731
FIG. 3019A-B: DNA328813, BAA76774.1,
217118_s_at
FIG. 3020: PRO84553
FIG. 3021: DNA227752, NM_001504, 217119_s_at
FIG. 3022: PRO38215
FIG. 3023A-B: DNA329269, BAA32292.2,
217122_s_at
FIG. 3024: PRO84865
FIG. 3025: DNA340209, NP_114093.1, 217123_x_at
FIG. 3026: PRO91704
FIG. 3027: DNA344687, NP_001893.2, 217127_at
FIG. 3028: PRO84866
FIG. 3029: DNA103549, M21624, 217143_s_at
FIG. 3030: PRO4876
FIG. 3031: DNA227786, NP_057472.1, 217147_s_at
FIG. 3032: PRO38249
FIG. 3033: DNA344688, NM_005949, 217165_x_at
FIG. 3034: PRO95283
FIG. 3035: DNA344689, NM_176786, 217212_s_at
FIG. 3036: PRO95284
FIG. 3037: DNA344690, D84140, 217235_x_at
FIG. 3038: DNA151105, NP_005601.1, 217301_x_at
FIG. 3039: PRO12857
FIG. 3040: DNA344691, X69383, 217381_s_at
FIG. 3041: PRO95286
FIG. 3042: DNA344692, D13079, 217394_at
FIG. 3043: PRO95287
FIG. 3044: DNA344693, BC047570, 217403_s_at
FIG. 3045: PRO95288
FIG. 3046: DNA344694, 7697666.21, 217523_at
FIG. 3047: PRO95289
FIG. 3048: DNA344695, 023453.1, 217540_at
FIG. 3049: PRO95290
FIG. 3050: DNA344696, 346253.1, 217550_at
FIG. 3051: PRO95291
FIG. 3052: DNA344697, AK074970, 217724_at
FIG. 3053: PRO95292
FIG. 3054: DNA323856, AL080119, 217725_x_at
FIG. 3055: PRO80599
FIG. 3056: DNA325832, NP_068839.1, 217731_s_at
FIG. 3057: PRO1869
FIG. 3058: DNA325832, NM_021999, 217732_s_at
FIG. 3059: PRO1869
FIG. 3060A-B: DNA327847, 142131.14, 217738_at
FIG. 3061: PRO2834
FIG. 3062: DNA88541, NP_005737.1, 217739_s_at
FIG. 3063: PRO2834
FIG. 3064: DNA227205, NP_071404.1, 217744_s_at
FIG. 3065: PRO37668
FIG. 3066: DNA344698, NP_057001.1, 217751_at
FIG. 3067: PRO95293
FIG. 3068: DNA325910, NR_057110.2, 217776_at
FIG. 3069: PRO82365
FIG. 3070: DNA328819, NP_057145.1, 217783_s_at
FIG. 3071: PRO84557
FIG. 3072: DNA325873, NP_006100.2, 217786_at
FIG. 3073: PRO82331
FIG. 3074A-B: DNA254292, NP_004472.1,
217787_s_at
FIG. 3075: PRO49403
FIG. 3076A-B: DNA254292, NM_004481,
217788_s_at
FIG. 3077: PRO49403
FIG. 3078: DNA344699, NP_005709.1, 217818_s_at
FIG. 3079: PRO80955
FIG. 3080: DNA344700, BC032643, 217832_at
FIG. 3081: PRO95294
FIG. 3082: DNA344701, BC040844, 217834_s_at
FIG. 3083: PRO95295
FIG. 3084: DNA328823, NP_057421.1, 217838_s_at
FIG. 3085: PRO84561
FIG. 3086: DNA344702, NP_066952.1, 217848_s_at
FIG. 3087: PRO11669
FIG. 3088A-B: DNA324921, NP_073585.6,
217853_at
FIG. 3089: PRO81523
FIG. 3090: DNA344703, NP_002686.2, 217854_s_at
FIG. 3091: PRO95296
FIG. 3092: DNA344704, NP_060904.1, 217865_at
FIG. 3093: PRO95297
FIG. 3094: DNA335592, NP_036237.2, 217867_x_at
FIG. 3095: PRO852
FIG. 3096: DNA344705, NP_001247.2, 217879_at
FIG. 3097: PRO95298
FIG. 3098: DNA255145, NP_060917.1, 217882_at
FIG. 3099: PRO50225
FIG. 3100A-B: DNA325652, NP_057441.1,
217892_s_at
FIG. 3101: PRO82143
FIG. 3102: DNA330345, NP_055130.1, 217906_at
FIG. 3103: PRO85566
FIG. 3104: DNA328826, NP_004272.2, 217911_s_at
FIG. 3105: PRO84564
FIG. 3106: DNA344706, NP_751918.1, 217919_s_at
FIG. 3107: PRO95299
FIG. 3108: DNA287241, NP_056991.1, 217933_s_at
FIG. 3109: PRO69516
FIG. 3110A-B: DNA225648, NP_061165.1,
217941_s_at
FIG. 3111: PRO36111
FIG. 3112: DNA326730, NP_057037.1, 217950_at
FIG. 3113: PRO83072
FIG. 3114: DNA329273, NP_037374.1, 217957_at
FIG. 3115: PRO84869
FIG. 3116A-B: DNA272661, NP_443198.1,
217966_s_at
FIG. 3117: PRO60787
FIG. 3118A-B: DNA272661, NM_052966,
217967_s_at
FIG. 3119: PRO60787
FIG. 3120: DNA329546, NP_055214.1, 217979_at
FIG. 3121: PRO296
FIG. 3122: DNA227218, NP_003721.2, 217983_s_at
FIG. 3123: PRO37681
FIG. 3124: DNA227218, NM_003730, 217984_at
FIG. 3125: PRO37681
FIG. 3126: DNA328831, NP_057329.1, 217989_at
FIG. 3127: PRO233
FIG. 3128: DNA344707, NP_663768.1, 217991_x_at
FIG. 3129: PRO95300
FIG. 3130: DNA328832, NP_067022.1, 217995_at
FIG. 3131: PRO84568
FIG. 3132: DNA328833, BC018929, 217996_at
FIG. 3133: PRO84569
FIG. 3134: DNA328834, AF220656, 217997_at
FIG. 3135: DNA287364, NP_031376.1, 218000_s_at
FIG. 3136: PRO69625
FIG. 3137: DNA326005, NP_057004.1, 218007_s_at
FIG. 3138: PRO82446
FIG. 3139: DNA273008, NP_003972.1, 218009_s_at
FIG. 3140: PRO61079
FIG. 3141: DNA339506, NP_060589.1, 218016_s_at
FIG. 3142: PRO91277
FIG. 3143: DNA325094, NP_079346.1, 218017_s_at
FIG. 3144: PRO81671
FIG. 3145: DNA328836, NP_054894.1, 218027_at
FIG. 3146: PRO84572
FIG. 3147A-B: DNA255183, NP_061900.1,
218035_s_at
FIG. 3148: PRO50262
FIG. 3149: DNA325978, NM_016359, 218039_at
FIG. 3150: PRO82423
FIG. 3151: DNA329276, NP_077001.1, 218069_at
FIG. 3152: PRO12104
FIG. 3153: DNA287261, NP_060344.1, 218081_at
FIG. 3154: PRO69533
FIG. 3155: DNA325169, NP_057494.2, 218085_at
FIG. 3156: PRO81734
FIG. 3157: DNA344708, NP_056207.2, 218086_at
FIG. 3158: PRO95301
FIG. 3159: DNA329278, NP_004495.1, 218092_s_at
FIG. 3160: PRO84871
FIG. 3161: DNA225639, NP_060831.1, 218096_at
FIG. 3162: PRO36102
FIG. 3163: DNA344709, NP_004540.1, 218101_s_at
FIG. 3164: PRO82036
FIG. 3165: DNA344710, NP_666499.1, 218105_s_at
FIG. 3166: PRO62669
FIG. 3167: DNA344711, NP_060699.2, 218139_s_at
FIG. 3168: PRO95302
FIG. 3169: DNA327857, NP_057386.1, 218142_s_at
FIG. 3170: PRO83799
FIG. 3171: DNA287235, NP_060598.1, 218156_s_at
FIG. 3172: PRO69514
FIG. 3173: DNA151377, NP_057132.1, 218170_at
FIG. 3174: PRO11754
FIG. 3175: DNA304470, NP_061100.1, 218172_s_at
FIG. 3176: PRO71046
FIG. 3177A-D: DNA340174, NP_064630.1,
218184_at
FIG. 3178: PRO91669
FIG. 3179: DNA344712, NP_036590.1, 218188_s_at
FIG. 3180: PRO82887
FIG. 3181A-C: DNA330360, NP_078789.1,
218204_s_at
FIG. 3182: PRO85576
FIG. 3183: DNA344713, NP_060641.2, 218218_at
FIG. 3184: PRO95303
FIG. 3185: DNA225650, NP_057246.1, 218234_at
FIG. 3186: PRO36113
FIG. 3187: DNA327858, NP_036473.1, 218238_at
FIG. 3188: PRO83800
FIG. 3189: DNA327858, NM_012341, 218239_s_at
FIG. 3190: PRO83800
FIG. 3191A-B: DNA344714, NP_037367.2,
218269_at
FIG. 3192: PRO95304
FIG. 3193: DNA329074, NP_064524.1, 218285_s_at
FIG. 3194: PRO21326
FIG. 3195A-B: DNA328853, NP_065702.2,
218319_at
FIG. 3196: PRO84584
FIG. 3197: DNA329281, NP_036526.2, 218336_at
FIG. 3198: PRO84874
FIG. 3199A-B: DNA344715, BAB47444.2,
218342_s_at
FIG. 3200: PRO95305
FIG. 3201: DNA328854, NP_056979.1, 218350_s_at
FIG. 3202: PRO84585
FIG. 3203A-B: DNA273415, NP_036442.2,
218355_at
FIG. 3204: PRO61414
FIG. 3205: DNA344716, NP_071921.1, 218373_at
FIG. 3206: PRO95306
FIG. 3207A-B: DNA330366, NP_073602.2,
218376_s_at
FIG. 3208: PRO85581
FIG. 3209: DNA328856, NP_068376.1, 218380_at
FIG. 3210: PRO84586
FIG. 3211: DNA327863, NP_055131.1, 218384_at
FIG. 3212: PRO83804
FIG. 3213: DNA255340, NP_060154.1, 218396_at
FIG. 3214: PRO50409
FIG. 3215: DNA344717, NP_663747.1, 218399_s_at
FIG. 3216: PRO95307
FIG. 3217A-B: DNA287192, NP_006178.1,
218400_at
FIG. 3218: PRO69478
FIG. 3219: DNA333245, NP_037454.2, 218404_at
FIG. 3220: PRO87952
FIG. 3221A-B: DNA344718, NP_076414.2,
218456_at
FIG. 3222: PRO95308
FIG. 3223: DNA328861, NP_057030.2, 218472_s_at
FIG. 3224: PRO84589
FIG. 3225: DNA327943, NP_055399.1, 218498_s_at
FIG. 3226: PRO865
FIG. 3227: DNA150648, NP_037464.1, 218507_at
FIG. 3228: PRO11576
FIG. 3229: DNA326550, NP_057663.1, 218529_at
FIG. 3230: PRO224
FIG. 3231: DNA327868, NP_060601.2, 218542_at
FIG. 3232: PRO83809
FIG. 3233: DNA255113, NP_073587.1, 218543_s_at
FIG. 3234: PRO50195
FIG. 3235: DNA330373, NP_060751.1, 218552_at
FIG. 3236: PRO85587
FIG. 3237: DNA344719, NP_059142.1, 218558_s_at
FIG. 3238: PRO85588
FIG. 3239: DNA329587, NP_036256.1, 218566_s_at
FIG. 3240: PRO85121
FIG. 3241: DNA325036, NP_060708.1, 218568_at
FIG. 3242: PRO81625
FIG. 3243A-B: DNA273435, NP_057532.1,
218585_s_at
FIG. 3244: PRO61430
FIG. 3245: DNA93548, NP_005758.1, 218589_at
FIG. 3246: PRO4929
FIG. 3247: DNA326916, NP_149061.1, 218592_s_at
FIG. 3248: PRO83235
FIG. 3249: DNA287642, NP_060934.1, 218597_s_at
FIG. 3250: PRO9902
FIG. 3251A-B: DNA254789, NP_057301.1,
218603_at
FIG. 3252: PRO49887
FIG. 3253A-B: DNA344720, NP_073600.2,
218618_s_at
FIG. 3254: PRO95309
FIG. 3255A-B: DNA339409, NP_057257.1,
218620_s_at
FIG. 3256: PRO91214
FIG. 3257: DNA327869, NP_057672.1, 218625_at
FIG. 3258: PRO1898
FIG. 3259: DNA339537, NP_060864.1, 218633_x_at
FIG. 3260: PRO91303
FIG. 3261: DNA344721, NP_057303.1, 218636_s_at
FIG. 3262: PRO1477
FIG. 3263A-B: DNA344722, NP_073606.1,
218648_at
FIG. 3264: PRO95310
FIG. 3265: DNA330378, NP_071741.2, 218663_at
FIG. 3266: PRO81126
FIG. 3267: DNA339660, NP_079491.1, 218670_at
FIG. 3268: PRO91402
FIG. 3269: DNA287291, NP_067036.1, 218676_s_at
FIG. 3270: PRO69561
FIG. 3271: DNA330379, NP_073562.1, 218689_at
FIG. 3272: PRO85591
FIG. 3273: DNA328873, NP_057041.1, 218698_at
FIG. 3274: PRO84600
FIG. 3275: DNA344723, NP_060320.1, 218712_at
FIG. 3276: PRO95311
FIG. 3277: DNA328874, NP_054778.1, 218723_s_at
FIG. 3278: PRO84601
FIG. 3279: DNA324251, NP_060880.2, 218726_at
FIG. 3280: PRO80935
FIG. 3281: DNA330382, NP_005724.1, 218755_at
FIG. 3282: PRO61907
FIG. 3283A-B: DNA344724, NP_054828.2,
218782_s_at
FIG. 3284: PRO95312
FIG. 3285: DNA335239, NP_060158.1, 218792_s_at
FIG. 3286: PRO89625
FIG. 3287: DNA344725, NP_060854.2, 218805_at
FIG. 3288: PRO95313
FIG. 3289: DNA256846, NP_059985.1, 218826_at
FIG. 3290: PRO51777
FIG. 3291: DNA255213, AK000364, 218829_s_at
FIG. 3292: PRO50292
FIG. 3293: DNA328879, NP_064570.1, 218845_at
FIG. 3294: PRO84606
FIG. 3295A-B: DNA344726, NP_004821.2,
218846_at
FIG. 3296: PRO95314
FIG. 3297: DNA330385, NP_057733.2, 218859_s_at
FIG. 3298: PRO85594
FIG. 3299: DNA330386, NP_057394.1, 218866_s_at
FIG. 3300: PRO85595
FIG. 3301: DNA344727, NP_060930.2, 218870_at
FIG. 3302: PRO95315
FIG. 3303: DNA330387, NP_036309.1, 218875_s_at
FIG. 3304: PRO85596
FIG. 3305: DNA327874, BC022791, 218880_at
FIG. 3306: PRO4805
FIG. 3307: DNA344728, NP_078806.1, 218881_s_at
FIG. 3308: PRO95316
FIG. 3309: DNA226633, NP_060376.1, 218886_at
FIG. 3310: PRO37096
FIG. 3311A-B: DNA335042, NP_060562.3,
218888_s_at
FIG. 3312: PRO4401
FIG. 3313: DNA344729, AK026953, 218889_at
FIG. 3314: PRO95317
FIG. 3315: DNA254380, NP_065112.1, 218918_at
FIG. 3316: PRO49490
FIG. 3317: DNA328364, NP_068577.1, 218921_at
FIG. 3318: PRO84223
FIG. 3319: DNA329333, NP_054886.1, 218936_s_at
FIG. 3320: PRO84917
FIG. 3321A-B: DNA344730, NP_055129.1,
218943_s_at
FIG. 3322: PRO69459
FIG. 3323: DNA334561, NP_068572.1, 218976_at
FIG. 3324: PRO89050
FIG. 3325: DNA329050, NP_057053.1, 218982_s_at
FIG. 3326: PRO84712
FIG. 3327A-B: DNA344731, NP_060101.1,
218986_s_at
FIG. 3328: PRO51309
FIG. 3329: DNA327211, NP_075053.2, 218989_x_at
FIG. 3330: PRO71052
FIG. 3331: DNA227194, NP_060765.1, 218999_at
FIG. 3332: PRO37657
FIG. 3333: DNA328884, NP_054884.1, 219006_at
FIG. 3334: PRO84609
FIG. 3335: DNA227187, NP_057703.1, 219014_at
FIG. 3336: PRO37650
FIG. 3337: DNA328885, NP_061108.2, 219017_at
FIG. 3338: PRO50294
FIG. 3339: DNA329293, NP_057136.1, 219037_at
FIG. 3340: PRO84883
FIG. 3341: DNA333718, NP_068595.2, 219066_at
FIG. 3342: PRO88346
FIG. 3343A-B: DNA344732, NP_060254.2,
219073_s_at
FIG. 3344: PRO90806
FIG. 3345: DNA327877, NP_065108.1, 219099_at
FIG. 3346: PRO83816
FIG. 3347: DNA344733, NP_079204.1, 219100_at
FIG. 3348: PRO95318
FIG. 3349: DNA287242, NP_127460.1, 219110_at
FIG. 3350: PRO69517
FIG. 3351: DNA304472, NP_057678.1, 219117_s_at
FIG. 3352: PRO535
FIG. 3353: DNA297191, NP_060962.2, 219148_at
FIG. 3354: PRO70808
FIG. 3355: DNA329295, NP_036549.1, 219155_at
FIG. 3356: PRO84885
FIG. 3357A-B: DNA331610, NM_025085,
219158_s_at
FIG. 3358: PRO86609
FIG. 3359: DNA328892, NM_021630, 219165_at
FIG. 3360: PRO84616
FIG. 3361: DNA330400, NP_078796.1, 219176_at
FIG. 3362: PRO85608
FIG. 3363A-B: DNA344734, NP_078914.1,
219178_at
FIG. 3364: PRO95319
FIG. 3365: DNA329223, NP_037517.1, 219183_s_at
FIG. 3366: PRO84831
FIG. 3367: DNA330401, NP_057377.1, 219191_s_at
FIG. 3368: PRO85609
FIG. 3369: DNA344735, NP_071451.1, 219209_at
FIG. 3370: PRO83818
FIG. 3371: DNA344736, NP_057614.1, 219210_s_at
FIG. 3372: PRO95320
FIG. 3373: DNA330403, NP_059110.1, 219211_at
FIG. 3374: PRO85611
FIG. 3375: DNA339627, NP_079000.1, 219221_at
FIG. 3376: PRO91378
FIG. 3377: DNA333832, NP_071411.1, 219222_at
FIG. 3378: PRO88449
FIG. 3379: DNA225594, NP_037404.1, 219229_at
FIG. 3380: PRO36057
FIG. 3381: DNA252224, NM_022073, 219232_s_at
FIG. 3382: PRO48216
FIG. 3383: DNA344737, NP_060796.1, 219243_at
FIG. 3384: PRO84617
FIG. 3385: DNA344738, NP_061195.2, 219255_x_at
FIG. 3386: PRO19612
FIG. 3387: DNA329296, NP_060328.1, 219258_at
FIG. 3388: PRO84886
FIG. 3389: DNA328895, NP_071762.2, 219259_at
FIG. 3390: PRO1317
FIG. 3391: DNA255020, NP_061918.1, 219297_at
FIG. 3392: PRO50109
FIG. 3393: DNA255939, NP_078876.1, 219315_s_at
FIG. 3394: PRO50991
FIG. 3395: DNA227784, NP_060383.1, 219343_at
FIG. 3396: PRO38247
FIG. 3397: DNA254710, NP_060382.1, 219352_at
FIG. 3398: PRO49810
FIG. 3399: DNA287174, AF161525, 219356_s_at
FIG. 3400: PRO69464
FIG. 3401A-B: DNA327885, NP_075601.1,
219369_s_at
FIG. 3402: PRO82377
FIG. 3403: DNA188342, NP_064510.1, 219386_s_at
FIG. 3404: PRO21718
FIG. 3405: DNA344739, NP_683866.1, 219423_x_at
FIG. 3406: PRO95321
FIG. 3407: DNA329014, NP_005746.2, 219424_at
FIG. 3408: PRO9998
FIG. 3409: DNA328902, NP_071750.1, 219452_at
FIG. 3410: PRO84623
FIG. 3411: DNA328367, NP_079108.2, 219456_s_at
FIG. 3412: PRO84226
FIG. 3413: DNA328367, NM_024832, 219457_s_at
FIG. 3414: PRO84226
FIG. 3415A-B: DNA199058, NP_060319.1,
219460_s_at
FIG. 3416: PRO28533
FIG. 3417: DNA325850, NP_076994.1, 219479_at
FIG. 3418: PRO82312
FIG. 3419: DNA344740, NP_D79021.2, 219493_at
FIG. 3420: PRO95322
FIG. 3421A-B: DNA344741, NP_059120.2,
219505_at
FIG. 3422: PRO95323
FIG. 3423A-C: DNA330409, NM_022898,
219528_s_at
FIG. 3424: PRO85617
FIG. 3425: DNA329299, NP_004660.1, 219529_at
FIG. 3426: PRO84888
FIG. 3427: DNA334311, NP_073563.1, 219532_at
FIG. 3428: PRO50477
FIG. 3429: DNA344742, NP_003405.2, 219540_at
FIG. 3430: PRO95324
FIG. 3431: DNA256737, NP_060276.1, 219541_at
FIG. 3432: PRO51671
FIG. 3433: DNA330410, NP_060925.1, 219555_s_at
FIG. 3434: PRO85618
FIG. 3435: DNA225636, NP_065696.1, 219557_s_at
FIG. 3436: PRO36099
FIG. 3437: DNA336133, NP_078852.1, 219582_at
FIG. 3438: PRO90333
FIG. 3439: DNA325053, NP_060230.2, 219588_s_at
FIG. 3440: PRO81637
FIG. 3441: DNA344743, NP_006125.2, 219600_s_at
FIG. 3442: PRO193
FIG. 3443: DNA331601, NP_071915.1, 219628_at
FIG. 3444: PRO85620
FIG. 3445: DNA327892, NP_060470.1, 219648_at
FIG. 3446: PRO83828
FIG. 3447: DNA328915, NP_055056.2, 219654_at
FIG. 3448: PRO84634
FIG. 3449: DNA344744, NP_079352.1, 219675_s_at
FIG. 3450: PRO95325
FIG. 3451: DNA255161, NP_071430.1, 219684_at
FIG. 3452: PRO50241
FIG. 3453: DNA339552, NP_061922.1, 219696_at
FIG. 3454: PRO91318
FIG. 3455A-B: DNA330297, NP_065138.2,
219700_at
FIG. 3456: PRO85524
FIG. 3457A-B: DNA227762, NP_060169.1,
219734_at
FIG. 3458: PRO38225
FIG. 3459: DNA256481, NP_060269.1, 219757_s_at
FIG. 3460: PRO51518
FIG. 3461: DNA344745, NP_078896.1, 219765_at
FIG. 3462: PRO95326
FIG. 3463: DNA344746, NP_078987.2, 219777_at
FIG. 3464: PRO95327
FIG. 3465A-B: DNA330418, NP_060568.3,
219787_s_at
FIG. 3466: PRO85623
FIG. 3467: DNA344747, NP_690049.1, 219793_at
FIG. 3468: PRO95328
FIG. 3469: DNA324981, NP_076975.1, 219812_at
FIG. 3470: PRO81575
FIG. 3471: DNA331378, NP_079020.12, 219834_at
FIG. 3472: PRO86449
FIG. 3473: DNA287295, NP_078784.1, 219836_at
FIG. 3474: PRO69564
FIG. 3475: DNA344748, NP_066358.1, 219854_at
FIG. 3476: PRO95329
FIG. 3477: DNA255255, NM_022154, 219869_s_at
FIG. 3478: PRO50332
FIG. 3479: DNA344749, NP_079273.1, 219870_at
FIG. 3480: PRO95330
FIG. 3481: DNA254838, NP_078904.1, 219874_at
FIG. 3482: PRO49933
FIG. 3483: DNA328923, NP_075379.1, 219892_at
FIG. 3484: PRO84640
FIG. 3485: DNA330421, NP_057438.2, 219911_s_at
FIG. 3486: PRO85626
FIG. 3487A-C: DNA344750, NP_060606.2,
219918_s_at
FIG. 3488: PRO95331
FIG. 3489: DNA328924, NP_057150.2, 219933_at
FIG. 3490: PRO84641
FIG. 3491: DNA344751, NP_037396.2, 219945_at
FIG. 3492: PRO95332
FIG. 3493: DNA256345, AK000925, 219957_at
FIG. 3494: PRO51387
FIG. 3495: DNA218280, NP_068570.1, 219971_at
FIG. 3496: PRO34332
FIG. 3497: DNA325979, NP_060924.4, 219978_s_at
FIG. 3498: PRO82424
FIG. 3499: DNA330425, NP_078956.1, 219990_at
FIG. 3500: PRO85630
FIG. 3501: DNA333765, AK000812, 219994_at
FIG. 3502: PRO88389
FIG. 3503: DNA256141, NP_060893.1, 220030_at
FIG. 3504: PRO51189
FIG. 3505A-B: DNA344752, NP_037389.3,
220038_at
FIG. 3506: PRO95333
FIG. 3507A-B: DNA221079, NP_071445.1,
220066_at
FIG. 3508: PRO34753
FIG. 3509: DNA256091, NP_071385.1, 220094_s_at
FIG. 3510: PRO51141
FIG. 3511: DNA330431, NP_055198.1, 220118_at
FIG. 3512: PRO85635
FIG. 3513: DNA256803, AK001445, 220121_at
FIG. 3514: PRO51734
FIG. 3515: DNA227302, NP_037401.1, 220132_s_at
FIG. 3516: PRO37765
FIG. 3517: DNA344753, AK000388, 220161_s_at
FIG. 3518: PRO95334
FIG. 3519: DNA335568, NP_076927.1, 220177_s_at
FIG. 3520: PRO89910
FIG. 3521: DNA330434, NP_060842.1, 220235_s_at
FIG. 3522: PRO85637
FIG. 3523: DNA344754, NP_036551.3, 220334_at
FIG. 3524: PRO95335
FIG. 3525: DNA287186, NP_061134.1, 220358_at
FIG. 3526: PRO69472
FIG. 3527: DNA255964, NP_079113.1, 220416_at
FIG. 3528: PRO51015
FIG. 3529: DNA339549, NP_061834.1, 220418_at
FIG. 3530: PRO91315
FIG. 3531: DNA330438, NP_061026.1, 220485_s_at
FIG. 3532: PRO50795
FIG. 3533: DNA327214, NP_078991.2, 220495_s_at
FIG. 3534: PRO83483
FIG. 3535: DNA344755, NP_620591.1, 220558_x_at
FIG. 3536: PRO95336
FIG. 3537: DNA255798, NP_079265.1, 220576_at
FIG. 3538: PRO50853
FIG. 3539: DNA344756, NP_079282.1, 220577_at
FIG. 3540: PRO95337
FIG. 3541: DNA344757, NP_071767.2, 220587_s_at
FIG. 3542: PRO95338
FIG. 3543A-B: DNA334963, NP_116561.1,
220613_s_at
FIG. 3544: PRO89395
FIG. 3545: DNA227368, NP_057371.1, 220633_s_at
FIG. 3546: PRO37831
FIG. 3547A-B: DNA327908, NP_060988.2,
220651_s_at
FIG. 3548: PRO83843
FIG. 3549: DNA329306, NP_079149.2, 220655_at
FIG. 3550: PRO84895
FIG. 3551A-B: DNA327909, NP_064568.2,
220658_s_at
FIG. 3552: PRO83844
FIG. 3553: DNA329307, NP_037483.1, 220684_at
FIG. 3554: PRO84896
FIG. 3555: DNA323756, NP_057267.2, 220688_s_at
FIG. 3556: PRO80512
FIG. 3557: DNA330443, NP_061086.1, 220702_at
FIG. 3558: PRO85644
FIG. 3559: DNA344758, NP_061033.1, 220704_at
FIG. 3560: PRO88381
FIG. 3561A-B: DNA329308, NP_065705.2,
220735_s_at
FIG. 3562: PRO84897
FIG. 3563: DNA344759, NP_065857.1, 220773_s_at
FIG. 3564: PRO50495
FIG. 3565: DNA344760, NP_065089.1, 220888_s_at
FIG. 3566: PRO95339
FIG. 3567: DNA288247, NP_478059.1, 220892_s_at
FIG. 3568: PRO70011
FIG. 3569: DNA338124, NP_079419.1, 220918_at
FIG. 3570: PRO90989
FIG. 3571: DNA328940, NP_078893.1, 220933_s_at
FIG. 3572: PRO84653
FIG. 3573: DNA344761, NP_065126.1, 220944_at
FIG. 3574: PRO95340
FIG. 3575: DNA324246, NP_112188.1, 221004_s_at
FIG. 3576: PRO80930
FIG. 3577: DNA336778, NP_110407.2, 221020_s_at
FIG. 3578: PRO90848
FIG. 3579: DNA254520, NP_060952.1, 221039_s_at
FIG. 3580: PRO49627
FIG. 3581: DNA328945, NP_079177.2, 221081_s_at
FIG. 3582: PRO84657
FIG. 3583: DNA344762, NP_036613.1, 221092_at
FIG. 3584: PRO89669
FIG. 3585: DNA226227, NP_060872.1, 221111_at
FIG. 3586: PRO36690
FIG. 3587: DNA344763, NP_659508.1, 221223_x_at
FIG. 3588: PRO86458
FIG. 3589A-C: DNA332533, NP_068585.1,
221234_s_at
FIG. 3590: PRO87347
FIG. 3591: DNA328948, NP_110437.1, 221253_s_at
FIG. 3592: PRO84659
FIG. 3593: DNA330452, NP_112494.2, 221258_s_at
FIG. 3594: PRO85653
FIG. 3595: DNA344764, BC000158, 221267_s_at
FIG. 3596: PRO95341
FIG. 3597: DNA295327, NP_068575.1, 221271_at
FIG. 3598: PRO70773
FIG. 3599: DNA329312, NP_005205.2, 221331_x_at
FIG. 3600: PRO84901
FIG. 3601: DNA256061, NP_112183.1, 221428_s_at
FIG. 3602: PRO51109
FIG. 3603: DNA344765, NP_112487.1, 221434_s_at
FIG. 3604: PRO70013
FIG. 3605: DNA344766, 1163161.25, 221471_at
FIG. 3606: PRO12237
FIG. 3607: DNA324282, NP_002939.2, 221475_s_at
FIG. 3608: PRO6360
FIG. 3609: DNA227303, NP_004322.1, 221479_s_at
FIG. 3610: PRO37766
FIG. 3611A-B: DNA344767, NP_004767.1,
221484_at
FIG. 3612: PRO59982
FIG. 3613: DNA330456, NP_060571.1, 221520_s_at
FIG. 3614: PRO85657
FIG. 3615: DNA328952, NP_067067.1, 221524_s_at
FIG. 3616: PRO84663
FIG. 3617: DNA328953, NP_004086.1, 221539_at
FIG. 3618: PRO70296
FIG. 3619: DNA327526, NM_020676, 221552_at
FIG. 3620: PRO83574
FIG. 3621: DNA304486, NP_115497.1, 221553_at
FIG. 3622: PRO71055
FIG. 3623: DNA329317, NP_057353.1, 221558_s_at
FIG. 3624: PRO81157
FIG. 3625: DNA329095, NP_057000.2, 221565_s_at
FIG. 3626: PRO77352
FIG. 3627: DNA334699, NP_003937.1, 221567_at
FIG. 3628: PRO89166
FIG. 3629: DNA329319, NP_005440.1, 221601_s_at
FIG. 3630: PRO1607
FIG. 3631: DNA329319, NM_005449, 221602_s_at
FIG. 3632: PRO1607
FIG. 3633: DNA344768, NP_057059.2, 221618_s_at
FIG. 3634: PRO95342
FIG. 3635: DNA344769, NP_036464.1, 221641_s_at
FIG. 3636: PRO95343
FIG. 3637: DNA218280, NM_021798, 221658_s_at
FIG. 3638: PRO34332
FIG. 3639: DNA327927, NP_037390.2, 221666_s_at
FIG. 3640: PRO57311
FIG. 3641A-B: DNA344770, NP_055140.1,
221676_s_at
FIG. 3642: PRO49875
FIG. 3643: DNA194468, AF225418, 221679_s_at
FIG. 3644: PRO23835
FIG. 3645: DNA344771, AF094508, 221681_s_at
FIG. 3646: DNA330460, NP_060255.2, 221685_s_at
FIG. 3647: PRO85660
FIG. 3648: DNA324690, NP_002511.1, 221691_x_at
FIG. 3649: PRO58993
FIG. 3650: DNA256141, NM_018423, 221696_s_at
FIG. 3651: PRO51189
FIG. 3652: DNA344772, NP_078943.1, 221704_s_at
FIG. 3653: PRO90809
FIG. 3654A-C: DNA328664, NM_007200,
221718_s_at
FIG. 3655: PRO84437
FIG. 3656A-B: DNA344773, 1505701.34, 221727_at
FIG. 3657: PRO95345
FIG. 3658: DNA328961, NP_443112.1, 221756_at
FIG. 3659: PRO84667
FIG. 3660: DNA328961, NM_052880, 221757_at
FIG. 3661: PRO84667
FIG. 3662A-C: DNA328965, BAB21809.1,
221778_at
FIG. 3663: PRO51878
FIG. 3664A-B: DNA344774, AL833316,
221824_s_at
FIG. 3665: PRO95346
FIG. 3666: DNA344775, NP_689501.1, 221864_at
FIG. 3667: PRO95347
FIG. 3668: DNA344776, 299937.3, 221897_at
FIG. 3669: PRO95348
FIG. 3670: DNA327933, 1452741.11, 221899_at
FIG. 3671: PRO83865
FIG. 3672A-B: DNA344777, AB020656, 221905_at
FIG. 3673: DNA328971, AK000472, 221923_s_at
FIG. 3674: PRO84674
FIG. 3675: DNA329321, NP_112493.1, 221931_s_at
FIG. 3676: PRO84906
FIG. 3677A-B: DNA336655, BAB85561.1,
221971_x_at
FIG. 3678: PRO90728
FIG. 3679: DNA344778, 7696429.33, 221973_at
FIG. 3680: PRO95350
FIG. 3681: DNA331384, AK026326, 221985_at
FIG. 3682: PRO86454
FIG. 3683: DNA254739, NP_068766.1, 221987_s_at
FIG. 3684: PRO49837
FIG. 3685: DNA344779, AF218023, 221989_at
FIG. 3686: PRO95351
FIG. 3687: DNA344780, 127586.70, 222001_x_at
FIG. 3688: PRO95352
FIG. 3689A-C: DNA344781, NM_006738,
222024_s_at
FIG. 3690: PRO95353
FIG. 3691: DNA344782, AAH44933.1, 222039_at
FIG. 3692: PRO95354
FIG. 3693: DNA325036, NM_018238, 222132_s_at
FIG. 3694: PRO81625
FIG. 3695A-B: DNA339979, BAA95990.1,
222139_at
FIG. 3696: PRO91487
FIG. 3697: DNA329916, 338326.15, 222142_at
FIG. 3698: PRO85231
FIG. 3699A-B: DNA344783, 027987.100, 222145_at
FIG. 3700: PRO95355
FIG. 3701: DNA331386, AL079297, 222150_s_at
FIG. 3702: DNA328975, NP_078807.1, 222155_s_at
FIG. 3703: PRO47688
FIG. 3704: DNA256784, NP_075069.1, 222209_s_at
FIG. 3705: PRO51716
FIG. 3706: DNA323915, NP_077306.1, 222217_s_at
FIG. 3707: PRO703
FIG. 3708: DNA287425, NP_060979.1, 222231_s_at
FIG. 3709: PRO69682
FIG. 3710: DNA344784, AAB26149.1, 222247_at
FIG. 3711: PRO95356
FIG. 3712: DNA344785, AL137750, 222262_s_at
FIG. 3713: PRO95357
FIG. 3714: DNA344786, 405457.25, 222303_at
FIG. 3715: PRO95358
FIG. 3716: DNA330470, 096828.1, 222307_at
FIG. 3717: PRO85668
FIG. 3718: DNA344787, 016338.1, 222371_at
FIG. 3719: PRO95359
FIG. 3720A-B: DNA324364, NP_037468.1,
222385_x_at
FIG. 3721: PRO1314
FIG. 3722: DNA335675, AJ251830, 222392_x_at
FIG. 3723: PRO90003
FIG. 3724: DNA227358, NP_057479.1, 222404_x_at
FIG. 3725: PRO37821
FIG. 3726: DNA344788, AK074898, 222405_at
FIG. 3727: PRO95360
FIG. 3728A-B: DNA344789, NM_014325,
222409_at
FIG. 3729: PRO49875
FIG. 3730: DNA327939, NP_060654.1, 222442_s_at
FIG. 3731: PRO83869
FIG. 3732: DNA344790, NM_005105, 222443_s_at
FIG. 3733: PRO37600
FIG. 3734A-B: DNA325652, NM_016357,
222457_s_at
FIG. 3735: PRO82143
FIG. 3736A-B: DNA256489, NP_079110.1,
222464_s_at
FIG. 3737: PRO51526
FIG. 3738: DNA331089, NP_057143.1, 222500_at
FIG. 3739: PRO4984
FIG. 3740: DNA329370, NP_060611.2, 222522_x_at
FIG. 3741: PRO84949
FIG. 3742A-B: DNA344791, AL834191, 222603_at
FIG. 3743: PRO95361
FIG. 3744: DNA330483, AK001472, 222608_s_at
FIG. 3745: PRO85679
FIG. 3746: DNA329330, NP_057130.1, 222609_s_at
FIG. 3747: PRO84914
FIG. 3748: DNA344792, BC035985, 222622_at
FIG. 3749: PRO95362
FIG. 3750: DNA329331, NP_005763.2, 222666_s_at
FIG. 3751: PRO84915
FIG. 3752: DNA344793, 1454336.17, 222669_s_at
FIG. 3753: PRO95363
FIG. 3754: DNA344794, NP_079170.1, 222684_s_at
FIG. 3755: PRO95364
FIG. 3756A-B: DNA344795, AF537091, 222685_at
FIG. 3757: PRO95365
FIG. 3758A-B: DNA344796, 998337.2, 222689_at
FIG. 3759: PRO95366
FIG. 3760: DNA339537, NM_018394, 222697_s_at
FIG. 3761: PRO91303
FIG. 3762: DNA323797, NP_078916.1, 222703_s_at
FIG. 3763: PRO80547
FIG. 3764: DNA344797, BC044575, 222734_at
FIG. 3765: PRO95367
FIG. 3766: DNA333586, 295181.4, 222735_at
FIG. 3767: PRO84603
FIG. 3768A-B: DNA344798, NM_014109,
222740_at
FIG. 3769: PRO95368
FIG. 3770: DNA335239, NM_017688, 222746_s_at
FIG. 3771: PRO89625
FIG. 3772A-B: DNA340168, NP_060163.2,
222761_at
FIG. 3773: PRO91663
FIG. 3774: DNA344799, BC005401, 222763_s_at
FIG. 3775: PRO95369
FIG. 3776A-B: DNA335042, NM_018092,
222774_s_at
FIG. 3777: PRO4401
FIG. 3778A-B: DNA344800, BC033901,
222787_s_at
FIG. 3779: PRO95370
FIG. 3780: DNA255044, DNA255044, 222833_at
FIG. 3781A-B: DNA329438, NP_476516.1,
222837_s_at
FIG. 3782: PRO85008
FIG. 3783: DNA339367, NP_037469.1, 222841_s_at
FIG. 3784: PRO91172
FIG. 3785: DNA344801, AL834387, 222843_at
FIG. 3786: PRO95371
FIG. 3787A-B: DNA333626, DNA333626,
222846_at
FIG. 3788: PRO88268
FIG. 3789: DNA335638, NP_203130.1, 222847_s_at
FIG. 3790: PRO48216
FIG. 3791: DNA331389, NP_071428.2, 222848_at
FIG. 3792: PRO81238
FIG. 3793A-B: DNA344802, NP_064547.2,
222875_at
FIG. 3794: PRO95372
FIG. 3795: DNA344803, 321334.4, 222900_at
FIG. 3796: PRO95373
FIG. 3797: DNA344804, NP_005012.1, 222938_x_at
FIG. 3798: PRO95374
FIG. 3799: DNA330501, AK022792, 222958_s_at
FIG. 3800: PRO85694
FIG. 3801: DNA330503, NP_038466.2, 222991_s_at
FIG. 3802: PRO85696
FIG. 3803: DNA330504, NP_057575.2, 222993_at
FIG. 3804: PRO84923
FIG. 3805: DNA324548, NP_110409.2, 223020_at
FIG. 3806: PRO81202
FIG. 3807A-B: DNA344805, NP_057308.1,
223027_at
FIG. 3808: PRO84924
FIG. 3809A-B: DNA344806, NM_016224,
223028_s_at
FIG. 3810: PRO84924
FIG. 3811: DNA324707, NP_037369.1, 223032_x_at
FIG. 3812: PRO81339
FIG. 3813A-B: DNA256347, NP_065801.1,
223055_s_at
FIG. 3814: PRO51389
FIG. 3815A-B: DNA256347, NM_020750,
223056_s_at
FIG. 3816: PRO51389
FIG. 3817: DNA325295, NP_113641.1, 223058_at
FIG. 3818: PRO81841
FIG. 3819: DNA287216, NM_021154, 223062_s_at
FIG. 3820: PRO69496
FIG. 3821: DNA304492, NP_114405.1, 223065_s_at
FIG. 3822: PRO1864
FIG. 3823A-B: DNA328934, NP_061936.2,
223068_at
FIG. 3824: PRO84649
FIG. 3825A-B: DNA328934, NM_019063,
223069_s_at
FIG. 3826: PRO84649
FIG. 3827: DNA344807, NP_036609.1, 223072_s_at
FIG. 3828: PRO95375
FIG. 3829: DNA227294, NP_060225.1, 223076_s_at
FIG. 3830: PRO37757
FIG. 3831A-B: DNA329316, AF158555,
223079_s_at
FIG. 3832: PRO84904
FIG. 3833: DNA329349, NP_054861.1, 223100_s_at
FIG. 3834: PRO84931
FIG. 3835A-C: DNA339662, NP_110433.1,
223125_s_at
FIG. 3836: PRO91404
FIG. 3837: DNA330445, NP_112174.1, 223132_s_at
FIG. 3838: PRO85646
FIG. 3839: DNA325557, NP_115675.1, 223151_at
FIG. 3840: PRO82060
FIG. 3841: DNA329352, NP_057154.2, 223156_at
FIG. 3842: PRO84932
FIG. 3843A-B: DNA339969, BAA86461.1,
223162_s_at
FIG. 3844: PRO91477
FIG. 3845: DNA324924, NP_113631.1, 223164_at
FIG. 3846: PRO81525
FIG. 3847A-B: DNA344808, NP_067028.1,
223168_at
FIG. 3848: PRO1200
FIG. 3849A-B: DNA344809, AAH23525.1,
223176_at
FIG. 3850: PRO95376
FIG. 3851: DNA344810, NP_113665.1, 223179_at
FIG. 3852: PRO84933
FIG. 3853: DNA254276, NP_054896.1, 223180_s_at
FIG. 3854: PRO49387
FIG. 3855: DNA344811, NP_113675.2, 223182_s_at
FIG. 3856: PRO95377
FIG. 3857: DNA344812, AF201944, 223193_x_at
FIG. 3858: PRO95378
FIG. 3859: DNA323792, NP_113647.1, 223195_s_at
FIG. 3860: PRO80542
FIG. 3861: DNA339535, NP_060855.1, 223200_s_at
FIG. 3862: PRO91301
FIG. 3863A-B: DNA257461, NP_113607.1,
223217_s_at
FIG. 3864: PRO52040
FIG. 3865A-B: DNA257461, NM_031419,
223218_s_at
FIG. 3866: PRO52040
FIG. 3867: DNA327954, NP_113646.1, 223220_s_at
FIG. 3868: PRO83879
FIG. 3869: DNA340182, NP_068380.1, 223222_at
FIG. 3870: PRO91677
FIG. 3871: DNA344813, NP_114091.2, 223227_at
FIG. 3872: PRO95379
FIG. 3873: DNA344814, NP_060019.1, 223253_at
FIG. 3874: PRO95380
FIG. 3875: DNA330517, NP_115879.1, 223273_at
FIG. 3876: PRO85707
FIG. 3877: DNA344815, NP_116565.1, 223276_at
FIG. 3878: PRO12050
FIG. 3879A-B: DNA330522, NP_116071.2,
223287_s_at
FIG. 3880: PRO85712
FIG. 3881: DNA326962, NP_064711.1, 223290_at
FIG. 3882: PRO83275
FIG. 3883: DNA330523, BC001220, 223294_at
FIG. 3884: PRO85713
FIG. 3885: DNA257363, NP_115691.1, 223296_at
FIG. 3886: PRO51950
FIG. 3887: DNA329355, NP_150596.1, 223299_at
FIG. 3888: PRO50434
FIG. 3889: DNA329356, NP_115671.1, 223304_at
FIG. 3890: PRO84935
FIG. 3891: DNA330454, NP_112589.1, 223307_at
FIG. 3892: PRO85655
FIG. 3893: DNA344816, NM_020806, 223319_at
FIG. 3894: PRO50495
FIG. 3895: DNA329358, NP_115649.1, 223334_at
FIG. 3896: PRO84937
FIG. 3897A-B: DNA255756, L12052, 223358_s_at
FIG. 3898: PRO50812
FIG. 3899: DNA344817, NM_145071, 223377_x_at
FIG. 3900: PRO86458
FIG. 3901A-B: DNA344818, NP_055387.1,
223380_s_at
FIG. 3902: PRO95381
FIG. 3903: DNA344819, NP_663735.1, 223381_at
FIG. 3904: PRO38881
FIG. 3905A-B: DNA344820, NP_115644.1,
223382_s_at
FIG. 3906: PRO84939
FIG. 3907A-B: DNA344821, NM_032268,
223383_at
FIG. 3908: PRO84939
FIG. 3909: DNA340216, NP_115686.2, 223398_at
FIG. 3910: PRO91711
FIG. 3911: DNA339511, NP_060635.1, 223400_s_at
FIG. 3912: PRO91282
FIG. 3913: DNA324156, NP_115588.1, 223403_s_at
FIG. 3914: PRO80856
FIG. 3915: DNA344822, NP_115514.2, 223412_at
FIG. 3916: PRO95382
FIG. 3917: DNA329362, NP_060286.1, 223413_s_at
FIG. 3918: PRO84941
FIG. 3919: DNA329362, NM_017816, 223414_s_at
FIG. 3920: PRO84941
FIG. 3921: DNA255676, NP_060754.1, 223434_at
FIG. 3922: PRO50738
FIG. 3923: DNA330533, NP_058647.1, 223451_s_at
FIG. 3924: PRO772
FIG. 3925: DNA344823, BAA92078.1, 223457_at
FIG. 3926: PRO95383
FIG. 3927: DNA273418, AAG01157.1, 223480_s_at
FIG. 3928: DNA327958, NP_115789.1, 223484_at
FIG. 3929: PRO23554
FIG. 3930: DNA329456, NP_057126.1, 223490_s_at
FIG. 3931: PRO85023
FIG. 3932: DNA338084, NP_006564.1, 223502_s_at
FIG. 3933: PRO738
FIG. 3934: DNA344824, AF255647, 223503_at
FIG. 3935: PRO95384
FIG. 3936: DNA333656, NP_115646.2, 223533_at
FIG. 3937: PRO88295
FIG. 3938: DNA330536, NP_115666.1, 223542_at
FIG. 3939: PRO85722
FIG. 3940A-B: DNA339971, BAA86587.1,
223617_x_at
FIG. 3941: PRO91479
FIG. 3942: DNA327028, NP_005291.1, 223620_at
FIG. 3943: PRO37083
FIG. 3944: DNA344825, BC002724, 223666_at
FIG. 3945: PRO83126
FIG. 3946: DNA344826, NP_006548.1, 223704_s_at
FIG. 3947: PRO51385
FIG. 3948: DNA344827, AF176013, 223722_at
FIG. 3949: PRO95385
FIG. 3950: DNA344828, NM_146388, 223743_s_at
FIG. 3951: PRO95386
FIG. 3952: DNA188735, NP_001506.1, 223758_s_at
FIG. 3953: PRO26224
FIG. 3954: DNA287253, NP_444268.1, 223774_at
FIG. 3955: PRO69527
FIG. 3956: DNA331132, NP_115524.1, 223798_at
FIG. 3957: PRO86273
FIG. 3958: DNA332645, NP_570138.1, 223809_at
FIG. 3959: PRO61997
FIG. 3960: DNA327200, NP_114156.1, 223836_at
FIG. 3961: PRO1065
FIG. 3962: DNA344829, NP_683699.1, 223851_s_at
FIG. 3963: PRO95387
FIG. 3964: DNA335398, AF132202, 223940_x_at
FIG. 3965A-B: DNA344830, NM_004830,
223947_s_at
FIG. 3966: PRO95388
FIG. 3967: DNA335568, NM_024022, 223948_s_at
FIG. 3968: PRO89910
FIG. 3969: DNA327213, NM_032405, 223949_at
FIG. 3970: PRO83482
FIG. 3971: DNA344831, NM_013324, 223961_s_at
FIG. 3972: PRO37588
FIG. 3973: DNA324248, NM_004509, 223980_s_at
FIG. 3974: PRO80932
FIG. 3975: DNA344832, AF130059, 223991_s_at
FIG. 3976: PRO95389
FIG. 3977: DNA344833, NP_002594.1, 224046_s_at
FIG. 3978: PRO95390
FIG. 3979: DNA344834, NM_172234, 224156_x_at
FIG. 3980: PRO95391
FIG. 3981A-C: DNA227619, NP_054831.1,
224218_s_at
FIG. 3982: PRO38082
FIG. 3983: DNA324707, NM_013237, 224232_s_at
FIG. 3984: PRO81339
FIG. 3985: DNA329370, NM_018141, 224247_s_at
FIG. 3986: PRO84949
FIG. 3987: DNA344835, NP_115942.1, 224285_at
FIG. 3988: PRO78450
FIG. 3989: DNA330558, NP_057588.1, 224330_s_at
FIG. 3990: PRO84950
FIG. 3991: DNA344836, NP_115868.1, 224331_s_at
FIG. 3992: PRO84951
FIG. 3993: DNA344837, BC015060, 224345_x_at
FIG. 3994: PRO86616
FIG. 3995: DNA344838, NM_018725, 224361_s_at
FIG. 3996: PRO19612
FIG. 3997: DNA335328, NP_116010.1, 224367_at
FIG. 3998: PRO89703
FIG. 3999: DNA330334, NP_114402.1, 224368_s_at
FIG. 4000: PRO85557
FIG. 4001: DNA328323, NP_114148.2, 224428_s_at
FIG. 4002: PRO69531
FIG. 4003: DNA344839, NP_113668.2, 224450_s_at
FIG. 4004: PRO95392
FIG. 4005: DNA328885, NM_018638, 224453_s_at
FIG. 4006: PRO50294
FIG. 4007: DNA344840, NP_116186.1, 224461_s_at
FIG. 4008: PRO95393
FIG. 4009: DNA329373, NP_115722.1, 224467_s_at
FIG. 4010: PRO84952
FIG. 4011: DNA323732, NP_057260.2, 224472_x_at
FIG. 4012: PRO80490
FIG. 4013: DNA344841, BC006236, 224480_s_at
FIG. 4014: PRO95394
FIG. 4015A-C: DNA344842, AJ314646, 224482_s_at
FIG. 4016: DNA344843, BC006384, 224507_s_at
FIG. 4017: PRO95396
FIG. 4018: DNA344844, 242250.1, 224508_at
FIG. 4019: PRO95397
FIG. 4020: DNA327977, NP_115886.1, 224518_s_at
FIG. 4021: PRO83898
FIG. 4022: DNA329374, NP_115735.1, 224523_s_at
FIG. 4023: PRO84953
FIG. 4024: DNA344845, NM_148902, 224553_s_at
FIG. 4025: PRO95398
FIG. 4026: DNA344846, 1453417.19, 224559_at
FIG. 4027: PRO95399
FIG. 4028A-E: DNA344847, AF001893, 224566_at
FIG. 4029: PRO95400
FIG. 4030: DNA334965, D87666, 224567_x_at
FIG. 4031: DNA330569, BC020516, 224572_s_at
FIG. 4032: DNA344848, NP_066972.1, 224583_at
FIG. 4033: PRO82633
FIG. 4034A-B: DNA334919, NP_536856.2,
224596_at
FIG. 4035: PRO89354
FIG. 4036: DNA344849, 1383705.7, 224601_at
FIG. 4037: PRO95401
FIG. 4038: DNA331396, 1357555.1, 224603_at
FIG. 4039: PRO86461
FIG. 4040: DNA255362, DNA255362, 224604_at
FIG. 4041: DNA344850, BC017399, 224605_at
FIG. 4042: PRO95402
FIG. 4043: DNA344851, AF070636, 224609_at
FIG. 4044: PRO95403
FIG. 4045: DNA344852, 348196.115, 224610_at
FIG. 4046: PRO95404
FIG. 4047: DNA329376, BAA91036.1, 224632_at
FIG. 4048: PRO84954
FIG. 4049A-B: DNA344853, 361207.5, 224634_at
FIG. 4050: PRO95405
FIG. 4051: DNA344854, AK093442, 224654_at
FIG. 4052: PRO95406
FIG. 4053A-B: DNA344855, BAB21782.1,
224674_at
FIG. 4054: PRO49364
FIG. 4055A-B: DNA344856, AL161973, 224685_at
FIG. 4056A-B: DNA330574, BAA86542.2,
224698_at
FIG. 4057: PRO85755
FIG. 4058: DNA329378, BC022990, 224714_at
FIG. 4059: PRO84956
FIG. 4060: DNA330577, NP_443076.1, 224715_at
FIG. 4061: PRO85758
FIG. 4062: DNA330579, NP_612434.1, 224719_s_at
FIG. 4063: PRO85760
FIG. 4064: DNA344857, NP_653202.1, 224733_at
FIG. 4065: PRO95408
FIG. 4066: DNA257352, DNA257352, 224739_at
FIG. 4067: PRO51940
FIG. 4068: DNA344858, 887619.58, 224741_x_at
FIG. 4069: PRO95409
FIG. 4070: DNA330581, NP_542399.1, 224753_at
FIG. 4071: PRO82014
FIG. 4072A-B: DNA344859, NP_065875.1,
224764_at
FIG. 4073: PRO95410
FIG. 4074: DNA336077, BC035511, 224783_at
FIG. 4075: PRO90299
FIG. 4076A-B: DNA333692, AB033075, 224790_at
FIG. 4077: DNA228087, DNA228087, 224793_s_at
FIG. 4078: PRO38550
FIG. 4079A-B: DNA287330, BAA86479.1,
224799_at
FIG. 4080: PRO69594
FIG. 4081A-B: DNA330584, NP_065881.1,
224800_at
FIG. 4082: PRO85764
FIG. 4083A-B: DNA287330, AB032991, 224801_at
FIG. 4084: DNA331397, AK001723, 224802_at
FIG. 4085: PRO23259
FIG. 4086: DNA344860, NP_699164.1, 224819_at
FIG. 4087: PRO95411
FIG. 4088A-B: DNA330559, BAB21791.1,
224832_at
FIG. 4089: PRO85741
FIG. 4090A-B: DNA330809, 336997.1, 224837_at
FIG. 4091: PRO85973
FIG. 4092A-B: DNA330522, NM_032682,
224838_at
FIG. 4093: PRO85712
FIG. 4094A-B: DNA344861, NP_597700.1,
224839_s_at
FIG. 4095: PRO95412
FIG. 4096A-B: DNA324748, NP_004108.1,
224840_at
FIG. 4097: PRO36841
FIG. 4098A-B: DNA344862, AF141346,
224841_x_at
FIG. 4099: DNA344863, BC027989, 224847_at
FIG. 4100: PRO95414
FIG. 4101A-C: DNA329379, 010205.2, 224848_at
FIG. 4102: PRO84957
FIG. 4103: DNA344864, NP_116199.1, 224850_at
FIG. 4104: PRO95415
FIG. 4105A-B: DNA324748, NM_004117,
224856_at
FIG. 4106: PRO36841
FIG. 4107: DNA329381, D28589, 224870_at
FIG. 4108A-B: DNA344865, NP_065871.1,
224909_s_at
FIG. 4109: PRO95416
FIG. 4110: DNA344866, AAH10736.1, 224913_s_at
FIG. 4111: PRO95417
FIG. 4112: DNA330591, NP_115865.1, 224919_at
FIG. 4113: PRO85771
FIG. 4114A-B: DNA344867, BC009948, 224925_at
FIG. 4115: PRO95418
FIG. 4116A-B: DNA228196, BAA92674.1,
224937_at
FIG. 4117: PRO38661
FIG. 4118: DNA336269, 346724.14, 224944_at
FIG. 4119: PRO90430
FIG. 4120: DNA344868, 7769724.1, 224989_at
FIG. 4121: PRO95419
FIG. 4122: DNA329384, NP_777581.1, 224990_at
FIG. 4123: PRO84960
FIG. 4124: DNA344869, BC034247, 225036_at
FIG. 4125: PRO95420
FIG. 4126: DNA344870, NP_061189.1, 225081_s_at
FIG. 4127: PRO95421
FIG. 4128: DNA330598, 1384569.2, 225086_at
FIG. 4129: PRO85776
FIG. 4130A-E: DNA329391, 233747.10, 225097_at
FIG. 4131: PRO84967
FIG. 4132A-B: DNA327993, 898436.7, 225133_at
FIG. 4133: PRO81138
FIG. 4134: DNA344871, BC037573, 225148_at
FIG. 4135: PRO95422
FIG. 4136: DNA344872, NP_079272.4, 225158_at
FIG. 4137: PRO84969
FIG. 4138: DNA344873, NM_024996, 225161_at
FIG. 4139: PRO84969
FIG. 4140: DNA330604, NP_277050.1, 225171_at
FIG. 4141: PRO85782
FIG. 4142: DNA330604, NM_033515, 225173_at
FIG. 4143: PRO85782
FIG. 4144: DNA344874, BC040556, 225175_s_at
FIG. 4145: PRO95423
FIG. 4146: DNA344875, AAH27990.1, 225178_at
FIG. 4147: PRO83914
FIG. 4148A-B: DNA344876, 335186.18, 225195_at
FIG. 4149: PRO95424
FIG. 4150: DNA336053, NP_110438.1, 225196_s_at
FIG. 4151: PRO90282
FIG. 4152: DNA344877, 233597.34, 225220_at
FIG. 4153: PRO95425
FIG. 4154: DNA344878, NP_542763.1, 225252_at
FIG. 4155: PRO95426
FIG. 4156A-B: DNA330605, 233102.7, 225265_at
FIG. 4157: PRO85783
FIG. 4158A-B: DNA258863, DNA258863,
225266_at
FIG. 4159A-B: DNA344879, 7771332.17, 225285_at
FIG. 4160: PRO95427
FIG. 4161A-B: DNA330606, 475590.1, 225290_at
FIG. 4162: PRO85784
FIG. 4163: DNA344880, NP_149100.1, 225291_at
FIG. 4164: PRO95428
FIG. 4165: DNA339708, NP_116147.1, 225309_at
FIG. 4166: PRO91438
FIG. 4167: DNA344881, 1455093.11, 225315_at
FIG. 4168: PRO95429
FIG. 4169: DNA324422, DNA324422, 225331_at
FIG. 4170: PRO81086
FIG. 4171A-B: DNA344882, 331507.16, 225342_at
FIG. 4172: PRO95430
FIG. 4173: DNA344883, 475538.46, 225351_at
FIG. 4174: PRO95431
FIG. 4175: DNA344884, 475309.4, 225356_at
FIG. 4176: PRO95432
FIG. 4177A-B: DNA330742, 476805.1, 225363_at
FIG. 4178: PRO85910
FIG. 4179: DNA327965, NP_060760.1, 225367_at
FIG. 4180: PRO83888
FIG. 4181: DNA329401, NP_612403.2, 225386_s_at
FIG. 4182: PRO84976
FIG. 4183: DNA344885, NM_173647, 225414_at
FIG. 4184: PRO95433
FIG. 4185: DNA344886, NP_116258.1, 225439_at
FIG. 4186: PRO52516
FIG. 4187A-B: DNA330617, 336147.2, 225447_at
FIG. 4188: PRO59923
FIG. 4189: DNA330618, CAB55990.1, 225458_at
FIG. 4190: PRO85793
FIG. 4191: DNA344887, BC022333, 225470_at
FIG. 4192: PRO95434
FIG. 4193A-B: DNA328006, 234824.7, 225478_at
FIG. 4194: PRO83924
FIG. 4195A-B: DNA334963, NM_032943,
225496_s_at
FIG. 4196: PRO89395
FIG. 4197A-B: DNA344888, AL833216, 225519_at
FIG. 4198: PRO95435
FIG. 4199: DNA331675, NP_056255.1, 225520_at
FIG. 4200: PRO86670
FIG. 4201A-B: DNA344889, BAB33341.1,
225525_at
FIG. 4202: PRO95436
FIG. 4203: DNA330621, AAF71051.1, 225535_s_at
FIG. 4204: PRO85795
FIG. 4205: DNA328010, NP_149016.1, 225557_at
FIG. 4206: PRO83928
FIG. 4207A-B: DNA344890, NM_057170,
225558_at
FIG. 4208: PRO95437
FIG. 4209A-B: DNA344891, AL832362, 225570_at
FIG. 4210: PRO95438
FIG. 4211A-B: DNA329407, 234687.2, 225606_at
FIG. 4212: PRO84980
FIG. 4213A-B: DNA344892, AK074072, 225608_at
FIG. 4214A-C: DNA344893, 197240.1, 225611_at
FIG. 4215: PRO95440
FIG. 4216: DNA331399, 994419.37, 225622_at
FIG. 4217: PRO86463
FIG. 4218A-B: DNA340041, AK024473, 225624_at
FIG. 4219A-B: DNA331400, NP_060910.2,
225626_at
FIG. 4220: PRO86464
FIG. 4221A-B: DNA344894, BAA96062.2,
225629_s_at
FIG. 4222: PRO95441
FIG. 4223: DNA344895, 473880.39, 225636_at
FIG. 4224: PRO95442
FIG. 4225: DNA344896, NM_148170, 225647_s_at
FIG. 4226: PRO95443
FIG. 4227A-B: DNA288261, NP_037414.2,
225655_at
FIG. 4228: PRO70021
FIG. 4229: DNA344897, NP_612496.1, 225657_at
FIG. 4230: PRO81096
FIG. 4231A-B: DNA344898, NM_133646,
225662_at
FIG. 4232: PRO95444
FIG. 4233A-B: DNA344899, AF480462, 225665_at
FIG. 4234: PRO95445
FIG. 4235: DNA332522, 235504.1, 225685_at
FIG. 4236: PRO87339
FIG. 4237: DNA328012, BC017873, 225686_at
FIG. 4238: PRO83930
FIG. 4239: DNA329410, DNA329410, 225699_at
FIG. 4240: PRO84982
FIG. 4241: DNA304821, AAH11254.1, 225706_at
FIG. 4242: PRO71227
FIG. 4243: DNA344900, NP_689735.1, 225707_at
FIG. 4244: PRO95446
FIG. 4245: DNA344901, 1383664.3, 225710_at
FIG. 4246: PRO95447
FIG. 4247: DNA344902, 040422.37, 225711_at
FIG. 4248: PRO95448
FIG. 4249A-B: DNA330634, 243208.1, 225725_at
FIG. 4250: PRO85806
FIG. 4251A-B: DNA255834, BAA86514.1,
225727_at
FIG. 4252: PRO50889
FIG. 4253: DNA325290, NP_116294.1, 225751_at
FIG. 4254: PRO81837
FIG. 4255A-B: DNA344903, 232693.1, 225752_at
FIG. 4256: PRO95449
FIG. 4257A-B: DNA344904, 344455.25,
225766_s_at
FIG. 4258: PRO60223
FIG. 4259: DNA344905, BC044244, 225775_at
FIG. 4260: PRO95450
FIG. 4261: DNA328016, NP_542409.1, 225783_at
FIG. 4262: PRO83934
FIG. 4263: DNA344906, 033730.20, 225796_at
FIG. 4264: PRO95451
FIG. 4265: DNA344907, BC009508, 225799_at
FIG. 4266: PRO84986
FIG. 4267A-B: DNA328001, 246799.1, 225801_at
FIG. 4268: PRO83920
FIG. 4269: DNA330637, NP_478136.1, 225803_at
FIG. 4270: PRO85809
FIG. 4271: DNA344908, BC046199, 225834_at
FIG. 4272: PRO95452
FIG. 4273: DNA335325, 199593.7, 225835_at
FIG. 4274: PRO89700
FIG. 4275: DNA329417, 411336.1, 225842_at
FIG. 4276: PRO84989
FIG. 4277: DNA329418, NP_660152.1, 225850_at
FIG. 4278: PRO19906
FIG. 4279: DNA344909, 001697.17, 225857_s_at
FIG. 4280: PRO95453
FIG. 4281A-B: DNA258903, DNA258903,
225864_at
FIG. 4282: DNA344910, BC035314, 225866_at
FIG. 4283: PRO81453
FIG. 4284A-B: DNA344911, NP_733837.1,
225887_at
FIG. 4285: PRO95454
FIG. 4286: DNA330642, NP_115494.1, 225898_at
FIG. 4287: PRO85814
FIG. 4288A-B: DNA331403, NP_150601.1,
225912_at
FIG. 4289: PRO86467
FIG. 4290: DNA344912, 232561.20, 225922_at
FIG. 4291: PRO95455
FIG. 4292A-B: DNA328790, 481415.9, 225927_at
FIG. 4293: PRO84535
FIG. 4294A-B: DNA344913, AL833201,
225929_s_at
FIG. 4295: PRO95456
FIG. 4296: DNA344914, BC032220, 225931_s_at
FIG. 4297: PRO95457
FIG. 4298A-B: DNA344915, AL390144,
225959_s_at
FIG. 4299: PRO95458
FIG. 4300: DNA344916, 202205.5, 225967_s_at
FIG. 4301: PRO95459
FIG. 4302A-B: DNA344917, BC037303, 225984_at
FIG. 4303: PRO95460
FIG. 4304A-B: DNA329423, BAB21799.1,
226003_at
FIG. 4305: PRO84994
FIG. 4306A-B: DNA335463, 246054.6, 226021_at
FIG. 4307: PRO89818
FIG. 4308A-B: DNA344918, 347857.19, 226025_at
FIG. 4309: PRO95461
FIG. 4310: DNA335659, 027830.2, 226034_at
FIG. 4311: PRO89988
FIG. 4312A-B: DNA344919, 331817.1, 226039_at
FIG. 4313: PRO95462
FIG. 4314: DNA344920, NP_079382.2, 226075_at
FIG. 4315: PRO95463
FIG. 4316A-B: DNA344921, 1500207.3, 226085_at
FIG. 4317: PRO95464
FIG. 4318A-B: DNA344922, NM_012081,
226099_at
FIG. 4319: PRO37794
FIG. 4320: DNA329425, BC008294, 226117_at
FIG. 4321A-B: DNA344923, AK027859, 226118_at
FIG. 4322: PRO95465
FIG. 4323: DNA257557, DNA257557, 226123_at
FIG. 4324: DNA330657, 198409.1, 226140_s_at
FIG. 4325: PRO85829
FIG. 4326: DNA344924, 243488.38, 226150_at
FIG. 4327: PRO95466
FIG. 4328A-B: DNA344925, BAB67795.1,
226184_at
FIG. 4329: PRO95467
FIG. 4330: DNA344926, 128514.91, 226193_x_at
FIG. 4331: PRO95468
FIG. 4332: DNA344927, NP_659489.1, 226199_at
FIG. 4333: PRO91821
FIG. 4334: DNA344928, AF306698, 226214_at
FIG. 4335: PRO95469
FIG. 4336A-B: DNA329428, 1446144.8, 226218_at
FIG. 4337: PRO84999
FIG. 4338A-B: DNA344929, 1445835.2, 226225_at
FIG. 4339: PRO95470
FIG. 4340: DNA344930, 7761926.1, 226233_at
FIG. 4341: PRO95471
FIG. 4342: DNA344931, BX248749, 226241_s_at
FIG. 4343A-C: DNA344932, 987122.2, 226251_at
FIG. 4344: PRO95473
FIG. 4345: DNA344933, NP_071931.1, 226264_at
FIG. 4346: PRO95474
FIG. 4347: DNA330666, 199829.14, 226272_at
FIG. 4348: PRO85838
FIG. 4349: DNA344934, BC036402, 226275_at
FIG. 4350: DNA344935, 347831.7, 226282_at
FIG. 4351: PRO95476
FIG. 4352: DNA328028, NP_005773.1, 226319_s_at
FIG. 4353: PRO83945
FIG. 4354: DNA328028, NM_005782, 226320_at
FIG. 4355: PRO83945
FIG. 4356: DNA344936, 7696668.2, 226333_at
FIG. 4357: PRO95477
FIG. 4358: DNA344937, 218237.1, 226350_at
FIG. 4359: PRO95478
FIG. 4360A-B: DNA331407, 198233.1, 226352_at
FIG. 4361: PRO86471
FIG. 4362: DNA329430, NP_116191.2, 226353_at
FIG. 4363: PRO38524
FIG. 4364A-B: DNA330675, 177663.2, 226372_at
FIG. 4365: PRO85847
FIG. 4366A-B: DNA344938, AL832599, 226390_at
FIG. 4367: DNA335613, NP_116178.1, 226401_at
FIG. 4368: PRO89948
FIG. 4369: DNA344939, BC044951, 226410_at
FIG. 4370: DNA344940, 407605.1, 226431_at
FIG. 4371: PRO95480
FIG. 4372A-B: DNA344941, 474795.3, 226438_at
FIG. 4373: PRO95481
FIG. 4374: DNA330678, 401430.1, 226444_at
FIG. 4375: PRO85850
FIG. 4376: DNA344942, AL390172, 226517_at
FIG. 4377: PRO95482
FIG. 4378: DNA344943, 334193.1, 226528_at
FIG. 4379: PRO95483
FIG. 4380: DNA304794, NP_115521.2, 226541_at
FIG. 4381: PRO71206
FIG. 4382: DNA344944, 978789.5, 226545_at
FIG. 4383: PRO95484
FIG. 4384A-B: DNA344945, 237667.2, 226568_at
FIG. 4385: PRO95485
FIG. 4386A-B: DNA328031, 331264.1, 226587_at
FIG. 4387: PRO83948
FIG. 4388: DNA344946, AK098194, 226609_at
FIG. 4389: PRO95486
FIG. 4390: DNA344947, AAM76703.1, 226610_at
FIG. 4391: PRO95487
FIG. 4392: DNA344948, AF514992, 226611_s_at
FIG. 4393: DNA328033, 1446419.1, 226625_at
FIG. 4394: PRO83949
FIG. 4395: DNA344949, NP_689775.1, 226661_at
FIG. 4396: PRO95489
FIG. 4397: DNA338349, NM_173626, 226679_at
FIG. 4398: PRO91021
FIG. 4399A-B: DNA328035, 336832.2, 226682_at
FIG. 4400: PRO83951
FIG. 4401A-B: DNA344950, 239418.7, 226683_at
FIG. 4402: PRO95490
FIG. 4403A-C: DNA329129, NM_007203,
226694_at
FIG. 4404: PRO84288
FIG. 4405: DNA328037, AAH16969.1, 226702_at
FIG. 4406: PRO83952
FIG. 4407: DNA344951, NP_660202.1, 226707_at
FIG. 4408: PRO95491
FIG. 4409: DNA344952, 7762613.1, 226736_at
FIG. 4410: PRO95492
FIG. 4411A-B: DNA344953, NP_689561.1,
226738_at
FIG. 4412: PRO95493
FIG. 4413A-B: DNA344954, 7762967.1, 226756_at
FIG. 4414: PRO95494
FIG. 4415: DNA338085, NP_001538.2, 226757_at
FIG. 4416: PRO90963
FIG. 4417: DNA344955, 232416.1, 226759_at
FIG. 4418: PRO95495
FIG. 4419A-B: DNA344956, 898708.1, 226760_at
FIG. 4420: PRO95496
FIG. 4421A-B: DNA344957, AL832206, 226782_at
FIG. 4422: PRO95497
FIG. 4423A-B: DNA332574, 1383798.8, 226789_at
FIG. 4424: PRO87370
FIG. 4425A-B: DNA330694, 481455.4, 226810_at
FIG. 4426: PRO85865
FIG. 4427: DNA328038, 216863.2, 226811_at
FIG. 4428: PRO83953
FIG. 4429A-B: DNA344958, NP_115939.1,
226829_at
FIG. 4430: PRO95498
FIG. 4431: DNA344959, 221888.1, 226832_at
FIG. 4432: PRO95499
FIG. 4433: DNA344960, 999400.45, 226864_at
FIG. 4434: PRO95500
FIG. 4435: DNA344961, 255540.3, 226867_at
FIG. 4436: PRO95501
FIG. 4437: DNA344962, Z99705, 226878_at
FIG. 4438: DNA344963, 366261.31, 226883_at
FIG. 4439: PRO95503
FIG. 4440: DNA330564, NP_115885.1, 226906_s_at
FIG. 4441: PRO85746
FIG. 4442: DNA328044, DNA328044, 226936_at
FIG. 4443: PRO83958
FIG. 4444: DNA154627, DNA154627, 226976_at
FIG. 4445: DNA344964, 7696742.1, 226982_at
FIG. 4446: PRO95504
FIG. 4447: DNA344965, 7769585.1, 226991_at
FIG. 4448: PRO95505
FIG. 4449: DNA339717, NP_150281.1, 227006_at
FIG. 4450: PRO91445
FIG. 4451A-B: DNA275168, DNA275168,
227013_at
FIG. 4452: PRO62870
FIG. 4453: DNA344966, NP_065170.1, 227014_at
FIG. 4454: PRO86261
FIG. 4455A-B: DNA330705, 198782.1, 227020_at
FIG. 4456: PRO85876
FIG. 4457: DNA344967, 350955.33, 227030_at
FIG. 4458: PRO95506
FIG. 4459A-C: DNA344968, AB055890, 227039_at
FIG. 4460: PRO95507
FIG. 4461: DNA344969, 7769752.1, 227052_at
FIG. 4462: PRO95508
FIG. 4463: DNA336061, NP_660322.1, 227066_at
FIG. 4464: PRO90288
FIG. 4465: DNA344970, 7698705.3, 227074_at
FIG. 4466: PRO95509
FIG. 4467A-B: DNA344971, 7697931.24, 227110_at
FIG. 4468: PRO95510
FIG. 4469: DNA330709, 7692923.1, 227117_at
FIG. 4470: PRO85880
FIG. 4471: DNA344972, 7698297.2, 227124_at
FIG. 4472: PRO95511
FIG. 4473: DNA333713, 407443.5, 227125_at
FIG. 4474: PRO88341
FIG. 4475: DNA344973, AK098237, 227141_at
FIG. 4476: PRO95512
FIG. 4477: DNA340090, AAH07902.1, 227161_at
FIG. 4478: PRO91590
FIG. 4479A-B: DNA344974, NP_689899.1,
227166_at
FIG. 4480: PRO38669
FIG. 4481: DNA344975, NP_612350.1, 227172_at
FIG. 4482: PRO95513
FIG. 4483: DNA344976, 332013.1, 227177_at
FIG. 4484: PRO95514
FIG. 4485: DNA267411, NP_659443.1, 227182_at
FIG. 4486: PRO57098
FIG. 4487A-B: DNA344977, 408890.1, 227210_at
FIG. 4488: PRO95515
FIG. 4489: DNA344978, AL834179, 227237_x_at
FIG. 4490: PRO95516
FIG. 4491A-B: DNA344979, AL833296, 227239_at
FIG. 4492: PRO95517
FIG. 4493: DNA330717, 232831.10, 227290_at
FIG. 4494: PRO85888
FIG. 4495: DNA344980, BC042036, 227291_s_at
FIG. 4496: PRO95518
FIG. 4497A-B: DNA344981, 337195.1, 227318_at
FIG. 4498: PRO95519
FIG. 4499: DNA329446, NM_078468, 227322_s_at
FIG. 4500: PRO85014
FIG. 4501: DNA344982, AK097987, 227353_at
FIG. 4502: PRO95520
FIG. 4503: DNA336553, AK095177, 227354_at
FIG. 4504: PRO90632
FIG. 4505: DNA344983, 211443.3, 227357_at
FIG. 4506: PRO95521
FIG. 4507: DNA344984, 163230.9, 227361_at
FIG. 4508: PRO95522
FIG. 4509: DNA344985, BC036414, 227369_at
FIG. 4510: PRO95523
FIG. 4511: DNA344986, BC045695, 227379_at
FIG. 4512: PRO95524
FIG. 4513: DNA344987, 244251.8, 227383_at
FIG. 4514: PRO95525
FIG. 4515: DNA332679, 335037.7, 227396_at
FIG. 4516: PRO87464
FIG. 4517: DNA226872, NP_001955.1, 227404_s_at
FIG. 4518: PRO37335
FIG. 4519: DNA344988, 200338.2, 227410_at
FIG. 4520: PRO95526
FIG. 4521: DNA344989, NP_659486.1, 227413_at
FIG. 4522: PRO95527
FIG. 4523A-C: DNA344990, 410523.22, 227426_at
FIG. 4524: PRO12910
FIG. 4525A-B: DNA340206, NP_079420.2,
227438_at
FIG. 4526: PRO91701
FIG. 4527A-B: DNA328054, 233014.1, 227458_at
FIG. 4528: PRO83968
FIG. 4529: DNA344991, NP_005222.2, 227473_at
FIG. 4530: PRO95528
FIG. 4531A-B: DNA344992, AL832945, 227478_at
FIG. 4532: PRO95529
FIG. 4533: DNA344993, 221804.1, 227489_at
FIG. 4534: PRO95530
FIG. 4535: DNA344994, 197788.1, 227491_at
FIG. 4536: PRO95531
FIG. 4537: DNA344995, 1449825.8, 227503_at
FIG. 4538: PRO95532
FIG. 4539: DNA344996, 887619.55, 227517_s_at
FIG. 4540: PRO95533
FIG. 4541A-B: DNA331401, 336865.4, 227525_at
FIG. 4542: PRO86465
FIG. 4543: DNA340229, NP_443070.1, 227552_at
FIG. 4544: PRO91724
FIG. 4545: DNA344997, AAM09645.1, 227560_at
FIG. 4546: PRO95534
FIG. 4547A-B: DNA287193, BAA92611.1,
227606_s_at
FIG. 4548: PRO69479
FIG. 4549: DNA330730, BC010846, 227607_at
FIG. 4550: PRO85899
FIG. 4551A-B: DNA344998, NM_170709,
227627_at
FIG. 4552: PRO95535
FIG. 4553A-B: DNA344999, BC028212, 227645_at
FIG. 4554: PRO95536
FIG. 4555A-B: DNA345000, 1081047.29, 227670_at
FIG. 4556: PRO95537
FIG. 4557: DNA330734, NP_116143.2, 227686_at
FIG. 4558: PRO85903
FIG. 4559: DNA345001, 020646.23, 227697_at
FIG. 4560: PRO95538
FIG. 4561: DNA323723, NP_060658.1, 227700_x_at
FIG. 4562: PRO80483
FIG. 4563: DNA345002, AJ420488, 227708_at
FIG. 4564: PRO95539
FIG. 4565A-B: DNA333658, 1454272.17, 227755_at
FIG. 4566: PRO88297
FIG. 4567A-B: DNA345003, 232924.7, 227767_at
FIG. 4568: PRO95540
FIG. 4569: DNA332527, 028115.17, 227769_at
FIG. 4570: PRO87344
FIG. 4571: DNA339728, NP_542382.1, 227787_s_at
FIG. 4572: PRO91456
FIG. 4573: DNA345004, 196714.3, 227798_at
FIG. 4574: PRO95541
FIG. 4575: DNA345005, AL137420, 227818_at
FIG. 4576: DNA345006, NP_689613.1, 227856_at
FIG. 4577: PRO95543
FIG. 4578: DNA260485, DNA260485, 227867_at
FIG. 4579: PRO54411
FIG. 4580: DNA336725, AY032883, 227877_at
FIG. 4581: PRO90794
FIG. 4582: DNA345007, 198947.2, 227889_at
FIG. 4583: PRO95544
FIG. 4584: DNA329481, NP_057234.2, 227915_at
FIG. 4585: PRO60949
FIG. 4586: DNA329456, NM_016042, 227916_x_at
FIG. 4587: PRO85023
FIG. 4588: DNA345008, 199363.8, 227930_at
FIG. 4589: PRO95545
FIG. 4590: DNA345009, 040316.1, 227944_at
FIG. 4591: PRO95546
FIG. 4592: DNA345010, 1101718.57, 227984_at
FIG. 4593: PRO95547
FIG. 4594: DNA150660, NP_057151.1, 228019_s_at
FIG. 4595: PRO12397
FIG. 4596: DNA345011, 241960.67, 228030_at
FIG. 4597: PRO95548
FIG. 4598: DNA345012, 156397.1, 228032_s_at
FIG. 4599: PRO95549
FIG. 4600: DNA334778, 1383803.1, 228049_x_at
FIG. 4601: PRO89231
FIG. 4602: DNA331655, 1449874.3, 228053_s_at
FIG. 4603: PRO86651
FIG. 4604: DNA330745, NP_612428.1, 228069_at
FIG. 4605: PRO85913
FIG. 4606: DNA345013, NP_694968.1, 228071_at
FIG. 4607: PRO23647
FIG. 4608: DNA345014, AAH25407.1, 228080_at
FIG. 4609: PRO95550
FIG. 4610: DNA345015, NP_694938.1, 228094_at
FIG. 4611: PRO95551
FIG. 4612: DNA330436, NP_037394.1, 228098_s_at
FIG. 4613: PRO85639
FIG. 4614: DNA151725, DNA151725, 228107_at
FIG. 4615: PRO12014
FIG. 4616A-C: DNA330747, 200650.1, 228109_at
FIG. 4617: PRO85915
FIG. 4618: DNA340579, BC040547, 228113_at
FIG. 4619: PRO92247
FIG. 4620A-B: DNA334022, NP_569713.1,
228167_at
FIG. 4621: PRO88589
FIG. 4622: DNA345016, CAD38596.1, 228245_s_at
FIG. 4623: PRO95552
FIG. 4624: DNA260948, DNA260948, 228273_at
FIG. 4625: PRO54700
FIG. 4626: DNA330755, BC020784, 228280_at
FIG. 4627: PRO85923
FIG. 4628: DNA345017, NP_659455.2, 228281_at
FIG. 4629: PRO95553
FIG. 4630: DNA340370, DNA340370, 228283_at
FIG. 4631: PRO91834
FIG. 4632: DNA339731, NP_612380.1, 228298_at
FIG. 4633: PRO91459
FIG. 4634: DNA345018, 333338.2, 228314_at
FIG. 4635: PRO95554
FIG. 4636A-B: DNA345019, 1453154.2, 228324_at
FIG. 4637: PRO95555
FIG. 4638: DNA345020, NM_174889, 228355_s_at
FIG. 4639: PRO95556
FIG. 4640: DNA336744, BC007609, 228361_at
FIG. 4641: PRO90814
FIG. 4642: DNA345021, 7769848.1, 228363_at
FIG. 4643: PRO95557
FIG. 4644: DNA345022, AF378122, 228376_at
FIG. 4645: PRO95558
FIG. 4646: DNA330759, 337444.1, 228390_at
FIG. 4647: PRO85926
FIG. 4648A-B: DNA330760, 330900.8, 228401_at
FIG. 4649: PRO85927
FIG. 4650A-B: DNA339727, NP_542179.1,
228410_at
FIG. 4651: PRO91455
FIG. 4652: DNA345023, NM_015975, 228483_s_at
FIG. 4653: PRO95559
FIG. 4654A-C: DNA330761, 388991.1, 228487_s_at
FIG. 4655: PRO85928
FIG. 4656A-B: DNA328454, NP_057525.1,
228496_s_at
FIG. 4657: PRO4330
FIG. 4658: DNA345024, 412954.22, 228532_at
FIG. 4659: PRO95560
FIG. 4660: DNA336376, 234038.1, 228560_at
FIG. 4661: PRO91061
FIG. 4662: DNA345025, 1453417.9, 228582_x_at
FIG. 4663: PRO95561
FIG. 4664: DNA150004, DNA150004, 228592_at
FIG. 4665: PRO4644
FIG. 4666: DNA345026, BC035088, 228654_at
FIG. 4667: PRO95562
FIG. 4668A-B: DNA345027, 7698079.3, 228658_at
FIG. 4669: PRO95563
FIG. 4670: DNA335393, 025911.1, 228708_at
FIG. 4671: PRO89758
FIG. 4672A-B: DNA345028, 7695185.17, 228722_at
FIG. 4673: PRO95564
FIG. 4674: DNA330772, 286623.2, 228729_at
FIG. 4675: PRO85937
FIG. 4676: DNA257559, NP_116272.1, 228737_at
FIG. 4677: PRO52129
FIG. 4678: DNA328082, BC014851, 228762_at
FIG. 4679: PRO83994
FIG. 4680: DNA345029, 998974.45, 228809_at
FIG. 4681: PRO95565
FIG. 4682: DNA260010, DNA260010, 228812_at
FIG. 4683: DNA330777, DNA330777, 228869_at
FIG. 4684: PRO85941
FIG. 4685: DNA345030, 7693726.1, 228879_at
FIG. 4686: PRO95566
FIG. 4687: DNA345031, 021903.1, 228910_at
FIG. 4688: PRO95567
FIG. 4689: DNA345032, 1087130.10, 228931_at
FIG. 4690: PRO95568
FIG. 4691: DNA329447, BC016981, 228948_at
FIG. 4692: PRO85015
FIG. 4693A-B: DNA345033, AY198415, 228964_at
FIG. 4694: PRO95569
FIG. 4695A-B: DNA340099, BC028424, 228980_at
FIG. 4696: PRO91599
FIG. 4697: DNA345034, AL137573, 229007_at
FIG. 4698: PRO95570
FIG. 4699A-B: DNA336693, NP_277037.1,
229016_s_at
FIG. 4700: PRO90766
FIG. 4701: DNA330786, 233085.1, 229029_at
FIG. 4702: PRO85950
FIG. 4703: DNA336085, DNA336085, 229041_s_at
FIG. 4704: PRO90304
FIG. 4705: DNA330777, 330848.1, 229045_at
FIG. 4706: PRO85941
FIG. 4707: DNA345035, BAC04479.1, 229065_at
FIG. 4708: PRO95571
FIG. 4709: DNA330790, NP_116133.1, 229070_at
FIG. 4710: PRO85954
FIG. 4711: DNA330791, 7697349.2, 229072_at
FIG. 4712: PRO85955
FIG. 4713: DNA332520, 344561.1, 229101_at
FIG. 4714: PRO87337
FIG. 4715A-B: DNA345036, 468481.1, 229116_at
FIG. 4716: PRO95572
FIG. 4717A-D: DNA345037, 903479.18, 229287_at
FIG. 4718: PRO95573
FIG. 4719: DNA333664, 237320.4, 229295_at
FIG. 4720: PRO88303
FIG. 4721A-B: DNA255352, AB033060, 229354_at
FIG. 4722: DNA345038, NM_024711, 229367_s_at
FIG. 4723: PRO95574
FIG. 4724: DNA345039, 199232.2, 229390_at
FIG. 4725: PRO57551
FIG. 4726: DNA255197, DNA255197, 229391_s_at
FIG. 4727: PRO50276
FIG. 4728: DNA335178, AF402776, 229437_at
FIG. 4729: PRO69678
FIG. 4730: DNA330797, 211332.1, 229442_at
FIG. 4731: PRO85961
FIG. 4732: DNA328090, 007911.2, 229450_at
FIG. 4733: PRO84001
FIG. 4734A-B: DNA237810, DNA237810,
229490_s_at
FIG. 4735: PRO38918
FIG. 4736: DNA338094, AK093350, 229521_at
FIG. 4737: PRO90970
FIG. 4738: DNA330799, 481875.1, 229551_x_at
FIG. 4739: PRO85963
FIG. 4740: DNA334937, BAB71227.1, 229553_at
FIG. 4741: PRO89370
FIG. 4742A-B: DNA345040, 451858.13, 229572_at
FIG. 4743: PRO95575
FIG. 4744A-B: DNA345041, AL834393, 229594_at
FIG. 4745: DNA345042, NP_689831.1, 229603_at
FIG. 4746: PRO95577
FIG. 4747: DNA345043, 401253.39, 229604_at
FIG. 4748: PRO95578
FIG. 4749: DNA345044, BC025714, 229606_at
FIG. 4750: PRO95579
FIG. 4751: DNA333760, 098138.1, 229629_at
FIG. 4752: PRO88384
FIG. 4753: DNA345045, BC034328, 229638_at
FIG. 4754: DNA345046, AL833184, 229686_at
FIG. 4755: PRO95581
FIG. 4756: DNA334491, 428695.5, 229725_at
FIG. 4757: PRO88993
FIG. 4758A-B: DNA227985, NP_055107.1,
229733_s_at
FIG. 4759: PRO38448
FIG. 4760: DNA345047, 979808.6, 229764_at
FIG. 4761: PRO95582
FIG. 4762: DNA330807, 334422.1, 229814_at
FIG. 4763: PRO85971
FIG. 4764: DNA345048, 7683061.1, 229841_at
FIG. 4765: PRO95583
FIG. 4766: DNA345049, NP_694579.1, 229901_at
FIG. 4767: PRO81858
FIG. 4768: DNA333743, 243761.3, 229937_x_at
FIG. 4769: PRO88368
FIG. 4770: DNA345050, 221062.1, 229954_at
FIG. 4771: PRO95584
FIG. 4772A-B: DNA345051, NP_722579.1,
229971_at
FIG. 4773: PRO6017
FIG. 4774: DNA345052, NP_689413.1, 229980_s_at
FIG. 4775: PRO69560
FIG. 4776: DNA330811, 1382987.2, 230000_at
FIG. 4777: PRO85975
FIG. 4778: DNA338348, BAC03808.1, 230012_at
FIG. 4779: PRO91019
FIG. 4780: DNA345053, AL834186, 230060_at
FIG. 4781: PRO95585
FIG. 4782: DNA332487, DNA332487, 230110_at
FIG. 4783: PRO87315
FIG. 4784: DNA345054, 064937.11, 230141_at
FIG. 4785: PRO95586
FIG. 4786: DNA345055, NP_065391.1, 230170_at
FIG. 4787: PRO88
FIG. 4788: DNA345056, AL831898, 230179_at
FIG. 4789: PRO95587
FIG. 4790A-B: DNA345057, AL713763, 230180_at
FIG. 4791: PRO95588
FIG. 4792: DNA345058, AL832695, 230192_at
FIG. 4793: DNA345059, 229293.16, 230206_at
FIG. 4794: PRO95590
FIG. 4795: DNA345060, 7692383.1, 230226_s_at
FIG. 4796: PRO95591
FIG. 4797: DNA345061, AK058039, 230292_at
FIG. 4798: PRO95592
FIG. 4799: DNA330818, 212282.1, 230304_at
FIG. 4800: PRO85982
FIG. 4801: DNA345062, 403834.1, 230383_x_at
FIG. 4802: PRO95593
FIG. 4803: DNA330822, 332195.1, 230391_at
FIG. 4804: PRO85986
FIG. 4805A-B: DNA345063, 234102.72, 230425_at
FIG. 4806: PRO95594
FIG. 4807: DNA345064, NP_653312.1, 230434_at
FIG. 4808: PRO95595
FIG. 4809: DNA330712, 1452648.12, 230466_s_at
FIG. 4810: PRO85883
FIG. 4811A-B: DNA330824, 333480.5, 230489_at
FIG. 4812: PRO85988
FIG. 4813: DNA332672, 335924.1, 230494_at
FIG. 4814: PRO87457
FIG. 4815: DNA332827, NP_660356.1, 230563_at
FIG. 4816: PRO87594
FIG. 4817: DNA345065, 234921.2, 230570_at
FIG. 4818: PRO95596
FIG. 4819A-C: DNA254793, NP_055987.1,
230618_s_at
FIG. 4820: PRO49890
FIG. 4821: DNA328098, 402974.1, 230653_at
FIG. 4822: PRO84008
FIG. 4823: DNA257789, NP_116219.1, 230656_s_at
FIG. 4824: PRO52338
FIG. 4825: DNA340247, DNA340247, 230753_at
FIG. 4826: PRO91742
FIG. 4827: DNA345066, AAH29505.1, 230756_at
FIG. 4828: PRO95597
FIG. 4829: DNA336379, 401125.10, 230795_at
FIG. 4830: PRO90514
FIG. 4831: DNA345067, 1132645.25, 230805_at
FIG. 4832: PRO95598
FIG. 4833: DNA332685, 234194.1, 230836_at
FIG. 4834: PRO87470
FIG. 4835: DNA338109, 211204.3, 230866_at
FIG. 4836: PRO90980
FIG. 4837: DNA336019, DNA336019, 230970_at
FIG. 4838: DNA345068, 407233.3, 231093_at
FIG. 4839: PRO95599
FIG. 4840: DNA329405, AL117452, 231094_s_at
FIG. 4841: DNA345069, 895820.1, 231106_at
FIG. 4842: PRO95600
FIG. 4843: DNA329473, 370473.13, 231124_x_at
FIG. 4844: PRO85038
FIG. 4845A-B: DNA226303, DNA226303,
231259_s_at
FIG. 4846: PRO36766
FIG. 4847A-B: DNA339703, NP_115970.2,
231396_s_at
FIG. 4848: PRO91433
FIG. 4849: DNA338354, DNA338354, 231576_at
FIG. 4850: PRO91025
FIG. 4851: DNA150808, M55542, 231577_s_at
FIG. 4852: PRO12478
FIG. 4853: DNA345070, NP_006630.1, 231747_at
FIG. 4854: PRO34958
FIG. 4855: DNA330839, NP_060908.1, 231769_at
FIG. 4856: PRO86002
FIG. 4857: DNA331119, NP_005433.2, 231776_at
FIG. 4858: PRO50745
FIG. 4859: DNA335123, AK027521, 231837_at
FIG. 4860: PRO89526
FIG. 4861: DNA345071, 1512952.7, 231866_at
FIG. 4862: PRO95601
FIG. 4863A-C: DNA339989, BAB21817.1,
231899_at
FIG. 4864: PRO91497
FIG. 4865A-B: DNA329476, 205127.1, 231929_at
FIG. 4866: PRO85040
FIG. 4867A-B: DNA256267, BAB13444.1,
231956_at
FIG. 4868: PRO51311
FIG. 4869: DNA345072, 978672.3, 232000_at
FIG. 4870: PRO95602
FIG. 4871: DNA345073, NP_056475.1, 232024_at
FIG. 4872: PRO95603
FIG. 4873: DNA323732, NM_016176, 232032_x_at
FIG. 4874: PRO80490
FIG. 4875: DNA330852, 1383611.1, 232138_at
FIG. 4876: PRO86015
FIG. 4877: DNA329094, NP_077285.1, 232160_s_at
FIG. 4878: PRO84746
FIG. 4879: DNA345074, 1077685.1, 232230_at
FIG. 4880: PRO95604
FIG. 4881: DNA345075, AJ278112, 232278_s_at
FIG. 4882: PRO95605
FIG. 4883: DNA329393, AF367998, 232296_s_at
FIG. 4884: PRO84969
FIG. 4885: DNA330862, 339154.9, 232304_at
FIG. 4886: PRO86025
FIG. 4887A-B: DNA340232, NP_443169.1,
232382_s_at
FIG. 4888: PRO91727
FIG. 4889: DNA328117, U25029, 232431_at
FIG. 4890: PRO84024
FIG. 4891: DNA340435, DNA340435, 232504_at
FIG. 4892: DNA329286, NP_005691.2, 232510_s_at
FIG. 4893: PRO69644
FIG. 4894: DNA330868, 337037.1, 232584_at
FIG. 4895: PRO86031
FIG. 4896: DNA340361, DNA340361, 232615_at
FIG. 4897: DNA345076, 143540.3, 232682_at
FIG. 4898: PRO95606
FIG. 4899: DNA330869, 406591.1, 232687_at
FIG. 4900: PRO86032
FIG. 4901: DNA270329, DNA270329, 232737_s_at
FIG. 4902: PRO58716
FIG. 4903: DNA330870, 227719.1, 232883_at
FIG. 4904: PRO86033
FIG. 4905: DNA325531, NM_032379, 232914_s_at
FIG. 4906: PRO82038
FIG. 4907: DNA345077, AK022251, 233089_at
FIG. 4908: PRO95607
FIG. 4909: DNA336161, NP_060857.2, 233252_s_at
FIG. 4910: PRO90356
FIG. 4911A-B: DNA340168, NM_017693,
233255_s_at
FIG. 4912: PRO91663
FIG. 4913: DNA324156, NM_032212, 233341_s_at
FIG. 4914: PRO80856
FIG. 4915: DNA331423, AF176071, 233467_s_at
FIG. 4916A-B: DNA331391, NP_065947.1,
233734_s_at
FIG. 4917: PRO49998
FIG. 4918: DNA335477, 209190.1, 233800_at
FIG. 4919: PRO89830
FIG. 4920A-B: DNA345078, 474673.14,
233849_s_at
FIG. 4921: PRO95608
FIG. 4922: DNA329481, NM_016150, 233857_s_at
FIG. 4923: PRO60949
FIG. 4924A-B: DNA338110, 1382987.31, 233880_at
FIG. 4925: PRO90981
FIG. 4926: DNA345079, NP_057023.2, 233970_s_at
FIG. 4927: PRO84916
FIG. 4928: DNA331687, D13078, 234013_at
FIG. 4929: PRO86682
FIG. 4930: DNA333607, 211626.1, 234151_at
FIG. 4931: PRO88251
FIG. 4932: DNA345080, 401293.1, 234260_at
FIG. 4933: PRO95609
FIG. 4934A-B: DNA345081, NP_057422.2,
234304_s_at
FIG. 4935: PRO95610
FIG. 4936: DNA330881, NP_067004.3, 234306_s_at
FIG. 4937: PRO1138
FIG. 4938: DNA329312, NM_005214, 234362_s_at
FIG. 4939: PRO84901
FIG. 4940: DNA345082, 1452291.29, 234398_at
FIG. 4941: PRO95611
FIG. 4942: DNA345083, S60795, 234402_at
FIG. 4943: PRO95612
FIG. 4944: DNA345084, NP_443104.1, 234408_at
FIG. 4945: PRO20110
FIG. 4946: DNA345085, AAA61109.1, 234440_at
FIG. 4947: PRO95613
FIG. 4948A-C: DNA339394, NP_055768.2,
234660_s_at
FIG. 4949: PRO91199
FIG. 4950: DNA345086, BAB15056.1, 234785_at
FIG. 4951: PRO95614
FIG. 4952: DNA345087, X04937, 234819_at
FIG. 4953: PRO95615
FIG. 4954: DNA345088, CAA29554.1, 234849_at
FIG. 4955: PRO95616
FIG. 4956A-C: DNA345089, AJ238394,
234928_x_at
FIG. 4957: PRO95617
FIG. 4958: DNA330882, 406739.1, 234974_at
FIG. 4959: PRO86044
FIG. 4960: DNA345090, NM_052913, 234994_at
FIG. 4961: PRO95618
FIG. 4962: DNA258761, DNA258761, 235019_at
FIG. 4963A-B: DNA345091, 135369.13, 235020_at
FIG. 4964: PRO95619
FIG. 4965: DNA339413, DNA339413, 235046_at
FIG. 4966A-B: DNA345092, 292261.1, 235048_at
FIG. 4967: PRO95620
FIG. 4968A-B: DNA340485, BAC56923.1,
235085_at
FIG. 4969: PRO92206
FIG. 4970: DNA345093, 337920.2, 235104_at
FIG. 4971: PRO95621
FIG. 4972: DNA328146, BC025376, 235117_at
FIG. 4973: PRO84051
FIG. 4974: DNA333752, 200228.1, 235199_at
FIG. 4975: PRO88377
FIG. 4976: DNA345094, 1384081.2, 235203_at
FIG. 4977: PRO95622
FIG. 4978: DNA330896, 250896.1, 235213_at
FIG. 4979: PRO86057
FIG. 4980: DNA345095, 131102.1, 235230_at
FIG. 4981: PRO95623
FIG. 4982: DNA324093, NP_620156.1, 235256_s_at
FIG. 4983: PRO80802
FIG. 4984: DNA336016, DNA336016, 235291_s_at
FIG. 4985: DNA345096, 237100.26, 235292_at
FIG. 4986: PRO95624
FIG. 4987: DNA330898, 227608.1, 235299_at
FIG. 4988: PRO86059
FIG. 4989A-B: DNA345097, NP_783161.1,
235306_at
FIG. 4990: PRO86060
FIG. 4991: DNA328151, 982500.1, 235352_at
FIG. 4992: PRO84056
FIG. 4993A-C: DNA345098, AL832877, 235410_at
FIG. 4994: PRO95625
FIG. 4995A-B: DNA345099, AF133211, 235421_at
FIG. 4996: PRO95626
FIG. 4997A-B: DNA345100, NP_689737.1,
235425_at
FIG. 4998: PRO95627
FIG. 4999A-B: DNA345101, 979268.1, 235440_at
FIG. 5000: PRO95628
FIG. 5001: DNA257872, DNA257872, 235457_at
FIG. 5002: DNA330906, NP_116171.2, 235458_at
FIG. 5003: PRO86067
FIG. 5004A-B: DNA345102, AAH30800.1,
235463_s_at
FIG. 5005: PRO95629
FIG. 5006: DNA345103, NP_689629.1, 235509_at
FIG. 5007: PRO95630
FIG. 5008: DNA330912, 984873.1, 235609_at
FIG. 5009: PRO86073
FIG. 5010A-B: DNA336026, AB095926, 235643_at
FIG. 5011: DNA345104, 1448915.1, 235680_at
FIG. 5012: PRO95631
FIG. 5013: DNA336165, AF368463, 235706_at
FIG. 5014: PRO84371
FIG. 5015: DNA345105, NP_689674.1, 235745_at
FIG. 5016: PRO95632
FIG. 5017A-B: DNA335175, DNA335175,
235971_at
FIG. 5018: PRO89566
FIG. 5019A-B: DNA345106, 244378.1, 236125_at
FIG. 5020: PRO49375
FIG. 5021: DNA336348, 1512910.2, 236203_at
FIG. 5022: PRO90492
FIG. 5023: DNA331211, 392245.1, 236226_at
FIG. 5024: PRO86341
FIG. 5025: DNA335691, DNA335691, 236280_at
FIG. 5026: PRO12646
FIG. 5027: DNA345107, AF488410, 236313_at
FIG. 5028A-B: DNA345108, AF318353, 236322_at
FIG. 5029: PRO95634
FIG. 5030: DNA329312, AF414120, 236341_at
FIG. 5031: PRO84901
FIG. 5032: DNA333653, 325998.1, 236435_at
FIG. 5033: PRO88292
FIG. 5034: DNA345109, 7763130.1, 236471_at
FIG. 5035: PRO95635
FIG. 5036: DNA328168, 179804.1, 236474_at
FIG. 5037: PRO84071
FIG. 5038: DNA345110, 7691553.11, 236488_s_at
FIG. 5039: PRO95636
FIG. 5040: DNA330934, DNA330934, 236595_at
FIG. 5041: PRO86095
FIG. 5042: DNA330935, 229915.1, 236610_at
FIG. 5043: PRO86096
FIG. 5044: DNA345111, 414146.8, 236717_at
FIG. 5045: PRO95637
FIG. 5046: DNA329491, DNA329491, 236787_at
FIG. 5047: DNA330939, 214517.1, 236796_at
FIG. 5048: PRO86100
FIG. 5049: DNA345112, AK074237, 236984_at
FIG. 5050: PRO95638
FIG. 5051: DNA330943, 1042935.2, 237009_at
FIG. 5052: PRO86104
FIG. 5053: DNA345113, 7762795.1, 237105_at
FIG. 5054: PRO95639
FIG. 5055A-B: DNA226536, NM_003234,
237215_s_at
FIG. 5056: PRO36999
FIG. 5057: DNA345114, BC032694, 237559_at
FIG. 5058: PRO78081
FIG. 5059: DNA328178, 985267.1, 237839_at
FIG. 5060: PRO84081
FIG. 5061: DNA330950, 983684.2, 237953_at
FIG. 5062: PRO86111
FIG. 5063A-B: DNA345115, 062186.18, 238002_at
FIG. 5064: PRO60111
FIG. 5065: DNA345116, BC033490, 238018_at
FIG. 5066: PRO95640
FIG. 5067A-B: DNA330952, 333610.10,
238021_s_at
FIG. 5068: PRO86113
FIG. 5069: DNA345117, 333610.2, 238022_at
FIG. 5070: PRO95641
FIG. 5071: DNA345118, 337083.5, 238075_at
FIG. 5072: PRO95642
FIG. 5073: DNA329492, 017295.1, 238156_at
FIG. 5074: PRO85053
FIG. 5075: DNA345119, 331249.6, 238520_at
FIG. 5076: PRO95643
FIG. 5077: DNA329495, 1447201.1, 238581_at
FIG. 5078: PRO85056
FIG. 5079: DNA329497, 232064.1, 238619_at
FIG. 5080: PRO85058
FIG. 5081A-B: DNA345120, 1400266.11, 238649_at
FIG. 5082: PRO95644
FIG. 5083: DNA334895, 172305.1, 238787_at
FIG. 5084: PRO89333
FIG. 5085: DNA328188, 7688626.1, 238875_at
FIG. 5086: PRO84091
FIG. 5087: DNA345121, 255109.1, 238900_at
FIG. 5088: PRO95645
FIG. 5089: DNA329500, 214454.1, 238950_at
FIG. 5090: PRO85061
FIG. 5091A-C: DNA345122, NM_018136,
239002_at
FIG. 5092: PRO95646
FIG. 5093A-B: DNA345123, 086440.4, 239151_at
FIG. 5094: PRO95647
FIG. 5095: DNA335753, 408088.2, 239179_at
FIG. 5096: PRO90062
FIG. 5097: DNA345124, 7685093.8, 239237_at
FIG. 5098: PRO95648
FIG. 5099: DNA345125, 401336.15, 239288_at
FIG. 5100: PRO95649
FIG. 5101: DNA333746, 332697.1, 239294_at
FIG. 5102: PRO88371
FIG. 5103: DNA345126, AL713733, 239412_at
FIG. 5104: PRO95650
FIG. 5105: DNA329502, 210572.1, 239427_at
FIG. 5106: PRO85063
FIG. 5107: DNA330983, 305289.1, 239448_at
FIG. 5108: PRO86142
FIG. 5109: DNA345127, 1397901.50, 239629_at
FIG. 5110: PRO95651
FIG. 5111: DNA333632, 247565.1, 240064_at
FIG. 5112: PRO88274
FIG. 5113: DNA330314, 026641.5, 240265_at
FIG. 5114: PRO85538
FIG. 5115: DNA340269, DNA340269, 240572_s_at
FIG. 5116: PRO91765
FIG. 5117A-B: DNA345128, NM_175571,
240646_at
FIG. 5118: PRO86060
FIG. 5119: DNA345129, 217952.1, 240789_at
FIG. 5120: PRO95652
FIG. 5121: DNA345130, 231676.2, 240951_at
FIG. 5122: PRO95653
FIG. 5123: DNA345131, NM_139273, 240983_s_at
FIG. 5124: PRO95654
FIG. 5125: DNA345132, 227682.1, 241393_at
FIG. 5126: PRO95655
FIG. 5127: DNA345133, BC016950, 241682_at
FIG. 5128: PRO95656
FIG. 5129: DNA345134, 212515.1, 241819_at
FIG. 5130: PRO24261
FIG. 5131: DNA331011, 979953.1, 241859_at
FIG. 5132: PRO86169
FIG. 5133: DNA345135, AK074645, 241869_at
FIG. 5134: PRO95657
FIG. 5135: DNA329506, NP_387510.1, 241937_s_at
FIG. 5136: PRO85067
FIG. 5137: DNA345136, 264653.1, 241956_at
FIG. 5138: PRO95658
FIG. 5139: DNA331015, 109159.1, 242031_at
FIG. 5140: PRO86173
FIG. 5141: DNA345137, 072859.8, 242146_at
FIG. 5142: PRO95659
FIG. 5143: DNA345138, 1502644.28, 242520_s_at
FIG. 5144: PRO95660
FIG. 5145A-B: DNA345139, AB067489, 242665_at
FIG. 5146: DNA331031, 405967.1, 242669_at
FIG. 5147: PRO86189
FIG. 5148A-B: DNA345140, NM_015979,
242706_s_at
FIG. 5149: PRO85734
FIG. 5150: DNA345141, 7698324.1, 242939_at
FIG. 5151: PRO95662
FIG. 5152: DNA329507, 407430.1, 242943_at
FIG. 5153: PRO85068
FIG. 5154: DNA335321, 350834.1, 243049_at
FIG. 5155: PRO89696
FIG. 5156: DNA345142, 011019.14, 243124_at
FIG. 5157: PRO95663
FIG. 5158: DNA345143, AL833716, 243166_at
FIG. 5159: PRO95664
FIG. 5160A-B: DNA329508, 142131.16, 243296_at
FIG. 5161: PRO85069
FIG. 5162: DNA345144, 407288.1, 243386_at
FIG. 5163: PRO95665
FIG. 5164: DNA345145, 994948.45, 243405_at
FIG. 5165: PRO95666
FIG. 5166: DNA331051, 306804.1, 243469_at
FIG. 5167: PRO86209
FIG. 5168A-B: DNA345146, 331965.1, 243495_s_at
FIG. 5169: PRO52796
FIG. 5170: DNA333748, 394811.1, 243602_at
FIG. 5171: PRO88373
FIG. 5172: DNA345147, 315972.1, 243788_at
FIG. 5173: PRO95667
FIG. 5174: DNA345148, 086440.19, 243937_x_at
FIG. 5175: PRO95668
FIG. 5176A-B: DNA329494, 978990.1, 243999_at
FIG. 5177: PRO85055
FIG. 5178: DNA345149, 1009940.1, 244042_x_at
FIG. 5179: PRO95669
FIG. 5180: DNA335678, 432509.1, 244044_at
FIG. 5181: PRO90006
FIG. 5182: DNA334339, DNA334339, 244267_at
FIG. 5183: PRO86220
FIG. 5184: DNA345150, 333325.3, 244308_at
FIG. 5185: PRO95670
FIG. 5186: DNA328237, 337066.49, 244383_at
FIG. 5187: PRO84140
FIG. 5188A-B: DNA345151, NP_689742.2,
244509_at
FIG. 5189: PRO95671
FIG. 5190: DNA334446, 207194.3, 244579_at
FIG. 5191: PRO88952
FIG. 5192: DNA333766, 215245.1, 244598_at
FIG. 5193: PRO88390
FIG. 5194: DNA345152, 032035.3, 244764_at
FIG. 5195: PRO95672
FIG. 5196: DNA331069, DNA331069, 244798_at
FIG. 5197: PRO86226
FIG. 5198A-B: DNA328729, BAA11496.1,
D80001_at
FIG. 5199: PRO38526
FIG. 5200: DNA328961, BC011049, DNA36995_at
FIG. 5201: PRO84667
FIG. 5202: DNA304492, NM_032016,
DNA45409_at
FIG. 5203: PRO1864
FIG. 5204: DNA327200, NM_031950,
DNA59602_at
FIG. 5205: PRO1065
FIG. 5206: DNA345153, BC031639, DNA61875_at
FIG. 5207: PRO83478
FIG. 5208: DNA345154, NP_002174.1,
DNA82348_at
FIG. 5209: PRO2021
FIG. 5210: DNA327667, NP_065392.1,
DNA84141_at
FIG. 5211: PRO83135
FIG. 5212: DNA325850, NM_024089,
DNA84917_at
FIG. 5213: PRO82312
FIG. 5214: DNA325654, NM_014033,
DNA92232_at
FIG. 5215: PRO4348
FIG. 5216A-B: DNA345155, NM_153837,
DNA96860_at
FIG. 5217: PRO6017
FIG. 5218: DNA96866, DNA96866, DNA96866_at
FIG. 5219: PRO6015
FIG. 5220: DNA331073, NP_112184.1,
DNA101926_at
FIG. 5221: PRO86229
FIG. 5222: DNA108681, DNA108681,
DNA108681_at
FIG. 5223: PRO6492
FIG. 5224: DNA329215, NM_012092,
DNA108917_at
FIG. 5225: PRO7424
FIG. 5226: DNA345156, BC047595, DNA119482_at
FIG. 5227: PRO9850
FIG. 5228A-B: DNA345157, BAA86515.1,
DNA132162_at
FIG. 5229: PRO95673
FIG. 5230: DNA345158, BC044246, DNA139546_at
FIG. 5231: PRO95674
FIG. 5232: DNA324246, NM_030926,
DNA143288_at
FIG. 5233: PRO80930
FIG. 5234A-B: DNA150956, D31887,
DNA150956_at
FIG. 5235: DNA304833, NP_443163.1,
DNA161000_at
FIG. 5236: PRO71240
FIG. 5237: DNA330417, NP_085144.1,
DNA164989_at
FIG. 5238: PRO21341
FIG. 5239: DNA345159, BC050675, P_Z93700_at
FIG. 5240: PRO95675
FIG. 5241: DNA329207, AL442092, P_X52226_at
FIG. 5242: PRO220
FIG. 5243: DNA345160, BC025407, P_X52238_at
FIG. 5244: PRO95676
FIG. 5245: DNA345161, BC009955, P_Z34109_at
FIG. 5246A-B: DNA330610, BAB15739.1,
P_A37063_at
FIG. 5247: PRO85787
FIG. 5248: DNA328250, NP_443164.1, P_Z65107_at
FIG. 5249: PRO82061
FIG. 5250: DNA304469, NP_149078.1, P_A37079_at
FIG. 5251: PRO71045
FIG. 5252: DNA345162, NM_153206, P_Z65110_at
FIG. 5253: PRO95678
FIG. 5254: DNA345163, NM_171846, P_A37128_at
FIG. 5255: PRO95679
FIG. 5256A-C: DNA345164, NM_020477,
NM_000037_at
FIG. 5257: PRO95680
FIG. 5258: DNA109234, NM_000074,
NM_000074_at
FIG. 5259: PRO6517
FIG. 5260: DNA325711, NM_000075,
NM_000075_at
FIG. 5261: PRO4873
FIG. 5262: DNA227514, NP_000152.1,
NM_000161_at
FIG. 5263: PRO37977
FIG. 5264: DNA287630, NM_000169,
NM_000169_at
FIG. 5265: PRO2154
FIG. 5266: DNA328612, NP_000166.2,
NM_000175_at
FIG. 5267: PRO84394
FIG. 5268: DNA76511, NP_000197.1,
NM_000206_at
FIG. 5269: PRO2539
FIG. 5270A-B: DNA220748, NM_000210,
NM_000210_at
FIG. 5271: PRO34726
FIG. 5272: DNA88450, NM_000235, NM_000235_at
FIG. 5273: PRO2795
FIG. 5274: DNA226014, NM_000239,
NM_000239_at
FIG. 5275: PRO36477
FIG. 5276: DNA227071, NM_000269,
NM_000269_at
FIG. 5277: PRO37534
FIG. 5278: DNA226078, NP_000296.1,
NM_000305_at
FIG. 5279: PRO36541
FIG. 5280: DNA226082, NP_000301.1,
NM_000310_at
FIG. 5281: PRO36545
FIG. 5282A-B: DNA226395, NM_000321,
NM_000321_at
FIG. 5283: PRO36858
FIG. 5284A-C: DNA345165, AF039704,
NM_000391_at
FIG. 5285: DNA227081, NP_000390.2,
NM_000399_at
FIG. 5286: PRO37544
FIG. 5287: DNA76514, NM_000418, NM_000418_at
FIG. 5288: PRO2540
FIG. 5289: DNA88549, M28526, NM_000442_at
FIG. 5290: PRO2408
FIG. 5291A-E: DNA226238, NM_000540,
NM_000540_at
FIG. 5292A-B: PRO36701
FIG. 5293: DNA83046, M31516, NM_000574_at
FIG. 5294: PRO2569
FIG. 5295A-B: DNA227659, NM_000579,
NM_000579_at
FIG. 5296: PRO38122
FIG. 5297: DNA345166, NM_000584,
NM_000584_at
FIG. 5298: PRO74
FIG. 5299: DNA345167, NM_000588,
NM_000588_at
FIG. 5300: PRO95682
FIG. 5301: DNA36717, NM_000590, NM_000590_at
FIG. 5302: PRO72
FIG. 5303: DNA345168, NM_000593,
NM_000593_at
FIG. 5304: PRO36996
FIG. 5305: DNA218655, M10988, NM_000594_at
FIG. 5306: PRO34451
FIG. 5307: DNA35629, NM_000595, NM_000595_at
FIG. 5308: PRO7
FIG. 5309: DNA225829, M59040, NM_000610_at
FIG. 5310: PRO36292
FIG. 5311: DNA345169, NP_000607.1,
NM_000616_at
FIG. 5312: PRO2222
FIG. 5313: DNA225528, NM_000619,
NM_000619_at
FIG. 5314: PRO35991
FIG. 5315: DNA227597, NM_000636,
NM_000636_at
FIG. 5316: PRO38060
FIG. 5317: DNA188234, NM_000639,
NM_000639_at
FIG. 5318: PRO21942
FIG. 5319: DNA331493, NM_000647,
NM_000647_at
FIG. 5320: PRO84690
FIG. 5321: DNA225993, NM_000655,
NM_000655_at
FIG. 5322: PRO36456
FIG. 5323: DNA89242, NM_000700, NM_000700_at
FIG. 5324: PRO2907
FIG. 5325: DNA88194, NM_000733, NM_000733_at
FIG. 5326: PRO2220
FIG. 5327: DNA90631, NM_000756, NM_000756_at
FIG. 5328: PRO2519
FIG. 5329: DNA345170, NM_000758,
NM_000758_at
FIG. 5330: PRO2055
FIG. 5331A-B: DNA226870, DNA226870,
NM_000791_at
FIG. 5332: PRO37333
FIG. 5333: DNA151820, NM_000860,
NM_000860_at
FIG. 5334: PRO12194
FIG. 5335A-B: DNA345171, NP_000868.1,
NM_000877_at
FIG. 5336: PRO2590
FIG. 5337A-B: DNA331484, NM_000878,
NM_000877_at
FIG. 5338: PRO3276
FIG. 5339: DNA345172, NM_000879,
NM_000879_at
FIG. 5340: PRO69
FIG. 5341A-B: DNA220746, NM_000885,
FIG. 5342: PRO34724
FIG. 5343: DNA220761, NM_000889,
NM_000889_at
FIG. 5344: PRO34739
FIG. 5345A-B: DNA345173, NM_138822,
NM_000919_at
FIG. 5346: PRO95683
FIG. 5347: DNA326011, NP_000933.1,
NM_000942_at
FIG. 5348: PRO2720
FIG. 5349: DNA227709, NM_000956,
NM_000956_at
FIG. 5350: PRO38172
FIG. 5351: DNA226195, NM_000958,
NM_000958_at
FIG. 5352: PRO36658
FIG. 5353A-B: DNA226070, NM_000963,
NM_000963_at
FIG. 5354: PRO36533
FIG. 5355A-B: DNA333708, NM_001066,
NM_001066_at
FIG. 5356: PRO21928
FIG. 5357A-B: DNA150748, NM_001114,
NM_001114_at
FIG. 5358: PRO12446
FIG. 5359: DNA225584, NM_001154,
NM_001154_at
FIG. 5360: PRO36047
FIG. 5361A-B: DNA325972, NM_001211,
NM_001211_at
FIG. 5362: PRO82417
FIG. 5363: DNA327718, NM_033307,
NM_001225_at
FIG. 5364: PRO83697
FIG. 5365: DNA287267, NP_001228.1,
NM_001237_at
FIG. 5366: PRO37015
FIG. 5367: DNA226177, NM_001295,
NM_001295_at
FIG. 5368: PRO36640
FIG. 5369: DNA331744, NM_001335,
NM_001335_at
FIG. 5370: PRO1574
FIG. 5371: DNA226182, NP_001391.2,
NM_001400_at
FIG. 5372: PRO36645
FIG. 5373: DNA227344, NP_001403.1,
NM_001412_at
FIG. 5374: PRO37807
FIG. 5375: DNA97300, NP_001407.1,
NM_001416_at
FIG. 5376: PRO3647
FIG. 5377: DNA188346, NM_001459,
NM_001459_at
FIG. 5378: PRO21766
FIG. 5379: DNA227752, X95876, NM_001504_at
FIG. 5380: PRO38215
FIG. 5381: DNA329941, NM_001552,
NM_001552_at
FIG. 5382: PRO85249
FIG. 5383A-B: DNA345174, NM_001558,
NM_001558_at
FIG. 5384: PRO2536
FIG. 5385A-B: DNA345175, NM_001559,
NM_001559_at
FIG. 5386: PRO23394
FIG. 5387: DNA218677, L12964, NM_001561_at
FIG. 5388: PRO34455
FIG. 5389: DNA82362, NM_001565, NM_001565_at
FIG. 5390: PRO1718
FIG. 5391A-B: DNA226364, NP_001612.1,
NM_001621_at
FIG. 5392: PRO36827
FIG. 5393: DNA88076, NM_001637, NM_001637_at
FIG. 5394: PRO2640
FIG. 5395: DNA188736, U00115, NM_001706_at
FIG. 5396: PRO26296
FIG. 5397A-B: DNA83031, NM_001746,
NM_001746_at
FIG. 5398: PRO2564
FIG. 5399: DNA150725, NM_001747,
NM_001747_at
FIG. 5400: PRO12792
FIG. 5401: DNA227480, NP_001739.1,
NM_001748_at
FIG. 5402: PRO37943
FIG. 5403: DNA345176, 348151.15, NM_001759_at
FIG. 5404: PRO95684
FIG. 5405: DNA103588, L27706, NM_001762_at
FIG. 5406: PRO4912
FIG. 5407: DNA75526, NM_001767, NM_001767_at
FIG. 5408: PRO2013
FIG. 5409: DNA328387, NM_001769,
NM_001769_at
FIG. 5410: PRO4769
FIG. 5411: DNA226380, NM_001774,
NM_001774_at
FIG. 5412: PRO4695
FIG. 5413: DNA226234, NM_001775,
NM_001775_at
FIG. 5414: PRO36697
FIG. 5415: DNA328522, NM_001778,
NM_001778_at
FIG. 5416: PRO2696
FIG. 5417: DNA226436, NM_001781,
NM_001781_at
FIG. 5418: PRO36899
FIG. 5419: DNA227573, NP_001780.1,
NM_001789_at
FIG. 5420: PRO38036
FIG. 5421: DNA329940, NM_001814,
NM_001814_at
FIG. 5422: PRO2679
FIG. 5423: DNA225671, NM_001831,
NM_001831_at
FIG. 5424: PRO36134
FIG. 5425: DNA196361, NM_001837,
NM_001837_at
FIG. 5426: PRO24864
FIG. 5427: DNA88224, NM_001838, NM_001838_at
FIG. 5428: PRO2236
FIG. 5429: DNA227606, NM_001881,
NM_001881_at
FIG. 5430: PRO38069
FIG. 5431: DNA225804, DNA225804,
NM_001908_at
FIG. 5432: PRO3344
FIG. 5433: DNA225661, NP_001944.1,
NM_001953_at
FIG. 5434: PRO36124
FIG. 5435: DNA226872, NM_001964,
NM_001964_at
FIG. 5436: PRO37335
FIG. 5437: DNA325595, NP_001966.1,
NM_001975_at
FIG. 5438: PRO38010
FIG. 5439: DNA226133, NM_001992,
NM_001992_at
FIG. 5440: PRO36596
FIG. 5441: DNA226892, DNA226892,
NM_002053_at
FIG. 5442: PRO12478
FIG. 5443: DNA88352, NM_002076, NM_002076_at
FIG. 5444: PRO2759
FIG. 5445: DNA88374, NM_002104, NM_002104_at
FIG. 5446: PRO2768
FIG. 5447: DNA151752, NM_002133,
NM_002133_at
FIG. 5448: PRO12886
FIG. 5449: DNA228014, NM_002162,
NM_002162_at
FIG. 5450: PRO38477
FIG. 5451A-B: DNA345177, NP_002173.1,
NM_002182_at
FIG. 5452: PRO6177
FIG. 5453: DNA345178, NM_002185,
NM_002185_at
FIG. 5454: PRO95685
FIG. 5455: DNA345179, NM_002186,
NM_002186_at
FIG. 5456: PRO64957
FIG. 5457: DNA345180, NM_002188,
NM_002188_at
FIG. 5458: PRO95686
FIG. 5459A-B: DNA220744, NP_002194.1,
NM_002203_at
FIG. 5460: PRO34722
FIG. 5461A-B: DNA88423, NP_002200.1,
NM_002209_at
FIG. 5462: PRO2784
FIG. 5463A-B: DNA325306, NM_002211,
NM_002211_at
FIG. 5464: PRO81851
FIG. 5465: DNA345181, NP_689926.1,
NM_002219_at
FIG. 5466: PRO95687
FIG. 5467A-C: DNA328811, D26070,
NM_002222_at
FIG. 5468: PRO84551
FIG. 5469: DNA226359, DNA226359,
NM_002228_at
FIG. 5470: PRO36822
FIG. 5471: DNA103320, NM_002229,
NM_002229_at
FIG. 5472: PRO4650
FIG. 5473: DNA345182, NM_002250,
NM_002250_at
FIG. 5474: PRO4787
FIG. 5475: DNA150971, NM_002258,
NM_002258_at
FIG. 5476: PRO12564
FIG. 5477: DNA226427, NM_002260,
NM_002260_at
FIG. 5478: PRO36890
FIG. 5479A-B: DNA345183, AJ000673,
NM_002262_at
FIG. 5480: DNA345184, BC036703, NM_002265_at
FIG. 5481: PRO82739
FIG. 5482: DNA288243, NM_002286,
NM_002286_at
FIG. 5483: PRO36451
FIG. 5484A-B: DNA188301, NM_002309,
NM_002309_at
FIG. 5485: PRO21834
FIG. 5486: DNA151012, NM_009588,
NM_002341_at
FIG. 5487: PRO11604
FIG. 5488A-B: DNA196641, NM_002349,
NM_002349_at
FIG. 5489: PRO25114
FIG. 5490: DNA103245, M16038, NM_002350_at
FIG. 5491: PRO4575
FIG. 5492: DNA227033, NM_002371,
NM_002371_at
FIG. 5493: PRO37496
FIG. 5494: DNA345185, NP_002380.3,
NM_002389_at
FIG. 5495: PRO95689
FIG. 5496: DNA103554, J03569, NM_002394_at
FIG. 5497: PRO4881
FIG. 5498: DNA97290, NM_002512, NM_002512_at
FIG. 5499: PRO3637
FIG. 5500: DNA88035, NM_002526, NM_002526_at
FIG. 5501: PRO2135
FIG. 5502: DNA345186, NM_175080,
NM_002561_at
FIG. 5503: PRO95690
FIG. 5504A-B: DNA329120, NM_002569,
NM_002569_at
FIG. 5505: PRO2752
FIG. 5506: DNA83130, NM_002674, NM_002674_at
FIG. 5507: PRO2096
FIG. 5508: DNA345187, NP_002698.1,
NM_002707_at
FIG. 5509: DNA227090, NP_002750.1,
NM_002759_at
FIG. 5510: PRO37553
FIG. 5511: DNA345188, NP_002795.2,
NM_002804_at
FIG. 5512: PRO81979
FIG. 5513A-B: DNA345189, NM_002844,
NM_002844_at
FIG. 5514: PRO95691
FIG. 5515: DNA227063, NM_002858,
NM_002858_at
FIG. 5516: PRO37526
FIG. 5517: DNA219225, NP_002874.1,
NM_002883_at
FIG. 5518: PRO34531
FIG. 5519: DNA88607, NP_002892.1,
NM_002901_at
FIG. 5520: PRO2863
FIG. 5521: DNA103281, NM_002908,
NM_002908_at
FIG. 5522: PRO4611
FIG. 5523: DNA216508, NM_002981,
NM_002981_at
FIG. 5524: PRO34260
FIG. 5525: DNA192060, NM_002983,
NM_002983_at
FIG. 5526: PRO21960
FIG. 5527: DNA216689, NM_002984,
NM_002984_at
FIG. 5528: PRO34276
FIG. 5529: DNA329241, NP_003002.1,
NM_003011_at
FIG. 5530: PRO84846
FIG. 5531: DNA329005, NM_003037,
NM_003037_at
FIG. 5532: PRO12612
FIG. 5533A-B: DNA326573, NP_003063.2,
NM_003072_at
FIG. 5534: PRO82935
FIG. 5535: DNA345190, NM_139276,
NM_003150_at
FIG. 5536: PRO95692
FIG. 5537: DNA227447, X59871, NM_003202_at
FIG. 5538: PRO37910
FIG. 5539A-B: DNA226536, X01060,
NM_003234_at
FIG. 5540: PRO36999
FIG. 5541A-B: DNA83176, NM_003243,
NM_003243_at
FIG. 5542: PRO2620
FIG. 5543: DNA227874, NM_003329,
NM_003329_at
FIG. 5544: PRO38337
FIG. 5545: DNA103421, NP_003366.1,
NM_003375_at
FIG. 5546: PRO4749
FIG. 5547: DNA345191, X71635, NM_003467_at
FIG. 5548: PRO4516
FIG. 5549: DNA304489, NM_003504,
NM_003504_at
FIG. 5550: PRO71058
FIG. 5551: DNA227239, NM_003506,
NM_003506_at
FIG. 5552: PRO37702
FIG. 5553: DNA150990, X84958, NM_003641_at
FIG. 5554: PRO12570
FIG. 5555: DNA333697, NM_003650,
NM_003650_at
FIG. 5556: PRO88328
FIG. 5557: DNA151802, AB004066, NM_003670_at
FIG. 5558: PRO12890
FIG. 5559: DNA227213, NP_003671.1,
NM_003680_at
FIG. 5560: PRO37676
FIG. 5561: DNA228010, NM_003688,
NM_003688_at
FIG. 5562: PRO38473
FIG. 5563: DNA345192, U88326, NM_003745_at
FIG. 5564: PRO12771
FIG. 5565: DNA345193, NM_148974,
NM_003790_at
FIG. 5566: PRO95693
FIG. 5567: DNA227921, NM_003798,
NM_003798_at
FIG. 5568: PRO38384
FIG. 5569: DNA345194, NP_003798.2,
NM_003807_at
FIG. 5570: PRO5810
FIG. 5571: DNA84130, U37518, NM_003810_at
FIG. 5572: PRO1096
FIG. 5573A-B: DNA200236, NP_003807.1,
NM_003816_at
FIG. 5574: PRO34137
FIG. 5575: DNA345195, NM_003839,
NM_003839_at
FIG. 5576: PRO20114
FIG. 5577: DNA345196, NM_003853,
NM_003853_at
FIG. 5578: PRO36013
FIG. 5579: DNA345197, NM_003855,
NM_003855_at
FIG. 5580: PRO4778
FIG. 5581: DNA325749, NP_003868.1,
NM_003877_at
FIG. 5582: PRO12839
FIG. 5583: DNA331776, NM_003897,
NM_003897_at
FIG. 5584: PRO84760
FIG. 5585: DNA227329, NP_004031.1,
NM_004040_at
FIG. 5586: PRO37792
FIG. 5587: DNA328570, NM_004049,
NM_004049_at
FIG. 5588: PRO37843
FIG. 5589: DNA88173, S93414, NM_004079_at
FIG. 5590: PRO2210
FIG. 5591: DNA103208, NM_004099,
NM_004099_at
FIG. 5592: PRO4538
FIG. 5593: DNA287620, NM_004131,
NM_004131_at
FIG. 5594: PRO2081
FIG. 5595: DNA227562, NP_004139.1,
NM_004148_at
FIG. 5596: PRO38025
FIG. 5597: DNA331392, NM_004195,
NM_004195_at
FIG. 5598: PRO364
FIG. 5599: DNA103394, U81800, NM_004207_at
FIG. 5600: PRO4722
FIG. 5601: DNA345198, NP_004212.3,
NM_004221_at
FIG. 5602: PRO95694
FIG. 5603: DNA345199, NP_004224.1,
NM_004233_at
FIG. 5604: PRO2225
FIG. 5605: DNA329130, NP_004286.2,
NM_004295_at
FIG. 5606: PRO20124
FIG. 5607: DNA287240, NM_004335,
NM_004335_at
FIG. 5608: PRO29371
FIG. 5609: DNA329008, NP_004337.2,
NM_004346_at
FIG. 5610: PRO12832
FIG. 5611: DNA226578, U47414, NM_004354_at
FIG. 5612: PRO37041
FIG. 5613: DNA345200, NP_620599.1,
NM_004357_at
FIG. 5614: PRO95695
FIG. 5615A-B: DNA151420, NM_004430,
NM_004430_at
FIG. 5616: PRO12876
FIG. 5617: DNA328541, NM_004512,
NM_004512_at
FIG. 5618: PRO4843
FIG. 5619A-C: DNA345201, NP_757366.1,
NM_004513_at
FIG. 5620: PRO95696
FIG. 5621: DNA328262, U57094, NM_004580_at
FIG. 5622: PRO84153
FIG. 5623: DNA226737, NM_004585,
NM_004585_at
FIG. 5624: PRO37200
FIG. 5625A-B: DNA345202, NM_033300,
NM_004631_at
FIG. 5626: PRO95697
FIG. 5627: DNA227700, NM_004778,
NM_004778_at
FIG. 5628: PRO38163
FIG. 5629: DNA151675, NM_004800,
NM_004800_at
FIG. 5630: PRO11975
FIG. 5631: DNA345203, NM_004810,
NM_004810_at
FIG. 5632: PRO12190
FIG. 5633: DNA345204, AJ420587, NM_004830_at
FIG. 5634: PRO95698
FIG. 5635: DNA345205, AL117422, NM_004844_at
FIG. 5636: PRO95699
FIG. 5637: DNA329010, NM_004951,
NM_004951_at
FIG. 5638: PRO23370
FIG. 5639: DNA227563, NP_004946.1,
NM_004955_at
FIG. 5640: PRO38026
FIG. 5641A-B: DNA103316, M54968,
NM_004985_at
FIG. 5642: PRO4646
FIG. 5643: DNA151043, NP_005004.1,
NM_005013_at
FIG. 5644: PRO12099
FIG. 5645: DNA227909, NP_005024.1,
NM_005033_at
FIG. 5646: PRO38372
FIG. 5647: DNA227124, NM_005127,
NM_005127_at
FIG. 5648: PRO37587
FIG. 5649: DNA328264, NM_005192,
NM_005192_at
FIG. 5650: PRO12087
FIG. 5651: DNA329159, NP_005195.2,
NM_005204_at
FIG. 5652: PRO4660
FIG. 5653: DNA88259, L15006, NM_005214_at
FIG. 5654: PRO2254
FIG. 5655: DNA189700, NM_005252,
NM_005252_at
FIG. 5656: PRO25619
FIG. 5657: DNA325989, NP_005304.3,
NM_005313_at
FIG. 5658: PRO2732
FIG. 5659: DNA225961, NM_005317,
NM_005317_at
FIG. 5660: PRO36424
FIG. 5661: DNA196628, NM_005327,
NM_005327_at
FIG. 5662: PRO25105
FIG. 5663: DNA227208, AF055377, NM_005360_at
FIG. 5664: PRO37671
FIG. 5665: DNA103269, NP_005366.1,
NM_005375_at
FIG. 5666: PRO4599
FIG. 5667: DNA188207, D28124, NM_005380_at
FIG. 5668: PRO21719
FIG. 5669: DNA153752, NP_005372.1,
NM_005381_at
FIG. 5670: PRO12926
FIG. 5671: DNA227376, NP_005393.1,
NM_005402_at
FIG. 5672: PRO37839
FIG. 5673A-B: DNA331302, NP_005424.1,
NM_005433_at
FIG. 5674: PRO12922
FIG. 5675: DNA88410, NM_005534, NM_005534_at
FIG. 5676: PRO2778
FIG. 5677: DNA226262, NM_005563,
NM_005563_at
FIG. 5678: PRO36725
FIG. 5679: DNA333671, NM_005601,
NM_005601_at
FIG. 5680: PRO37543
FIG. 5681: DNA150427, NM_005608,
NM_005608_at
FIG. 5682: PRO12243
FIG. 5683: DNA345206, NM_005627,
NM_005627_at
FIG. 5684: PRO86741
FIG. 5685: DNA226500, NM_005628,
NM_005628_at
FIG. 5686: PRO36963
FIG. 5687: DNA329013, NM_005658,
NM_005658_at
FIG. 5688: PRO20128
FIG. 5689: DNA226610, M80254, NM_005729_at
FIG. 5690: PRO37073
FIG. 5691A-B: DNA345207, NM_133482,
NM_005732_at
FIG. 5692: PRO95700
FIG. 5693: DNA88541, NM_005746, NM_005746_at
FIG. 5694: PRO2834
FIG. 5695: DNA93548, NM_005767, NM_005767_at
FIG. 5696: PRO4929
FIG. 5697: DNA227695, AF097358, NM_005810_at
FIG. 5698: PRO38158
FIG. 5699: DNA150959, NM_005822,
NM_005822_at
FIG. 5700: PRO11599
FIG. 5701: DNA328516, NM_005842,
NM_005842_at
FIG. 5702: PRO12323
FIG. 5703: DNA151825, NM_005900,
NM_005900_at
FIG. 5704: PRO12900
FIG. 5705: DNA345208, NM_130439,
NM_005962_at
FIG. 5706: PRO95701
FIG. 5707: DNA328266, NM_006002,
NM_006002_at
FIG. 5708: PRO12125
FIG. 5709: DNA225959, NM_006144,
NM_006144_at
FIG. 5710: PRO36422
FIG. 5711: DNA28759, NM_006159, NM_006159_at
FIG. 5712: PRO2520
FIG. 5713: DNA329015, NP_006155.2,
NM_006164_at
FIG. 5714: PRO84691
FIG. 5715A-B: DNA151841, M59465,
NM_006290_at
FIG. 5716: PRO12904
FIG. 5717: DNA103371, NP_006361.1,
NM_006370_at
FIG. 5718: PRO4701
FIG. 5719: DNA189708, AF155568, NM_006372_at
FIG. 5720: PRO23166
FIG. 5721: DNA150430, NM_006396,
NM_006396_at
FIG. 5722: PRO12770
FIG. 5723: DNA227112, NM_006406,
NM_006406_at
FIG. 5724: PRO37575
FIG. 5725: DNA227795, NM_006429,
NM_006429_at
FIG. 5726: PRO38258
FIG. 5727: DNA329225, NM_006495,
NM_006495_at
FIG. 5728: PRO84833
FIG. 5729: DNA226277, X91790, NM_006499_at
FIG. 5730: PRO36740
FIG. 5731: DNA103253, NP_006507.1,
NM_006516_at
FIG. 5732: PRO4583
FIG. 5733A-B: DNA331802, AF012108,
NM_006534_at
FIG. 5734: PRO86743
FIG. 5735: DNA93439, Y13248, NM_006564_at
FIG. 5736: PRO4515
FIG. 5737: DNA227751, NM_006566,
NM_006566_at
FIG. 5738: PRO38214
FIG. 5739A-B: DNA345209, NP_006697.2,
NM_006706_at
FIG. 5740: PRO95702
FIG. 5741: DNA225836, U66142, NM_006725_at
FIG. 5742: PRO36299
FIG. 5743: DNA226260, NP_006760.1,
NM_006769_at
FIG. 5744: PRO36723
FIG. 5745: DNA227190, NP_006830.1,
NM_006839_at
FIG. 5746: PRO37653
FIG. 5747: DNA324897, NM_006854,
NM_006854_at
FIG. 5748: PRO12468
FIG. 5749A-B: DNA103449, NM_006931,
NM_006931_at
FIG. 5750: PRO4776
FIG. 5751: DNA324805, NM_007047,
NM_007047_at
FIG. 5752: PRO81419
FIG. 5753: DNA328271, NM_007057,
NM_007057_at
FIG. 5754: PRO81868
FIG. 5755: DNA329189, NM_007208,
NM_007208_at
FIG. 5756: PRO4911
FIG. 5757: DNA103440, NM_007360,
NM_007360_at
FIG. 5758: PRO4767
FIG. 5759A-B: DNA345210, BC028412,
NM_012081_at
FIG. 5760: PRO37794
FIG. 5761: DNA326809, NM_012112,
NM_012112_at
FIG. 5762: PRO83142
FIG. 5763A-B: DNA151707, NP_036273.1,
NM_012141_at
FIG. 5764: PRO12884
FIG. 5765: DNA345211, NM_012449,
NM_012449_at
FIG. 5766: PRO28528
FIG. 5767: DNA150621, NM_012463,
NM_012463_at
FIG. 5768: PRO12374
FIG. 5769: DNA331485, NM_012483,
NM_012483_at
FIG. 5770: PRO86529
FIG. 5771: DNA331519, NM_012485,
NM_012484_at
FIG. 5772: PRO86551
FIG. 5773: DNA227302, NM_013269,
NM_013269_at
FIG. 5774: PRO37765
FIG. 5775: DNA225594, NM_013272,
NM_013272_at
FIG. 5776: PRO36057
FIG. 5777: DNA103481, NP_037417.1,
NM_013285_at
FIG. 5778: PRO4808
FIG. 5779: DNA196426, NM_013308,
NM_013308_at
FIG. 5780: PRO24924
FIG. 5781: DNA227125, AF132297, NM_013324_at
FIG. 5782: PRO37588
FIG. 5783: DNA150648, NM_013332,
NM_013332_at
FIG. 5784: PRO11576
FIG. 5785: DNA345212, AB025219, NM_013416_at
FIG. 5786: PRO84354
FIG. 5787: DNA345213, NM_014044,
NM_014044_at
FIG. 5788: PRO95703
FIG. 5789A-C: DNA227619, NM_014112,
NM_014112_at
FIG. 5790: PRO38082
FIG. 5791: DNA331817, NM_014339,
NM_014339_at
FIG. 5792: PRO86240
FIG. 5793: DNA227351, AF191020, NM_014367_at
FIG. 5794: PRO37814
FIG. 5795: DNA329546, NM_014399,
NM_014399_at
FIG. 5796: PRO296
FIG. 5797: DNA330084, NM_014450,
NM_014450_at
FIG. 5798: PRO9895
FIG. 5799: DNA227252, U96628, NM_014456_at
FIG. 5800: PRO37715
FIG. 5801A-B: DNA277809, D87465,
NM_014767_at
FIG. 5802: PRO64556
FIG. 5803A-B: DNA151685, NP_055610.1,
NM_014795_at
FIG. 5804: PRO12883
FIG. 5805A-B: DNA227353, NM_014822,
NM_014822_at
FIG. 5806: PRO37816
FIG. 5807: DNA150805, NM_014888,
NM_014888_at
FIG. 5808: PRO11583
FIG. 5809: DNA103333, NM_014890,
NM_014890_at
FIG. 5810: PRO4663
FIG. 5811: DNA328274, NM_014891,
NM_014891_at
FIG. 5812: PRO12912
FIG. 5813A-B: DNA304464, NM_014918,
NM_014918_at
FIG. 5814: PRO71042
FIG. 5815A-B: DNA345214, NP_619520.1,
NM_014966_at
FIG. 5816: PRO12282
FIG. 5817: DNA330103, NM_015364,
NM_015364_at
FIG. 5818: PRO19671
FIG. 5819: DNA345215, NM_015392,
NM_015392_at
FIG. 5820: PRO95704
FIG. 5821: DNA226662, NP_057043.1,
NM_015959_at
FIG. 5822: PRO37125
FIG. 5823: DNA330096, NM_015967,
NM_015967_at
FIG. 5824: PRO37163
FIG. 5825A-B: DNA345216, AF077041,
NM_016081_at
FIG. 5826: PRO95705
FIG. 5827: DNA328831, NM_016245,
NM_016245_at
FIG. 5828: PRO233
FIG. 5829: DNA227352, AF1110777, NM_016283_at
FIG. 5830: PRO37815
FIG. 5831: DNA330421, NM_016354,
NM_016354_at
FIG. 5832: PRO85626
FIG. 5833A-B: DNA328454, NM_016441,
NM_016441_at
FIG. 5834: PRO4330
FIG. 5835: DNA345217, NP_057546.1,
NM_016462_at
FIG. 5836: PRO23604
FIG. 5837: DNA227364, NP_057635.1,
NM_016551_at
FIG. 5838: PRO37827
FIG. 5839: DNA326550, NM_016579,
NM_016579_at
FIG. 5840: PRO224
FIG. 5841: DNA327869, NM_016588,
NM_016588_at
FIG. 5842: PRO1898
FIG. 5843: DNA227187, NM_016619,
NM_016619_at
FIG. 5844: PRO37650
FIG. 5845: DNA326078, NM_016641,
NM_016641_at
FIG. 5846: PRO38464
FIG. 5847: DNA227294, NM_017755,
NM_017755_at
FIG. 5848: PRO37757
FIG. 5849: DNA226633, NM_017906,
NM_017906_at
FIG. 5850: PRO37096
FIG. 5851: DNA336491, AK027630, NM_018092_at
FIG. 5852: PRO4401
FIG. 5853A-B: DNA345218, BC034607,
NM_018123_at
FIG. 5854: PRO95706
FIG. 5855: DNA227194, NM_018295,
NM_018295_at
FIG. 5856: PRO37657
FIG. 5857: DNA226227, NM_018402,
NM_018402_at
FIG. 5858: PRO36690
FIG. 5859: DNA287642, NM_018464,
NM_018464_at
FIG. 5860: PRO9902
FIG. 5861: DNA345219, AF116708, NM_018630_at
FIG. 5862: DNA304494, AF212365, NM_018725_at
FIG. 5863: PRO71061
FIG. 5864: DNA227929, NP_061932.1,
NM_019059_at
FIG. 5865: PRO38392
FIG. 5866: DNA227268, NP_061955.1,
NM_019082_at
FIG. 5867: PRO37731
FIG. 5868: DNA226256, J00194, NM_019111_at
FIG. 5869: PRO36719
FIG. 5870: DNA329552, NM_019895,
NM_019895_at
FIG. 5871: PRO85097
FIG. 5872: DNA329074, NM_020139,
NM_020139_at
FIG. 5873: PRO21326
FIG. 5874: DNA329553, NM_020150,
NM_020150_at
FIG. 5875: PRO38313
FIG. 5876: DNA227280, NP_064615.1,
NM_020230_at
FIG. 5877: PRO37743
FIG. 5878: DNA227720, NP_065161.1,
NM_020428_at
FIG. 5879: PRO38183
FIG. 5880: DNA225636, NM_020645,
NM_020645_at
FIG. 5881: PRO36099
FIG. 5882: DNA150992, NP_066362.1,
NM_021034_at
FIG. 5883: PRO12572
FIG. 5884: DNA329023, NM_021102,
NM_021102_at
FIG. 5885: PRO209
FIG. 5886: DNA227121, NM_021105,
NM_021105_at
FIG. 5887: PRO37584
FIG. 5888: DNA345220, NM_021129,
NM_021129_at
FIG. 5889: PRO11669
FIG. 5890A-B: DNA333179, AF231512,
NM_021618_at
FIG. 5891: PRO87901
FIG. 5892: DNA326379, NP_067639.1,
NM_021626_at
FIG. 5893: PRO302
FIG. 5894: DNA345221, BC004348, NM_021798_at
FIG. 5895: PRO10273
FIG. 5896: DNA331834, AF246221, NM_021999_at
FIG. 5897: PRO86760
FIG. 5898: DNA304835, NP_071327.1,
NM_022044_at
FIG. 5899: PRO71242
FIG. 5900: DNA330378, NM_022346,
NM_022346_at
FIG. 5901: PRO81126
FIG. 5902: DNA328902, NM_022355,
NM_022355_at
FIG. 5903: PRO84623
FIG. 5904: DNA328895, NM_022367,
NM_022367_at
FIG. 5905: PRO1317
FIG. 5906A-B: DNA329024, BAA25532.2,
AB011178_at
FIG. 5907: PRO84696
FIG. 5908: DNA345222, NP_612213.2,
AF007152_at
FIG. 5909: PRO95708
FIG. 5910: DNA66487, NM_002467, HSMYC1_at
FIG. 5911: PRO1213
FIG. 5912A-B: DNA325227, NP_005338.1,
HSRNABIP_at
FIG. 5913: PRO81785
FIG. 5914: DNA345223, Y00790, HSTCRGR_at
FIG. 5915: PRO95709
FIG. 5916: DNA103258, DNA103258,
HSINTASA_at
FIG. 5917: PRO4588
FIG. 5918: DNA288259, NP_114172.1,
HUMCYCB_at
FIG. 5919: PRO4676
FIG. 5920A-B: DNA227134, NP_000918.1,
HUMMDR1_at
FIG. 5921: PRO37597
FIG. 5922: DNA329025, NM_006208,
HUMPC1Q1_at
FIG. 5923: PRO4860
FIG. 5924: DNA345224, X15260, HUMTCRGC_at
FIG. 5925: DNA150552, AAB97011.1,
AF040965_at
FIG. 5926: PRO12326
FIG. 5927: DNA331095, NP_005216.1, HUME2F_at
FIG. 5928: PRO86245
FIG. 5929: DNA151041, DNA151041, P_V84330_at
FIG. 5930: PRO12849
FIG. 5931: DNA329276, NM_024096, AK024843_at
FIG. 5932: PRO12104
FIG. 5933: DNA151120, DNA151120,
HUMP13KIN_at
FIG. 5934: PRO12179
FIG. 5935: DNA345225, NM_138341, P_Z29229_at
FIG. 5936: PRO95710
FIG. 5937: DNA345226, NP_663781.1,
AK024570_at
FIG. 5938: PRO11652
FIG. 5939: DNA287190, AL049943, HSM800284_at
FIG. 5940: DNA345227, NP_005660.1,
HUMPOLLA_at
FIG. 5941: PRO95711
FIG. 5942: DNA151434, DNA151434, P_X04382_at
FIG. 5943: PRO11802
FIG. 5944: DNA345228, NP_079522.1, P_V61478_at
FIG. 5945: PRO95712
FIG. 5946A-C: DNA345229, NM_015293,
AB018339_at
FIG. 5947: PRO95713
FIG. 5948: DNA345230, M12886, HUMTCBYY_at
FIG. 5949: PRO95714
FIG. 5950A-C: DNA302013, NM_023037,
HSU50534_at
FIG. 5951: PRO71030
FIG. 5952A-B: DNA328284, NP_056356.1,
P_X37553_at
FIG. 5953: PRO84160
FIG. 5954A-B: DNA345231, 331792.1,
HSM801131_at
FIG. 5955: PRO24965
FIG. 5956: DNA151774, DNA151774, P_X85042_at
FIG. 5957: PRO12052
FIG. 5958A-B: DNA169926, DNA169926,
AB032991_at
FIG. 5959: PRO23259
FIG. 5960A-B: DNA345232, NM_006996,
HSA237724_at
FIG. 5961: PRO23299
FIG. 5962A-B: DNA329269, AB007916,
AB007916_at
FIG. 5963A-B: DNA193917, AL050367,
HSM800541_at
FIG. 5964: DNA330906, NM_032782, P_A51904_at
FIG. 5965: PRO86067
FIG. 5966: DNA193996, DNA193996, P_A40502_at
FIG. 5967: PRO23400
FIG. 5968: DNA194141, DNA194141, P_X37431_at
FIG. 5969: PRO23535
FIG. 5970: DNA228132, AK027031, AK027031_at
FIG. 5971: PRO38595
FIG. 5972: DNA345233, AL136919, P_Z51682_at
FIG. 5973: PRO95715
FIG. 5974: DNA328288, BC020517, AK022938_at
FIG. 5975: PRO69876
FIG. 5976: DNA345234, AK026962, AK026962_at
FIG. 5977: PRO95716
FIG. 5978: DNA331098, AY052405, AX047348_at
FIG. 5979: PRO86248
FIG. 5980: DNA345235, 221966.14,
AI984778_RC_at
FIG. 5981: PRO95717
FIG. 5982: DNA345236, 330869.67, AV762213_at
FIG. 5983: PRO95718
FIG. 5984: DNA210194, DNA210194,
HSM802254_at
FIG. 5985: DNA331856, BC022522, 237658.8_at
FIG. 5986: PRO71209
FIG. 5987: DNA194527, DNA194527, 399617.1_at
FIG. 5988: PRO23884
FIG. 5989: DNA345237, 196714.4, 196714.2_at
FIG. 5990: PRO95719
FIG. 5991: DNA345238, 001697.46, 001697.5_at
FIG. 5992: PRO95720
FIG. 5993: DNA345239, AAH35779.1, 399901.2_at
FIG. 5994: PRO95721
FIG. 5995: DNA338349, BC035900, 428335.22_at
FIG. 5996: PRO91021
FIG. 5997: DNA164635, DNA164635,
DNA164635_at
FIG. 5998: DNA326749, NP_116101.1,
DNA167237_at
FIG. 5999: PRO23238
FIG. 6000: DNA210622, NM_015925,
NN_015925_at
FIG. 6001: PRO35016
FIG. 6002: DNA345240, 098138.2, P_Q74306_at
FIG. 6003: PRO95722
FIG. 6004: DNA330438, NM_018556,
NM_018556_at
FIG. 6005: PRO50795
FIG. 6006: DNA345241, NM_018384,
NM_018384_at
FIG. 6007: PRO95723
FIG. 6008: DNA254520, NM_018482,
NM_018482_at
FIG. 6009: PRO49627
FIG. 6010: DNA254470, NM_002497,
NM_002497_at
FIG. 6011: PRO49578
FIG. 6012A-B: DNA331400, NM_018440,
NM_018440_at
FIG. 6013: PRO86464
FIG. 6014: DNA254414, NP_054898.1,
NM_014179_at
FIG. 6015: PRO49524
FIG. 6016: DNA255340, NM_017684,
NM_017684_at
FIG. 6017: PRO50409
FIG. 6018: DNA253811, NP_004410.2,
NM_004419_at
FIG. 6019: PRO49214
FIG. 6020: DNA255921, NM_000734,
NM_000734_at
FIG. 6021: PRO50974
FIG. 6022: DNA345242, BC002342, NM_014325_at
FIG. 6023: PRO49875
FIG. 6024: DNA255161, NM_022147,
NM_022147_at
FIG. 6025: PRO50241
FIG. 6026: DNA330123, NM_007053,
NM_007053_at
FIG. 6027: PRO35080
FIG. 6028: DNA327812, NM_006417,
NM_006417_at
FIG. 6029: PRO83773
FIG. 6030: DNA304717, NM_000389,
NM_000389_at
FIG. 6031: PRO71143
FIG. 6032: DNA328431, NM_001826,
NM_001826_at
FIG. 6033: PRO45093
FIG. 6034A-B: DNA333574, NM_002829,
NM_002829_at
FIG. 6035: PRO88221
FIG. 6036: DNA345243, L38616, NM_004899_at
FIG. 6037: PRO95724
FIG. 6038: DNA287207, NM_006325,
NM_006325_at
FIG. 6039: PRO39268
FIG. 6040: DNA329172, NM_005263,
NM_005263_at
FIG. 6041: PRO84796
FIG. 6042: DNA345244, NP_036229.1,
NM_012097_at
FIG. 6043: PRO71114
FIG. 6044: DNA256257, NM_014398,
NM_014398_at
FIG. 6045: PRO51301
FIG. 6046A-B: DNA221079, NM_022162,
NM_022162_at
FIG. 6047: PRO34753
FIG. 6048: DNA255454, NP_060834.1,
NM_018364_at
FIG. 6049: PRO50521
FIG. 6050A-B: DNA254789, NM_016217,
NM_016217_at
FIG. 6051: PRO49887
FIG. 6052A-B: DNA254376, NM_014963,
NM_014963_at
FIG. 6053: PRO49486
FIG. 6054: DNA254214, NM_001698,
NM_001698_at
FIG. 6055: PRO49326
FIG. 6056: DNA345245, BC015815, NM_006994_at
FIG. 6057: PRO49242
FIG. 6058: DNA253802, NP_055569.1,
NM_014754_at
FIG. 6059: PRO49207
FIG. 6060: DNA255269, AL110271, NM_015462_at
FIG. 6061: PRO50346
FIG. 6062: DNA256521, NM_013431,
NM_013431_at
FIG. 6063: PRO51556
FIG. 6064A-B: DNA345246, NM_138292,
NM_000051_at
FIG. 6065: PRO95725
FIG. 6066: DNA256533, NM_006114,
NM_006114_at
FIG. 6067: PRO51565
FIG. 6068A-B: DNA287273, NM_006444,
NM_006444_at
FIG. 6069: PRO69545
FIG. 6070: DNA330223, NP_001790.1,
NM_001799_at
FIG. 6071: PRO49730
FIG. 6072: DNA254350, NM_004052,
NM_004052_at
FIG. 6073: PRO49461
FIG. 6074: DNA254163, S73813, NM_001776_at
FIG. 6075: PRO49277
FIG. 6076: DNA328876, NP_060582.1,
NM_018112_at
FIG. 6077: PRO84603
FIG. 6078: DNA329900, M87338, NM_002914_at
FIG. 6079: PRO81549
FIG. 6080: DNA330040, NM_078626,
NM_001262_at
FIG. 6081: PRO59546
FIG. 6082: DNA339592, NP_071401.2,
NM_022118_at
FIG. 6083: PRO91353
FIG. 6084: DNA329575, NP_004699.1,
NM_004708_at
FIG. 6085: PRO61403
FIG. 6086: DNA277083, M84489, NM_002745_at
FIG. 6087: PRO64127
FIG. 6088: DNA327690, NM_004031,
NM_004031_at
FIG. 6089: PRO83673
FIG. 6090: DNA272066, NM_002940,
NM_002940_at
FIG. 6091: PRO60337
FIG. 6092: DNA345247, BC012125, NM_022154_at
FIG. 6093: PRO50332
FIG. 6094A-B: DNA254616, NM_004482,
NM_004482_at
FIG. 6095: PRO49718
FIG. 6096: DNA255402, NM_014473,
NM_014473_at
FIG. 6097: PRO50469
FIG. 6098: DNA328296, NP_061059.1,
NM_018589_at
FIG. 6099: PRO51817
FIG. 6100: DNA345248, NM_006639,
NM_006639_at
FIG. 6101: PRO34958
FIG. 6102: DNA287241, NM_015907,
NM_015907_at
FIG. 6103: PRO69516
FIG. 6104: DNA254380, NM_020379,
NM_020379_at
FIG. 6105: PRO49490
FIG. 6106A-B: DNA345249, AAH38115.1,
NM_017631_at
FIG. 6107: PRO95726
FIG. 6108: DNA287221, NP_057407.1,
NM_016323_at
FIG. 6109: PRO69500
FIG. 6110: DNA252224, AK025273, NM_022073_at
FIG. 6111: PRO48216
FIG. 6112A-B: DNA254218, NP_001914.2,
NM_001923_at
FIG. 6113: PRO49330
FIG. 6114: DNA329033, NM_005384,
NM_005384_at
FIG. 6115: PRO84700
FIG. 6116A-C: DNA345250, NP_002751.1,
NM_002760_at
FIG. 6117: PRO59148
FIG. 6118: DNA273060, NM_001255,
NM_001255_at
FIG. 6119: PRO61125
FIG. 6120: DNA345251, NP_694858.1,
NM_002270_at
FIG. 6121: PRO60223
FIG. 6122: DNA269750, NP_002919.1,
NM_002928_at
FIG. 6123: PRO58159
FIG. 6124: DNA327927, NM_013258,
NM_013258_at
FIG. 6125: PRO57311
FIG. 6126: DNA330057, NM_005950,
NM_005950_at
FIG. 6127: PRO85337
FIG. 6128A-B: DNA345252, AL136911,
NM_016357_at
FIG. 6129: PRO82143
FIG. 6130: DNA329118, NM_021874,
NM_021874_at
FIG. 6131: PRO83123
FIG. 6132A-B: DNA345253, NM_174956,
NM_005173_at
FIG. 6133: PRO95727
FIG. 6134: DNA256737, NM_017806,
NM_017806_at
FIG. 6135: PRO51671
FIG. 6136: DNA329253, NM_006137,
NM_006137_at
FIG. 6137: PRO84853
FIG. 6138: DNA254570, NP_055484.1,
NM_014669_at
FIG. 6139: PRO49673
FIG. 6140: DNA254416, NM_060915.1,
NM_018445_at
FIG. 6141: PRO49526
FIG. 6142A-C: DNA328497, NM_005502,
NM_005502_at
FIG. 6143: PRO84319
FIG. 6144A-B: DNA330366, NM_022765,
NM_022765_at
FIG. 6145: PRO85581
FIG. 6146: DNA328471, NP_005848.2,
NM_005857_at
FIG. 6147: PRO84297
FIG. 6148: DNA324742, NM_001760,
NM_001760_at
FIG. 6149: PRO81367
FIG. 6150A-B: DNA255183, NM_019027,
NM_019027_at
FIG. 6151: PRO50262
FIG. 6152: DNA256141, AL353940, NM_018423_at
FIG. 6153: PRO51189
FIG. 6154: DNA255145, NM_018447,
NM_018447_at
FIG. 6155: PRO50225
FIG. 6156: DNA256762, AK022882, NM_022451_at
FIG. 6157: PRO51695
FIG. 6158: DNA345254, NM_020437,
NM_020437_at
FIG. 6159: PRO86261
FIG. 6160: DNA329584, NP_005032.1,
NM_005041_at
FIG. 6161: PRO85118
FIG. 6162: DNA345255, AY184205, NM_015180_at
FIG. 6163: PRO95728
FIG. 6164: DNA327521, NM_002201,
NM_002201_at
FIG. 6165: PRO58320
FIG. 6166: DNA331323, NM_001259,
NM_001259_at
FIG. 6167: PRO86412
FIG. 6168: DNA272655, NM_001827,
NM_001827_at
FIG. 6169: PRO60781
FIG. 6170A-B: DNA345256, NP_665702.1,
NM_004619_at
FIG. 6171: PRO20111
FIG. 6172: DNA345257, NM_003835,
NM_003835_at
FIG. 6173: PRO95729
FIG. 6174: DNA345258, NM_002925,
NM_002925_at
FIG. 6175: PRO63255
FIG. 6176: DNA345259, NM_006538,
NM_006538_at
FIG. 6177: PRO84980
FIG. 6178: DNA270717, U31382, NM_004485_at
FIG. 6179: PRO59080
FIG. 6180: DNA152786, NP_057215.1,
NM_016131_at
FIG. 6181: PRO10928
FIG. 6182: DNA345260, NM_022168,
NM_022168_at
FIG. 6183: PRO95730
FIG. 6184A-B: DNA327674, NM_002748,
NM_002748_at
FIG. 6185: PRO83661
FIG. 6186: DNA325648, NP_037409.2,
NM_013277_at
FIG. 6187: PRO82139
FIG. 6188: DNA256561, NM_019604,
NM_019604_at
FIG. 6189: PRO51592
FIG. 6190: DNA329585, NP_005499.1,
NM_005508_at
FIG. 6191: PRO85119
FIG. 6192: DNA345261, NM_005290,
NM_005290_at
FIG. 6193: PRO54695
FIG. 6194: DNA328915, NM_014241,
NM_014241_at
FIG. 6195: PRO84634
FIG. 6196: DNA256089, D88308, NM_003645_at
FIG. 6197: PRO51139
FIG. 6198: DNA255215, AF207600, NM_018638_at
FIG. 6199: PRO50294
FIG. 6200A-B: DNA256807, NM_016255,
NM_016255_at
FIG. 6201: PRO51738
FIG. 6202: DNA255213, DNA255213,
NM_017780_at
FIG. 6203: PRO50292
FIG. 6204: DNA255386, NP_037518.1,
NM_013386_at
FIG. 6205: PRO50454
FIG. 6206A-B: DNA254292, DNA254292,
NM_004481_at
FIG. 6207: PRO49403
FIG. 6208: DNA260974, NM_006074,
NM_006074_at
FIG. 6209: PRO54720
FIG. 6210: DNA345262, NP_055118.1,
NM_014303_at
FIG. 6211: PRO49256
FIG. 6212: DNA331119, NM_005442,
NM_005442_at
FIG. 6213: PRO50745
FIG. 6214: DNA345263, NM_022468,
NM_022468_at
FIG. 6215: PRO51432
FIG. 6216: DNA254543, NP_006799.1,
NM_006808_at
FIG. 6217: PRO49648
FIG. 6218: DNA255088, NP_003249.1,
NM_003258_at
FIG. 6219: PRO50174
FIG. 6220: DNA253798, NP_002632.1,
NM_002641_at
FIG. 6221: PRO49203
FIG. 6222: DNA287425, NM_018509,
NM_018509_at
FIG. 6223: PRO69682
FIG. 6224: DNA295327, NM_021803,
NM_021803_at
FIG. 6225: PRO70773
FIG. 6226: DNA273523, NP_002154.1,
NM_002163_at
FIG. 6227: PRO61504
FIG. 6228: DNA271189, L22075, NM_006572_at
FIG. 6229: PRO59506
FIG. 6230: DNA333731, NP_055165.1,
NM_014350_at
FIG. 6231: PRO88357
FIG. 6232: DNA325507, NP_005842.1,
NM_005851_at
FIG. 6233: PRO69461
FIG. 6234: DNA294794, NM_002870,
NM_002870_at
FIG. 6235: PRO70754
FIG. 6236: DNA328303, NP_056525.1,
NM_015710_at
FIG. 6237: PRO84173
FIG. 6238: DNA345264, AL137399, NM_006785_at
FIG. 6239: DNA327858, AF120334, NM_012341_at
FIG. 6240: PRO83800
FIG. 6241: DNA331122, NP_005728.2,
NM_005737_at
FIG. 6242: PRO86265
FIG. 6243: DNA289528, NM_004311,
NM_004311_at
FIG. 6244: PRO70286
FIG. 6245: DNA329123, NM_002882,
NM_002882_at
FIG. 6246: PRO84765
FIG. 6247: DNA339428, NP_057604.1,
NM_016520_at
FIG. 6248: PRO91233
FIG. 6249: DNA329038, NP_055704.1,
NM_014889_at
FIG. 6250: PRO84705
FIG. 6251: DNA345265, NP_004216.1,
NM_004225_at
FIG. 6252: PRO95732
FIG. 6253: DNA329587, NM_012124,
NM_012124_at
FIG. 6254: PRO85121
FIG. 6255A-B: DNA329248, AB002359,
AB002359_at
FIG. 6256A-B: DNA255619, DNA255619,
AF054589_at
FIG. 6257: PRO50682
FIG. 6258A-B: DNA330255, AK025499,
HSM800958_at
FIG. 6259: PRO85488
FIG. 6260A-B: DNA255050, AL136883,
HSM801851_at
FIG. 6261: PRO50138
FIG. 6262: DNA328529, NM_001629, P_Z36336_at
FIG. 6263: PRO49814
FIG. 6264A-B: DNA329039, NP_056250.2,
AK027070_at
FIG. 6265: PRO84706
FIG. 6266: DNA328509, NM_006748, HSU44403_at
FIG. 6267: PRO57996
FIG. 6268: DNA345266, AF067023, NM_001363_at
FIG. 6269A-B: DNA345267, NM_020453,
AB040920_at
FIG. 6270: PRO95734
FIG. 6271A-B: DNA331898, AF058925,
AF058925_at
FIG. 6272: PRO86787
FIG. 6273: DNA345268, NM_032479, AF151109_at
FIG. 6274: PRO84951
FIG. 6275: DNA331901, AL117515, AB029015_at
FIG. 6276: DNA256422, AJ227900, HSA227900_at
FIG. 6277: DNA254610, Z48633, HSHRTPSN_at
FIG. 6278: DNA345269, NM_015660,
HSM800796_at
FIG. 6279: PRO95735
FIG. 6280: DNA256846, NM_017515, AK023080_at
FIG. 6281: PRO51777
FIG. 6282: DNA331902, NP_619634.1,
HSSOM172M_at
FIG. 6283: PRO86790
FIG. 6284: DNA329040, NP_005524.1,
HSU72882_at
FIG. 6285: PRO84707
FIG. 6286: DNA256796, AF083127, AF083127_at
FIG. 6287: DNA345270, AAH06437.1,
AK024476_at
FIG. 6288: PRO82523
FIG. 6289A-B: DNA256299, BAB21793.1,
AB051489_at
FIG. 6290: PRO51343
FIG. 6291: DNA330259, NP_008944.1,
HSM801707_at
FIG. 6292: PRO49366
FIG. 6293: DNA331132, NM_032148,
HSM801796_at
FIG. 6294: PRO86273
FIG. 6295: DNA255964, NM_024837, AK025125_at
FIG. 6296: PRO51015
FIG. 6297: DNA256061, NM_030921, AF267864_at
FIG. 6298: PRO51109
FIG. 6299: DNA329078, NP_112200.2,
HSM801679_at
FIG. 6300: PRO23253
FIG. 6301: DNA345271, NP_001275.1,
NM_001284_at
FIG. 6302: PRO22838
FIG. 6303: DNA304710, NM_001540,
NM_001540_at
FIG. 6304: PRO71136
FIG. 6305: DNA330023, NM_001924,
NM_001924_at
FIG. 6306: PRO85308
FIG. 6307: DNA275385, NM_002094,
NM_002094_at
FIG. 6308: PRO63048
FIG. 6309: DNA328418, NM_003407,
NM_003407_at
FIG. 6310: PRO84261
FIG. 6311: DNA345272, NM_004128,
NM_004128_at
FIG. 6312: PRO95736
FIG. 6313: DNA331133, U63830, NM_004180_at
FIG. 6314: PRO86274
FIG. 6315: DNA287203, NP_006182.1,
NM_006191_at
FIG. 6316: PRO69487
FIG. 6317: DNA325920, NM_012111,
NM_012111_at
FIG. 6318: PRO82373
FIG. 6319: DNA253807, NM_020529,
NM_020529_at
FIG. 6320: PRO49210
FIG. 6321: DNA329925, NM_001537,
NM_001537_at
FIG. 6322: PRO85239
FIG. 6323: DNA289526, NM_004024,
NM_004024_at
FIG. 6324: PRO70282
FIG. 6325: DNA269766, NP_005646.1,
NM_005655_at
FIG. 6326: PRO58175
FIG. 6327: DNA329047, NM_006399,
NM_006399_at
FIG. 6328: PRO58425
FIG. 6329: DNA274167, AF026166, NM_006431_at
FIG. 6330: PRO62097
FIG. 6331: DNA254572, NM_006585,
NM_006585_at
FIG. 6332: PRO49675
FIG. 6333: DNA328591, NP_006635.1,
NM_006644_at
FIG. 6334: PRO84376
FIG. 6335: DNA255289, NM_014791,
NM_014791_at
FIG. 6336: PRO50363
FIG. 6337: DNA345273, X15183, HSHSP90R_at
FIG. 6338: PRO95737
FIG. 6339: DNA271847, NM_001539,
NM_001539_at
FIG. 6340: PRO60127
FIG. 6341: DNA270929, M88279, NM_002014_at
FIG. 6342: PRO59262
FIG. 6343: DNA329106, AF042081, NM_003022_at
FIG. 6344: PRO83360
FIG. 6345: DNA345274, NM_174886,
NM_003244_at
FIG. 6346: PRO95738
FIG. 6347: DNA253585, NM_004418,
NM_004418_at
FIG. 6348: PRO49183
FIG. 6349A-B: DNA275334, NP_112162.1,
NM_004749_at
FIG. 6350: PRO63009
FIG. 6351A-B: DNA270923, NM_004817,
NM_004817_at
FIG. 6352: PRO59256
FIG. 6353: DNA345275, NM_005572,
NM_005572_at
FIG. 6354: PRO80660
FIG. 6355A-B: DNA328473, NP_006473.1,
NM_006482_at
FIG. 6356: PRO84299
FIG. 6357: DNA326736, NM_006666,
NM_006666_at
FIG. 6358: PRO83076
FIG. 6359: DNA290235, NP_057121.1,
NM_016037_at
FIG. 6360: PRO70335
FIG. 6361: DNA331135, D43950, HUMKG1DD_at
FIG. 6362: DNA273498, DNA273498,
HUMHSP70H_at
FIG. 6363: PRO61480
FIG. 6364: DNA270689, X58072, NM_002051_at
FIG. 6365: PRO59053
FIG. 6366: DNA271973, NM_002731,
NM_002731_at
FIG. 6367: PRO60248
FIG. 6368A-B: DNA345276, S65186,
NM_005546_at
FIG. 6369: PRO95739
FIG. 6370: DNA274202, NP_006804.1,
NM_006813_at
FIG. 6371: PRO62131
FIG. 6372: DNA328601, NM_015675,
NM_015675_at
FIG. 6373: PRO84384
FIG. 6374: DNA329050, NM_015969,
NM_015969_at
FIG. 6375: PRO84712
FIG. 6376: DNA326116, NM_016292,
NM_016292_at
FIG. 6377: PRO82542
FIG. 6378A-B: DNA329122, D87119,
NM_021643_at
FIG. 6379: PRO84764
FIG. 6380: DNA255418, L43575, HUMUNKN_at
FIG. 6381: DNA345277, AK026038, AB046774_at
FIG. 6382: PRO95740
FIG. 6383: DNA339707, NP_116119.1, P_T31854_at
FIG. 6384: PRO91437
FIG. 6385: DNA328923, NM_023003, AF255922_at
FIG. 6386: PRO84640
FIG. 6387: DNA345278, NM_025006, AK023435_at
FIG. 6388: PRO95741
FIG. 6389: DNA255219, NP_078936.1,
AK026226_at
FIG. 6390: PRO50298
FIG. 6391: DNA345279, AAH14655.1,
IR1875335_at
FIG. 6392: PRO84549
FIG. 6393: DNA256091, NM_022102, AK024611_at
FIG. 6394: PRO51141
FIG. 6395: DNA254838, NM_024628, AK026841_at
FIG. 6396: PRO49933
FIG. 6397: DNA330548, AK025645, AK025645_at
FIG. 6398: PRO85732
FIG. 6399: DNA329355, NM_033280, P_V40521_at
FIG. 6400: PRO50434
FIG. 6401A-B: DNA256267, AB046838,
AB046838_at
FIG. 6402: DNA327954, NM_031458, P_D00629_at
FIG. 6403: PRO83879
FIG. 6404: DNA255798, NM_024989, AK022439_at
FIG. 6405: PRO50853
FIG. 6406: DNA329384, NM_174921, P_Z33372_at
FIG. 6407: PRO84960
FIG. 6408: DNA345280, AB089319, P_Z24893_at
FIG. 6409: PRO95742
FIG. 6410: DNA255913, AL050125, HSM800425_at
FIG. 6411: PRO50966
FIG. 6412: DNA325379, NP_116136.1,
HSM800835_at
FIG. 6413: PRO81913
FIG. 6414: DNA254596, DNA254596, AF026941_at
FIG. 6415: PRO49699
FIG. 6416A-B: DNA254801, AL080209,
HSM800735_at
FIG. 6417: PRO49897
FIG. 6418: DNA255700, DNA255700,
HSM801128_at
FIG. 6419A-B: DNA328853, NM_020651,
AF302505_at
FIG. 6420: PRO84584
FIG. 6421: DNA330854, AK023113, AK023113_at
FIG. 6422: PRO86017
FIG. 6423A-B: DNA345281, 198947.4,
AK023271_at
FIG. 6424: PRO6012
FIG. 6425: DNA345282, 154551.19, 154551.10_at
FIG. 6426: PRO95743
FIG. 6427A-B: DNA345283, 1327517.49,
994387.65_at
FIG. 6428: PRO95744
FIG. 6429: DNA257363, NM_032315, 203633.4_at
FIG. 6430: PRO51950
FIG. 6431: DNA345284, NM_145810, 475113.7_at
FIG. 6432: PRO69531
FIG. 6433: DNA345285, 200333.3,
200333.3_CON_at
FIG. 6434: PRO95745
FIG. 6435: DNA304068, NP_653250.1,
1091656.1_at
FIG. 6436: PRO71035
FIG. 6437A-B: DNA338079, AL831953,
337352.17_at
FIG. 6438: PRO90959
FIG. 6439: DNA258677, DNA258677, 404505.1_at
FIG. 6440: DNA345286, 1452432.11, 359193.13_at
FIG. 6441: PRO95746
FIG. 6442A-B: DNA345287, NM_032550,
481857.16_at
FIG. 6443: PRO95747
FIG. 6444: DNA259902, DNA259902, 475431.4_at
FIG. 6445: PRO53832
FIG. 6446: DNA345288, 1499607.2, 210883.2_at
FIG. 6447: PRO95748
FIG. 6448: DNA345289, 1449133.1, 109254.1_at
FIG. 6449: PRO95749
FIG. 6450: DNA345290, 332730.8, 332730.8_at
FIG. 6451: PRO95750
FIG. 6452: DNA345291, 407233.2, 407233.2_at
FIG. 6453: PRO95751
FIG. 6454: DNA345292, NM_144601, 197670.7_at
FIG. 6455: PRO95752
FIG. 6456: DNA259663, DNA259663, 215119.2_at
FIG. 6457: DNA345293, 408339.15, 221433.12_at
FIG. 6458: PRO95753
FIG. 6459: DNA287258, NP_542786.1,
228321.19_at
FIG. 6460: PRO52174
FIG. 6461: DNA329626, 1089565.1, 1089565.1_at
FIG. 6462: PRO85155
FIG. 6463: DNA259852, DNA259852, 099349.1_at
FIG. 6464: PRO53782

What is claimed:

1. Isolated nucleic acid comprising at least 80% nucleic acid sequence identity to a nucleotide sequence encoding the polypeptide as shown in any one of the SEQ ID NOs 1-6464.

2. Isolated nucleic acid comprising at least 80% nucleic acid sequence identity to a nucleotide sequence comprising the full-length coding sequence of the nucleotide sequence as shown in any one of the SEQ ID NOs 1-6464.

3. A vector comprising the nucleic acid of claim 1.

4. The vector of claim 3 operably linked to control sequences recognized by a host cell transformed with the vector.

5. A host cell comprising the vector of claim 3.

6. The host cell of claim 5, wherein said cell is a CHO cell, an E.coli cell or a yeast cell.

7. A process for producing a PRO polypeptide comprising culturing the host cell of claim 6 under conditions suitable for expression of said PRO polypeptide and recovering said PRO polypeptide from the cell culture.

8. An isolated polypeptide comprising at least 80% amino acid sequence identity to an amino acid sequence of the polypeptide as shown in any one of the SEQ ID NOs 1-6464.

9. A chimeric molecule comprising a polypeptide according to claim 8 fused to a heterologous amino acid sequence.

10. The chimeric molecule of claim 9, wherein said heterologous amino acid sequence is an epitope tag sequence or an Fc region of an immunoglobulin.

11. An antibody which specifically binds to a polypeptide according to claim 8.

12. The antibody of claim 11, wherein said antibody is a monoclonal antibody, a humanized antibody or a single-chain antibody.

13. A composition of matter comprising (a) a polypeptide of claim 8, (b) an agonist of said polypeptide, (c) an antagonist of said polypeptide, or (d) an antibody that binds to said polypeplide, in combination with a carrier.

14. The composition of matter of claim 13, wherein said carrier is a pharmaceutically acceptable carrier.

15. The composition of matter of claim 14 comprising a therapeutically effective amount of (a), (b), (c) or (d).

16. An article of manufacture, comprising:
a container;
a label on said container; and
a composition of matter comprising (a) a polypeptide of claim 8, (b) an agonist of said polypeptide, (c) an antagonist of said polypeptide, or (d) an antibody that binds to said polypeptide, contained within said container, wherein label on said container indicates that said composition of matter can be used for treating an immune related disease.

17. A method of treating an immune related disorder in a mammal in need thereof comprising administering to said mammal a therapeutically effective amount of (a) a polypeptide of claim 8, (b) an agonist of said polypeptide, (c) an antagonist of said polypeptide, or (d) an antibody that binds to said polypeptide.

18. The method of claim 17, wherein the immune related disorder is systemic lupus erythematosis, rheumatoid arthritis, osteoarthritis, juvenile chronic arthritis, a spondyloarthropathy, systemic sclerosis, an idiopathic inflammatory myopathy, Sjögren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central or peripheral nervous system, idiopathic demyelinating polyneuropathy, Guillain-Barré syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, contact dermatitis, psoriasis, an allergic disease, asthma, allergic rhinitis, atopic dermatitis, food hypersensitivity, urticaria, an immunologic disease of the lung, eosinophilic pneumonias, idiopathic pulmonary fibrosis, hypersensitivity pneumonitis, a transplantation associated disease, graft rejection or graft-versus-host-disease.

19. A method for determining the presence of a PRO polypeptide of the invention as described in any one of SEQ ID NOs 1-6464, in a sample suspected of containing said polypeptide, said method comprising exposing said sample to an anti-PRO antibody, where the and determining binding of said antibody to a component of said sample.

20. A method of diagnosing an immune related disease in a mammal, said method comprising detecting the level of expression of a gene encoding a PRO polypeptide of the invention as described in any one of SEQ ID NOs 1-6464, (a) in a test sample of tissue cells obtained from the mammal, and (b) in a control sample of known normal tissue cells of the same cell type, wherein a higher or lower level of expression of said gene in the test sample as compared to the control sample is indicative of the presence of an immune related disease in the mammal from which the test tissue cells were obtained.

21. A method of diagnosing an immune related disease in a mammal, said method comprising (a) contacting a PRO polypeptide of the invention as described in any one of SEQ ID NOs 1-6464, anti-PRO antibody with a test sample of tissue cells obtained from said mammal and (b) detecting the formation of a complex between the antibody and the polypeptide in the test sample, wherein formation of said complex is indicative of the presence of an immune related disease in the mammal from which the test tissue cells were obtained.

22. A method of identifying a compound that inhibits the activity of a PRO polypeptide of the invention as described in any one of SEQ ID NOs 1-6464, said method comprising contacting cells which normally respond to said polypeptide with (a) said polypeptide and (b) a candidate compound, and determining the lack responsiveness by said cell to (a).

23. A method of identifying a compound that inhibits the expression of a gene encoding a PRO polypeptide of the invention as described in any one of SEQ ID NOs 1-6464, said method comprising contacting cells which normally express said polypeptide with a candidate compound, and determining the lack of expression said gene.

24. The method of claim 23, wherein said candidate compound is an antisense nucleic acid.

25 . A method of identifying a compound that mimics the activity of a PRO polypeptide of the invention as described in any one of SEQ ID NOs 1-6464, said method comprising contacting cells which normally respond to said polypeptide with a candidate compound, and determining the responsiveness by said cell to said candidate compound.

26. A method of stimulating the immune response in a mammal, said method comprising administering to said mammal an effective amount of a PRO polypeptide of the invention as described in any one of SEQ ID NOs 1-6464, antagonist, wherein said immune response is stimulated.

27. A method of diagnosing an inflammatory immune response in a mammal, said method comprising detecting the level of expression of a gene encoding a PRO polypeptide of the invention as described in any one of SEQ ID NOs 1-6464, (a) in a test sample of tissue cells obtained from the mammal, and (b) in a control sample of known normal tissue cells of the same cell type, wherein a higher or lower level of expression of said gene in the test sample as compared to the control sample is indicative of the presence of an inflammatory immune response in the mammal from which the test tissue cells were obtained.
